(19) United States
(12) Patent Application Publication
(10) Pub. No.: US 2018/0029983 A1
(43) Pub. Date: Feb. 1, 2018

(54) COMPOUND, ORGANIC ELECTROLUMINESCENT ELEMENT MATERIAL, ORGANIC ELECTROLUMINESCENT ELEMENT, AND ELECTRONIC DEVICE

(71) Applicant: IDEMITSU KOSAN CO., LTD., Chiyoda-ku (JP)

(72) Inventors: Tomoki KATO, Ichihara-shi (JP); Masahiro KAWAMURA, Chiba-shi (JP)

(73) Assignee: IDEMITSU KOSAN CO., LTD., Chiyoda-ku (JP)

(21) Appl. No.: 15/550,568

(22) PCT Filed: Feb. 16, 2016
(86) PCT No.: PCT/JP2016/054476

§ 371 Date: Aug. 11, 2017

(30) Foreign Application Priority Data
Feb. 16, 2015 (JP) 2015-028082

(51) Int. Cl. C07D 209/82; C07D 491/048; C07D 519/00; H01L 51/50; C09K 11/06; C09K 11/87

(52) U.S. Cl. CPC C07D 209/82; C09K 11/06; C09K 11/87; C07D 519/00; H01L 51/50; C07D 491/048

(57) ABSTRACT

Disclosed herein is a compound represented by formula (1): A-L-B, wherein: A is a group represented by formula (1-A); B is a group represented by formula (1-B); L is a single bond, a substituted or unsubstituted arylene group having 6 to 50 ring carbon atoms, or a substituted or unsubstituted heteroarylene group having 5 to 50 ring atoms; and L is bonded to one of R¹ to R⁴ of formula (1-A) and R¹¹ to R¹⁴ of formula (1-B);

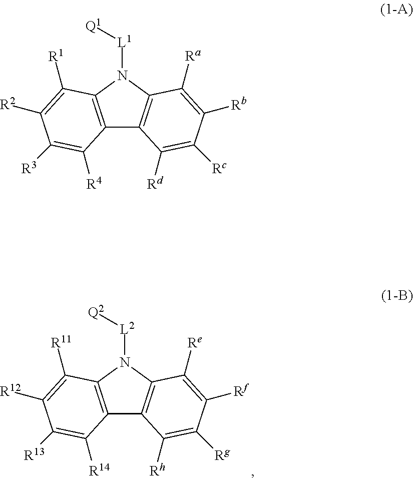

in which the groups Q¹, Q², L¹, L², R¹-R⁴, R¹¹-R¹⁴, R^{a}-R^{d} and R^{e}-R^{h} defined herein. Also disclosed herein are materials and devices containing the compound represented by formula (1).

### TECHNICAL FIELD

The present invention relates to compounds, materials for organic electroluminescence device comprising the compounds, organic electroluminescence devices comprising the compounds, and electronic devices comprising the organic electroluminescence devices.

### BACKGROUND ART

An organic electroluminescence device (also referred to as “organic EL device”) is generally composed of an anode, a cathode, and one or more organic thin film layers which comprise a light emitting layer and are sandwiched between the anode and the cathode. When a voltage is applied between the electrodes, electrons are injected from the cathode and holes are injected from the anode into a light emitting region. The injected electrons recombine with the injected holes in the light emitting region to form excited states. When the excited states return to the ground state, the energy is released as light. Therefore, it is important for increasing the efficiency of an organic EL device to develop a compound which transports electrons or holes into the light emitting region efficiently and facilitates the recombination of electrons and holes. Therefore, a host material to be used in the light emitting layer have been still continuously sought.

In addition, a charge transporting material having a high electron and/or hole mobility and a charge transporting material having an ionization potential level and/or a affinity level (electron affinity) balanced with the energy level of light emitting layer are required for improving the light emission efficiency and the device lifetime. Therefore, various proposals have been made for such a charge transporting material.

### CITATION LIST

### Patent Literature

### Patent Literature 1: WO 2010/136109

### Patent Literature 2: WO 2012/067425

### Patent Literature 3: JP 2013-528929A

### SUMMARY OF INVENTION

### Technical Problem

An object of the invention is to provide an organic EL device having a high emission efficiency and a long lifetime and a material for organic EL device for realizing such an organic EL device.

### Solution to Problem

As a result of extensive research to achieve the above object, the inventors have found that the compound represented by formula (1) has a large excitation stability because of its small energy gap between HOMO (Highest Occupied Molecular Orbital) and LUMO (Lowest Unoccupied Molecular Orbital) and a high electron resistance, thereby improving the lifetime of an organic EL device. It has been further found that a high emission efficiency is obtained because the compound represented by formula (1) has a large ionization potential. It has been still further found that an organic EL device having a high emission efficiency and a long lifetime is obtained by using the compound.

In an aspect of the invention, the following (1) to (4) are provided.

(1) A compound represented by formula (1):

A-L-B (1)

wherein:

A is a group represented by formula (1-A);

B is a group represented by formula (1-B);

L is a single bond, a substituted or unsubstituted arylene group having 6 to 50 ring carbon atoms, or a substituted or unsubstituted heteroarylene group having 5 to 50 ring atoms; and

L is bonded to one of R¹ to R⁴ of formula (i-A) and R¹¹ to R¹⁴ of formula (1-B);

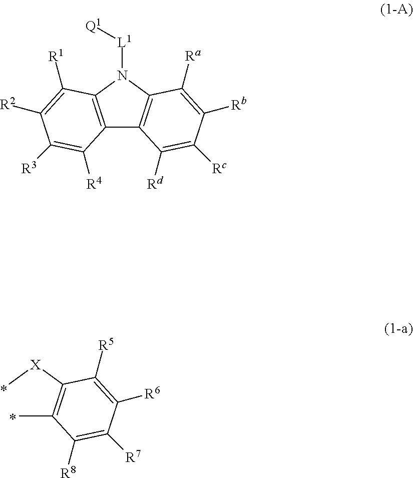

wherein:

R^{a} and R^{b}, R^{b} and R^{c}, or R^{c} and R^{d} are direct bonds which are respectively bonded to sites * of formula (1-a) to form a ring structure;

the others of R^{a} to R^{d} are each independently a hydrogen atom or a substituent, or adjacent two thereof are bonded to each other to form a ring structure;

one of R¹ to R⁸ is bonded to L;

R¹ to R⁸ not bonded to L are each independently a hydrogen atom or a substituent, or adjacent two thereof are bonded to each other to form a ring structure;

L¹ is a single bond, a substituted or unsubstituted arylene group having 6 to 50 ring carbon atoms, or a substituted or unsubstituted heteroarylene group having 5 to 50 ring atoms;

Q¹ is a substituted or unsubstituted aryl group having 6 to 50 ring carbon atoms or a substituted or unsubstituted heteroaryl group having 5 to 40 ring atoms; and

X is an oxygen atom or a sulfur atom;

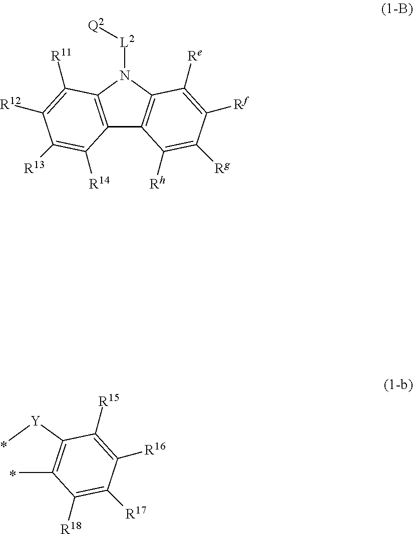

wherein:

R^{e} and R^{f}, R^{f} and R^{g}, or R^{g} and R^{h} are direct bonds which are respectively bonded to sites * of formula (1-b) to form a ring structure;

the others of R^{e} to R^{h} are each independently a hydrogen atom or a substituent, or adjacent two thereof are bonded to each other to form a ring structure;

one of R¹¹ to R¹⁸ is bonded to L;

R¹¹ to R¹⁸ not bonded to L are each independently a hydrogen atom or a substituent, or adjacent two thereof are bonded to each other to form a ring structure;

L² is a single bond, a substituted or unsubstituted arylene group having 6 to 50 ring carbon atoms, or a substituted or unsubstituted heteroarylene group having 5 to 50 ring atoms;

Q² is a substituted or unsubstituted aryl group having 6 to 50 ring carbon atoms, or a substituent or unsubstituted heteroarylene group having 5 to 40 ring atoms;

Y is C(R¹⁹)(R²⁰), N(R²¹), an oxygen atom, or a sulfur atom;

each of R¹⁹ and R²⁰ is independently a hydrogen atom, a substituted or unsubstituted alkyl group having 1 to 20 carbon atoms, a substituted or unsubstituted aryl group having 6 to 50 ring carbon atoms, a substituted or unsubstituted heteroaryl group having 5 to 50 ring atoms, a substituted or unsubstituted fluoroalkyl group having 1 to 20 carbon atoms, or a cyano group, or adjacent two thereof are bonded to each other to form a ring structure; and

R²¹ is a substituted or unsubstituted alkyl group having 1 to 20 carbon atoms, a substituted or unsubstituted aryl group having 6 to 50 ring carbon atoms, a substituted or unsubstituted heteroaryl group having 5 to 50 ring atoms, or a substituted or unsubstituted fluoroalkyl group having 1 to 20 carbon atoms.

(2) A material for organic electroluminescence device comprising the compound described in (1).
(3) An organic electroluminescence device comprising an organic thin film layer between a cathode and an anode, wherein the organic thin film layer comprises one or more layers comprising a light emitting layer and at least one layer of the organic thin film layer comprises the compound described in (1).
(4) An electronic device comprising the organic electroluminescence device described in (3).

### Advantageous Effects of Invention

An organic EL device having a high emission efficiency and a long lifetime can be obtained by using the compound represented by formula (1) as a material for organic EL device.

### BRIEF DESCRIPTION OF DRAWINGS

FIG. 1 is a schematic illustration showing the structure of an organic EL device in an aspect of the invention.

### DESCRIPTION OF EMBODIMENTS

The term of “XX to YY carbon atoms” referred to by “a substituted or unsubstituted group ZZ having XX to YY carbon atoms” used herein is the number of carbon atoms of the unsubstituted group ZZ and does not include any carbon atom in the substituent of the substituted group ZZ.

The term of “XX to YY atoms” referred to by “a substituted or unsubstituted group ZZ having XX to YY atoms” used herein is the number of atoms of the unsubstituted group ZZ and does not include any atom in the substituent of the substituted group ZZ.

The number of “ring carbon atoms” referred to herein means the number of the carbon atoms included in the atoms which are members forming the ring itself of a compound in which a series of atoms is bonded to form a ring (for example, a monocyclic compound, a fused ring compound, a cross-linked compound, a carbocyclic compound, and a heterocyclic compound). If the ring has a substituent, the carbon atom in the substituent is not included in the ring carbon atom. The same applies to the number of “ring carbon atom” described below, unless otherwise noted. For example, a benzene ring has 6 ring carbon atoms, a naphthalene ring has 10 ring carbon atoms, a pyridinyl group has 5 ring carbon atoms, and a furanyl group has 4 ring carbon atoms. If a benzene ring or a naphthalene ring has, for example, an alkyl substituent, the carbon atom in the alkyl substituent is not counted as the ring carbon atom of the benzene or naphthalene ring. In case of a fluorene ring to which a fluorene substituent is bonded (inclusive of a spirofluorene ring), the carbon atom in the fluorene substituent is not counted as the ring carbon atom of the fluorene ring.

The number of “ring atom” referred to herein means the number of the atoms which are members forming the ring itself (for example, a monocyclic ring, a fused ring, and a ring assembly) of a compound in which a series of atoms is bonded to form the ring (for example, a monocyclic compound, a fused ring compound, a cross-linked compound, a carbocyclic compound, and a heterocyclic compound). The atom not forming the ring (for example, hydrogen atom(s) for saturating the valence of the atom which forms the ring) and the atom in a substituent, if the ring is substituted, are not counted as the ring atom. The same applies to the number of “ring atoms” described below, unless otherwise noted. For example, a pyridine ring has 6 ring atoms, a quinazoline ring has 10 ring atoms, and a furan ring has 5 ring atoms. The hydrogen atom on the ring carbon atom of a pyridine ring or a quinazoline ring and the atom in a substituent are not counted as the ring atom. In case of a fluorene ring to which a fluorene substituent is bonded (inclusive of a spirofluorene ring), the atom in the fluorene substituent is not counted as the ring atom of the fluorene ring.

The definition of “hydrogen atom” used herein includes isotopes different in the neutron numbers, i.e., light hydrogen (protium), heavy hydrogen (deuterium), and tritium.

The terms of “heteroaryl group” and “heteroarylene group” used herein means a group having at least one hetero atom as a ring atom. The hetero atom is preferably at least one selected from a nitrogen atom, an oxygen atom, a sulfur atom, a silicon atom, and a selenium atom.

The substituent and the optional substituent referred to by “substituted or unsubstituted” used herein is preferably selected from the group consisting of an alkyl group having 1 to 50, preferably 1 to 18, more preferably 1 to 8 carbon atoms; a cycloalkyl group having 3 to 50, preferably 3 to 10, more preferably 3 to 8, still more preferably 5 or 6 ring carbon atoms; an aryl group having 6 to 50, preferably 6 to 25, more preferably 6 to 18 ring carbon atoms; an aralkyl group having 7 to 51, preferably 7 to 30, more preferably 7 to 20 carbon atoms which includes an aryl group having 6 to 50, preferably 6 to 25, more preferably 6 to 18 ring carbon atoms; an alkoxy group having an alkyl group having 1 to 50, preferably 1 to 18, more preferably 1 to 8 carbon atoms; an aryloxy group having an aryl group having 6 to 50, preferably 6 to 25, more preferably 6 to 18 ring carbon atoms; a disubstituted amino group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; a mono-, di- or tri-substituted silyl group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; a heteroaryl group having 5 to 50, preferably 5 to 24, more preferably 5 to 13 ring atoms; a haloalkyl group having 1 to 50, preferably 1 to 18, more preferably 1 to 8 carbon atoms; a halogen atom selected from a fluorine atom, a chlorine atom, a bromine atom and an iodine atom; a cyano group; a nitro group; a substituted sulfonyl group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; a di-substituted phosphoryl group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; an alkylsulfonyloxy group; an arylsulfonyloxy group; an alkylcarbonyloxy group; an arylcarbonyloxy group; a boron-containing group; a zinc-containing group; a tin-containing group; a silicon-containing group; a magnesium-containing group; a lithium-containing group; a hydroxyl group; an alkyl-substituted or aryl-substituted carbonyl group; a carboxyl group; a vinyl group; a (meth)acryloyl group; an epoxy group; and an oxetanyl group.

The above substituent may further has the substituent mentioned above. The substituents may be bonded to each other to form a ring.

The term of “unsubstituted” referred to by “substituted or unsubstituted” used herein means that no hydrogen atom in the group is substituted by a substituent.

Of the above substituents, more preferred are a substituted or unsubstituted alkyl group having 1 to 50, preferably 1 to 18, more preferably 1 to 8 carbon atoms; a substituted or unsubstituted aryl group having 6 to 50, preferably 6 to 25, more preferably 6 to 18 ring carbon atoms; a substituted or unsubstituted heteroaryl group having 5 to 50, preferably 5 to 24, more preferably 5 to 13 ring atoms; a halogen atom; a cyano group; a substituted or unsubstituted fluoroalkyl group having 1 to 50, preferably 1 to 20, more preferably 1 to 10, still more preferably 1 to 5 carbon atoms; a substituted or unsubstituted alkoxy group having 1 to 50, preferably 1 to 20, more preferably 1 to 10, still more preferably 1 to 5 carbon atoms; a substituted or unsubstituted fluoroalkoxy group having 1 to 50, preferably 1 to 20, more preferably 1 to 10, still more preferably 1 to 5 carbon atoms; a substituted or unsubstituted aryloxy group having 6 to 50, preferably 6 to 25, more preferably 6 to 18, still more preferably 6 to 12 ring carbon atoms; a disubstituted amino group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; and a trisubstituted silyl group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms

Of the above substituents, still more preferred are a substituted or unsubstituted aryl group having 6 to 50, preferably 6 to 25, more preferably 6 to 18 ring carbon atoms; a substituted or unsubstituted heteroaryl group having 5 to 50, preferably 5 to 24, more preferably 5 to 13 ring atoms; a halogen atom; a cyano group; a substituted or unsubstituted fluoroalkyl group having 1 to 50, preferably 1 to 20, more preferably 1 to 10, still more preferably 1 to 5 carbon atoms; a substituted or unsubstituted alkoxy group having 1 to 50, preferably 1 to 20, more preferably 1 to 10, still more preferably 1 to 5 carbon atoms; a substituted or unsubstituted fluoroalkoxy group having 1 to 50, preferably 1 to 20, more preferably 1 to 10, still more preferably 1 to 5 carbon atoms; and a substituted or unsubstituted aryloxy group having 6 to 50, preferably 6 to 25, more preferably 6 to 18, still more preferably 6 to 12 ring carbon atoms.

### Examples of the Substituent

Examples of the alkyl group having 1 to 50 carbon atoms include a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), and a dodecyl group (inclusive of isomeric groups).

Examples of the aryl group having 6 to 50 ring carbon atoms include a phenyl group, a naphthylphenyl group, a biphenylyl group, a terphenylyl group, a biphenylenyl group, a naphthyl group, a phenylnaphthyl group, an acenaphthylenyl group, an anthryl group, a benzanthryl group, an aceanthryl group, a phenanthryl group, a benzophenanthryl group, a phenalenyl group, a fluorenyl group, a 9,9-dimethylfluorenyl group, a 7-phenyl-9,9-dimethylfluorenyl group, a pentacenyl group, a picenyl group, a pentaphenyl group, a pyrenyl group, a chrysenyl group, a benzochrysenyl group, a s-indanyl group, an as-indanyl group, a fluoranthenyl group, and a perylenyl group.

The heteroaryl group having 5 to 50 ring atoms comprises at least one preferably 1 to 3 same or different hetero atoms, for example, a nitrogen atom, a sulfur atom, and an oxygen atom.

Examples of the heteroaryl group include a pyrrolyl group, a furyl group, a thienyl group, a pyridyl group, a pyridazinyl group, a pyrimidinyl group, a pyridinyl group, a triazinyl group, an imidazolyl group, an xazolyl group, a thiazolyl group, a pyrazolyl group, an isoxazolyl group, an isothiazolyl group, an oxadiazolyl group, a thiadiazolyl group, a triazolyl group, an indolyl group, an isoindolyl group, a benzofuranyl group, an isobenzofuranyl group, a benzothiophenyl group, an indolizinyl group, a quinolizinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an indazolyl group, a benzisoxazolyl group, a benzisothiazolyl group, a dibenzofuranyl group, a dibenzothiophenyl group, a phenanthridinyl group, an acridinyl group, a phenanthrolinyl group, a phenazinyl group, a phenothiazinyl group, a phenoxazinyl group, and a xanthenyl group.

Examples of the fluoroalkyl group having 1 to 50 carbon atoms include those derived from the above alkyl group having 1 to 50 carbon atoms by replacing at least one hydrogen atom, preferably 1 to 7 hydrogen atoms or all hydrogen atoms with a fluorine atom or fluorine atoms.

Preferred examples of the fluoroalkyl group include a heptafluoropropyl group, a pentafluoroethyl group, a 2,2,2-trifluoroethyl group, and a trifluoromethyl group.

The alkoxy group having 1 to 50 carbon atoms is represented by —OR^{X}, wherein R^{X} is the above alkyl group having 1 to 50 carbon atoms.

Examples thereof include a t-butoxy group, a propoxy group, an ethoxy group, and a methoxy group.

The fluoroalkoxy group having 1 to 50 carbon atoms is represented by —OR^{Y}, wherein R^{Y} is the above fluoroalkyl group having 1 to 50 carbon atoms.

Examples of the fluoroalkoxy group include a heptafluoropropropoxy group, a pentafluoroethoxy group, a 2,2,2-trifluoroethoxy group, and a trifluoromethoxyl group.

The aryloxy group having 6 to 50 ring carbon atoms is represented by —OR^{Z}, wherein R^{Z} is the above aryl group having 6 to 50 ring carbon atoms.

Examples of the aryloxy group include a phenyloxy group, a 1-naphthyloxy group, a 2-naphthyloxy group, a 4-biphenylyloxy group, a p-terphenyl-4-yloxy group, and a p-tollyloxy group.

The alkyl group and the aryl group of the disubstituted amino group having the substituents selected from an alkyl group having 1 to 50 carbon atoms and an aryl group having 6 to 50 ring carbon atoms are selected from the alkyl group having 1 to 50 carbon atoms and the aryl group having 6 to 50 ring carbon atoms each mentioned above.

Examples of the disubstituted amino group include a dialkylamino group, such as a dimethylamino group, a diethylamino group, a diisopropylamino group, and a di-t-butylamino group, a diphenylamino group, a di(methylphenyl)amino group, a dinaphthylamino group, and a dibiphenylylamino group.

The alkyl group and the aryl group of the trisubstituted silyl group having the substituents selected from an alkyl group having 1 to 50 carbon atoms and an aryl group having 6 to 50 ring carbon atoms are selected from the alkyl group having 1 to 50 carbon atoms and the aryl group having 6 to 50 ring carbon atoms each mentioned above.

The trisubstituted silyl group is preferably a trialkylsilyl group wherein the alkyl group is as mentioned above and a triarylsilyl group wherein the aryl group is as mentioned above. Examples of the trialkylsilyl group include a trimethylsilyl group, a triethylsilyl group, a triisopropylsilyl group, a tri-t-butylsilyl group, and a tri-n-butylsilyl group. Examples of the triarylsilyl group include a triphenylsilyl group and a tri(methylphenyl)silyl group.

The present invention will be described below in detail.

The descriptions of preferred embodiments herein may be arbitrarily selected.

### Compound

In an aspect of the invention, the compound represented by formula (1) (hereinafter also referred to as “compound (1)”) is provided. The compound is useful as a material for organic electroluminescence device.

A-L-B (1)

wherein:

A is a group represented by formula (1-A);

B is a group represented by formula (1-B);

L is a single bond, a substituted or unsubstituted arylene group having 6 to 50 ring carbon atoms, or a substituted or unsubstituted heteroarylene group having 5 to 50 ring atoms; and

L is bonded to one of R¹ to R⁴ of formula (1-A) and R¹¹ to R¹⁴ of formula (1-B).

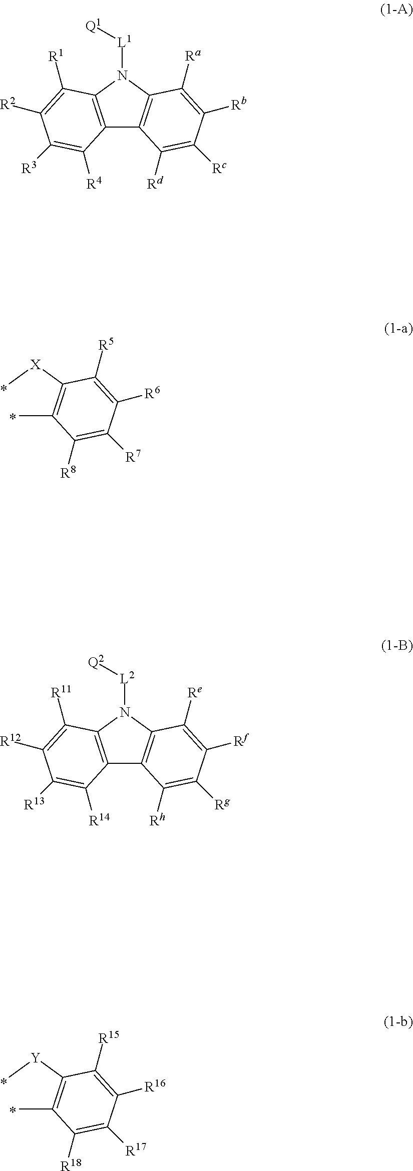

R^{a} to R^{d} of Formula (1-A)

R^{a} and R^{b}, R^{b} and R^{c}, or R^{c} and R^{d} are direct bonds which are respectively bonded to sites * of formula (1-a) to form a ring structure. The others of R^{a} to R^{d} are each independently a hydrogen atom or a substituent, or adjacent two thereof are bonded to each other to form a ring structure, and preferably all hydrogen atoms. When R^{a} and R^{b} are direct bonds which are respectively bonded to the sites * of formula (1-a), thereby forming a ring structure, it is possible for only R^{c} and R^{d} to form a ring structure by being bonded to each other. When R^{c} and R^{d} are direct bonds which are respectively bonded to the sites * of formula (1-a), thereby forming a ring structure, it is possible only for R^{a} and R^{b} to form a ring structure by being bonded to each other.

The substituent and preferred examples thereof are as described above, and more preferred are a substituted or unsubstituted alkyl group having 1 to 50, preferably 1 to 18, more preferably 1 to 8 carbon atoms, a halogen atom, a cyano group, and a substituted or unsubstituted alkoxy group having 1 to 20, preferably 1 to 5, more preferably 1 to 4 carbon atoms.

R¹ to R⁴ of Formula (1-A) and R⁵ to R⁸ of Formula (1-a)

One of R¹ to R⁸ is bonded to L of formula (1), preferably one of R¹ to R⁴ is bonded to L of formula (1), thus, it represents a bond directly bonded to B of formula (1), if L is a single bond.

R¹ to R⁸ not bonded to L are each independently a hydrogen atom or a substituent, or adjacent two thereof are bonded to each other to form a ring structure. The ring structure may be an aromatic ring or a partly saturated ring. R¹ to R⁸ not bonded to L are preferably all hydrogen atoms. The substituent is as described above with respect to R^{a} to R^{d}.

### L¹ of Formula (1-A)

L¹ is a single bond, a substituted or unsubstituted arylene group having 6 to 50 ring carbon atoms, or a substituted or unsubstituted heteroarylene group having 5 to 50 ring atoms.

Examples of the arylene group for L¹ include a phenylene group (1,2-phenylene group, 1,3-phenylene group, 1,4-phenylene group), a naphthylene group (for example, 1,4-naphthylene group and 1,5-naphthylene group), a biphenylylene group, a fluorenylene group (for example, 2,7-fluorenylene group), a 9,9-disubstituted fluorenylene group (for example, 9,9-dimethyl-2,7-fluorenylene group and 9,9-diphenyl-2,7-fluorenylene group), a benzofluorenylene group, a dibenzofluorenylene group, a picenylene group, a tetracenylene group, a pentacenylene group, a pyrenylene group, a chrysenylene group, a benzochrysenylene group, a s-indanylene group, an as-indanylene group, a fluoranthenylene group, a benzofluoranthenyl group, a triphenylenylene group, a benzotriphenylenyl group, a perylenylene group, a coronenylene group, and dibenzoanthrylene group.

In view of the emission efficiency and the lifetime, the arylene group is preferably a substituted or unsubstituted arylene group having 6 to 30 ring carbon atoms, more preferably a substituted or unsubstituted arylene group having 6 to 12 ring carbon atoms, still more preferably a substituted or unsubstituted arylene group having 6 to 10 ring carbon atoms, and particularly preferably a phenylene group.

Examples of the heteroarylene group for L¹ include a pyrrolylene group, a furylene group, a thienylene group, a pyridylene group, an imidazopyridylene group, a pyridazinylene group, a pyrimidinylene group, a pyrazinylene group, a triazinylene group, an imidazolylene group, an oxazolylene group, a thiazolylene group, a pyrazolylene group, an isoxazolylene group, an isothiazolylene group, an oxadiazolylene group, a thiadiazolylene group, a triazolylene group, a tetrazolylen group, an indolylene group, an isoindolylene group, a benzofuranylene group, an isobenzofuranylene group, a benzothiophenylene group, an isobenzothiophenylene group, an indolizinylene group, a quinolizinylene group, a quinolylene group, an isoquinolylene group, a cinnolylene group, a phthalazinylene group, a quinazolinylene group, a quinoxalinylene group, a benzimidazolylene group, a benzoxazolylene group, a benzothiazolylene group, an indazolylene group, a benzisoxazolylene group, a benzisothiazolylene group, a dibenzofuranylene group, a dibenzothiophenylene group, a phenanthridinylene group, an acridinylene group, a phenanthrolinylene group, a phenazinylene group, a phenothiazinylene group, a phenoxazinylene group, and a xanthenylene group. In view of the emission efficiency and the lifetime, the heteroarylene group is preferably a substituted or unsubstituted heteroarylene group having 5 to 30 ring atoms, more preferably a substituted or unsubstituted heteroarylene group having 5 to 15 ring atoms, still more preferably a substituted or unsubstituted heteroarylene group having 5 to 10 ring atoms, and particularly preferably a substituted or unsubstituted heteroarylene group having 6 ring atoms.

In view of the emission efficiency and the lifetime, examples of the heteroarylene group are preferably a furylene group, a thienylene group, a pyridylene group, an imidazopyridylene group, a pyridazinylene group, a pyrimidinylene group, a pyrazinylene group, a triazinylene group, a benzimidazolylene group, a dibenzofuranylene group, a dibenzothiophenylene group, and a phenanthrolinylene group each being substituted or unsubstituted, and more preferably a substituted or unsubstituted pyrimidinylene group and a substituted or unsubstituted triazinylene group.

In view of the emission efficiency and the lifetime, L¹ is preferably a single bond or a substituted or unsubstituted arylene group having 6 to 50 ring carbon atoms and more preferably a single bond.

### Q¹ of Formula (1-A)

Q¹ is a substituted or unsubstituted aryl group having 6 to 50 ring carbon atoms or a substituted or unsubstituted heteroaryl group having 5 to 40 ring atoms.

Examples of the aryl group for Q¹ include a phenyl group, a naphthyl group (1-naphthyl group, 2-naphthyl group), an anthryl group (for example, 1-anthryl group and 2-anthryl group), a benzanthryl group, a phenanthryl group (for example, 1-phenanthryl group, 2-phenanthryl group, 3-phenanthryl group, and 9-phenanthryl group), a benzophenanthryl group, a fluorenyl group, a 9,9-disubstituted fluorenyl group (for example, 9,9-dimethyl-2-fluorenyl group and 9,9-diphenyl-2-fluorenyl group), a spirobifluorenyl group, a benzofluorenyl group, a dibenzofluorenyl group, a picenyl group, a tetracenyl group, a pentacenyl group, a pyrenyl group, a chrysenyl group, a benzochrysenyl group, a s-indanyl group, an as-indanyl group, a fluoranthenyl group, a benzofluoranthenyl group, a triphenylenyl group, a benzotriphenylenyl group, a perylenyl group, a coronenyl group, and a dibenzanthryl group. In view of the emission efficiency and the lifetime, the aryl group is preferably a substituted or unsubstituted aryl group having 6 to 30 ring carbon atoms and more preferably a substituted or unsubstituted aryl group having 6 to 20 ring carbon atoms.

In view of the emission efficiency and the lifetime, examples of the aryl group are preferably a substituted or unsubstituted phenyl group and a substituted or unsubstituted biphenylyl group.

Examples of the heteroaryl group for Q¹ include a pyrrolyl group, a furyl group, a thienyl group, a pyridyl group, an imidazopyridyl group, a pyridazinyl group, a pyrimidinyl group, a pyridinyl group, a triazinyl group, an imidazolyl group, an xazolyl group, a thiazolyl group, a pyrazolyl group, an isoxazolyl group, an isothiazolyl group, an oxadiazolyl group, a thiadiazolyl group, a triazolyl group, a tetrazolyl group, an indolyl group, an isoindolyl group, a benzofuranyl group, an isobenzofuranyl group, a benzothiophenyl group, an isobenzothiophenyl group, an indolizinyl group, a quinolizinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an indazolyl group, a benzisoxazolyl group, a benzisothiazolyl group, a dibenzofuranyl group, a dibenzothiophenyl group, a phenanthridinyl group, an acridinyl group, a phenanthrolinyl group, a phenazinyl group, a phenothiazinyl group, a phenoxazinyl group, and a xanthenyl group. In view of the emission efficiency and the lifetime, the heteroaryl group is preferably a substituted or unsubstituted heteroaryl group having 5 to 30 ring atoms and more preferably a substituted or unsubstituted heteroaryl group having 5 to 20 ring atoms.

In view of the emission efficiency and the lifetime, examples of the heteroaryl group are preferably a furyl group, a thienyl group, a pyridyl group, an imidazopyridyl group, a pyridazinyl group, a pyrimidinyl group, a pyridinyl group, a triazinyl group, a benzimidazolyl group, a dibenzofuranyl group, a dibenzothiophenyl group, and a phenanthrolinyl group each being substituted or unsubstituted, more preferably a substituted or unsubstituted pyrimidinyl group and a substituted or unsubstituted triazinyl group, and still more preferably a substituted pyrimidinyl group and a substituted triazinyl group, and particularly preferably a disubstituted pyrimidinyl group and a disubstituted triazinyl group.

In view of the emission efficiency and the lifetime, Q¹ is preferably a substituted or unsubstituted aryl group having 6 to 50 ring carbon atoms and particularly preferably a substituted or unsubstituted phenyl group, a substituted or unsubstituted biphenylyl group, a naphthyl group, a phenanthryl group, a benzophenanthryl group, a fluorenyl group, a 9,9-disubstituted fluorenyl group, a spirobifluorenyl group, a benzofluorenyl group, a benzochrysenyl group, a fluoranthenyl group, a benzofluoranthenyl group, or a triphenylenyl group.

In view of the emission efficiency and the lifetime, Q¹ is also preferably a group represented by formula (3), more preferably a group represented by formula (3-1), and still more preferably a group represented by any of formulae (3-2) to (3-4):

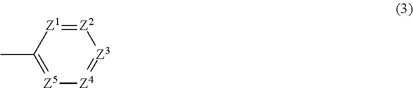

wherein:

at least one selected from Z¹ to Z⁵ is a nitrogen atom and the others are each independently C(R³⁰), wherein R³⁰ is a hydrogen atom or a substituent; and

when two or more of Z¹ to Z⁵ are C(R³⁰), variables R³⁰ may be the same or different and variables R³⁰ may be bonded to each other to form a ring structure;

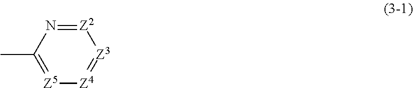

wherein:

each of Z² to Z⁵ is independently a nitrogen atom or C(R³⁰), wherein R³⁰ is a hydrogen atom or a substituent; and

when two or more of Z² to Z⁵ are C(R³⁰), variables R³⁰ may be the same or different and variables R³⁰ may be bonded to each other to form a ring structure;

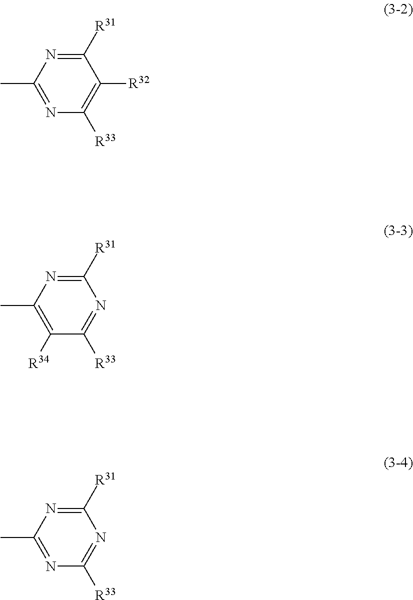

wherein:

each of R³¹ to R³⁴ is independently a hydrogen atom or a substituent;

R³¹ and R³², and R³² and R³³ in formula (3-2) may be bonded to each other to form a ring structure; and

R³³ and R³⁴ in formula (3-3) may be bonded to each other to form a ring structure.

### Formulae (3) and (3-1)

The substituent represented by R³⁰ is as described above with respect to R^{a} to R^{d}, and preferably a substituted or unsubstituted aryl group having 6 to 50 ring carbon atoms, more preferably a substituted or unsubstituted aryl group having 6 to 30 ring carbon atoms, still more preferably a substituted or unsubstituted aryl group having 6 to 20 ring carbon atoms, and particularly preferably a substituted or unsubstituted phenyl group.

When variables R³⁰ are bonded to each other to form a ring structure, formula (3) represents the following groups. The ring structure may be an aromatic ring as shown below or a partly saturated ring.

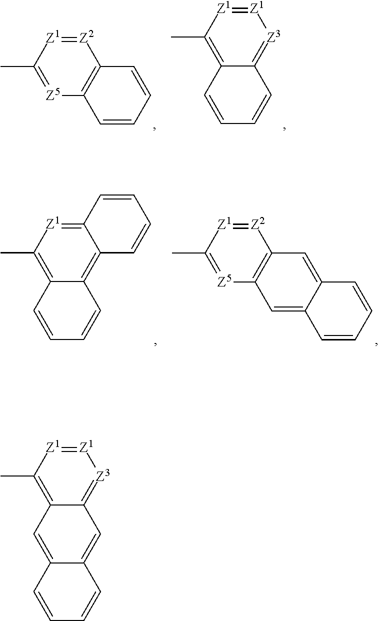

### Formulae (3-2) to (3-4)

The substituent represented by R¹ to R³⁴ and preferred examples thereof are as described above with respect to R³⁰.

When R³¹ and R³² or R³² and R³³ of formula (3-2) are bonded to each other to form a ring structure, or R³³ and R³⁴ of formula (3-3) are bonded to each other to form a ring structure, formulae (3-2) and (3-3) represent, for example, the following groups. The ring structure may be an aromatic ring as shown below or a partly saturated ring.

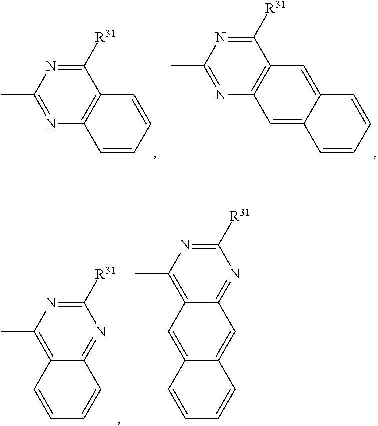

### X of Formula (1-a)

X is an oxygen atom or a sulfur atom. In a preferred embodiment, X is an oxygen atom, and in another preferred embodiment, X is a sulfur atom. X is more preferably an oxygen atom.

R^{e} to R^{h} of Formula (1-B)

R^{e} and R^{f}, R^{f} and R^{g}, or R^{g} and R^{h} are direct bonds which are respectively bonded to sites * of formula (1-b) to form a ring structure. The others of R^{e} to R^{h} are each independently a hydrogen atom or a substituent, or adjacent two thereof are bonded to each other to form a ring structure. When R^{e} and R^{f} are direct bonds which are respectively bonded to the sites * of formula (1-b), thereby forming a ring structure, it is possible for only R^{g} and R^{h} to form a ring structure by being bonded to each other. When R^{g} and R^{h} are direct bonds which are respectively bonded to the sites * of formula (1-b), thereby forming a ring structure, it is possible for only R^{e} and R^{f} to form a ring structure by being bonded to each other.

The substituent and preferred examples thereof are as described above, and more preferred are a substituted or unsubstituted alkyl group having 1 to 50, preferably 1 to 18, more preferably 1 to 8 carbon atoms, a halogen atom, a cyano group, and a substituted or unsubstituted alkoxy group having 1 to 20, preferably 1 to 5, more preferably 1 to 4 carbon atoms.

R¹¹ to R¹⁴ of Formula (1-B) and R¹⁵ to R¹⁸ of Formula (1-b)

One of R¹¹ to R¹⁸ is bonded to L of formula (1), preferably one of R¹¹ to R¹⁴ is bonded to L of formula (1), thus, it represents a bond directly bonded to A of formula (1), if L is a single bond.

When one of R¹ to R⁴ of formula (i-A) is bonded to L of formula (1), one of R¹⁵ to R¹⁸ of formula (1-b) is preferably bonded to L. Particularly preferably, one of R¹ to R⁴ of formula (1-A) and one of R¹¹ to R¹⁴ of formula (1-B) are bonded to L.

R¹¹ to R¹⁸ not bonded to L are each independently a hydrogen atom or a substituent, or adjacent two thereof are bonded to each other to form a ring structure. R¹¹ to R¹⁸ not bonded to L are preferably all hydrogen atoms. The substituent is as described above with respect to R^{e} to R^{h}.

### L² of Formula (1-B)

L² is a single bond, a substituted or unsubstituted arylene group having 6 to 50 ring carbon atoms, or a substituted or unsubstituted heteroarylene group having 5 to 50 ring atoms.

The arylene group for L² is the same as that for L¹ mentioned above. In view of the emission efficiency and the lifetime, the arylene group is preferably a substituted or unsubstituted arylene group having 6 to 30 ring carbon atoms, more preferably a substituted or unsubstituted arylene group having 6 to 12 ring carbon atoms, still more preferably a substituted or unsubstituted arylene group having 6 to 10 ring carbon atoms, and particularly preferably a phenylene group.

The heteroarylene group for L² is the same as that for L¹ mentioned above. In view of the emission efficiency and the lifetime, the heteroarylene group is preferably a substituted or unsubstituted heteroarylene group having 5 to 30 ring atoms, more preferably a substituted or unsubstituted heteroarylene group having 5 to 15 ring atoms, still more preferably a substituted or unsubstituted heteroarylene group having 5 to 10 ring atoms, and particularly preferably a substituted or unsubstituted heteroarylene group having 6 ring atoms.

In view of the emission efficiency and the lifetime, examples of the heteroarylene group are preferably a furylene group, a thienylene group, a pyridylene group, an imidazopyridylene group, a pyridazinylene group, a pyrimidinylene group, a pyrazinylene group, a triazinylene group, a benzimidazolylene group, a dibenzofuranylene group, a dibenzothiophenylene group, and a phenanthrolinylene group each being substituted or unsubstituted, and more preferably a substituted or unsubstituted pyrimidinylene group and a substituted or unsubstituted triazinylene group.

In view of the emission efficiency and the lifetime, L² is preferably a single bond or a substituted or unsubstituted arylene group having 6 to 50 ring carbon atoms and more preferably a single bond.

### Q² of Formula (1-B)

Q² is a substituted or unsubstituted aryl group having 6 to 50 ring carbon atoms or a substituted or unsubstituted heteroaryl group having 5 to 40 ring atoms.

The aryl group for Q² is the same as that for Q¹ mentioned above. In view of the emission efficiency and the lifetime, the aryl group is preferably a substituted or unsubstituted aryl group having 6 to 30 ring carbon atoms and more preferably a substituted or unsubstituted aryl group having 6 to 20 ring carbon atoms.

In view of the emission efficiency and the lifetime, examples of the aryl group are preferably a substituted or unsubstituted phenyl group, a substituted or unsubstituted biphenylyl group, a naphthyl group, a phenanthryl group, a benzophenanthryl group, a fluorenyl group, a 9,9-disubstituted fluorenyl group, a spirobifluorenyl group, a benzofluorenyl group, a benzochrysenyl group, a fluoranthenyl group, a benzofluoranthenyl group, and a triphenylenyl group.

The heteroaryl group for Q² is the same as that for Q¹ mentioned above. In view of the emission efficiency and the lifetime, the heteroaryl group is preferably a substituted or unsubstituted heteroaryl group having 5 to 30 ring atoms and more preferably a substituted or unsubstituted heteroaryl group having 5 to 20 ring atoms.

In view of the emission efficiency and the lifetime, examples of the heteroaryl group are preferably a furyl group, a thienyl group, a pyridyl group, an imidazopyridyl group, a pyridazinyl group, a pyrimidinyl group, a pyridinyl group, a triazinyl group, a benzimidazolyl group, a dibenzofuranyl group, a dibenzothiophenyl group, and a phenanthrolinyl group each being substituted or unsubstituted, more preferably a substituted or unsubstituted pyrimidinyl group and a substituted or unsubstituted triazinyl group, still more preferably a substituted pyrimidinyl group and a substituted triazinyl group, and particularly preferably a disubstituted pyrimidinyl group and a disubstituted triazinyl group.

Q² is preferably a substituted or unsubstituted aryl group having 6 to 50 ring carbon atoms.

The compound (1) of the invention, wherein Q² is a substituted or unsubstituted aryl group having 6 to 50 ring carbon atoms and Q¹ is a substituted or unsubstituted heteroaryl group having 5 to 40 ring atoms, is suitable as a host material (particularly a phosphorescent host material) of a light emitting layer.

The compound (1) of the invention, wherein Q² is a substituted or unsubstituted aryl group having 6 to 50 ring carbon atoms and Q¹ is a substituted or unsubstituted aryl group having 6 to 50 ring carbon atoms, is suitable as a material for an anode-side organic thin film layer (a hole transporting layer, a hole injecting layer, etc.) between an anode and a light emitting layer or a host material (particularly a phosphorescent host material) of a light emitting layer, particularly, as a material for an anode-side organic thin film layer (a hole transporting layer, a hole injecting layer, etc.)

### Y of Formula (1-b)

Y is C(R¹⁹)(R²⁰), N(R²¹), an oxygen atom or a sulfur atom.

Each of R¹⁹ and R²⁰ is independently a hydrogen atom, a substituted or unsubstituted alkyl group having 1 to 20 carbon atoms, a substituted or unsubstituted aryl group having 6 to 50 ring carbon atoms, a substituted or unsubstituted heteroaryl group having 5 to 50 ring atoms, a substituted or unsubstituted fluoroalkyl group having 1 to 20 carbon atoms, or a cyano group, or R¹⁹ and R²⁰ are bonded to each other to form a ring structure.

R²¹ is a substituted or unsubstituted alkyl group having 1 to 20 carbon atoms, a substituted or unsubstituted aryl group having 6 to 50 ring carbon atoms, a substituted or unsubstituted heteroaryl group having 5 to 50 ring atoms, or a substituted or unsubstituted fluoroalkyl group having 1 to 20 carbon atoms.

Preferred examples of the substituent represented by each of R¹⁹, R²⁰, and R²¹ include a substituted or unsubstituted alkyl group having 1 to 50, preferably 1 to 18, more preferably 1 to 8 carbon atoms; a substituted or unsubstituted aryl group having 6 to 50, preferably 6 to 25, more preferably 6 to 18 ring carbon atoms; and a substituted or unsubstituted heteroaryl group having 5 to 50, preferably 5 to 24, more preferably 5 to 13 ring atoms. More preferably, the substituent represented by R¹⁹ and R²⁰ is independently a substituted or unsubstituted alkyl group having 1 to 50, preferably 1 to 18, more preferably 1 to 8 carbon atoms or a substituted or unsubstituted aryl group having 6 to 50, preferably 6 to 25, more preferably 6 to 18 ring carbon atoms, with a substituted or unsubstituted aryl group having 6 to 50, preferably 6 to 25, more preferably 6 to 18 ring carbon atoms being still more preferred. The substituent represented by R²¹ is more preferably a substituted or unsubstituted aryl group having 6 to 50, preferably 6 to 25, more preferably 6 to 18 ring carbon atoms.

The ring structure formed when R¹⁹ and R²⁰ of C(R¹⁹)(R²⁰) are bonded to each other is, for example, the following structure:

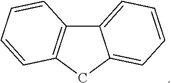 [SMILES: c1ccc2c(c1)Cc1ccccc1-2]

Y is preferably C(R¹⁹)(R²⁰), an oxygen atom, or a sulfur atom and more preferably an oxygen atom.

### L of Formula (1)

L is a single bond, a substituted or unsubstituted arylene group having 6 to 50 ring carbon atoms, or a substituted or unsubstituted heteroarylene group having 5 to 50 ring atoms.

The arylene group and the heteroarylene group for L and preferred examples thereof are the same as those for L¹ mentioned above.

In view of the emission efficiency and the lifetime, L is preferably a single bond or a substituted or unsubstituted arylene group having 6 to 50 ring carbon atoms and more preferably a single bond.

L is preferably bonded to one of R¹ to R⁴ of formula (1-A) and R¹¹ to R¹⁴ of formula (1-B). Namely, as described above, although one of R¹ to R⁸ of formula (1-A) can be bonded to L and one of R¹¹ to R¹⁸ of formula (1-B) can be bonded to L, one of R¹ to R⁴ and R¹¹ to R¹⁴ is necessarily bonded to L in a preferred embodiment.

For example, a compound wherein L is bonded to R³ of formula (1-A) and R¹³ of formula (1-B) is shown below.

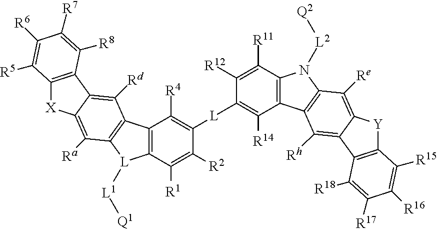

### Example of A of Formula (1)

In view of the emission efficiency and the lifetime, A is preferably a group represented by any of formulae (1-A-1) to (1-A-6):

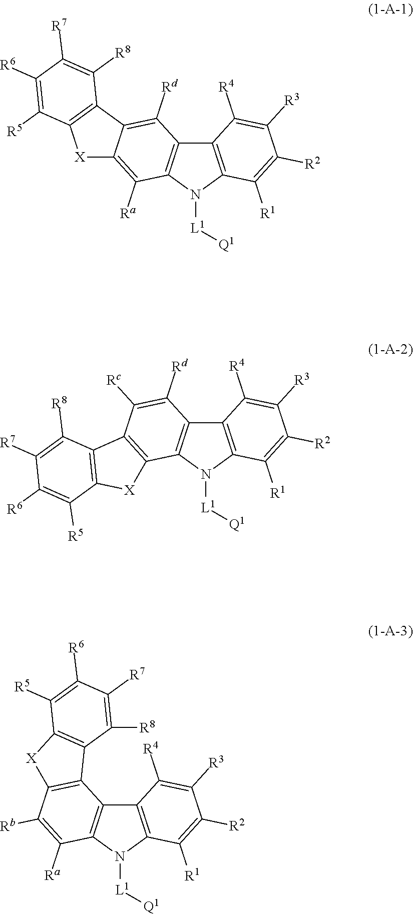

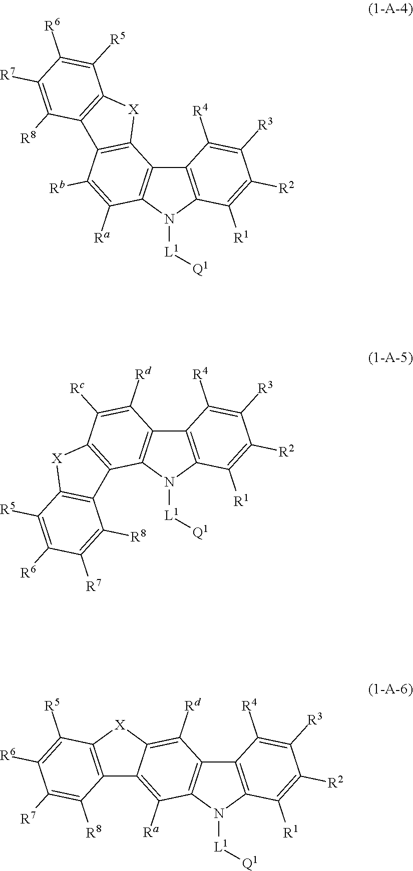

wherein:

R¹ to R⁸, X, L¹, Q¹, and preferred examples thereof are as defined above; and

each of R^{a} to R^{d} is independently a hydrogen atom or a substituent, wherein the substituent is as described above with respect to R^{a} to R^{d}.

### Example of B of Formula (1)

In view of the emission efficiency and the lifetime, B is preferably a group represented by any of formulae (1-B-1) to (1-B-6):

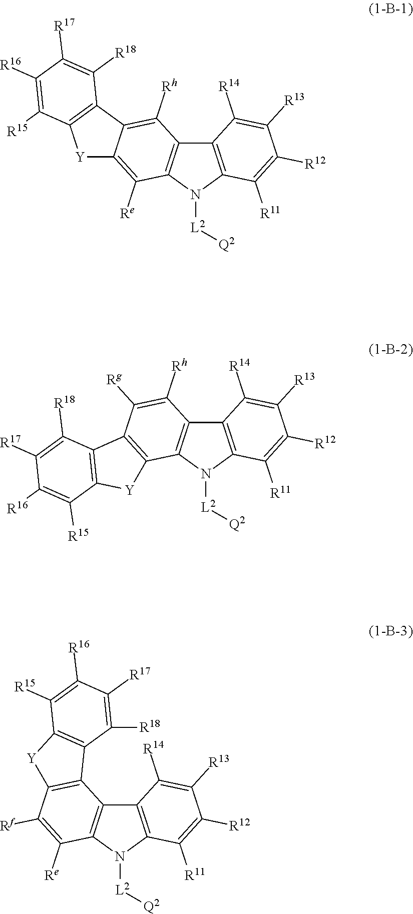

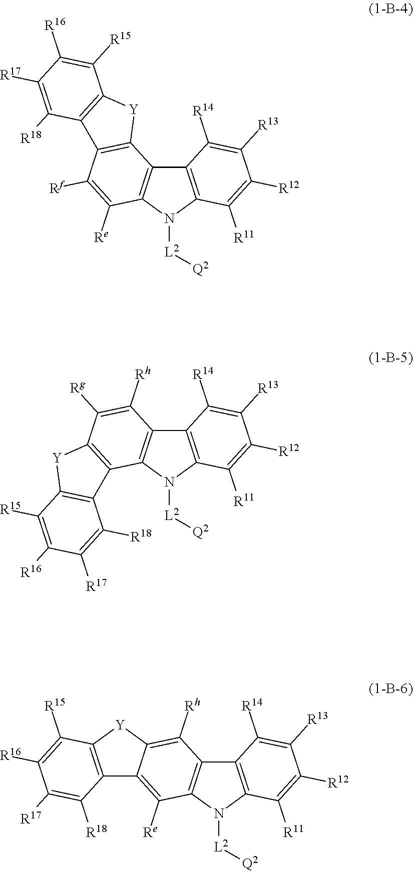

wherein:

R¹¹ to R¹⁸, Y, L², Q², and preferred examples thereof are as defined above; and

each of R^{e} to R^{h} is independently a hydrogen atom or a substituent, wherein the substituent is as described above with respect to R^{e} to R^{h}.

In view of the emission efficiency and the lifetime, in formula (1), A is bonded to L preferably via any of R¹ to R⁴, more preferably via R² or R³, and still more preferably via R³ of formulae (i-A) and (1-A-1) to (1-A-6).

In view of the emission efficiency and the lifetime, B is bonded to L preferably via any of R¹¹ to R¹⁴, more preferably via R¹² or R¹³, and still more preferably via R¹³ of formulae (1-B) and (1-B-1) to (1-B-6).

### Preferred Embodiment of the Compound Represented by Formula (1)

In view of the emission efficiency and the lifetime, the compound represented by formula (1) is preferably a compound represented by formula (2):

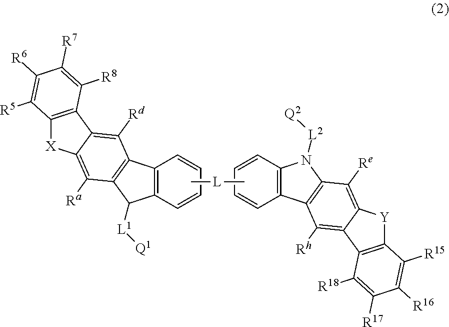

wherein R^{a}, R^{d}, R^{e}, R^{h}, R⁵ to R⁸, R¹⁵ to R¹⁸, X, Y, L, L¹, L², Q¹, Q², and preferred examples thereof are as defined above.

Two benzene rings to which L is bonded may have a substituent, wherein the substituent is as described above with respect to R¹ to R⁴ and R¹¹ to R¹⁴. The substituents may be bonded to each other to form a ring structure. Preferably, two benzene rings to which L is bonded have no substituent.

In view of the emission efficiency and the lifetime, the compound represented by formula (1) is more preferably a compound represented by any of formulae (2-1) to (2-4):

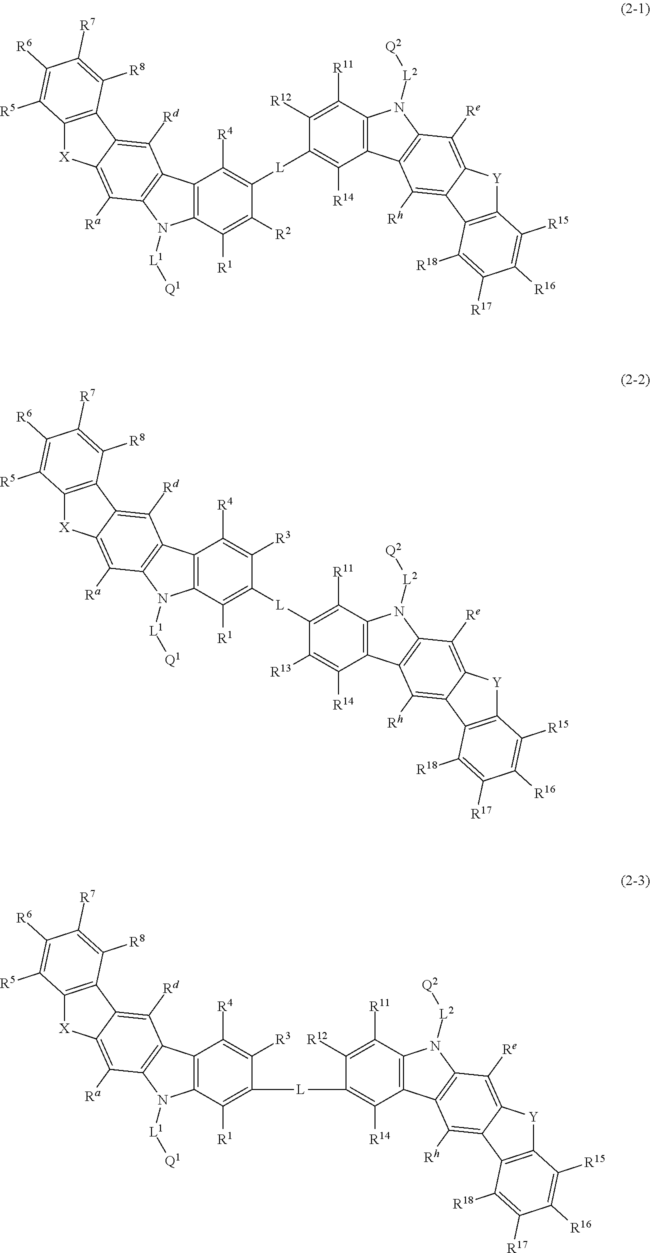

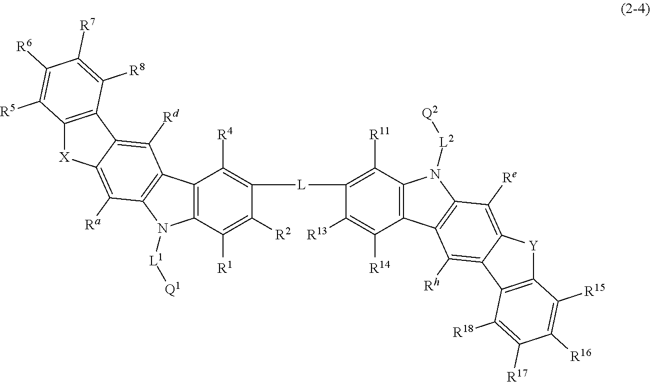

wherein R^{a}, R^{d}, R^{e}, R^{h}, R⁵ to R⁸, R¹⁵ to R¹⁸, X, Y, L, L¹, L², Q¹, Q², and preferred examples thereof are as defined above; and

each of R¹ to R⁴ and R¹¹ to R¹⁴ is independently a hydrogen atom or a substituent, and adjacent substituents may be bonded to each other to form a ring structure.

The substituent represented by R¹ to R⁴ and R¹¹ to R¹⁴ is as defined above. Preferably, adjacent substituents do not form a ring structure.

R¹ to R⁴ and R¹¹ to R¹⁴ are preferably all hydrogen atoms.

In view of the emission efficiency and the lifetime, the compound represented by formula (1) is still more preferably a compound represented by any of formulae (2-1′) to (2-4′) and further still more preferably a compound represented by any of formulae (2-1′), (2-3′), and (2-4′):

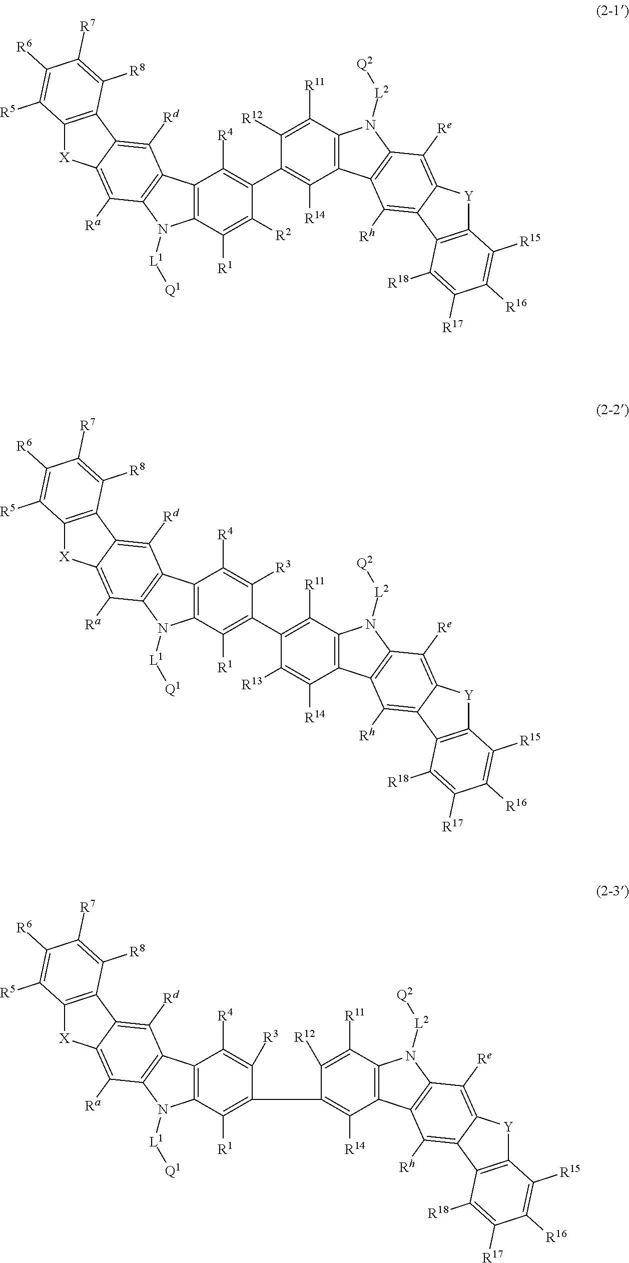

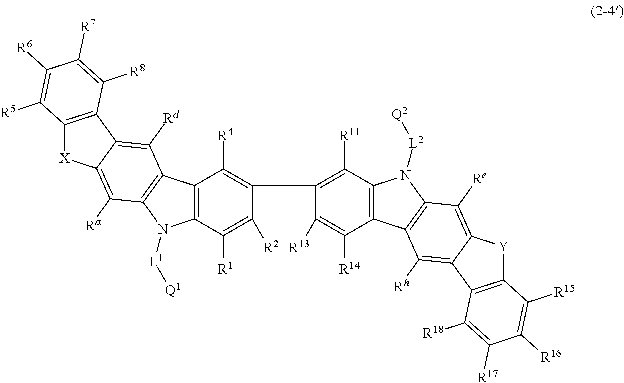

wherein R^{a}, R^{d}, R^{e}, R^{h}, R⁵ to R⁸, R¹⁵ to R¹⁸, X, Y, L¹, L², Q¹, Q², and preferred examples thereof are as defined above; and

each of R¹ to R⁴ and R¹¹ to R¹⁴ is independently a hydrogen atom or a substituent, and adjacent substituents may be bonded to each other to form a ring structure.

The substituent represented by R¹ to R⁴ and R¹¹ to R¹⁴ is as defined above. Preferably, adjacent substituents do not form a ring structure.

R¹ to R⁴ and R¹¹ to R¹⁴ are preferably all hydrogen atoms.

In view of the emission efficiency and the lifetime, the compound represented by formula (1) is still more preferably a compound represented by any of formulae (2-1′-i) to (2-4′-i) and further still more preferably a compound represented by formula (2-1′-i) or (2-4′-i):

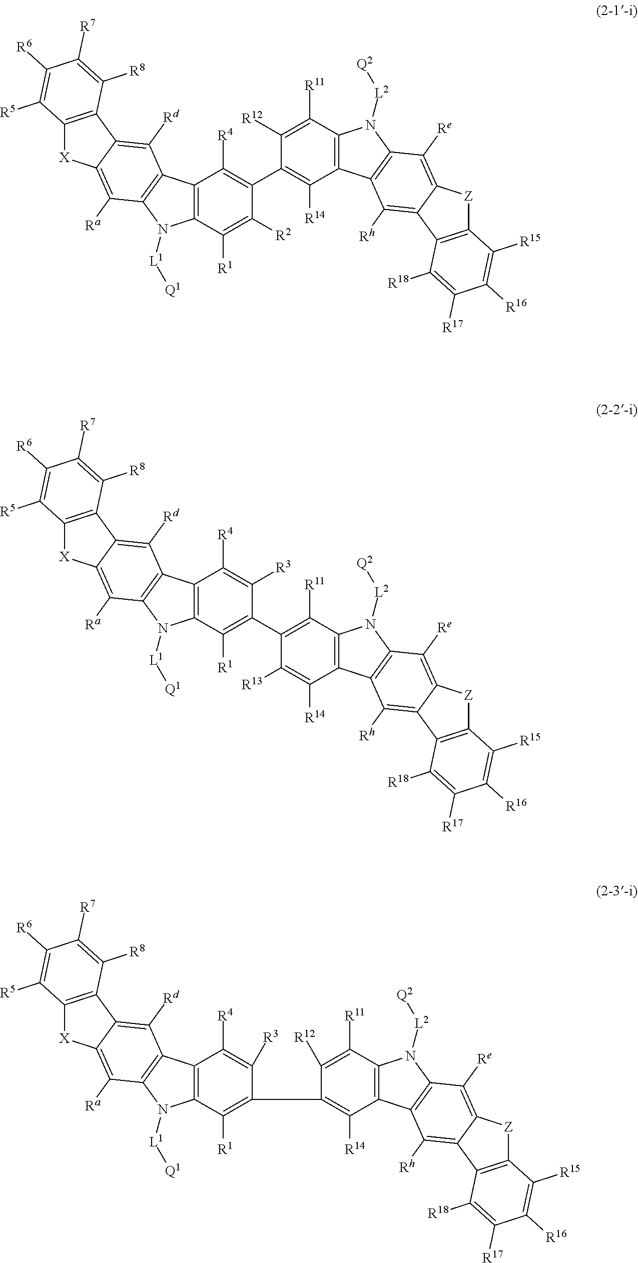

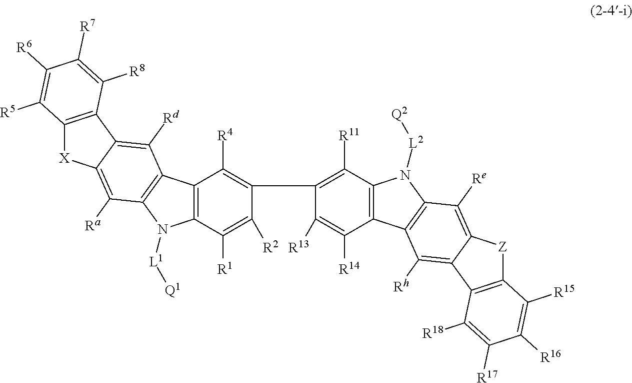

wherein R^{a}, R^{d}, R^{e}, R^{h}, R⁵ to R⁸, R¹⁵ to R¹⁸, X, L¹, L², Q¹, Q², and preferred examples thereof are as defined above; and

each of R¹ to R⁴ and R¹¹ to R¹⁴ is independently a hydrogen atom or a substituent, and adjacent substituents may be bonded to each other to form a ring structure.

The substituent represented by R¹ to R⁴ and R¹¹ to R¹⁴ is as defined above. Preferably, adjacent substituents do not form a ring structure.

R¹ to R⁴ and R¹¹ to R¹⁴ are preferably all hydrogen atoms.

Z is an oxygen atom or a sulfur atom. In view of the emission efficiency and the lifetime, Z is preferably an oxygen atom.

In view of the emission efficiency and the lifetime, the compound represented by formula (1) is still more preferably a compound represented by any of formulae (2-1′-i-O) to (2-4′-i-O) and further still more preferably a compound represented by (2-1′-i-O) or (2-4′-i-O):

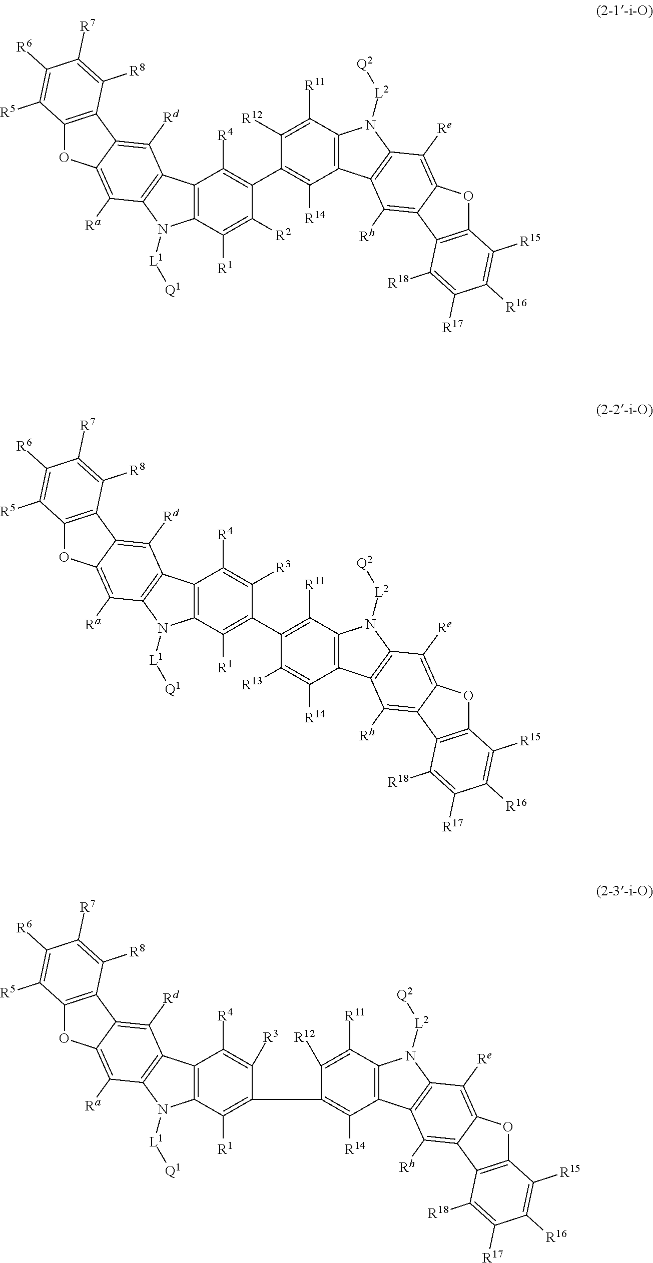

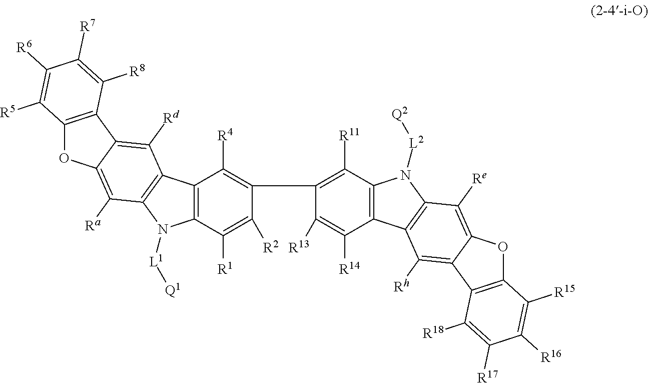

wherein R^{a}, R^{d}, R^{e}, R^{h}, R⁵ to R⁸, R¹⁵ to R¹⁸, L¹, L², Q¹, Q², and preferred examples thereof are as defined above; and

each of R¹ to R⁴ and R¹¹ to R¹⁴ is independently a hydrogen atom or a substituent, and adjacent substituents may be bonded to each other to form a ring structure.

The substituent represented by R¹ to R⁴ and R¹¹ to R¹⁴ is as defined above. Preferably, adjacent substituents do not form a ring structure.

R¹ to R⁴ and R¹¹ to R¹⁴ are preferably all hydrogen atoms.

In view of the emission efficiency and the lifetime, the compound represented by any of the formulae mentioned above wherein Q¹ and Q² is independently a substituted or unsubstituted aryl group having 6 to 50 ring carbon atoms is also preferred. Such a compound (1) of the invention is suitable as a material for an anode-side organic thin film layer (a hole transporting layer, a hole injecting layer, etc.) between an anode and a light emitting layer or a host material (particularly a phosphorescent host material) of a light emitting layer, particularly as a material for an anode-side organic thin film layer (a hole transporting layer, a hole injecting layer, etc.).

In view of the emission efficiency and the lifetime, the compound represented by any of the formulae mentioned above wherein Q¹ is a group represented by any of formulae (3) and (3-1) to (3-4) and Q² is a substituted or unsubstituted aryl group having 6 to 50 ring carbon atoms is also preferred. Such a compound (1) of the invention is suitable as a host material (particularly a phosphorescent material) of a light emitting layer.

In view of the emission efficiency and the lifetime, in any of the formulae mentioned above, R¹ to R⁸ not bonded to L, R¹¹ to R¹⁸ not bonded to L, R^{a} to R^{d} not bonded to the site * of formula (1-a), thereby failing to form a ring structure, and R^{e} to R^{h} not bonded to the site * of formula (1-b), thereby failing to form a ring structure are preferably all hydrogen atoms.

Examples of the compound in an aspect of the invention are shown below, although not limited thereto.

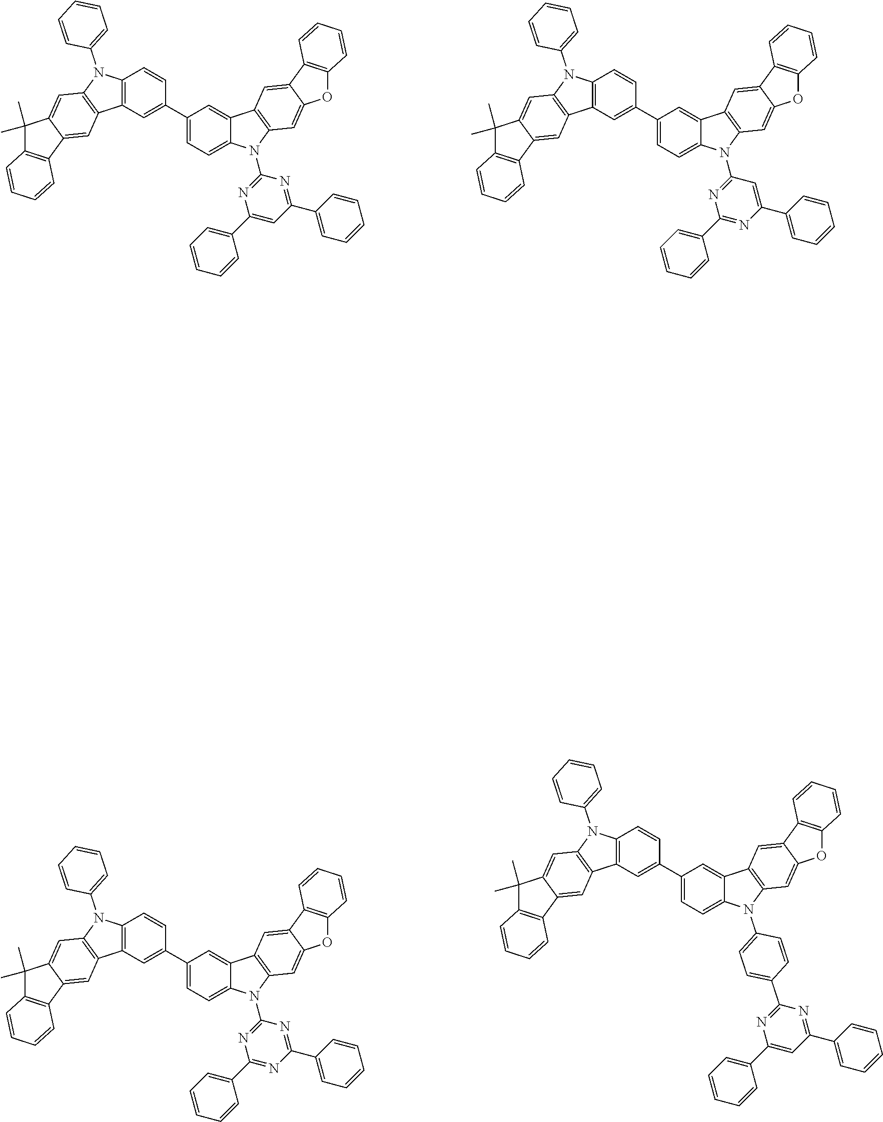 [SMILES: CC1(C)c2ccccc2-c2cc3c4cc(-c5ccc6c(c5)c5cc7c(cc5n6-c5nc(-c6ccccc6)cc(-c6ccccc6)n5)oc5ccccc57)ccc4n(-c4ccccc4)c3cc21 ; CC1(C)c2ccccc2-c2cc3c4cc(-c5ccc6c(c5)c5cc7c(cc5n6-c5cc(-c6ccccc6)nc(-c6ccccc6)n5)oc5ccccc57)ccc4n(-c4ccccc4)c3cc21 ; CC1(C)c2ccccc2-c2cc3c4cc(-c5ccc6c(c5)c5cc7c(cc5n6-c5nc(-c6ccccc6)nc(-c6ccccc6)n5)oc5ccccc57)ccc4n(-c4ccccc4)c3cc21 ; CC1(C)c2ccccc2-c2cc3c4cc(-c5ccc6c(c5)c5cc7c(cc5n6-c5ccc(-c6nc(-c8ccccc8)cc(-c8ccccc8)n6)cc5)oc5ccccc57)ccc4n(-c4ccccc4)c3cc21]

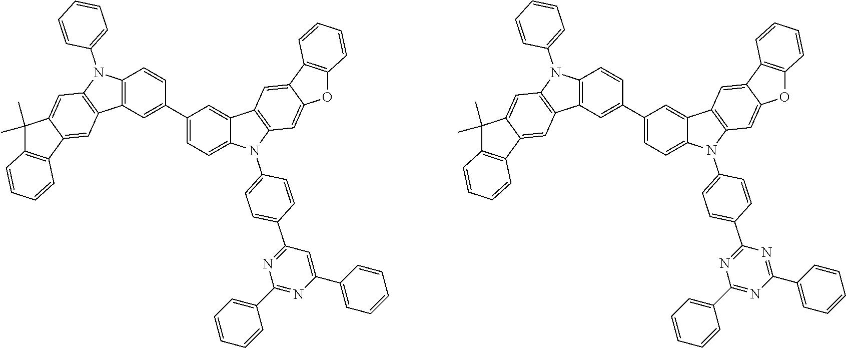 [SMILES: CC1(C)c2ccccc2-c2cc3c4cc(-c5ccc6c(c5)c5cc7c(cc5n6-c5ccc(-c6cc(-c8ccccc8)nc(-c8ccccc8)n6)cc5)oc5ccccc57)ccc4n(-c4ccccc4)c3cc21 ; CC1(C)c2ccccc2-c2cc3c4cc(-c5ccc6c(c5)c5cc7c(cc5n6-c5ccc(-c6nc(-c8ccccc8)nc(-c8ccccc8)n6)cc5)oc5ccccc57)ccc4n(-c4ccccc4)c3cc21]

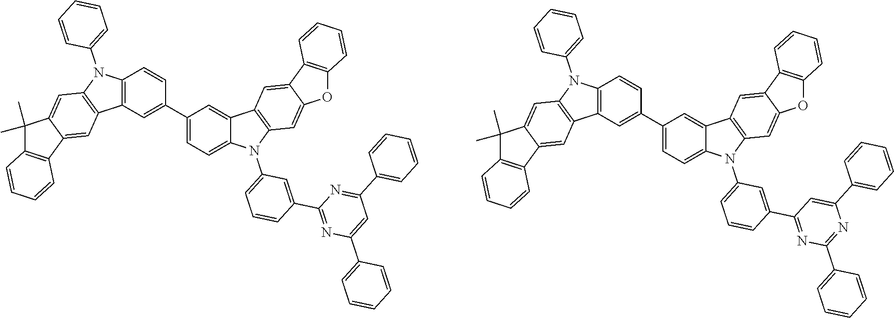 [SMILES: CC1(C)c2ccccc2-c2cc3c4cc(-c5ccc6c(c5)c5cc7c(cc5n6-c5cccc(-c6nc(-c8ccccc8)cc(-c8ccccc8)n6)c5)oc5ccccc57)ccc4n(-c4ccccc4)c3cc21 ; CC1(C)c2ccccc2-c2cc3c4cc(-c5ccc6c(c5)c5cc7c(cc5n6-c5cccc(-c6cc(-c8ccccc8)nc(-c8ccccc8)n6)c5)oc5ccccc57)ccc4n(-c4ccccc4)c3cc21]

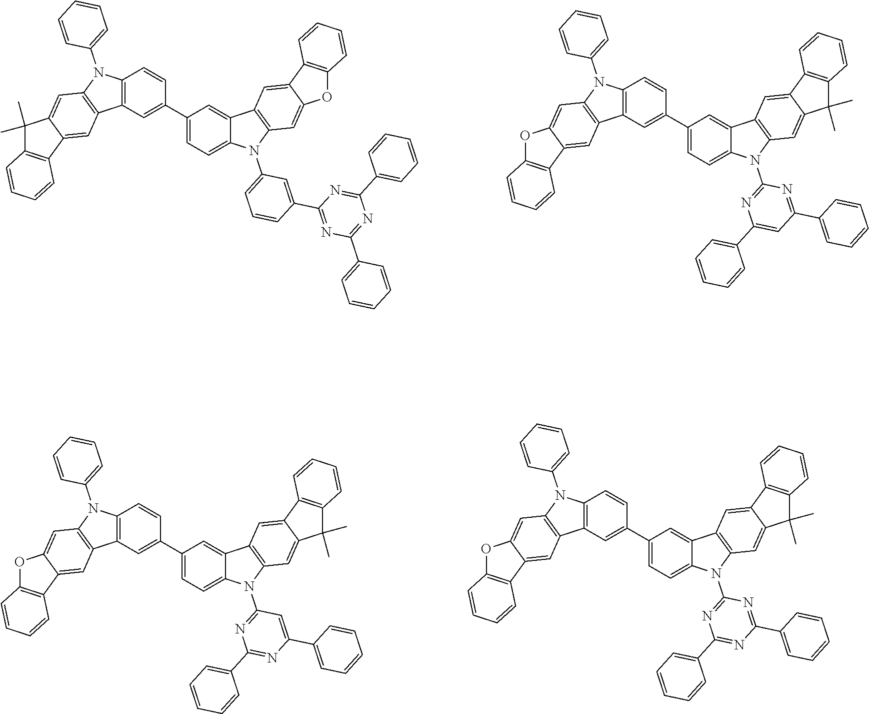 [SMILES: CC1(C)c2ccccc2-c2cc3c4cc(-c5ccc6c(c5)c5cc7c(cc5n6-c5cccc(-c6nc(-c8ccccc8)nc(-c8ccccc8)n6)c5)oc5ccccc57)ccc4n(-c4ccccc4)c3cc21 ; CC1(C)c2ccccc2-c2cc3c4cc(-c5ccc6c(c5)c5cc7c(cc5n6-c5ccccc5)oc5ccccc57)ccc4n(-c4nc(-c5ccccc5)cc(-c5ccccc5)n4)c3cc21 ; CC1(C)c2ccccc2-c2cc3c4cc(-c5ccc6c(c5)c5cc7c(cc5n6-c5ccccc5)oc5ccccc57)ccc4n(-c4cc(-c5ccccc5)nc(-c5ccccc5)n4)c3cc21 ; CC1(C)c2ccccc2-c2cc3c4cc(-c5ccc6c(c5)c5cc7c(cc5n6-c5ccccc5)oc5ccccc57)ccc4n(-c4nc(-c5ccccc5)nc(-c5ccccc5)n4)c3cc21]

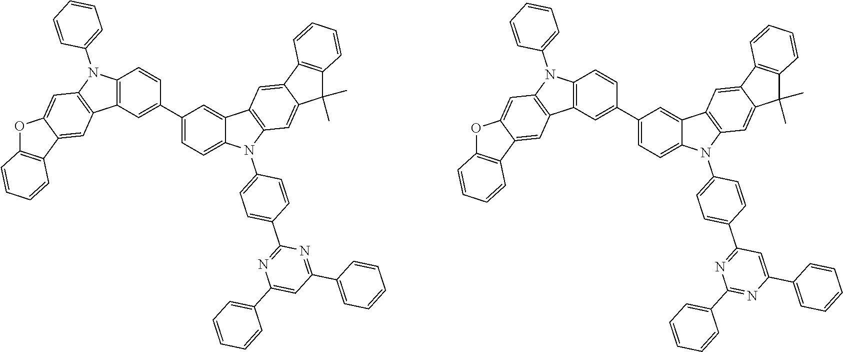 [SMILES: CC1(C)c2ccccc2-c2cc3c4cc(-c5ccc6c(c5)c5cc7c(cc5n6-c5ccccc5)oc5ccccc57)ccc4n(-c4ccc(-c5nc(-c6ccccc6)cc(-c6ccccc6)n5)cc4)c3cc21 ; CC1(C)c2ccccc2-c2cc3c4cc(-c5ccc6c(c5)c5cc7c(cc5n6-c5ccccc5)oc5ccccc57)ccc4n(-c4ccc(-c5cc(-c6ccccc6)nc(-c6ccccc6)n5)cc4)c3cc21]

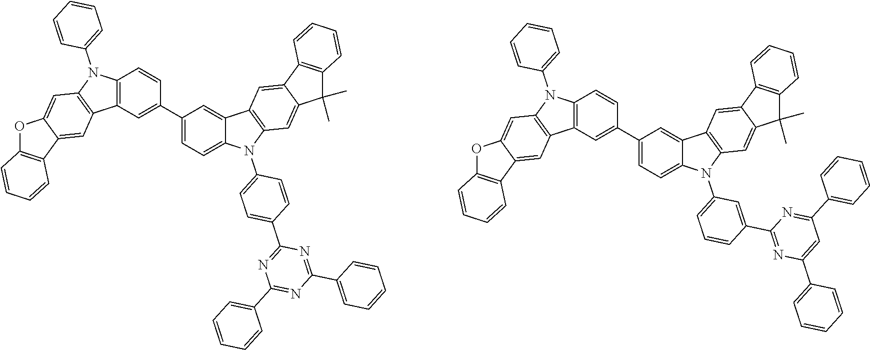 [SMILES: CC1(C)c2ccccc2-c2cc3c4cc(-c5ccc6c(c5)c5cc7c(cc5n6-c5ccccc5)oc5ccccc57)ccc4n(-c4ccc(-c5nc(-c6ccccc6)nc(-c6ccccc6)n5)cc4)c3cc21 ; CC1(C)c2ccccc2-c2cc3c4cc(-c5ccc6c(c5)c5cc7c(cc5n6-c5ccccc5)oc5ccccc57)ccc4n(-c4cccc(-c5nc(-c6ccccc6)cc(-c6ccccc6)n5)c4)c3cc21]

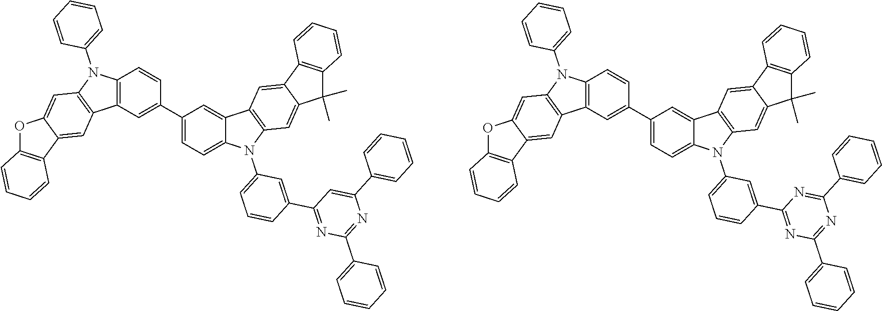 [SMILES: CC1(C)c2ccccc2-c2cc3c4cc(-c5ccc6c(c5)c5cc7c(cc5n6-c5ccccc5)oc5ccccc57)ccc4n(-c4cccc(-c5cc(-c6ccccc6)nc(-c6ccccc6)n5)c4)c3cc21 ; CC1(C)c2ccccc2-c2cc3c4cc(-c5ccc6c(c5)c5cc7c(cc5n6-c5ccccc5)oc5ccccc57)ccc4n(-c4cccc(-c5nc(-c6ccccc6)nc(-c6ccccc6)n5)c4)c3cc21]

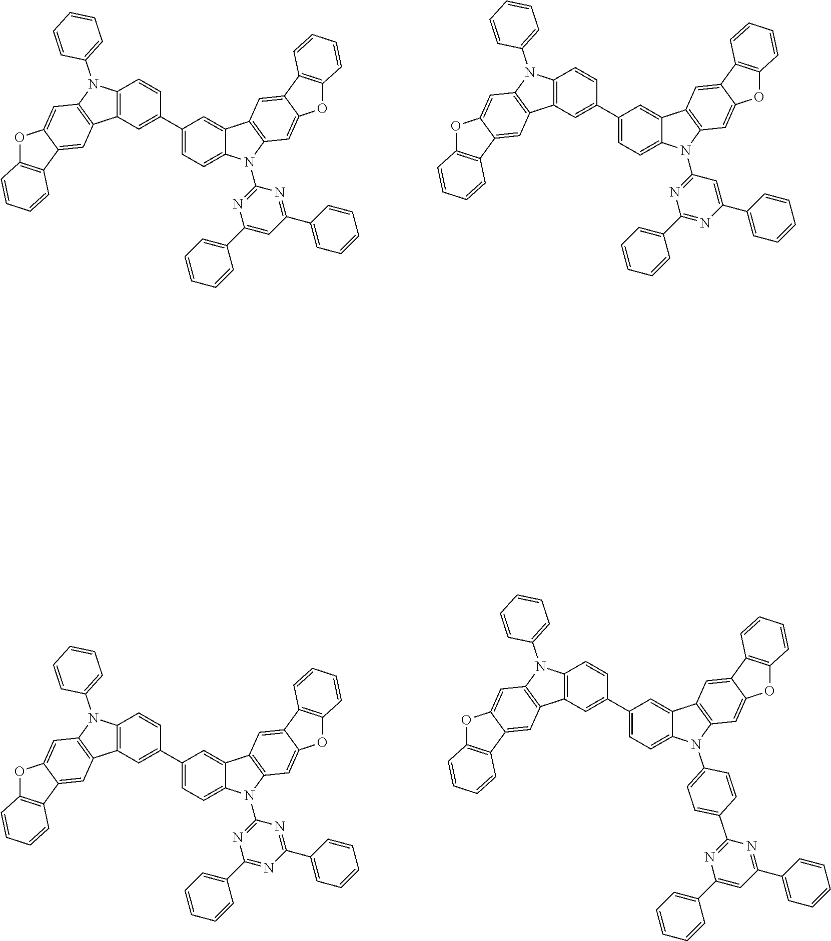 [SMILES: c1ccc(-c2cc(-c3ccccc3)nc(-n3c4ccc(-c5ccc6c(c5)c5cc7c(cc5n6-c5ccccc5)oc5ccccc57)cc4c4cc5c(cc43)oc3ccccc35)n2)cc1 ; c1ccc(-c2cc(-n3c4ccc(-c5ccc6c(c5)c5cc7c(cc5n6-c5ccccc5)oc5ccccc57)cc4c4cc5c(cc43)oc3ccccc35)nc(-c3ccccc3)n2)cc1 ; c1ccc(-c2nc(-c3ccccc3)nc(-n3c4ccc(-c5ccc6c(c5)c5cc7c(cc5n6-c5ccccc5)oc5ccccc57)cc4c4cc5c(cc43)oc3ccccc35)n2)cc1 ; c1ccc(-c2cc(-c3ccccc3)nc(-c3ccc(-n4c5ccc(-c6ccc7c(c6)c6cc8c(cc6n7-c6ccccc6)oc6ccccc68)cc5c5cc6c(cc54)oc4ccccc46)cc3)n2)cc1]

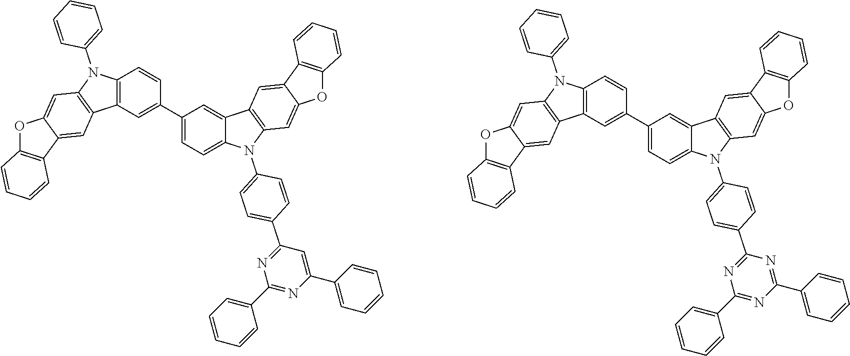 [SMILES: c1ccc(-c2cc(-c3ccc(-n4c5ccc(-c6ccc7c(c6)c6cc8c(cc6n7-c6ccccc6)oc6ccccc68)cc5c5cc6c(cc54)oc4ccccc46)cc3)nc(-c3ccccc3)n2)cc1 ; c1ccc(-c2nc(-c3ccccc3)nc(-c3ccc(-n4c5ccc(-c6ccc7c(c6)c6cc8c(cc6n7-c6ccccc6)oc6ccccc68)cc5c5cc6c(cc54)oc4ccccc46)cc3)n2)cc1]

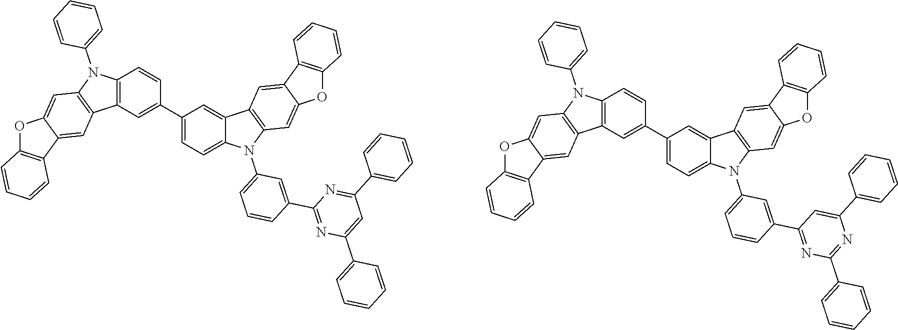 [SMILES: c1ccc(-c2cc(-c3ccccc3)nc(-c3cccc(-n4c5ccc(-c6ccc7c(c6)c6cc8c(cc6n7-c6ccccc6)oc6ccccc68)cc5c5cc6c(cc54)oc4ccccc46)c3)n2)cc1 ; c1ccc(-c2cc(-c3cccc(-n4c5ccc(-c6ccc7c(c6)c6cc8c(cc6n7-c6ccccc6)oc6ccccc68)cc5c5cc6c(cc54)oc4ccccc46)c3)nc(-c3ccccc3)n2)cc1]

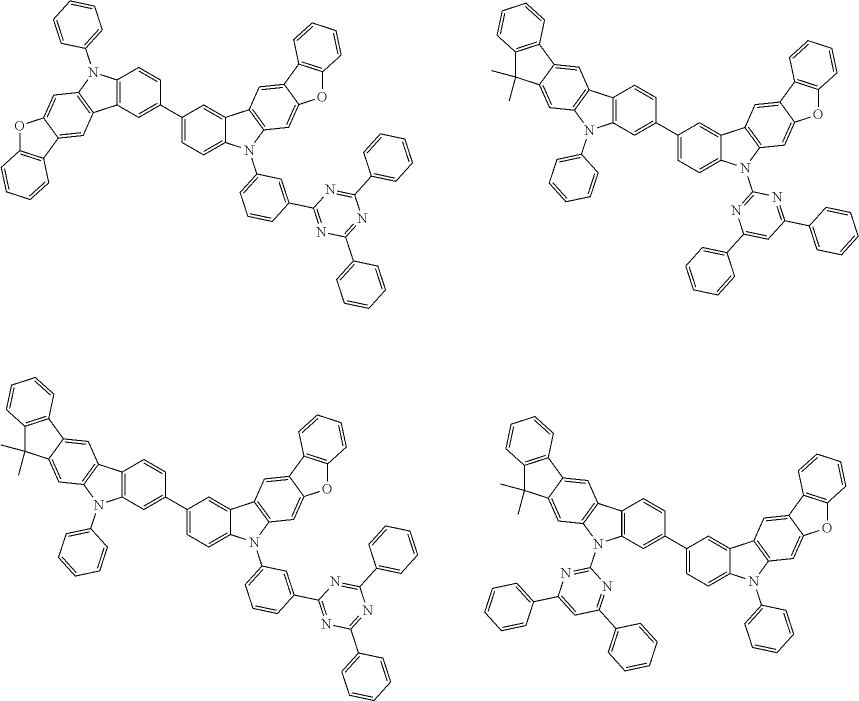 [SMILES: c1ccc(-c2nc(-c3ccccc3)nc(-c3cccc(-n4c5ccc(-c6ccc7c(c6)c6cc8c(cc6n7-c6ccccc6)oc6ccccc68)cc5c5cc6c(cc54)oc4ccccc46)c3)n2)cc1 ; CC1(C)c2ccccc2-c2cc3c4ccc(-c5ccc6c(c5)c5cc7c(cc5n6-c5nc(-c6ccccc6)cc(-c6ccccc6)n5)oc5ccccc57)cc4n(-c4ccccc4)c3cc21 ; CC1(C)c2ccccc2-c2cc3c4ccc(-c5ccc6c(c5)c5cc7c(cc5n6-c5cccc(-c6nc(-c8ccccc8)nc(-c8ccccc8)n6)c5)oc5ccccc57)cc4n(-c4ccccc4)c3cc21 ; CC1(C)c2ccccc2-c2cc3c4ccc(-c5ccc6c(c5)c5cc7c(cc5n6-c5ccccc5)oc5ccccc57)cc4n(-c4nc(-c5ccccc5)cc(-c5ccccc5)n4)c3cc21]

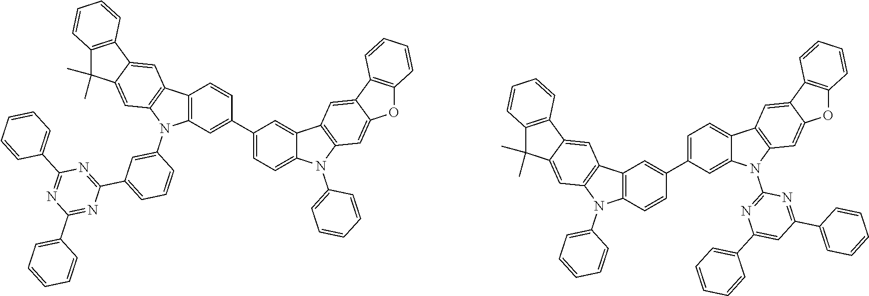 [SMILES: CC1(C)c2ccccc2-c2cc3c4ccc(-c5ccc6c(c5)c5cc7c(cc5n6-c5ccccc5)oc5ccccc57)cc4n(-c4cccc(-c5nc(-c6ccccc6)nc(-c6ccccc6)n5)c4)c3cc21 ; CC1(C)c2ccccc2-c2cc3c4cc(-c5ccc6c7cc8c(cc7n(-c7nc(-c9ccccc9)cc(-c9ccccc9)n7)c6c5)oc5ccccc58)ccc4n(-c4ccccc4)c3cc21]

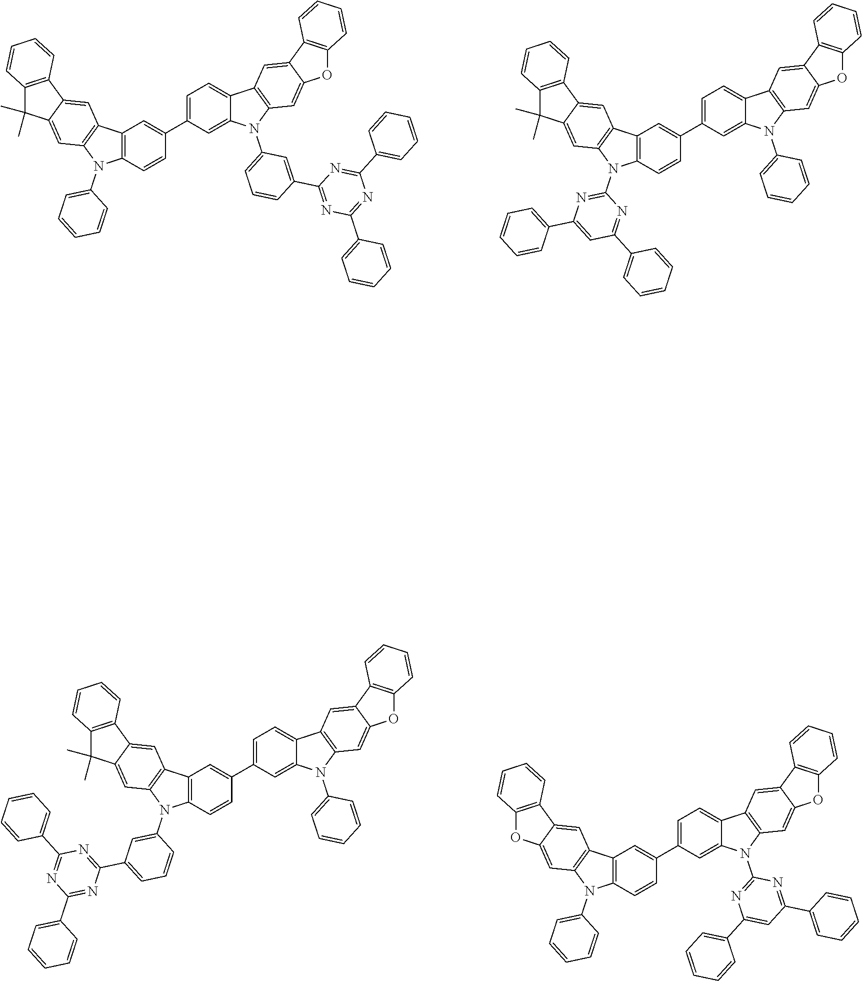 [SMILES: CC1(C)c2ccccc2-c2cc3c4cc(-c5ccc6c7cc8c(cc7n(-c7cccc(-c9nc(-c%10ccccc%10)nc(-c%10ccccc%10)n9)c7)c6c5)oc5ccccc58)ccc4n(-c4ccccc4)c3cc21 ; CC1(C)c2ccccc2-c2cc3c4cc(-c5ccc6c7cc8c(cc7n(-c7ccccc7)c6c5)oc5ccccc58)ccc4n(-c4nc(-c5ccccc5)cc(-c5ccccc5)n4)c3cc21 ; CC1(C)c2ccccc2-c2cc3c4cc(-c5ccc6c7cc8c(cc7n(-c7ccccc7)c6c5)oc5ccccc58)ccc4n(-c4cccc(-c5nc(-c6ccccc6)nc(-c6ccccc6)n5)c4)c3cc21 ; c1ccc(-c2cc(-c3ccccc3)nc(-n3c4cc(-c5ccc6c(c5)c5cc7c(cc5n6-c5ccccc5)oc5ccccc57)ccc4c4cc5c(cc43)oc3ccccc35)n2)cc1]

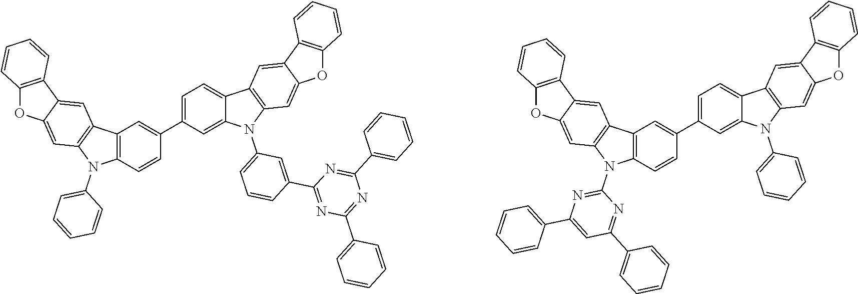 [SMILES: c1ccc(-c2nc(-c3ccccc3)nc(-c3cccc(-n4c5cc(-c6ccc7c(c6)c6cc8c(cc6n7-c6ccccc6)oc6ccccc68)ccc5c5cc6c(cc54)oc4ccccc46)c3)n2)cc1 ; c1ccc(-c2cc(-c3ccccc3)nc(-n3c4ccc(-c5ccc6c7cc8c(cc7n(-c7ccccc7)c6c5)oc5ccccc58)cc4c4cc5c(cc43)oc3ccccc35)n2)cc1]

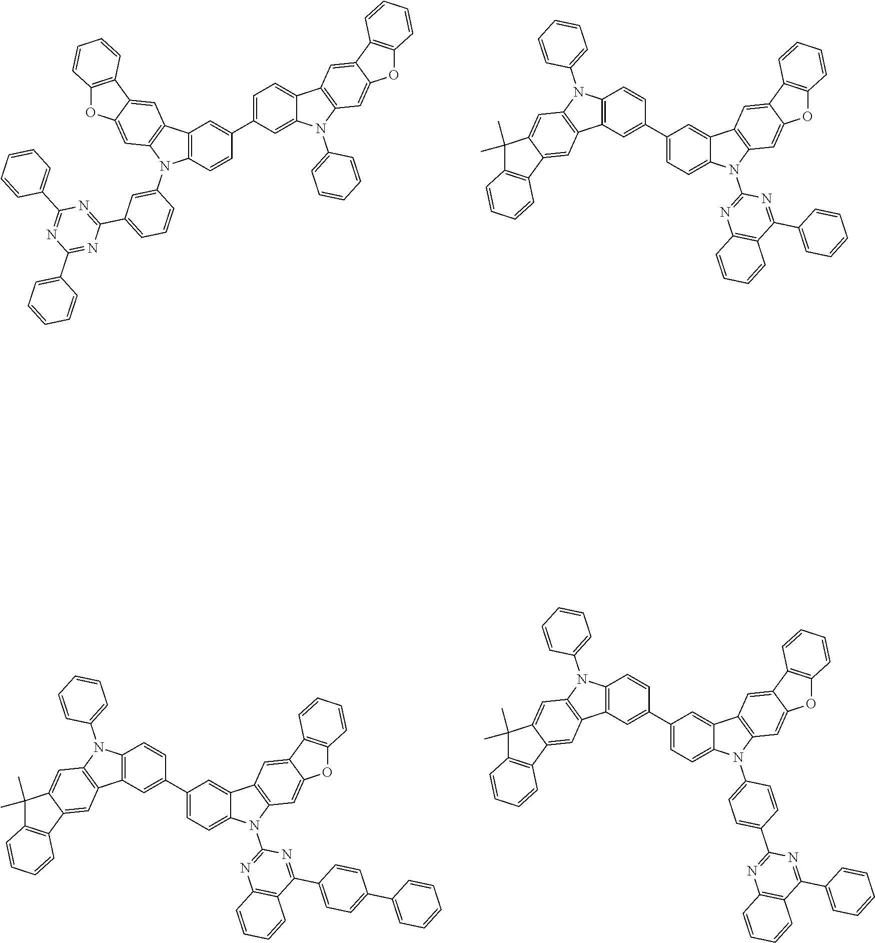 [SMILES: c1ccc(-c2nc(-c3ccccc3)nc(-c3cccc(-n4c5ccc(-c6ccc7c8cc9c(cc8n(-c8ccccc8)c7c6)oc6ccccc69)cc5c5cc6c(cc54)oc4ccccc46)c3)n2)cc1 ; CC1(C)c2ccccc2-c2cc3c4cc(-c5ccc6c(c5)c5cc7c(cc5n6-c5nc(-c6ccccc6)c6ccccc6n5)oc5ccccc57)ccc4n(-c4ccccc4)c3cc21 ; CC1(C)c2ccccc2-c2cc3c4cc(-c5ccc6c(c5)c5cc7c(cc5n6-c5nc(-c6ccc(-c8ccccc8)cc6)c6ccccc6n5)oc5ccccc57)ccc4n(-c4ccccc4)c3cc21 ; CC1(C)c2ccccc2-c2cc3c4cc(-c5ccc6c(c5)c5cc7c(cc5n6-c5ccc(-c6nc(-c8ccccc8)c8ccccc8n6)cc5)oc5ccccc57)ccc4n(-c4ccccc4)c3cc21]

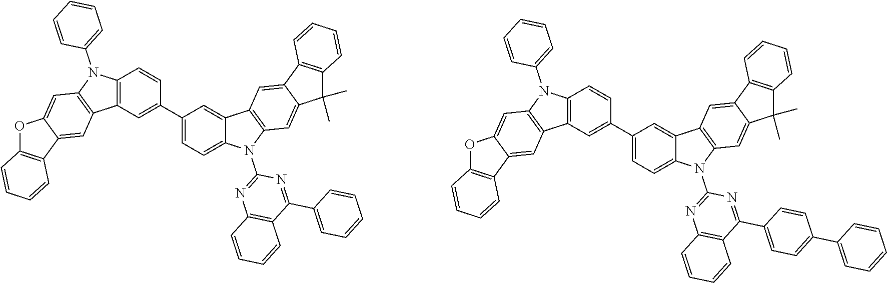 [SMILES: CC1(C)c2ccccc2-c2cc3c4cc(-c5ccc6c(c5)c5cc7c(cc5n6-c5ccccc5)oc5ccccc57)ccc4n(-c4nc(-c5ccccc5)c5ccccc5n4)c3cc21 ; CC1(C)c2ccccc2-c2cc3c4cc(-c5ccc6c(c5)c5cc7c(cc5n6-c5ccccc5)oc5ccccc57)ccc4n(-c4nc(-c5ccc(-c6ccccc6)cc5)c5ccccc5n4)c3cc21]

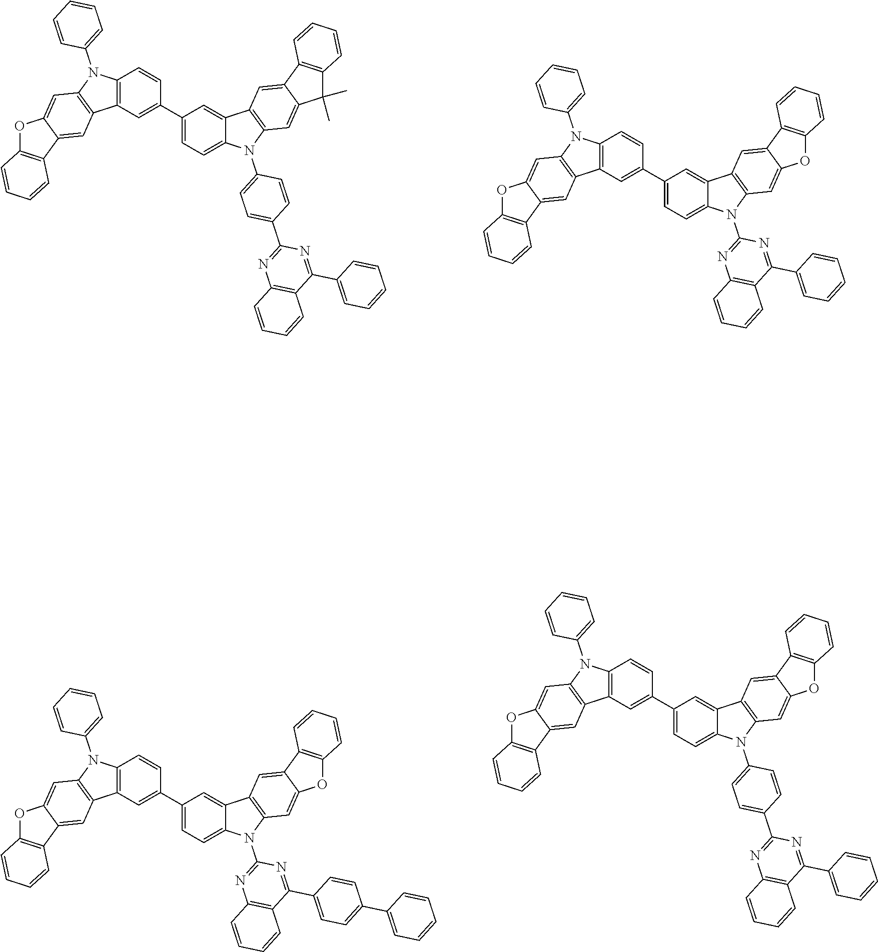 [SMILES: CC1(C)c2ccccc2-c2cc3c4cc(-c5ccc6c(c5)c5cc7c(cc5n6-c5ccccc5)oc5ccccc57)ccc4n(-c4ccc(-c5nc(-c6ccccc6)c6ccccc6n5)cc4)c3cc21 ; c1ccc(-c2nc(-n3c4ccc(-c5ccc6c(c5)c5cc7c(cc5n6-c5ccccc5)oc5ccccc57)cc4c4cc5c(cc43)oc3ccccc35)nc3ccccc23)cc1 ; c1ccc(-c2ccc(-c3nc(-n4c5ccc(-c6ccc7c(c6)c6cc8c(cc6n7-c6ccccc6)oc6ccccc68)cc5c5cc6c(cc54)oc4ccccc46)nc4ccccc34)cc2)cc1 ; c1ccc(-c2nc(-c3ccc(-n4c5ccc(-c6ccc7c(c6)c6cc8c(cc6n7-c6ccccc6)oc6ccccc68)cc5c5cc6c(cc54)oc4ccccc46)cc3)nc3ccccc23)cc1]

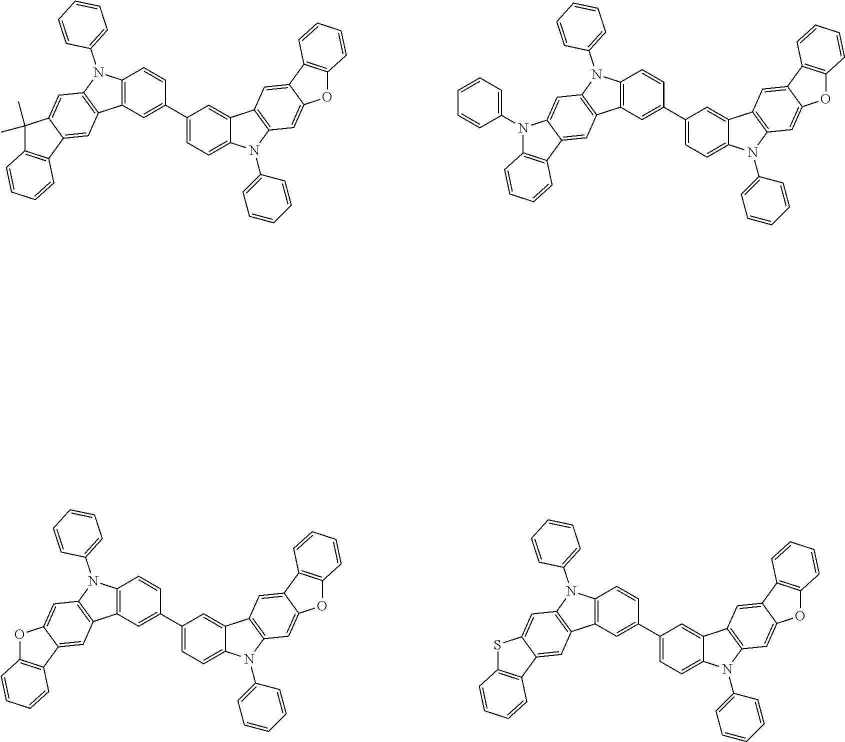 [SMILES: CC1(C)c2ccccc2-c2cc3c4cc(-c5ccc6c(c5)c5cc7c(cc5n6-c5ccccc5)oc5ccccc57)ccc4n(-c4ccccc4)c3cc21 ; c1ccc(-n2c3ccc(-c4ccc5c(c4)c4cc6c7ccccc7n(-c7ccccc7)c6cc4n5-c4ccccc4)cc3c3cc4c(cc32)oc2ccccc24)cc1 ; c1ccc(-n2c3ccc(-c4ccc5c(c4)c4cc6c(cc4n5-c4ccccc4)oc4ccccc46)cc3c3cc4c(cc32)oc2ccccc24)cc1 ; c1ccc(-n2c3ccc(-c4ccc5c(c4)c4cc6c(cc4n5-c4ccccc4)sc4ccccc46)cc3c3cc4c(cc32)oc2ccccc24)cc1]

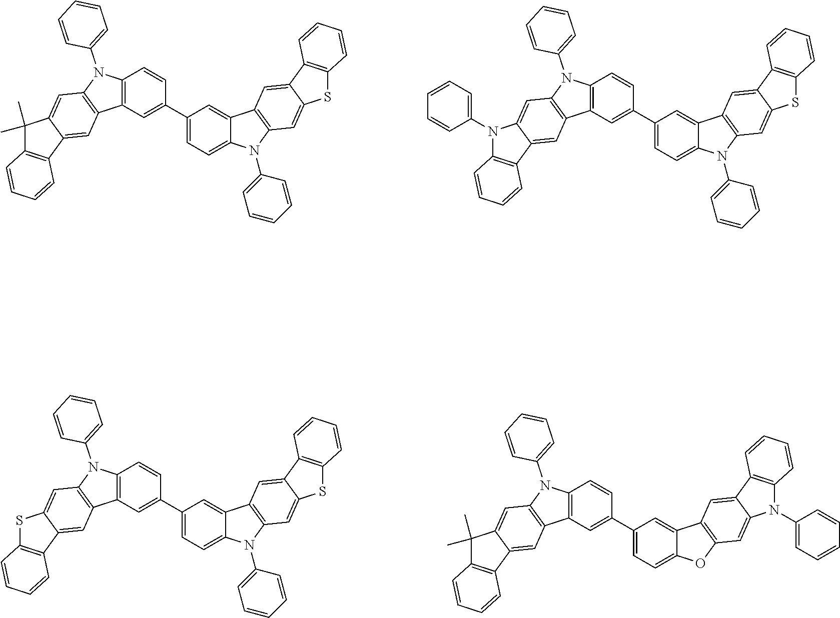 [SMILES: CC1(C)c2ccccc2-c2cc3c4cc(-c5ccc6c(c5)c5cc7c(cc5n6-c5ccccc5)sc5ccccc57)ccc4n(-c4ccccc4)c3cc21 ; c1ccc(-n2c3ccc(-c4ccc5c(c4)c4cc6c7ccccc7n(-c7ccccc7)c6cc4n5-c4ccccc4)cc3c3cc4c(cc32)sc2ccccc24)cc1 ; c1ccc(-n2c3ccc(-c4ccc5c(c4)c4cc6c(cc4n5-c4ccccc4)sc4ccccc46)cc3c3cc4c(cc32)sc2ccccc24)cc1 ; CC1(C)c2ccccc2-c2cc3c4cc(-c5ccc6oc7cc8c(cc7c6c5)c5ccccc5n8-c5ccccc5)ccc4n(-c4ccccc4)c3cc21]

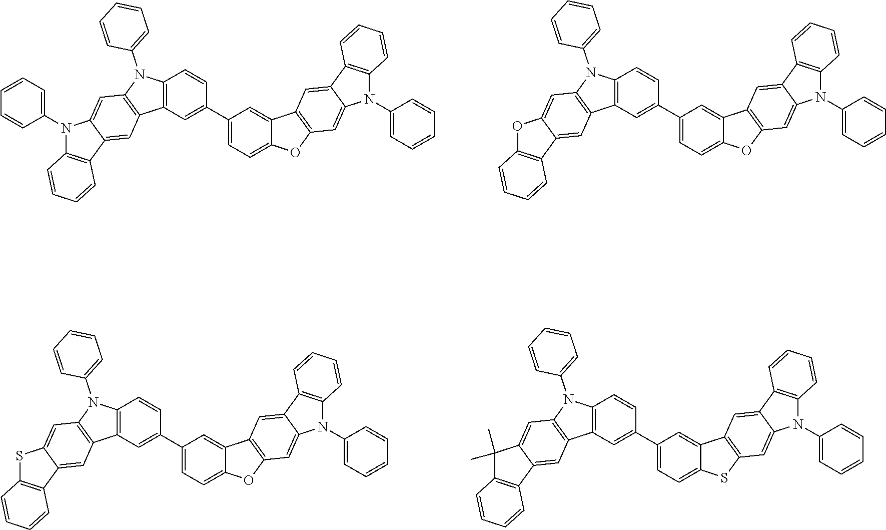 [SMILES: c1ccc(-n2c3ccccc3c3cc4c(cc32)oc2ccc(-c3ccc5c(c3)c3cc6c7ccccc7n(-c7ccccc7)c6cc3n5-c3ccccc3)cc24)cc1 ; c1ccc(-n2c3ccccc3c3cc4c(cc32)oc2ccc(-c3ccc5c(c3)c3cc6c(cc3n5-c3ccccc3)oc3ccccc36)cc24)cc1 ; c1ccc(-n2c3ccccc3c3cc4c(cc32)oc2ccc(-c3ccc5c(c3)c3cc6c(cc3n5-c3ccccc3)sc3ccccc36)cc24)cc1 ; CC1(C)c2ccccc2-c2cc3c4cc(-c5ccc6sc7cc8c(cc7c6c5)c5ccccc5n8-c5ccccc5)ccc4n(-c4ccccc4)c3cc21]

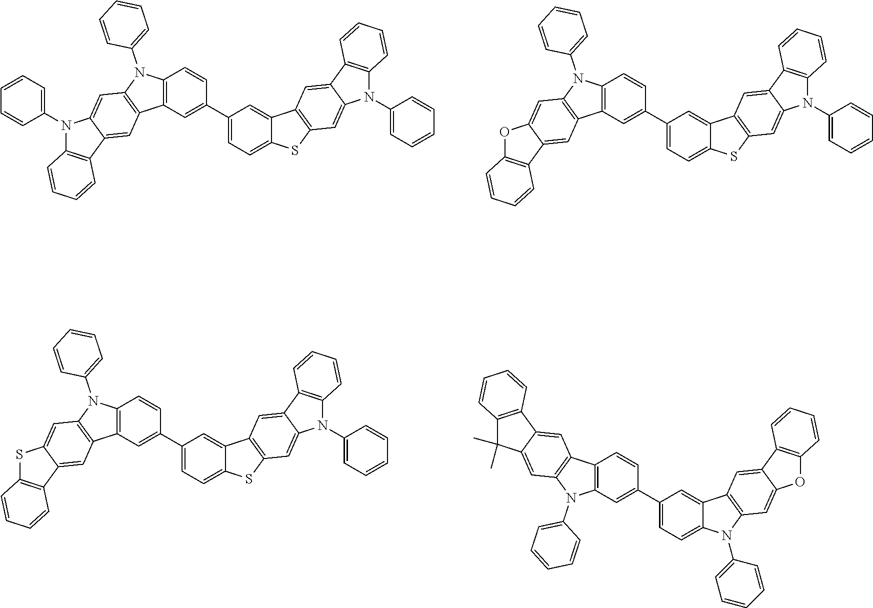 [SMILES: c1ccc(-n2c3ccccc3c3cc4c(cc32)sc2ccc(-c3ccc5c(c3)c3cc6c7ccccc7n(-c7ccccc7)c6cc3n5-c3ccccc3)cc24)cc1 ; c1ccc(-n2c3ccc(-c4ccc5sc6cc7c(cc6c5c4)c4ccccc4n7-c4ccccc4)cc3c3cc4c(cc32)oc2ccccc24)cc1 ; c1ccc(-n2c3ccccc3c3cc4c(cc32)sc2ccc(-c3ccc5c(c3)c3cc6c(cc3n5-c3ccccc3)sc3ccccc36)cc24)cc1 ; CC1(C)c2ccccc2-c2cc3c4ccc(-c5ccc6c(c5)c5cc7c(cc5n6-c5ccccc5)oc5ccccc57)cc4n(-c4ccccc4)c3cc21]

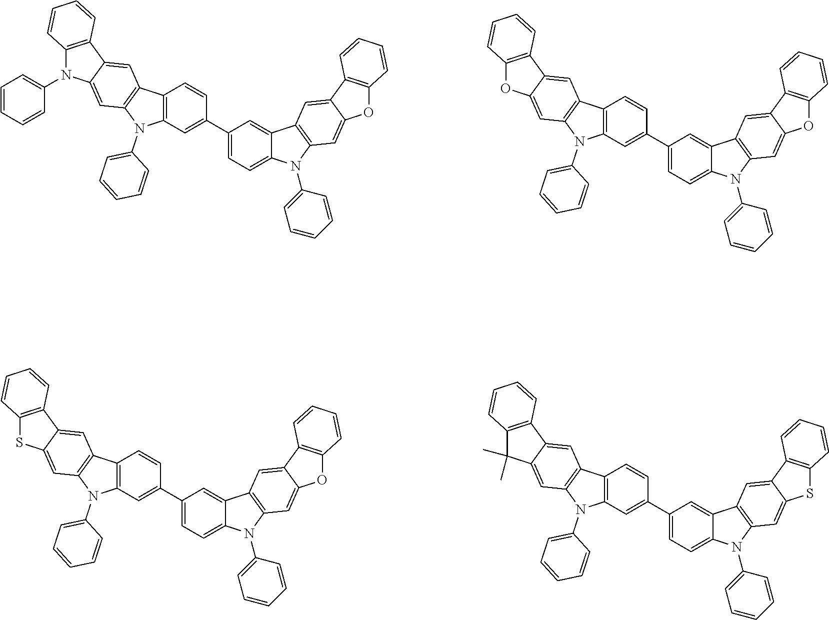 [SMILES: c1ccc(-n2c3ccc(-c4ccc5c6cc7c8ccccc8n(-c8ccccc8)c7cc6n(-c6ccccc6)c5c4)cc3c3cc4c(cc32)oc2ccccc24)cc1 ; c1ccc(-n2c3ccc(-c4ccc5c6cc7c(cc6n(-c6ccccc6)c5c4)oc4ccccc47)cc3c3cc4c(cc32)oc2ccccc24)cc1 ; c1ccc(-n2c3ccc(-c4ccc5c6cc7c(cc6n(-c6ccccc6)c5c4)sc4ccccc47)cc3c3cc4c(cc32)oc2ccccc24)cc1 ; CC1(C)c2ccccc2-c2cc3c4ccc(-c5ccc6c(c5)c5cc7c(cc5n6-c5ccccc5)sc5ccccc57)cc4n(-c4ccccc4)c3cc21]

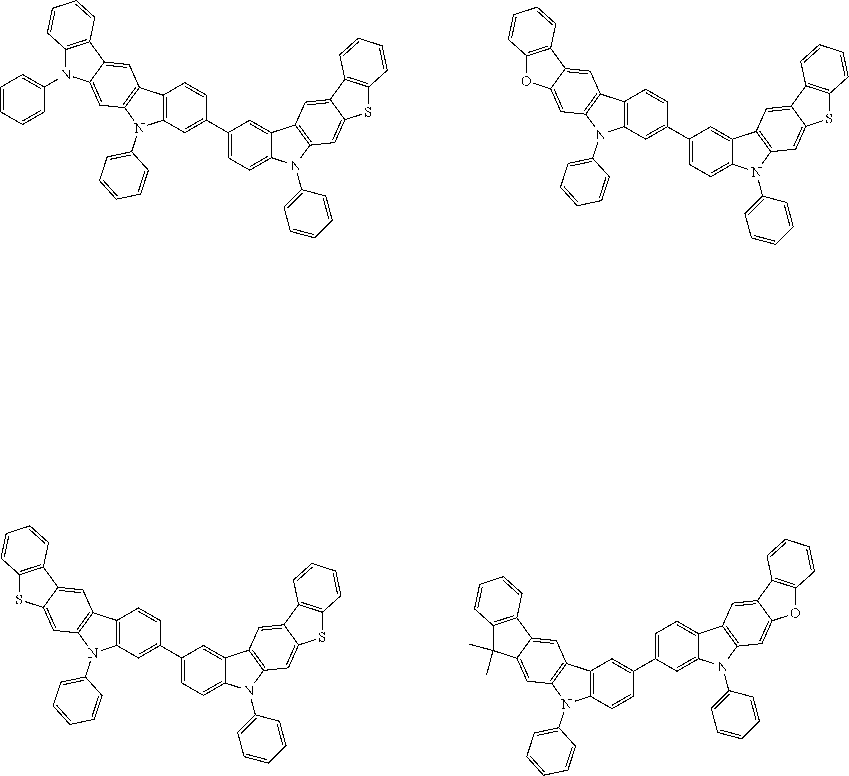 [SMILES: c1ccc(-n2c3ccc(-c4ccc5c6cc7c8ccccc8n(-c8ccccc8)c7cc6n(-c6ccccc6)c5c4)cc3c3cc4c(cc32)sc2ccccc24)cc1 ; c1ccc(-n2c3cc(-c4ccc5c(c4)c4cc6c(cc4n5-c4ccccc4)sc4ccccc46)ccc3c3cc4c(cc32)oc2ccccc24)cc1 ; c1ccc(-n2c3ccc(-c4ccc5c6cc7c(cc6n(-c6ccccc6)c5c4)sc4ccccc47)cc3c3cc4c(cc32)sc2ccccc24)cc1 ; CC1(C)c2ccccc2-c2cc3c4cc(-c5ccc6c7cc8c(cc7n(-c7ccccc7)c6c5)oc5ccccc58)ccc4n(-c4ccccc4)c3cc21]

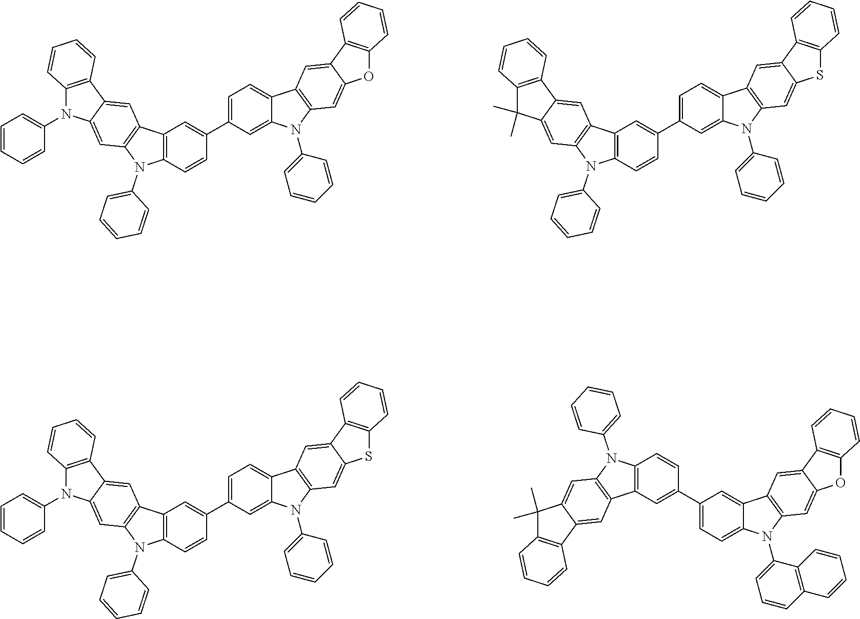 [SMILES: c1ccc(-n2c3cc(-c4ccc5c(c4)c4cc6c7ccccc7n(-c7ccccc7)c6cc4n5-c4ccccc4)ccc3c3cc4c(cc32)oc2ccccc24)cc1 ; CC1(C)c2ccccc2-c2cc3c4cc(-c5ccc6c7cc8c(cc7n(-c7ccccc7)c6c5)sc5ccccc58)ccc4n(-c4ccccc4)c3cc21 ; c1ccc(-n2c3cc(-c4ccc5c(c4)c4cc6c7ccccc7n(-c7ccccc7)c6cc4n5-c4ccccc4)ccc3c3cc4c(cc32)sc2ccccc24)cc1 ; CC1(C)c2ccccc2-c2cc3c4cc(-c5ccc6c(c5)c5cc7c(cc5n6-c5cccc6ccccc56)oc5ccccc57)ccc4n(-c4ccccc4)c3cc21]

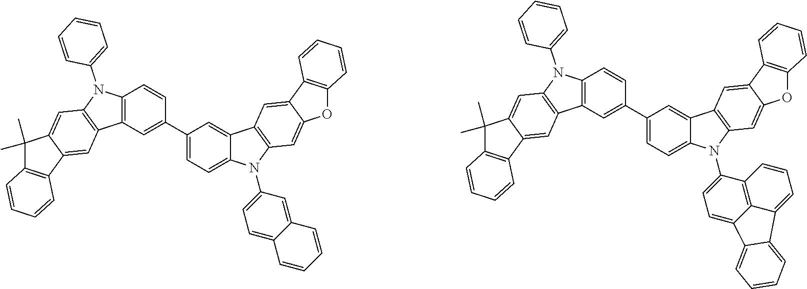 [SMILES: CC1(C)c2ccccc2-c2cc3c4cc(-c5ccc6c(c5)c5cc7c(cc5n6-c5ccc6ccccc6c5)oc5ccccc57)ccc4n(-c4ccccc4)c3cc21 ; CC1(C)c2ccccc2-c2cc3c4cc(-c5ccc6c(c5)c5cc7c(cc5n6-c5ccc6c8c(cccc58)-c5ccccc5-6)oc5ccccc57)ccc4n(-c4ccccc4)c3cc21]

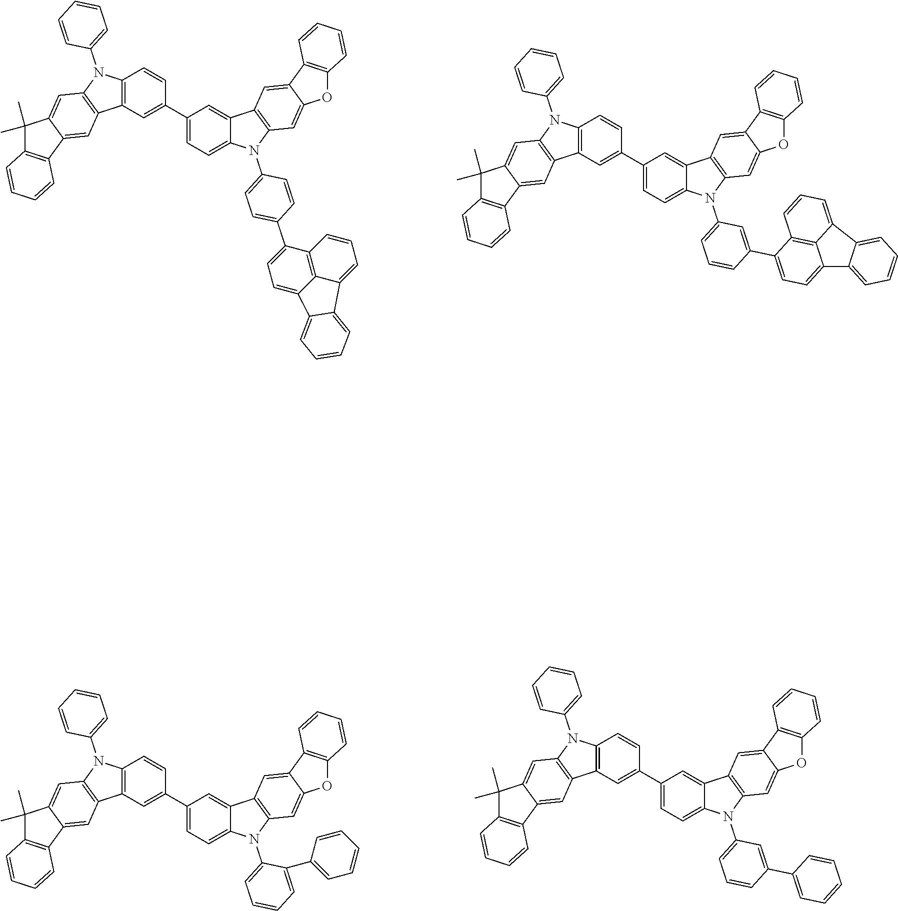 [SMILES: CC1(C)c2ccccc2-c2cc3c4cc(-c5ccc6c(c5)c5cc7c(cc5n6-c5ccc(-c6ccc8c9c(cccc69)-c6ccccc6-8)cc5)oc5ccccc57)ccc4n(-c4ccccc4)c3cc21 ; CC1(C)c2ccccc2-c2cc3c4cc(-c5ccc6c(c5)c5cc7c(cc5n6-c5cccc(-c6ccc8c9c(cccc69)-c6ccccc6-8)c5)oc5ccccc57)ccc4n(-c4ccccc4)c3cc21 ; CC1(C)c2ccccc2-c2cc3c4cc(-c5ccc6c(c5)c5cc7c(cc5n6-c5ccccc5-c5ccccc5)oc5ccccc57)ccc4n(-c4ccccc4)c3cc21 ; CC1(C)c2ccccc2-c2cc3c4cc(-c5ccc6c(c5)c5cc7c(cc5n6-c5cccc(-c6ccccc6)c5)oc5ccccc57)ccc4n(-c4ccccc4)c3cc21]

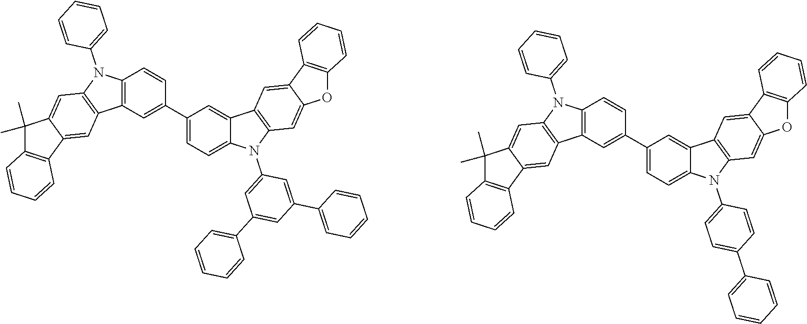 [SMILES: CC1(C)c2ccccc2-c2cc3c4cc(-c5ccc6c(c5)c5cc7c(cc5n6-c5cc(-c6ccccc6)cc(-c6ccccc6)c5)oc5ccccc57)ccc4n(-c4ccccc4)c3cc21 ; CC1(C)c2ccccc2-c2cc3c4cc(-c5ccc6c(c5)c5cc7c(cc5n6-c5ccc(-c6ccccc6)cc5)oc5ccccc57)ccc4n(-c4ccccc4)c3cc21]

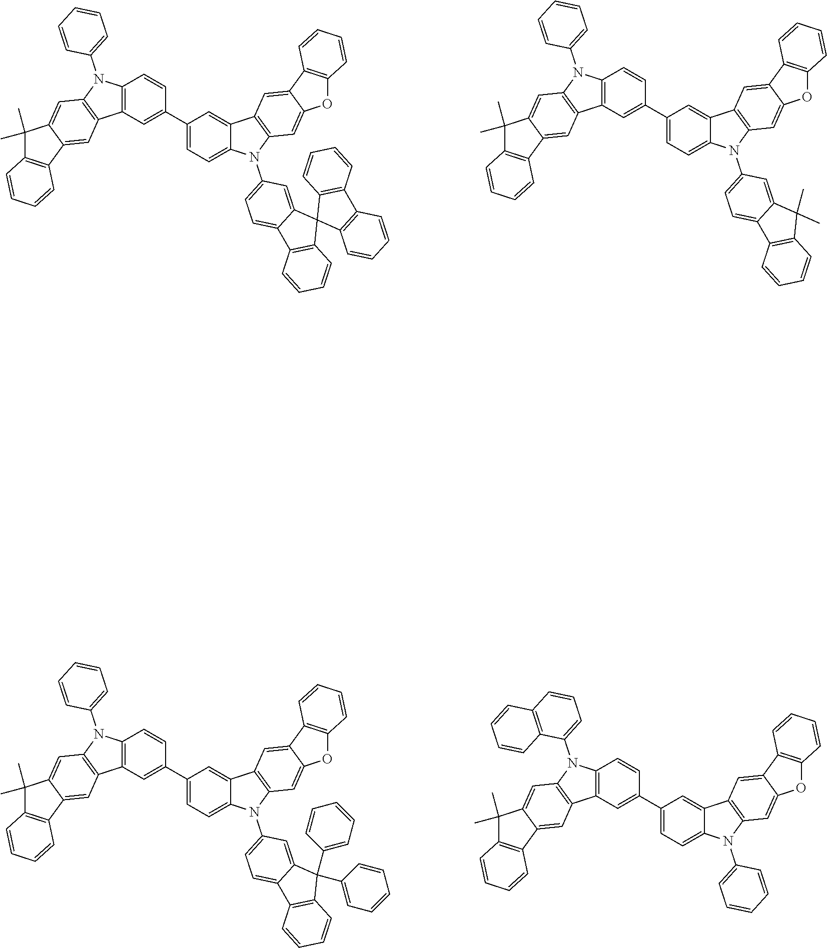 [SMILES: CC1(C)c2ccccc2-c2cc3c4cc(-c5ccc6c(c5)c5cc7c(cc5n6-c5ccc6c(c5)C5(c8ccccc8-c8ccccc85)c5ccccc5-6)oc5ccccc57)ccc4n(-c4ccccc4)c3cc21 ; CC1(C)c2ccccc2-c2ccc(-n3c4ccc(-c5ccc6c(c5)c5cc7c(cc5n6-c5ccccc5)C(C)(C)c5ccccc5-7)cc4c4cc5c(cc43)oc3ccccc35)cc21 ; CC1(C)c2ccccc2-c2cc3c4cc(-c5ccc6c(c5)c5cc7c(cc5n6-c5ccc6c(c5)C(c5ccccc5)(c5ccccc5)c5ccccc5-6)oc5ccccc57)ccc4n(-c4ccccc4)c3cc21 ; CC1(C)c2ccccc2-c2cc3c4cc(-c5ccc6c(c5)c5cc7c(cc5n6-c5ccccc5)oc5ccccc57)ccc4n(-c4cccc5ccccc45)c3cc21]

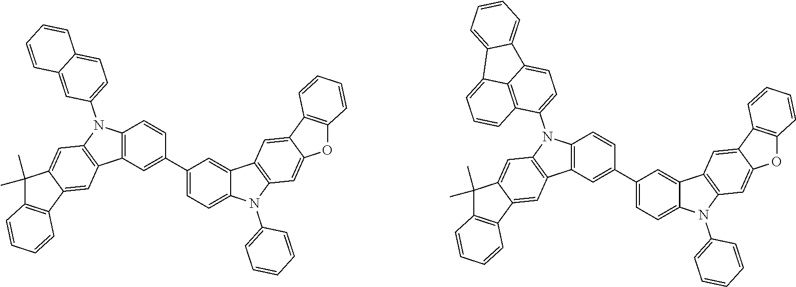 [SMILES: CC1(C)c2ccccc2-c2cc3c4cc(-c5ccc6c(c5)c5cc7c(cc5n6-c5ccccc5)oc5ccccc57)ccc4n(-c4ccc5ccccc5c4)c3cc21 ; CC1(C)c2ccccc2-c2cc3c4cc(-c5ccc6c(c5)c5cc7c(cc5n6-c5ccccc5)oc5ccccc57)ccc4n(-c4ccc5c6c(cccc46)-c4ccccc4-5)c3cc21]

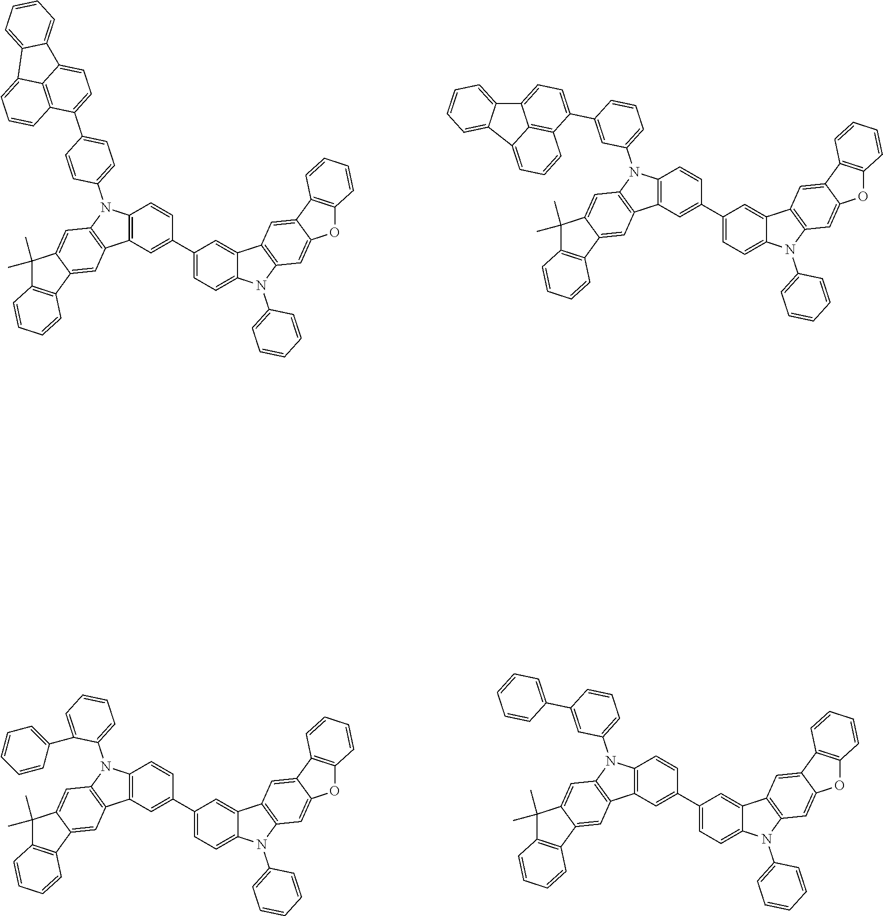 [SMILES: CC1(C)c2ccccc2-c2cc3c4cc(-c5ccc6c(c5)c5cc7c(cc5n6-c5ccccc5)oc5ccccc57)ccc4n(-c4ccc(-c5ccc6c7c(cccc57)-c5ccccc5-6)cc4)c3cc21 ; CC1(C)c2ccccc2-c2cc3c4cc(-c5ccc6c(c5)c5cc7c(cc5n6-c5ccccc5)oc5ccccc57)ccc4n(-c4cccc(-c5ccc6c7c(cccc57)-c5ccccc5-6)c4)c3cc21 ; CC1(C)c2ccccc2-c2cc3c4cc(-c5ccc6c(c5)c5cc7c(cc5n6-c5ccccc5)oc5ccccc57)ccc4n(-c4ccccc4-c4ccccc4)c3cc21 ; CC1(C)c2ccccc2-c2cc3c4cc(-c5ccc6c(c5)c5cc7c(cc5n6-c5ccccc5)oc5ccccc57)ccc4n(-c4cccc(-c5ccccc5)c4)c3cc21]

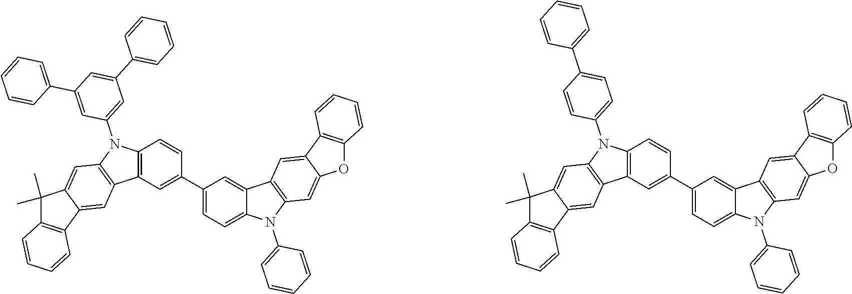 [SMILES: CC1(C)c2ccccc2-c2cc3c4cc(-c5ccc6c(c5)c5cc7c(cc5n6-c5ccccc5)oc5ccccc57)ccc4n(-c4cc(-c5ccccc5)cc(-c5ccccc5)c4)c3cc21 ; CC1(C)c2ccccc2-c2cc3c4cc(-c5ccc6c(c5)c5cc7c(cc5n6-c5ccccc5)oc5ccccc57)ccc4n(-c4ccc(-c5ccccc5)cc4)c3cc21]

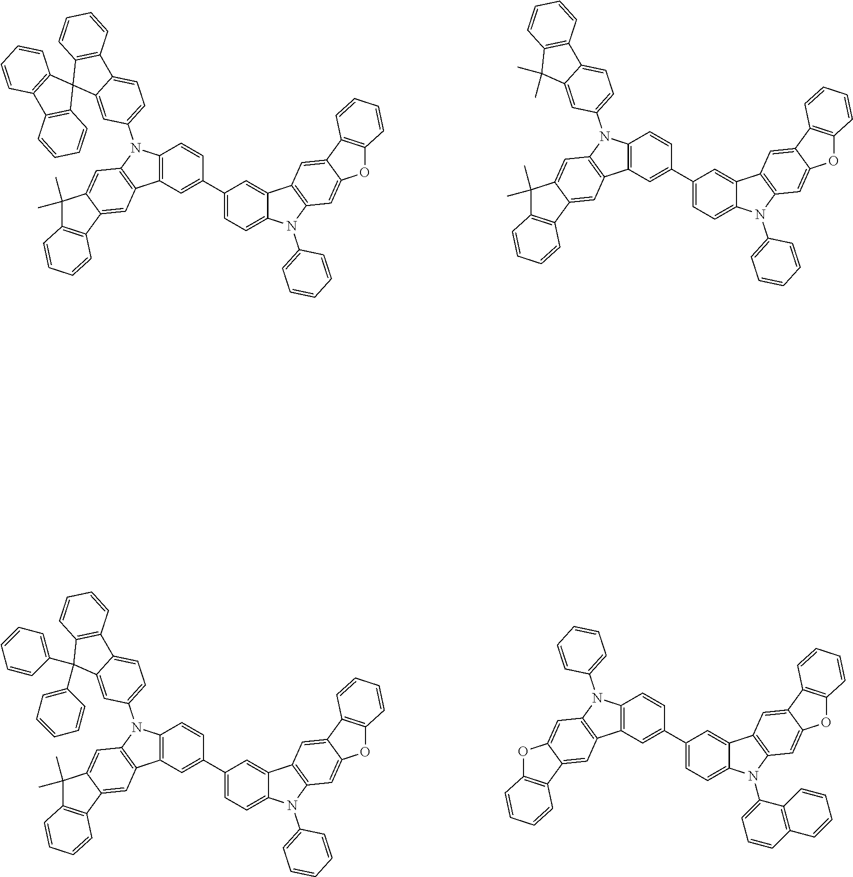 [SMILES: CC1(C)c2ccccc2-c2cc3c4cc(-c5ccc6c(c5)c5cc7c(cc5n6-c5ccccc5)oc5ccccc57)ccc4n(-c4ccc5c(c4)C4(c6ccccc6-c6ccccc64)c4ccccc4-5)c3cc21 ; CC1(C)c2ccccc2-c2ccc(-n3c4ccc(-c5ccc6c(c5)c5cc7c(cc5n6-c5ccccc5)oc5ccccc57)cc4c4cc5c(cc43)C(C)(C)c3ccccc3-5)cc21 ; CC1(C)c2ccccc2-c2cc3c4cc(-c5ccc6c(c5)c5cc7c(cc5n6-c5ccccc5)oc5ccccc57)ccc4n(-c4ccc5c(c4)C(c4ccccc4)(c4ccccc4)c4ccccc4-5)c3cc21 ; c1ccc(-n2c3ccc(-c4ccc5c(c4)c4cc6c(cc4n5-c4cccc5ccccc45)oc4ccccc46)cc3c3cc4c(cc32)oc2ccccc24)cc1]

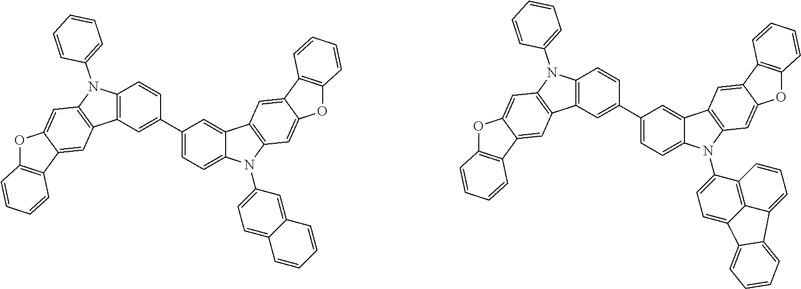 [SMILES: c1ccc(-n2c3ccc(-c4ccc5c(c4)c4cc6c(cc4n5-c4ccc5ccccc5c4)oc4ccccc46)cc3c3cc4c(cc32)oc2ccccc24)cc1 ; c1ccc(-n2c3ccc(-c4ccc5c(c4)c4cc6c(cc4n5-c4ccc5c7c(cccc47)-c4ccccc4-5)oc4ccccc46)cc3c3cc4c(cc32)oc2ccccc24)cc1]

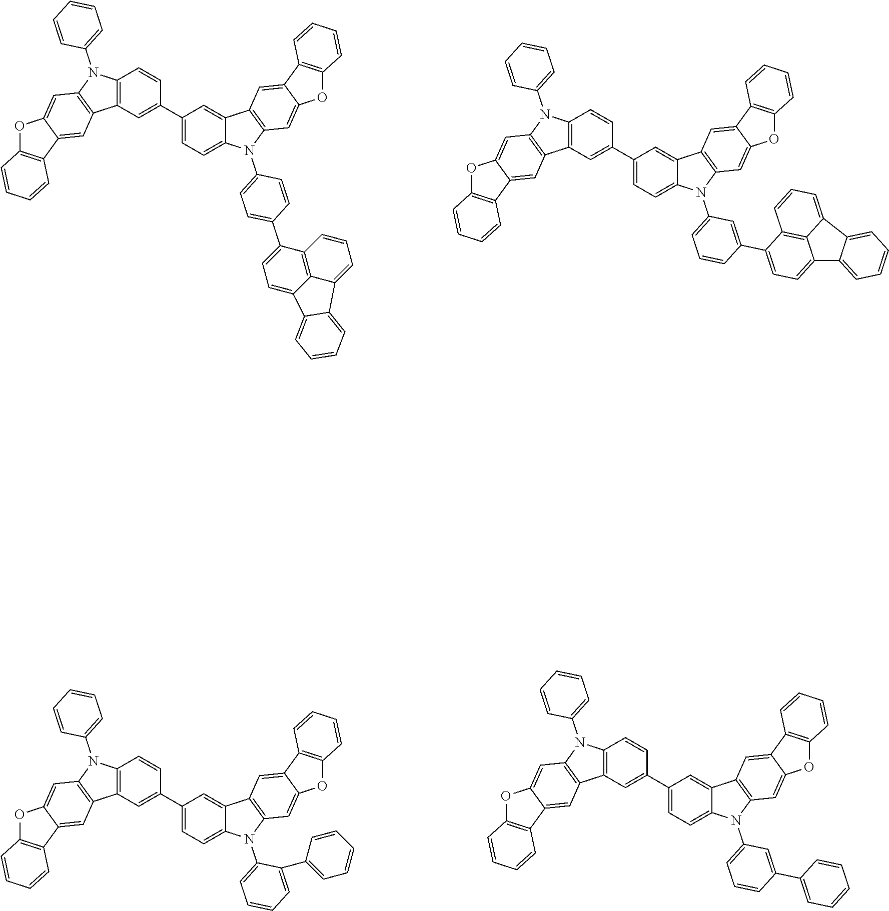 [SMILES: c1ccc(-n2c3ccc(-c4ccc5c(c4)c4cc6c(cc4n5-c4ccc(-c5ccc7c8c(cccc58)-c5ccccc5-7)cc4)oc4ccccc46)cc3c3cc4c(cc32)oc2ccccc24)cc1 ; c1ccc(-n2c3ccc(-c4ccc5c(c4)c4cc6c(cc4n5-c4cccc(-c5ccc7c8c(cccc58)-c5ccccc5-7)c4)oc4ccccc46)cc3c3cc4c(cc32)oc2ccccc24)cc1 ; c1ccc(-c2ccccc2-n2c3ccc(-c4ccc5c(c4)c4cc6c(cc4n5-c4ccccc4)oc4ccccc46)cc3c3cc4c(cc32)oc2ccccc24)cc1 ; c1ccc(-c2cccc(-n3c4ccc(-c5ccc6c(c5)c5cc7c(cc5n6-c5ccccc5)oc5ccccc57)cc4c4cc5c(cc43)oc3ccccc35)c2)cc1]

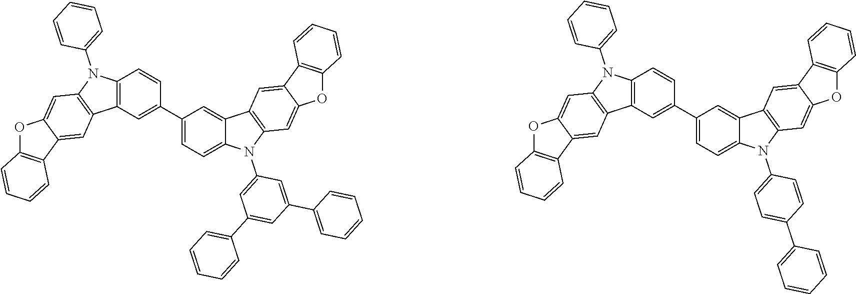 [SMILES: c1ccc(-c2cc(-c3ccccc3)cc(-n3c4ccc(-c5ccc6c(c5)c5cc7c(cc5n6-c5ccccc5)oc5ccccc57)cc4c4cc5c(cc43)oc3ccccc35)c2)cc1 ; c1ccc(-c2ccc(-n3c4ccc(-c5ccc6c(c5)c5cc7c(cc5n6-c5ccccc5)oc5ccccc57)cc4c4cc5c(cc43)oc3ccccc35)cc2)cc1]

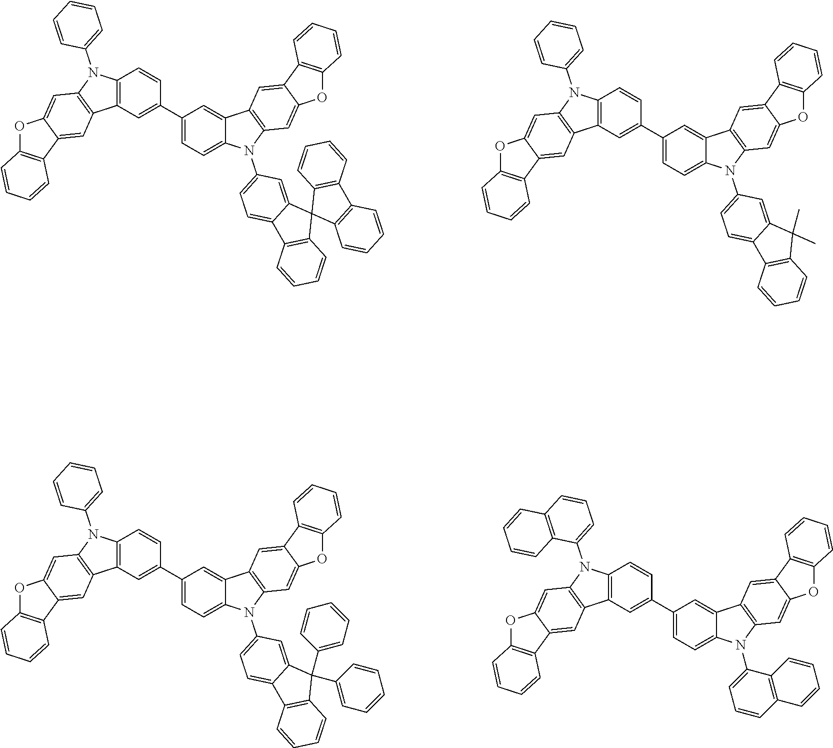 [SMILES: c1ccc(-n2c3ccc(-c4ccc5c(c4)c4cc6c(cc4n5-c4ccc5c(c4)C4(c7ccccc7-c7ccccc74)c4ccccc4-5)oc4ccccc46)cc3c3cc4c(cc32)oc2ccccc24)cc1 ; CC1(C)c2ccccc2-c2ccc(-n3c4ccc(-c5ccc6c(c5)c5cc7c(cc5n6-c5ccccc5)oc5ccccc57)cc4c4cc5c(cc43)oc3ccccc35)cc21 ; c1ccc(-n2c3ccc(-c4ccc5c(c4)c4cc6c(cc4n5-c4ccc5c(c4)C(c4ccccc4)(c4ccccc4)c4ccccc4-5)oc4ccccc46)cc3c3cc4c(cc32)oc2ccccc24)cc1 ; c1ccc2c(-n3c4ccc(-c5ccc6c(c5)c5cc7c(cc5n6-c5cccc6ccccc56)oc5ccccc57)cc4c4cc5c(cc43)oc3ccccc35)cccc2c1]

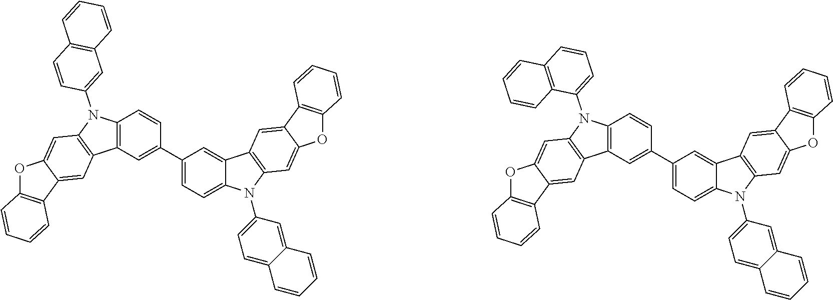 [SMILES: c1ccc2cc(-n3c4ccc(-c5ccc6c(c5)c5cc7c(cc5n6-c5ccc6ccccc6c5)oc5ccccc57)cc4c4cc5c(cc43)oc3ccccc35)ccc2c1 ; c1ccc2cc(-n3c4ccc(-c5ccc6c(c5)c5cc7c(cc5n6-c5cccc6ccccc56)oc5ccccc57)cc4c4cc5c(cc43)oc3ccccc35)ccc2c1]

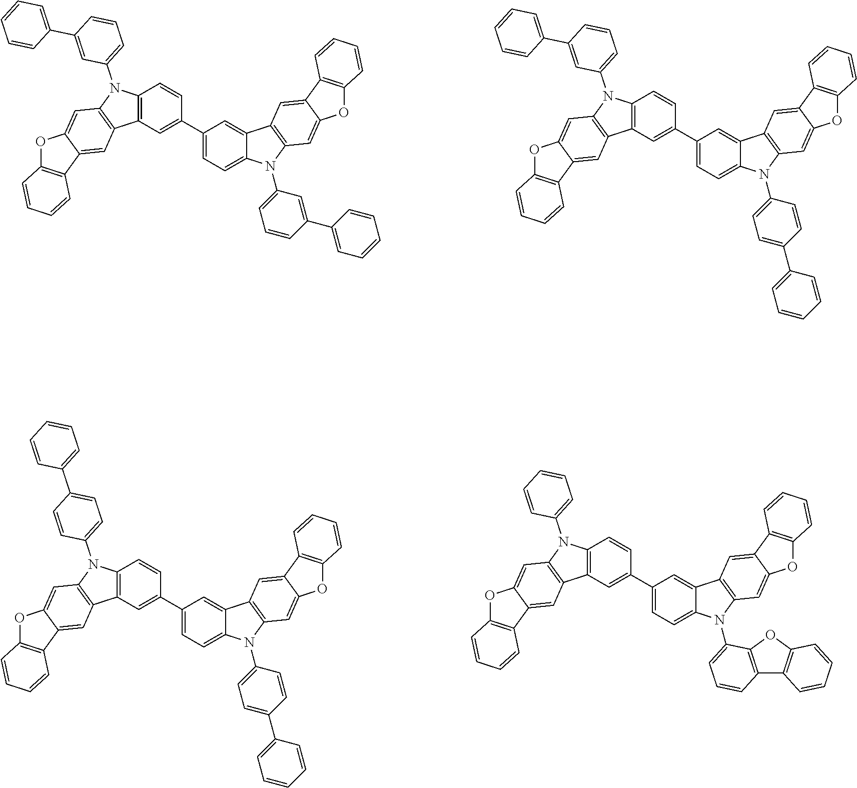 [SMILES: c1ccc(-c2cccc(-n3c4ccc(-c5ccc6c(c5)c5cc7c(cc5n6-c5cccc(-c6ccccc6)c5)oc5ccccc57)cc4c4cc5c(cc43)oc3ccccc35)c2)cc1 ; c1ccc(-c2ccc(-n3c4ccc(-c5ccc6c(c5)c5cc7c(cc5n6-c5cccc(-c6ccccc6)c5)oc5ccccc57)cc4c4cc5c(cc43)oc3ccccc35)cc2)cc1 ; c1ccc(-c2ccc(-n3c4ccc(-c5ccc6c(c5)c5cc7c(cc5n6-c5ccc(-c6ccccc6)cc5)oc5ccccc57)cc4c4cc5c(cc43)oc3ccccc35)cc2)cc1 ; c1ccc(-n2c3ccc(-c4ccc5c(c4)c4cc6c(cc4n5-c4cccc5c4oc4ccccc45)oc4ccccc46)cc3c3cc4c(cc32)oc2ccccc24)cc1]

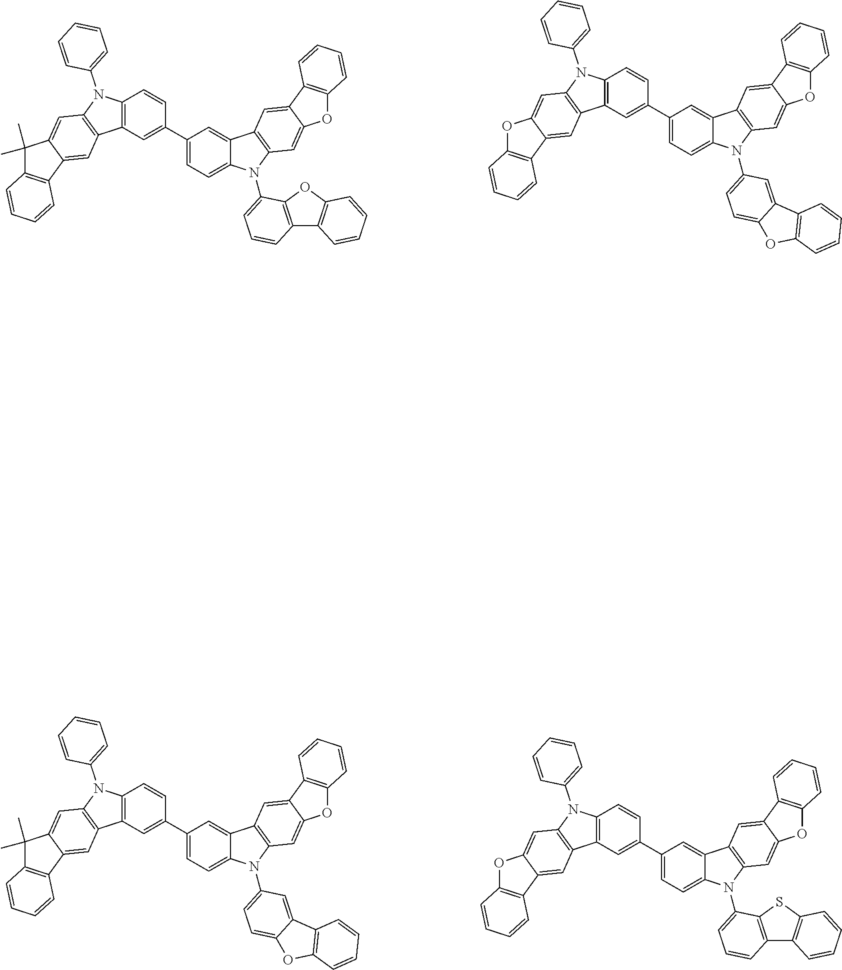 [SMILES: CC1(C)c2ccccc2-c2cc3c4cc(-c5ccc6c(c5)c5cc7c(cc5n6-c5cccc6c5oc5ccccc56)oc5ccccc57)ccc4n(-c4ccccc4)c3cc21 ; c1ccc(-n2c3ccc(-c4ccc5c(c4)c4cc6c(cc4n5-c4ccc5oc7ccccc7c5c4)oc4ccccc46)cc3c3cc4c(cc32)oc2ccccc24)cc1 ; CC1(C)c2ccccc2-c2cc3c4cc(-c5ccc6c(c5)c5cc7c(cc5n6-c5ccc6oc8ccccc8c6c5)oc5ccccc57)ccc4n(-c4ccccc4)c3cc21 ; c1ccc(-n2c3ccc(-c4ccc5c(c4)c4cc6c(cc4n5-c4cccc5c4sc4ccccc45)oc4ccccc46)cc3c3cc4c(cc32)oc2ccccc24)cc1]

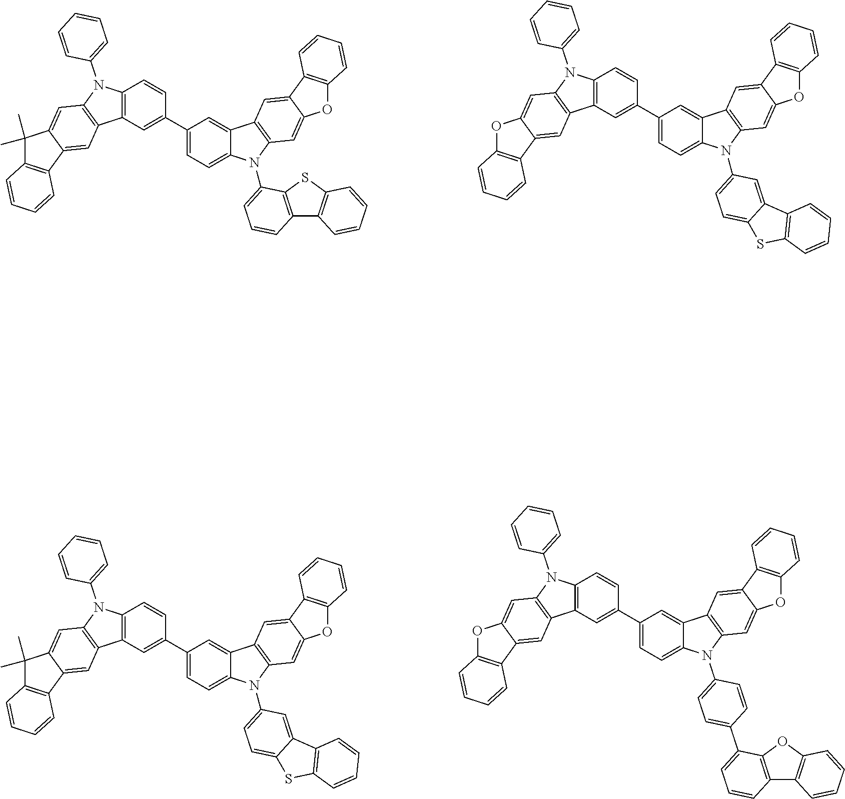 [SMILES: CC1(C)c2ccccc2-c2cc3c4cc(-c5ccc6c(c5)c5cc7c(cc5n6-c5cccc6c5sc5ccccc56)oc5ccccc57)ccc4n(-c4ccccc4)c3cc21 ; c1ccc(-n2c3ccc(-c4ccc5c(c4)c4cc6c(cc4n5-c4ccc5sc7ccccc7c5c4)oc4ccccc46)cc3c3cc4c(cc32)oc2ccccc24)cc1 ; CC1(C)c2ccccc2-c2cc3c4cc(-c5ccc6c(c5)c5cc7c(cc5n6-c5ccc6sc8ccccc8c6c5)oc5ccccc57)ccc4n(-c4ccccc4)c3cc21 ; c1ccc(-n2c3ccc(-c4ccc5c(c4)c4cc6c(cc4n5-c4ccc(-c5cccc7c5oc5ccccc57)cc4)oc4ccccc46)cc3c3cc4c(cc32)oc2ccccc24)cc1]

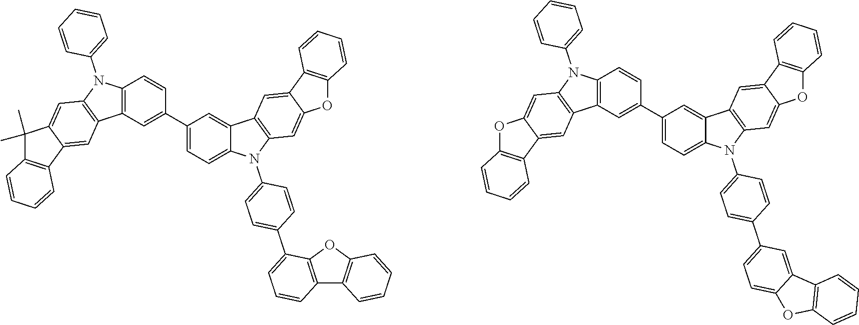 [SMILES: CC1(C)c2ccccc2-c2cc3c4cc(-c5ccc6c(c5)c5cc7c(cc5n6-c5ccc(-c6cccc8c6oc6ccccc68)cc5)oc5ccccc57)ccc4n(-c4ccccc4)c3cc21 ; c1ccc(-n2c3ccc(-c4ccc5c(c4)c4cc6c(cc4n5-c4ccc(-c5ccc7oc8ccccc8c7c5)cc4)oc4ccccc46)cc3c3cc4c(cc32)oc2ccccc24)cc1]

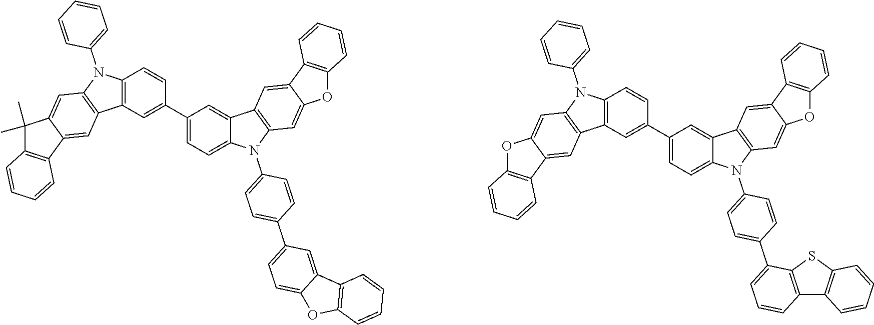 [SMILES: CC1(C)c2ccccc2-c2cc3c4cc(-c5ccc6c(c5)c5cc7c(cc5n6-c5ccc(-c6ccc8oc9ccccc9c8c6)cc5)oc5ccccc57)ccc4n(-c4ccccc4)c3cc21 ; c1ccc(-n2c3ccc(-c4ccc5c(c4)c4cc6c(cc4n5-c4ccc(-c5cccc7c5sc5ccccc57)cc4)oc4ccccc46)cc3c3cc4c(cc32)oc2ccccc24)cc1]

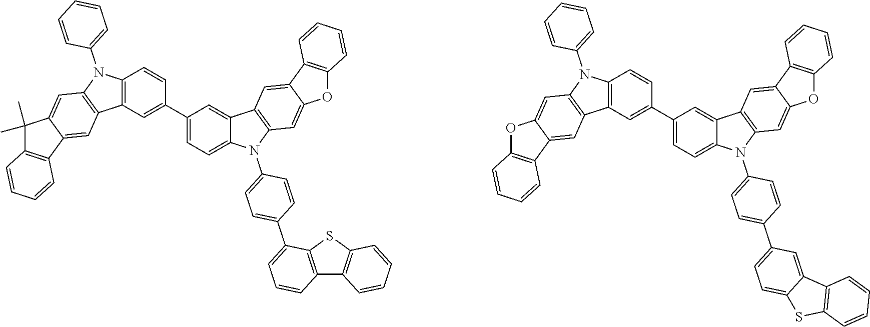 [SMILES: CC1(C)c2ccccc2-c2cc3c4cc(-c5ccc6c(c5)c5cc7c(cc5n6-c5ccc(-c6cccc8c6sc6ccccc68)cc5)oc5ccccc57)ccc4n(-c4ccccc4)c3cc21 ; c1ccc(-n2c3ccc(-c4ccc5c(c4)c4cc6c(cc4n5-c4ccc(-c5ccc7sc8ccccc8c7c5)cc4)oc4ccccc46)cc3c3cc4c(cc32)oc2ccccc24)cc1]

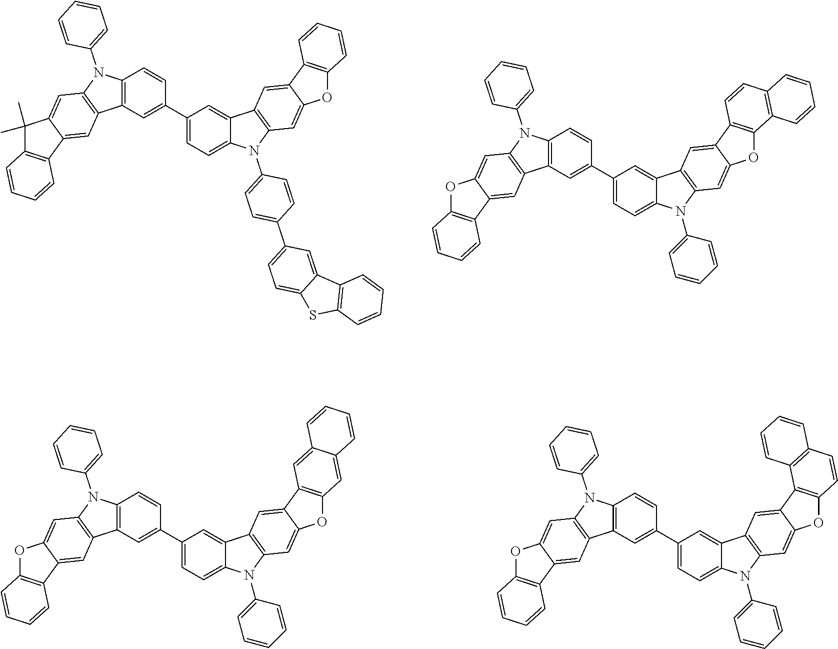 [SMILES: CC1(C)c2ccccc2-c2cc3c4cc(-c5ccc6c(c5)c5cc7c(cc5n6-c5ccc(-c6ccc8sc9ccccc9c8c6)cc5)oc5ccccc57)ccc4n(-c4ccccc4)c3cc21 ; c1ccc(-n2c3ccc(-c4ccc5c(c4)c4cc6c(cc4n5-c4ccccc4)oc4c5ccccc5ccc64)cc3c3cc4c(cc32)oc2ccccc24)cc1 ; c1ccc(-n2c3ccc(-c4ccc5c(c4)c4cc6c(cc4n5-c4ccccc4)oc4cc5ccccc5cc46)cc3c3cc4c(cc32)oc2ccccc24)cc1 ; c1ccc(-n2c3ccc(-c4ccc5c(c4)c4cc6c(cc4n5-c4ccccc4)oc4ccc5ccccc5c46)cc3c3cc4c(cc32)oc2ccccc24)cc1]

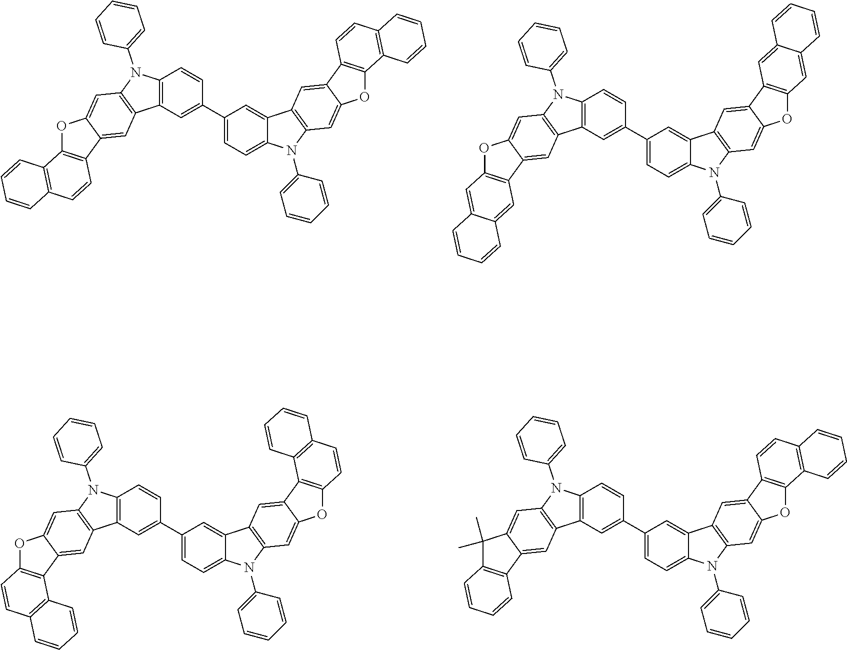 [SMILES: c1ccc(-n2c3ccc(-c4ccc5c(c4)c4cc6c(cc4n5-c4ccccc4)oc4c5ccccc5ccc64)cc3c3cc4c(cc32)oc2c3ccccc3ccc42)cc1 ; c1ccc(-n2c3ccc(-c4ccc5c(c4)c4cc6c(cc4n5-c4ccccc4)oc4cc5ccccc5cc46)cc3c3cc4c(cc32)oc2cc3ccccc3cc24)cc1 ; c1ccc(-n2c3ccc(-c4ccc5c(c4)c4cc6c(cc4n5-c4ccccc4)oc4ccc5ccccc5c46)cc3c3cc4c(cc32)oc2ccc3ccccc3c24)cc1 ; CC1(C)c2ccccc2-c2cc3c4cc(-c5ccc6c(c5)c5cc7c(cc5n6-c5ccccc5)oc5c6ccccc6ccc75)ccc4n(-c4ccccc4)c3cc21]

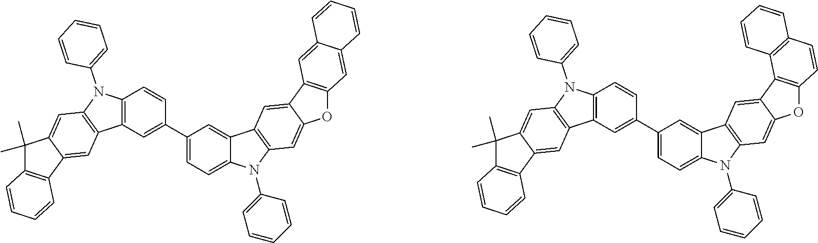 [SMILES: CC1(C)c2ccccc2-c2cc3c4cc(-c5ccc6c(c5)c5cc7c(cc5n6-c5ccccc5)oc5cc6ccccc6cc57)ccc4n(-c4ccccc4)c3cc21 ; CC1(C)c2ccccc2-c2cc3c4cc(-c5ccc6c(c5)c5cc7c(cc5n6-c5ccccc5)oc5ccc6ccccc6c57)ccc4n(-c4ccccc4)c3cc21]

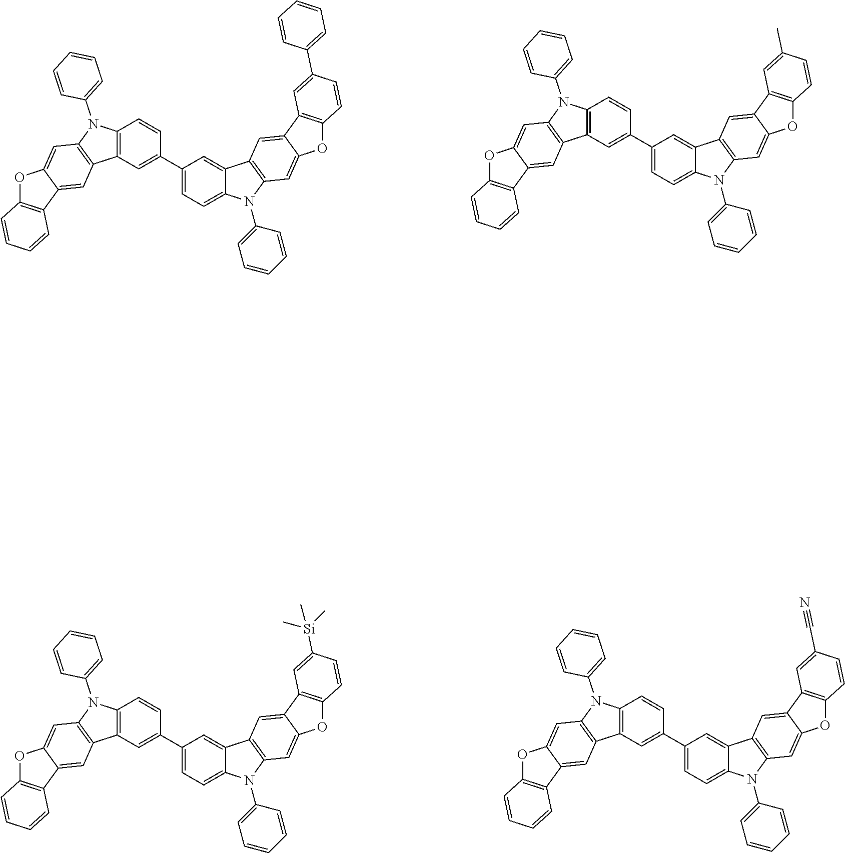 [SMILES: c1ccc(-c2ccc3oc4cc5c(cc4c3c2)c2cc(-c3ccc4c(c3)c3cc6c(cc3n4-c3ccccc3)oc3ccccc36)ccc2n5-c2ccccc2)cc1 ; Cc1ccc2oc3cc4c(cc3c2c1)c1cc(-c2ccc3c(c2)c2cc5c(cc2n3-c2ccccc2)oc2ccccc25)ccc1n4-c1ccccc1 ; C[Si](C)(C)c1ccc2oc3cc4c(cc3c2c1)c1cc(-c2ccc3c(c2)c2cc5c(cc2n3-c2ccccc2)oc2ccccc25)ccc1n4-c1ccccc1 ; N#Cc1ccc2oc3cc4c(cc3c2c1)c1cc(-c2ccc3c(c2)c2cc5c(cc2n3-c2ccccc2)oc2ccccc25)ccc1n4-c1ccccc1]

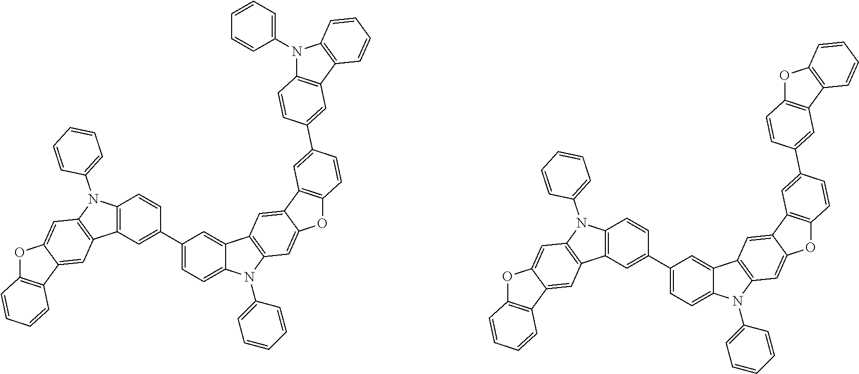 [SMILES: c1ccc(-n2c3ccccc3c3cc(-c4ccc5oc6cc7c(cc6c5c4)c4cc(-c5ccc6c(c5)c5cc8c(cc5n6-c5ccccc5)oc5ccccc58)ccc4n7-c4ccccc4)ccc32)cc1 ; c1ccc(-n2c3ccc(-c4ccc5c(c4)c4cc6c(cc4n5-c4ccccc4)oc4ccc(-c5ccc7oc8ccccc8c7c5)cc46)cc3c3cc4c(cc32)oc2ccccc24)cc1]

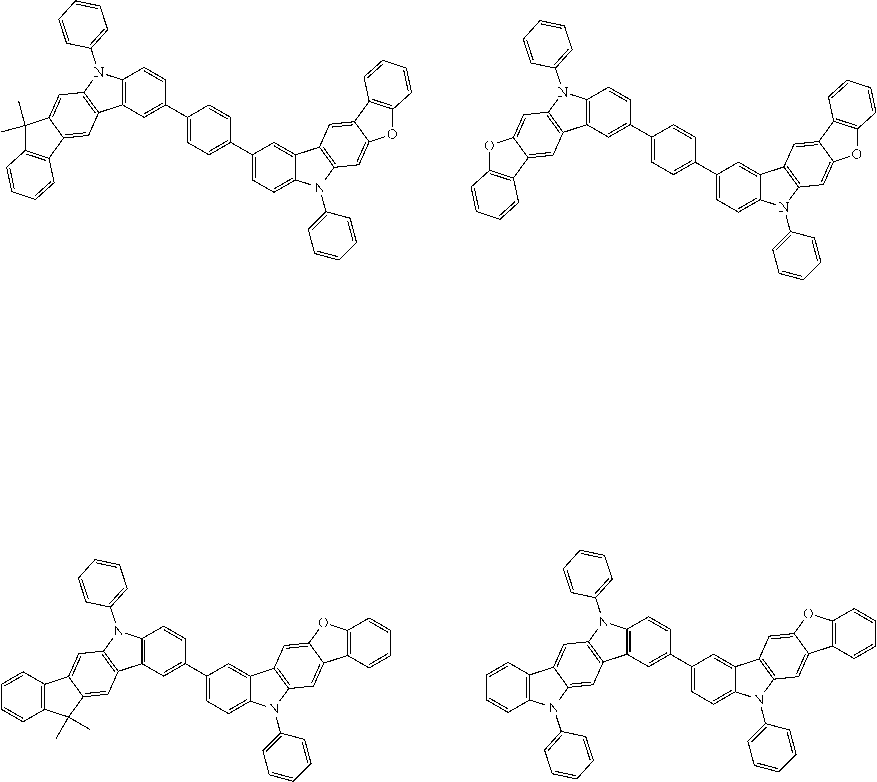 [SMILES: CC1(C)c2ccccc2-c2cc3c4cc(-c5ccc(-c6ccc7c(c6)c6cc8c(cc6n7-c6ccccc6)oc6ccccc68)cc5)ccc4n(-c4ccccc4)c3cc21 ; c1ccc(-n2c3ccc(-c4ccc(-c5ccc6c(c5)c5cc7c(cc5n6-c5ccccc5)oc5ccccc57)cc4)cc3c3cc4c(cc32)oc2ccccc24)cc1 ; CC1(C)c2ccccc2-c2cc3c(cc21)c1cc(-c2ccc4c(c2)c2cc5oc6ccccc6c5cc2n4-c2ccccc2)ccc1n3-c1ccccc1 ; c1ccc(-n2c3ccc(-c4ccc5c(c4)c4cc6c(cc4n5-c4ccccc4)c4ccccc4n6-c4ccccc4)cc3c3cc4oc5ccccc5c4cc32)cc1]

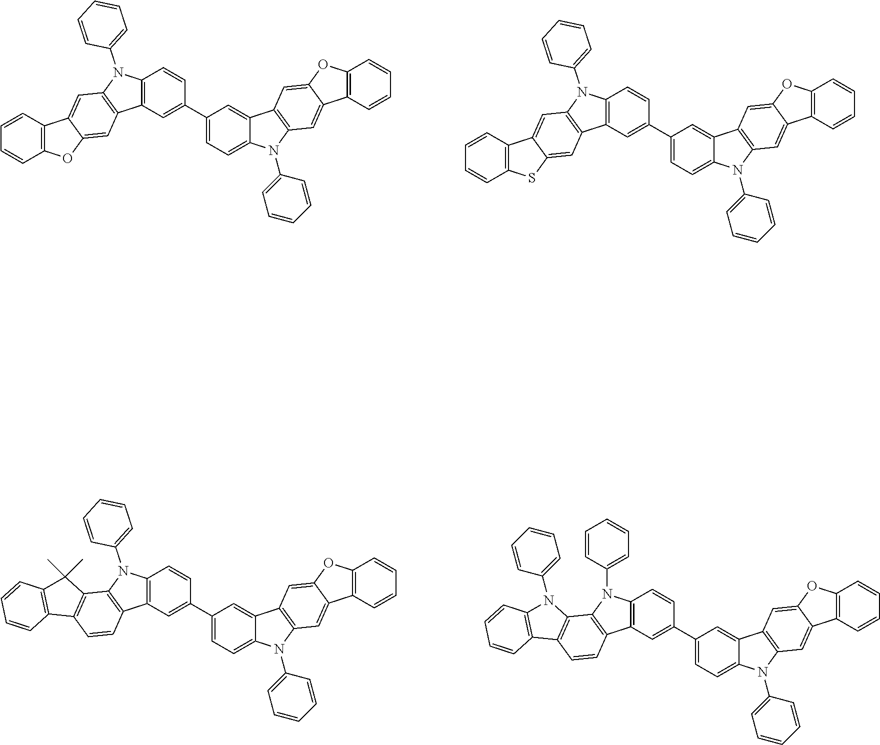 [SMILES: c1ccc(-n2c3ccc(-c4ccc5c(c4)c4cc6oc7ccccc7c6cc4n5-c4ccccc4)cc3c3cc4oc5ccccc5c4cc32)cc1 ; c1ccc(-n2c3ccc(-c4ccc5c(c4)c4cc6sc7ccccc7c6cc4n5-c4ccccc4)cc3c3cc4oc5ccccc5c4cc32)cc1 ; CC1(C)c2ccccc2-c2ccc3c4cc(-c5ccc6c(c5)c5cc7oc8ccccc8c7cc5n6-c5ccccc5)ccc4n(-c4ccccc4)c3c21 ; c1ccc(-n2c3ccc(-c4ccc5c(c4)c4ccc6c7ccccc7n(-c7ccccc7)c6c4n5-c4ccccc4)cc3c3cc4oc5ccccc5c4cc32)cc1]

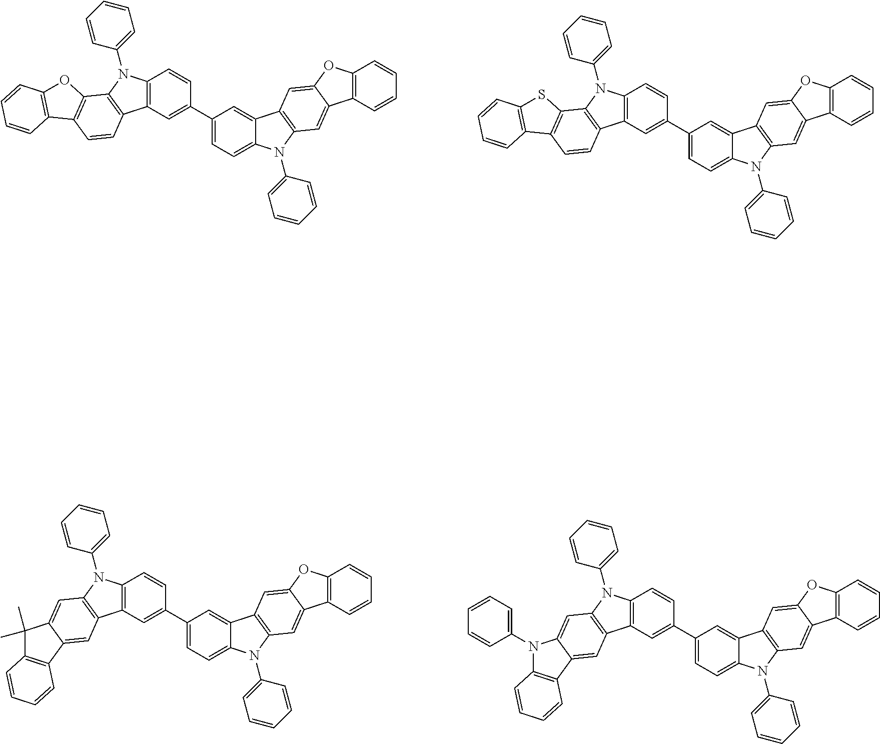 [SMILES: c1ccc(-n2c3ccc(-c4ccc5c(c4)c4ccc6c7ccccc7oc6c4n5-c4ccccc4)cc3c3cc4oc5ccccc5c4cc32)cc1 ; c1ccc(-n2c3ccc(-c4ccc5c(c4)c4ccc6c7ccccc7sc6c4n5-c4ccccc4)cc3c3cc4oc5ccccc5c4cc32)cc1 ; CC1(C)c2ccccc2-c2cc3c4cc(-c5ccc6c(c5)c5cc7oc8ccccc8c7cc5n6-c5ccccc5)ccc4n(-c4ccccc4)c3cc21 ; c1ccc(-n2c3ccc(-c4ccc5c(c4)c4cc6c7ccccc7n(-c7ccccc7)c6cc4n5-c4ccccc4)cc3c3cc4oc5ccccc5c4cc32)cc1]

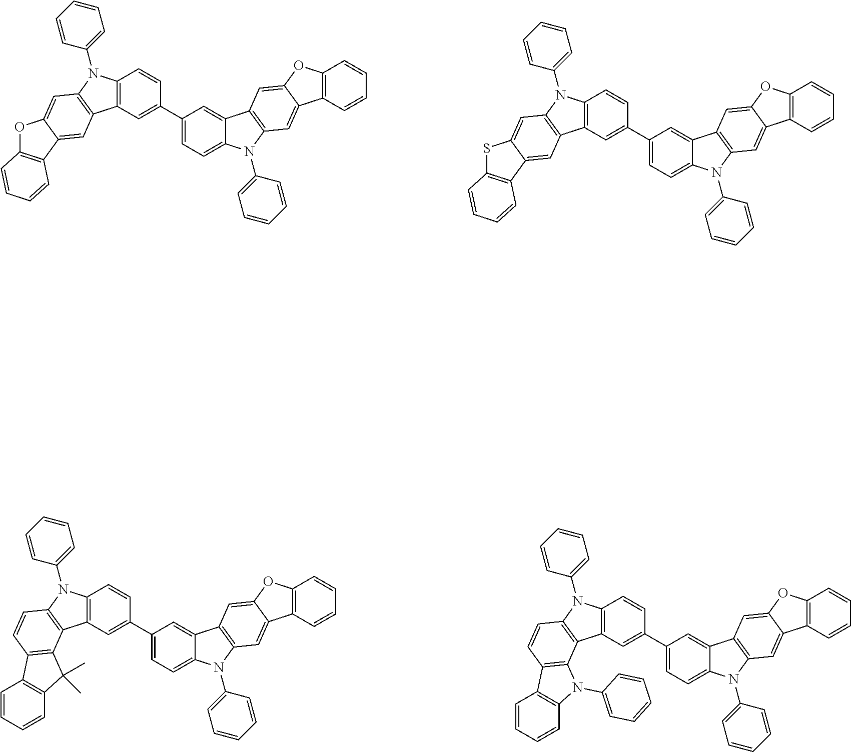 [SMILES: c1ccc(-n2c3ccc(-c4ccc5c(c4)c4cc6oc7ccccc7c6cc4n5-c4ccccc4)cc3c3cc4c(cc32)oc2ccccc24)cc1 ; c1ccc(-n2c3ccc(-c4ccc5c(c4)c4cc6oc7ccccc7c6cc4n5-c4ccccc4)cc3c3cc4c(cc32)sc2ccccc24)cc1 ; CC1(C)c2ccccc2-c2ccc3c(c21)c1cc(-c2ccc4c(c2)c2cc5oc6ccccc6c5cc2n4-c2ccccc2)ccc1n3-c1ccccc1 ; c1ccc(-n2c3ccc(-c4ccc5c(c4)c4c(ccc6c7ccccc7n(-c7ccccc7)c64)n5-c4ccccc4)cc3c3cc4oc5ccccc5c4cc32)cc1]

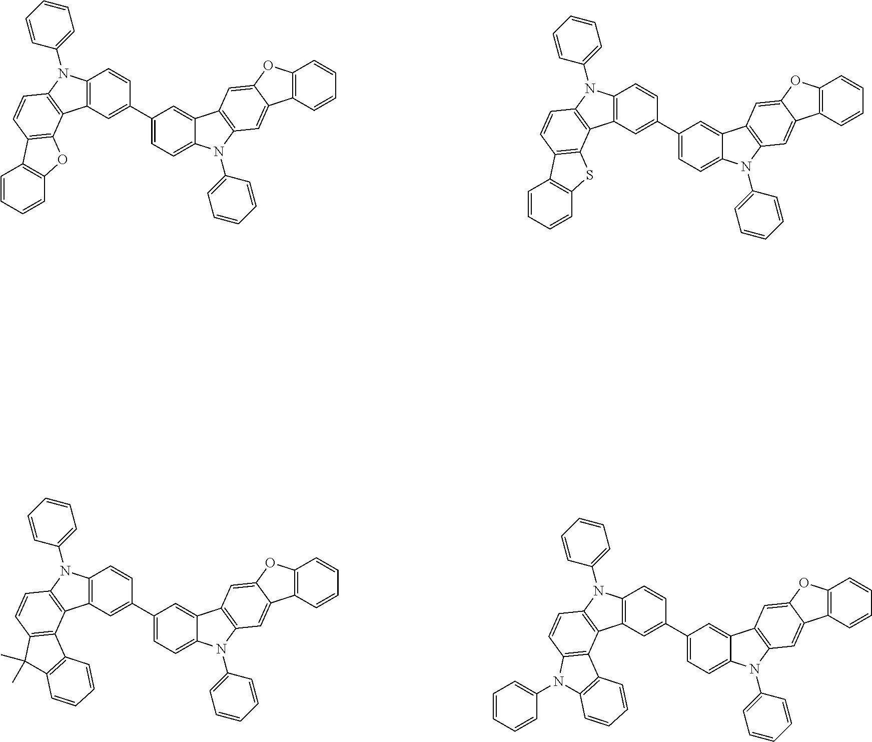 [SMILES: c1ccc(-n2c3ccc(-c4ccc5c(c4)c4c6oc7ccccc7c6ccc4n5-c4ccccc4)cc3c3cc4oc5ccccc5c4cc32)cc1 ; c1ccc(-n2c3ccc(-c4ccc5c(c4)c4c6sc7ccccc7c6ccc4n5-c4ccccc4)cc3c3cc4oc5ccccc5c4cc32)cc1 ; CC1(C)c2ccccc2-c2c1ccc1c2c2cc(-c3ccc4c(c3)c3cc5oc6ccccc6c5cc3n4-c3ccccc3)ccc2n1-c1ccccc1 ; c1ccc(-n2c3ccc(-c4ccc5c(c4)c4c6c7ccccc7n(-c7ccccc7)c6ccc4n5-c4ccccc4)cc3c3cc4oc5ccccc5c4cc32)cc1]

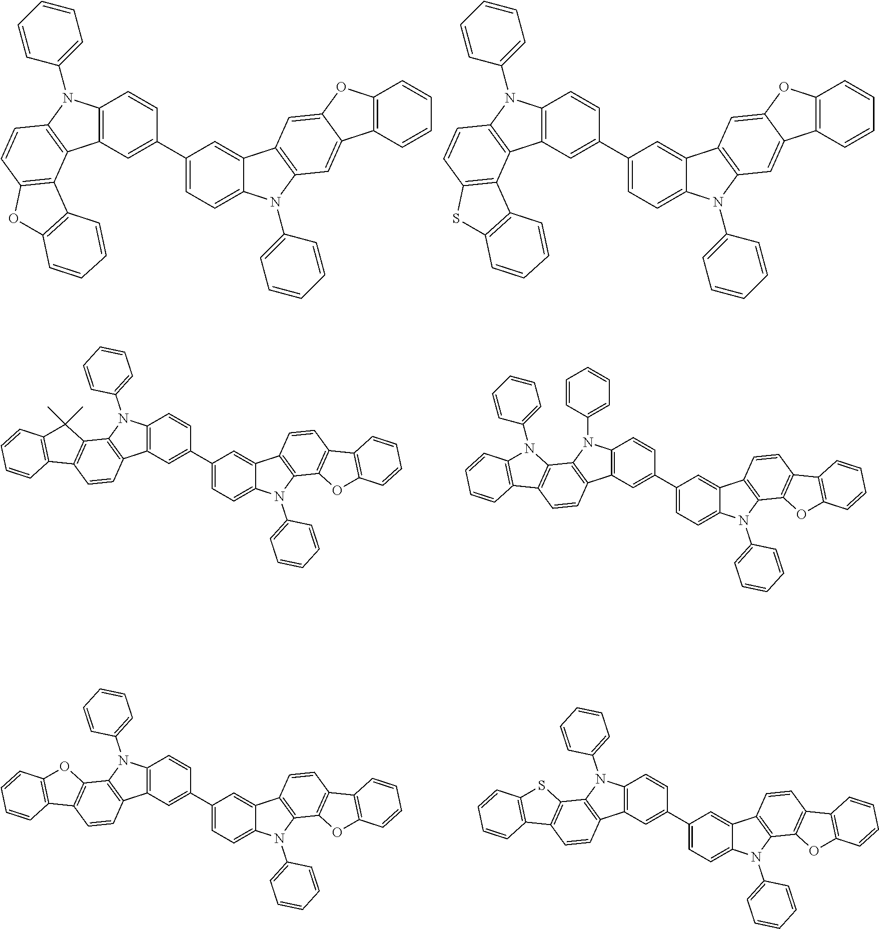

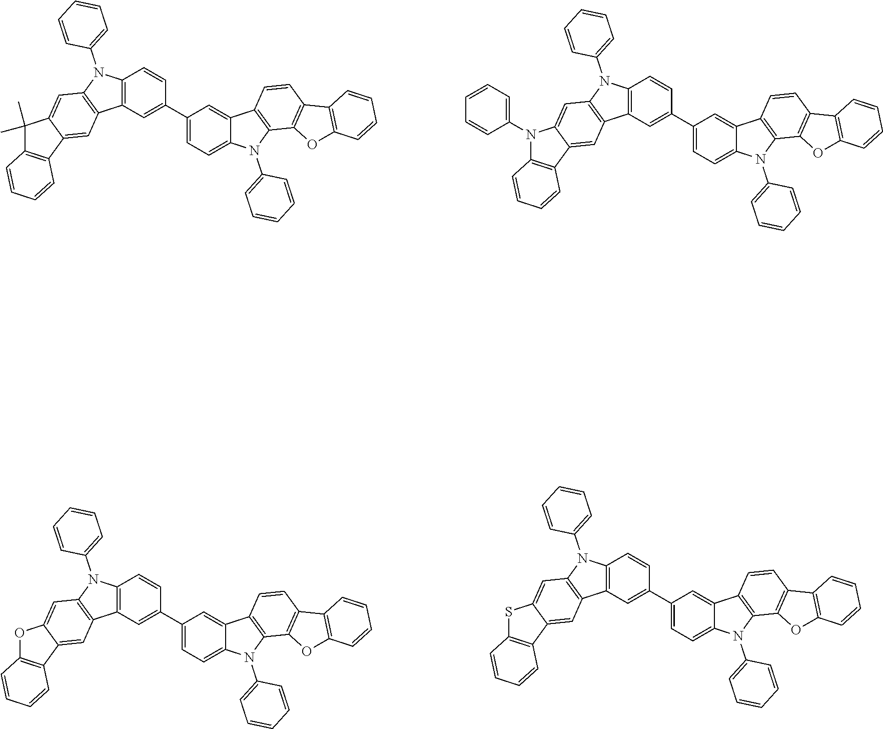 [SMILES: CC1(C)c2ccccc2-c2cc3c4cc(-c5ccc6c(c5)c5ccc7c8ccccc8oc7c5n6-c5ccccc5)ccc4n(-c4ccccc4)c3cc21 ; c1ccc(-n2c3ccccc3c3cc4c5cc(-c6ccc7c(c6)c6ccc8c9ccccc9oc8c6n7-c6ccccc6)ccc5n(-c5ccccc5)c4cc32)cc1 ; c1ccc(-n2c3ccc(-c4ccc5c(c4)c4ccc6c7ccccc7oc6c4n5-c4ccccc4)cc3c3cc4c(cc32)oc2ccccc24)cc1 ; c1ccc(-n2c3ccc(-c4ccc5c(c4)c4ccc6c7ccccc7oc6c4n5-c4ccccc4)cc3c3cc4c(cc32)sc2ccccc24)cc1]

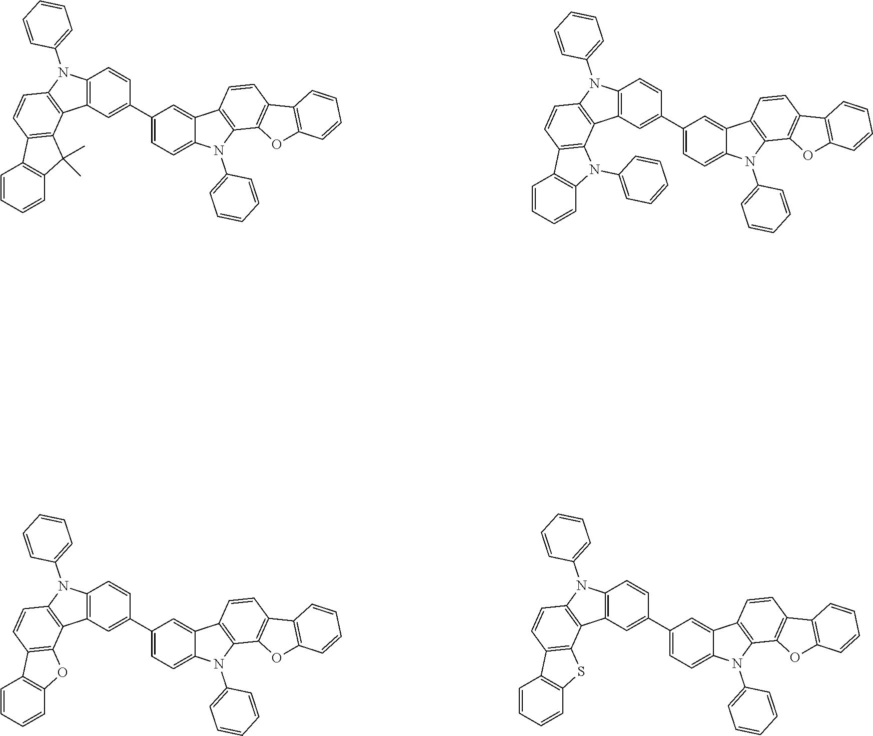 [SMILES: CC1(C)c2ccccc2-c2ccc3c(c21)c1cc(-c2ccc4c(c2)c2ccc5c6ccccc6oc5c2n4-c2ccccc2)ccc1n3-c1ccccc1 ; c1ccc(-n2c3ccc(-c4ccc5c(c4)c4ccc6c7ccccc7oc6c4n5-c4ccccc4)cc3c3c2ccc2c4ccccc4n(-c4ccccc4)c23)cc1 ; c1ccc(-n2c3ccc(-c4ccc5c(c4)c4ccc6c7ccccc7oc6c4n5-c4ccccc4)cc3c3c4oc5ccccc5c4ccc32)cc1 ; c1ccc(-n2c3ccc(-c4ccc5c(c4)c4ccc6c7ccccc7oc6c4n5-c4ccccc4)cc3c3c4sc5ccccc5c4ccc32)cc1]

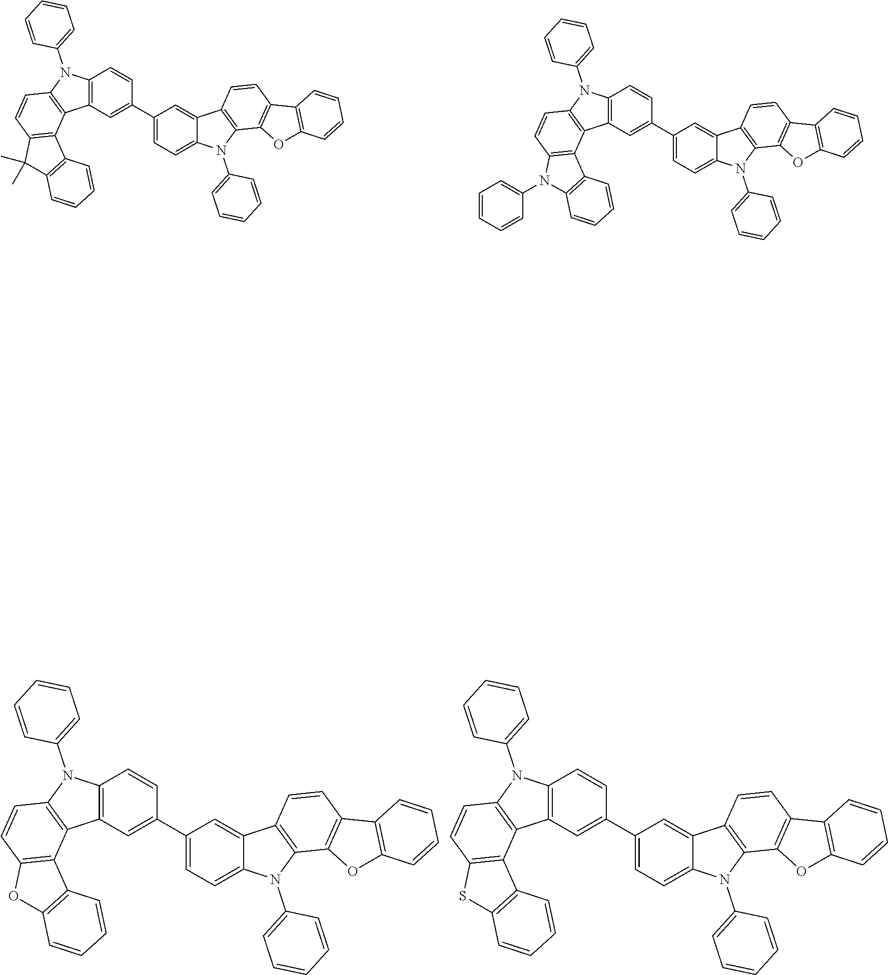

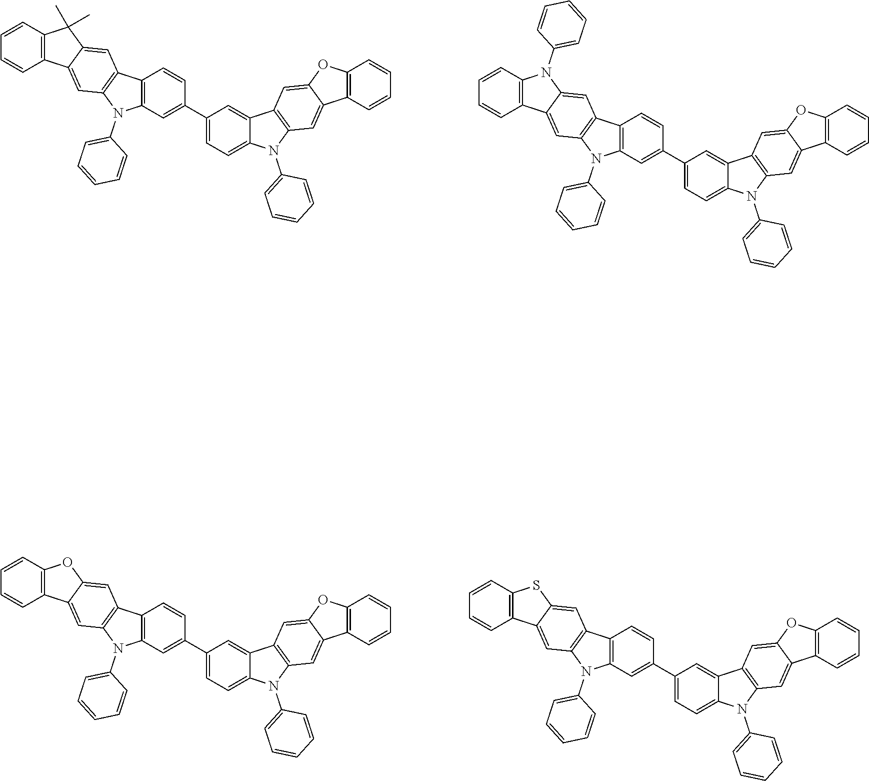 [SMILES: CC1(C)c2ccccc2-c2cc3c(cc21)c1ccc(-c2ccc4c(c2)c2cc5oc6ccccc6c5cc2n4-c2ccccc2)cc1n3-c1ccccc1 ; c1ccc(-n2c3ccc(-c4ccc5c6cc7c(cc6n(-c6ccccc6)c5c4)c4ccccc4n7-c4ccccc4)cc3c3cc4oc5ccccc5c4cc32)cc1 ; c1ccc(-n2c3ccc(-c4ccc5c6cc7oc8ccccc8c7cc6n(-c6ccccc6)c5c4)cc3c3cc4oc5ccccc5c4cc32)cc1 ; c1ccc(-n2c3ccc(-c4ccc5c6cc7sc8ccccc8c7cc6n(-c6ccccc6)c5c4)cc3c3cc4oc5ccccc5c4cc32)cc1]

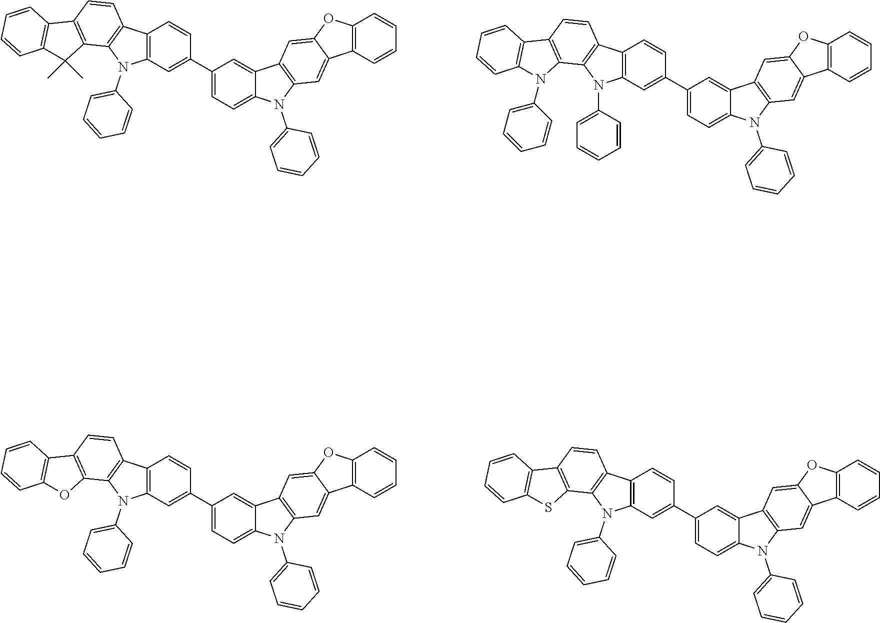 [SMILES: CC1(C)c2ccccc2-c2ccc3c4ccc(-c5ccc6c(c5)c5cc7oc8ccccc8c7cc5n6-c5ccccc5)cc4n(-c4ccccc4)c3c21 ; c1ccc(-n2c3ccc(-c4ccc5c6ccc7c8ccccc8n(-c8ccccc8)c7c6n(-c6ccccc6)c5c4)cc3c3cc4oc5ccccc5c4cc32)cc1 ; c1ccc(-n2c3ccc(-c4ccc5c6ccc7c8ccccc8oc7c6n(-c6ccccc6)c5c4)cc3c3cc4oc5ccccc5c4cc32)cc1 ; c1ccc(-n2c3ccc(-c4ccc5c6ccc7c8ccccc8sc7c6n(-c6ccccc6)c5c4)cc3c3cc4oc5ccccc5c4cc32)cc1]

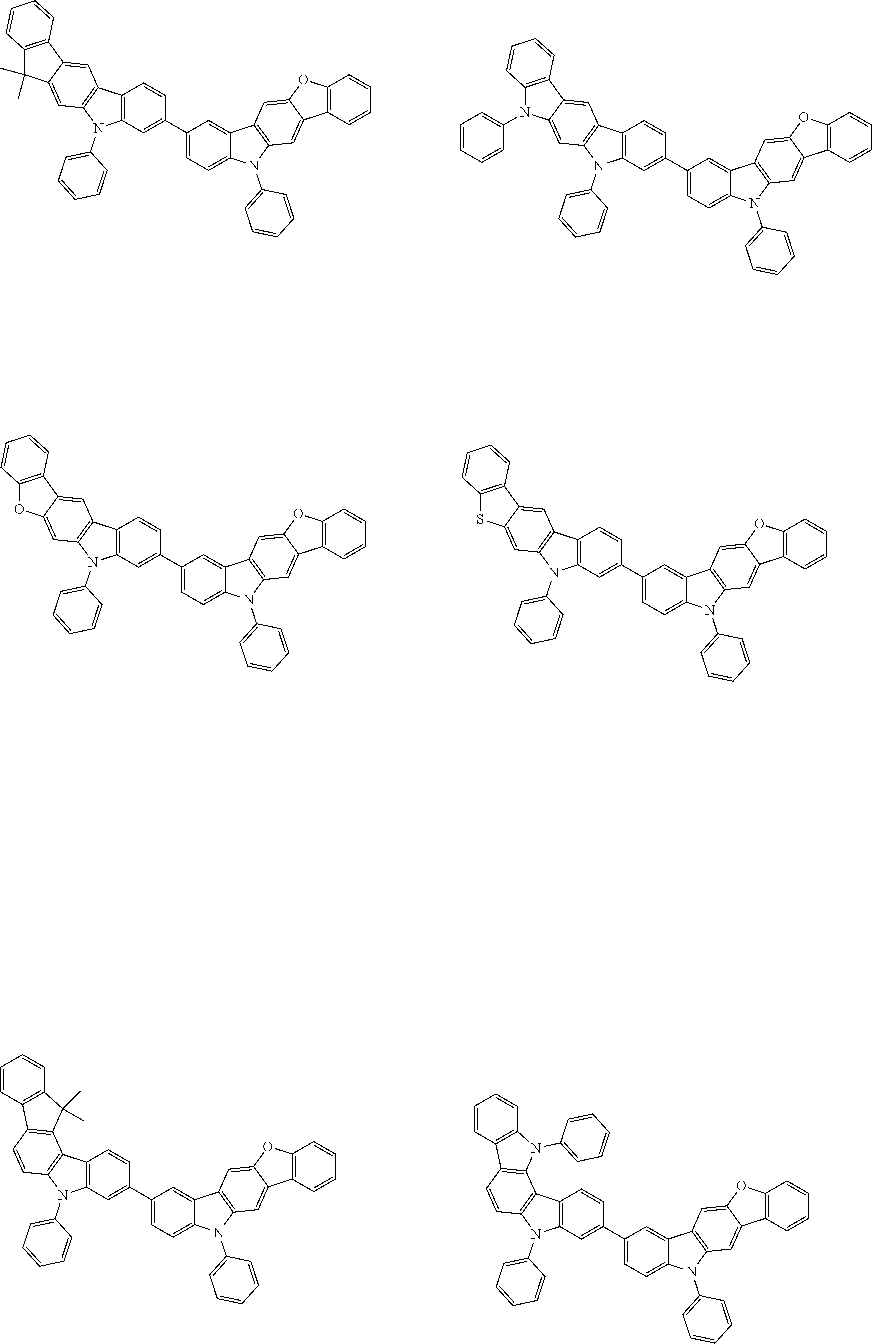

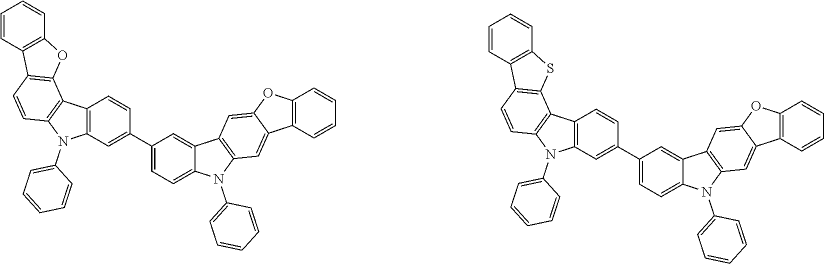 [SMILES: c1ccc(-n2c3ccc(-c4ccc5c6c7oc8ccccc8c7ccc6n(-c6ccccc6)c5c4)cc3c3cc4oc5ccccc5c4cc32)cc1 ; c1ccc(-n2c3ccc(-c4ccc5c6c7sc8ccccc8c7ccc6n(-c6ccccc6)c5c4)cc3c3cc4oc5ccccc5c4cc32)cc1]

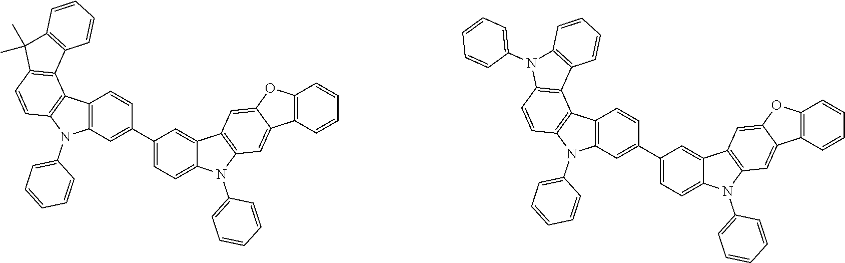 [SMILES: CC1(C)c2ccccc2-c2c1ccc1c2c2ccc(-c3ccc4c(c3)c3cc5oc6ccccc6c5cc3n4-c3ccccc3)cc2n1-c1ccccc1 ; c1ccc(-n2c3ccc(-c4ccc5c6c7c8ccccc8n(-c8ccccc8)c7ccc6n(-c6ccccc6)c5c4)cc3c3cc4oc5ccccc5c4cc32)cc1]

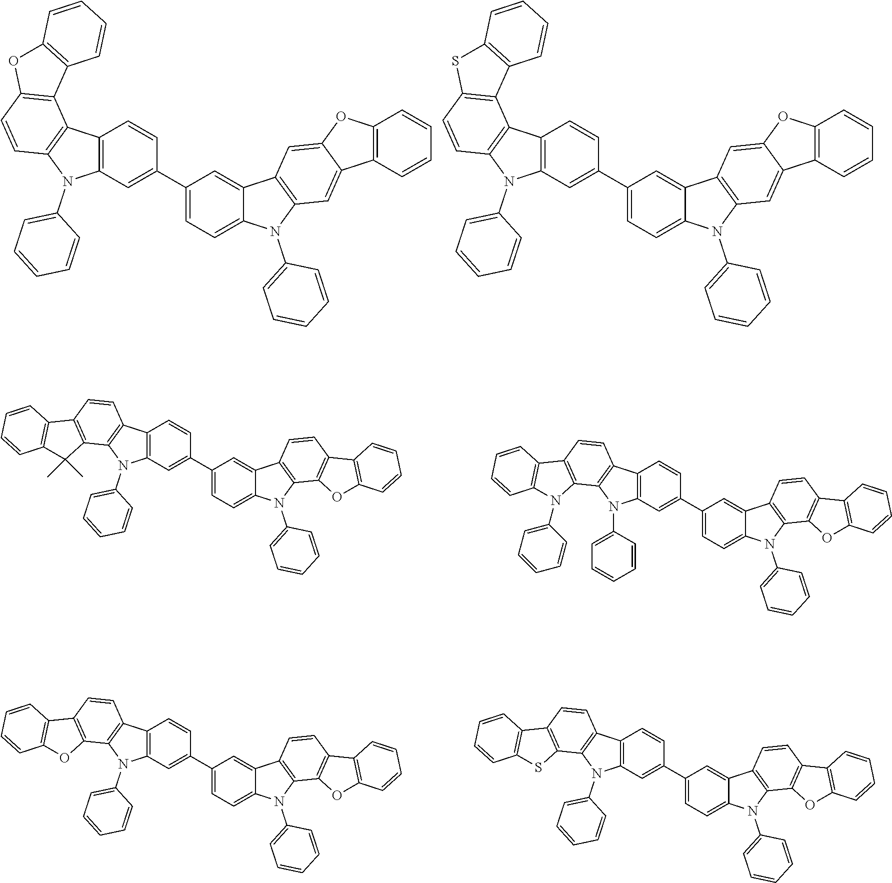

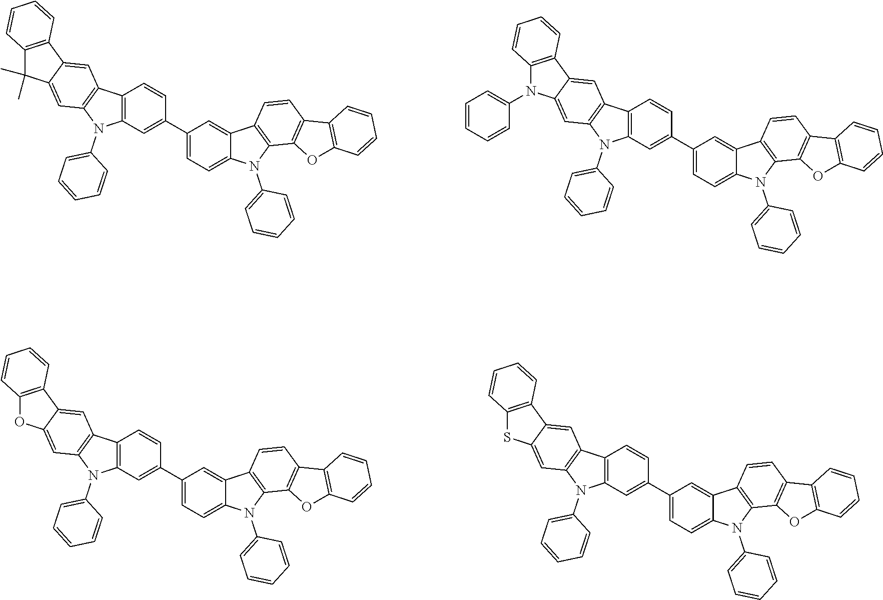 [SMILES: CC1(C)c2ccccc2-c2cc3c4ccc(-c5ccc6c(c5)c5ccc7c8ccccc8oc7c5n6-c5ccccc5)cc4n(-c4ccccc4)c3cc21 ; c1ccc(-n2c3ccccc3c3cc4c5ccc(-c6ccc7c(c6)c6ccc8c9ccccc9oc8c6n7-c6ccccc6)cc5n(-c5ccccc5)c4cc32)cc1 ; c1ccc(-n2c3cc(-c4ccc5c(c4)c4ccc6c7ccccc7oc6c4n5-c4ccccc4)ccc3c3cc4c(cc32)oc2ccccc24)cc1 ; c1ccc(-n2c3cc(-c4ccc5c(c4)c4ccc6c7ccccc7oc6c4n5-c4ccccc4)ccc3c3cc4c(cc32)sc2ccccc24)cc1]

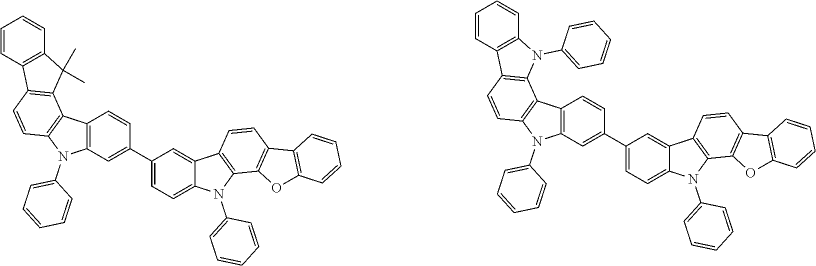 [SMILES: CC1(C)c2ccccc2-c2ccc3c(c21)c1ccc(-c2ccc4c(c2)c2ccc5c6ccccc6oc5c2n4-c2ccccc2)cc1n3-c1ccccc1 ; c1ccc(-n2c3cc(-c4ccc5c(c4)c4ccc6c7ccccc7oc6c4n5-c4ccccc4)ccc3c3c2ccc2c4ccccc4n(-c4ccccc4)c23)cc1]

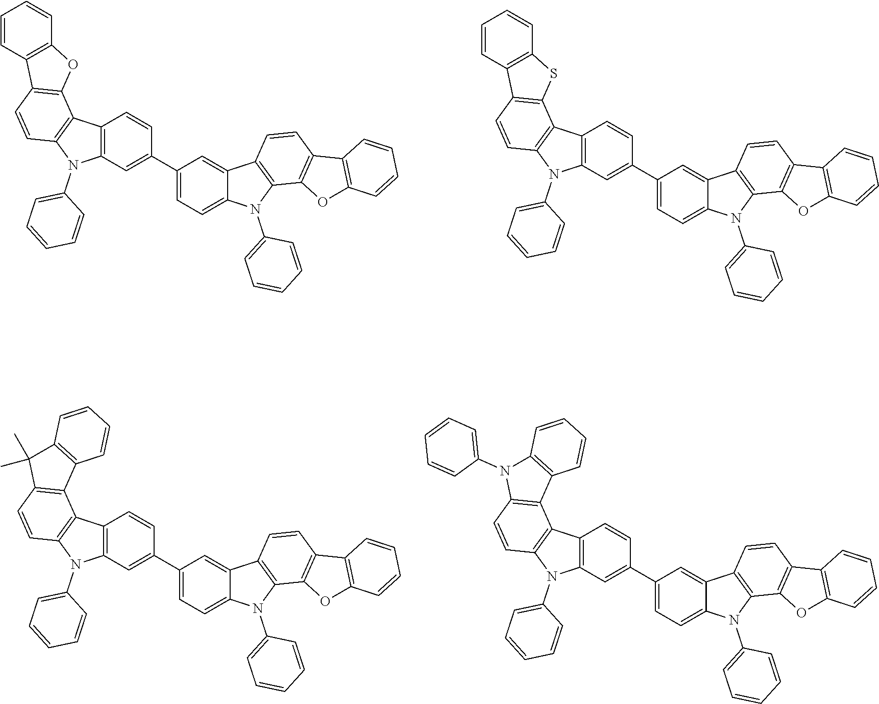 [SMILES: c1ccc(-n2c3cc(-c4ccc5c(c4)c4ccc6c7ccccc7oc6c4n5-c4ccccc4)ccc3c3c4oc5ccccc5c4ccc32)cc1 ; c1ccc(-n2c3cc(-c4ccc5c(c4)c4ccc6c7ccccc7oc6c4n5-c4ccccc4)ccc3c3c4sc5ccccc5c4ccc32)cc1 ; CC1(C)c2ccccc2-c2c1ccc1c2c2ccc(-c3ccc4c(c3)c3ccc5c6ccccc6oc5c3n4-c3ccccc3)cc2n1-c1ccccc1 ; c1ccc(-n2c3ccccc3c3c4c5ccc(-c6ccc7c(c6)c6ccc8c9ccccc9oc8c6n7-c6ccccc6)cc5n(-c5ccccc5)c4ccc32)cc1]

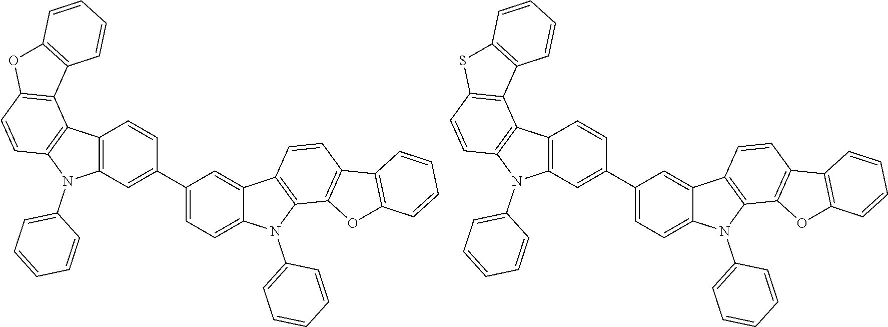

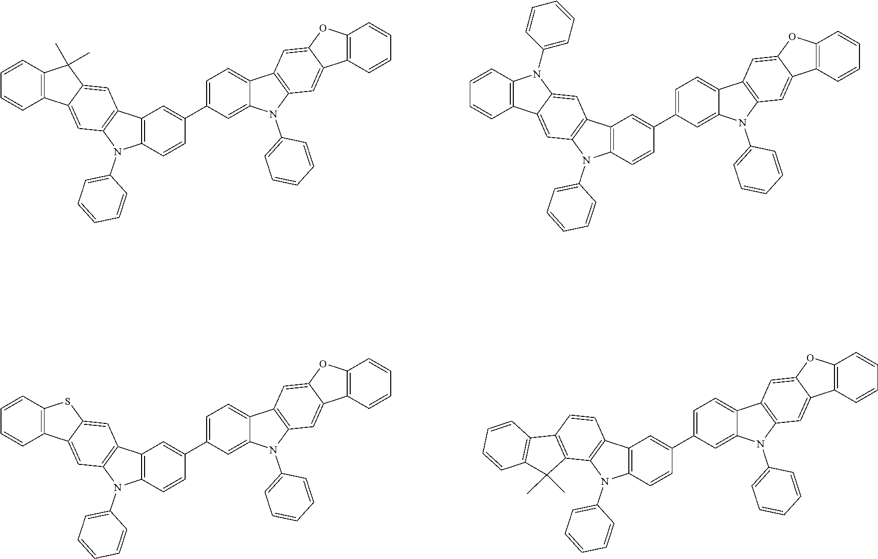 [SMILES: CC1(C)c2ccccc2-c2cc3c(cc21)c1cc(-c2ccc4c5cc6oc7ccccc7c6cc5n(-c5ccccc5)c4c2)ccc1n3-c1ccccc1 ; c1ccc(-n2c3cc(-c4ccc5c(c4)c4cc6c(cc4n5-c4ccccc4)c4ccccc4n6-c4ccccc4)ccc3c3cc4oc5ccccc5c4cc32)cc1 ; c1ccc(-n2c3cc(-c4ccc5c(c4)c4cc6sc7ccccc7c6cc4n5-c4ccccc4)ccc3c3cc4oc5ccccc5c4cc32)cc1 ; CC1(C)c2ccccc2-c2ccc3c4cc(-c5ccc6c7cc8oc9ccccc9c8cc7n(-c7ccccc7)c6c5)ccc4n(-c4ccccc4)c3c21]

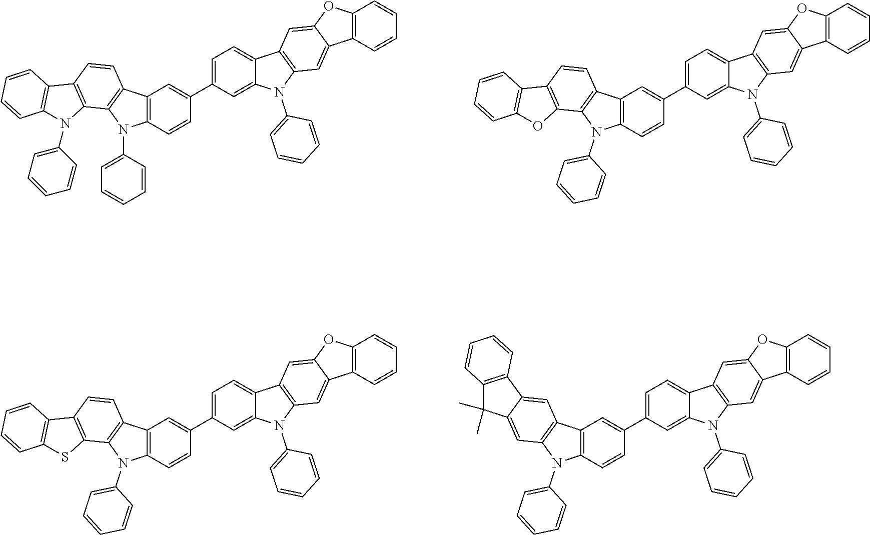 [SMILES: c1ccc(-n2c3cc(-c4ccc5c(c4)c4ccc6c7ccccc7n(-c7ccccc7)c6c4n5-c4ccccc4)ccc3c3cc4oc5ccccc5c4cc32)cc1 ; c1ccc(-n2c3cc(-c4ccc5c(c4)c4ccc6c7ccccc7oc6c4n5-c4ccccc4)ccc3c3cc4oc5ccccc5c4cc32)cc1 ; c1ccc(-n2c3cc(-c4ccc5c(c4)c4ccc6c7ccccc7sc6c4n5-c4ccccc4)ccc3c3cc4oc5ccccc5c4cc32)cc1 ; CC1(C)c2ccccc2-c2cc3c4cc(-c5ccc6c7cc8oc9ccccc9c8cc7n(-c7ccccc7)c6c5)ccc4n(-c4ccccc4)c3cc21]

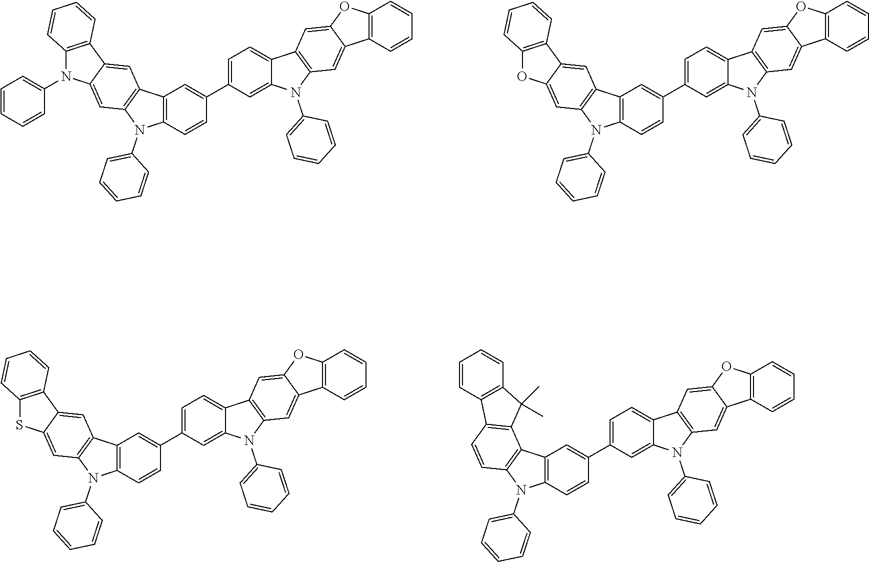 [SMILES: c1ccc(-n2c3cc(-c4ccc5c(c4)c4cc6c7ccccc7n(-c7ccccc7)c6cc4n5-c4ccccc4)ccc3c3cc4oc5ccccc5c4cc32)cc1 ; c1ccc(-n2c3ccc(-c4ccc5c6cc7oc8ccccc8c7cc6n(-c6ccccc6)c5c4)cc3c3cc4c(cc32)oc2ccccc24)cc1 ; c1ccc(-n2c3ccc(-c4ccc5c6cc7oc8ccccc8c7cc6n(-c6ccccc6)c5c4)cc3c3cc4c(cc32)sc2ccccc24)cc1 ; CC1(C)c2ccccc2-c2ccc3c(c21)c1cc(-c2ccc4c5cc6oc7ccccc7c6cc5n(-c5ccccc5)c4c2)ccc1n3-c1ccccc1]

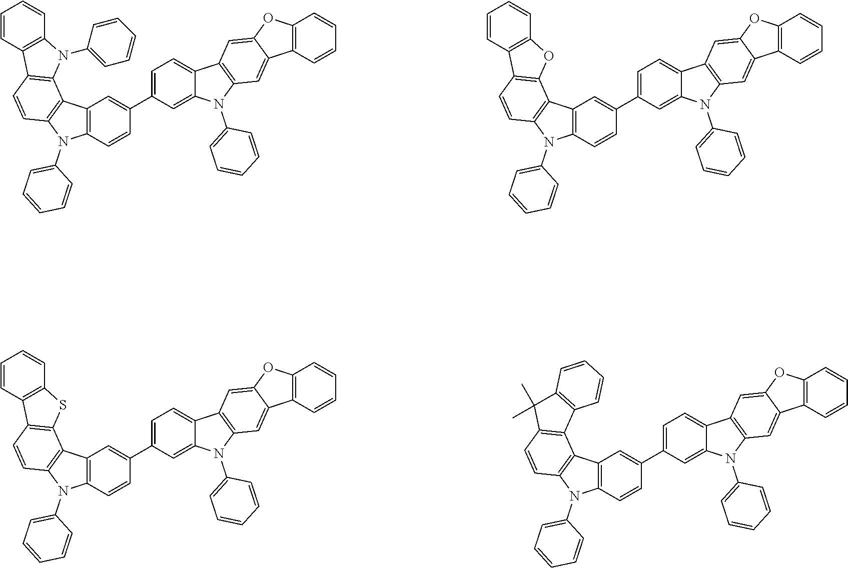 [SMILES: c1ccc(-n2c3cc(-c4ccc5c(c4)c4c(ccc6c7ccccc7n(-c7ccccc7)c64)n5-c4ccccc4)ccc3c3cc4oc5ccccc5c4cc32)cc1 ; c1ccc(-n2c3cc(-c4ccc5c(c4)c4c6oc7ccccc7c6ccc4n5-c4ccccc4)ccc3c3cc4oc5ccccc5c4cc32)cc1 ; c1ccc(-n2c3cc(-c4ccc5c(c4)c4c6sc7ccccc7c6ccc4n5-c4ccccc4)ccc3c3cc4oc5ccccc5c4cc32)cc1 ; CC1(C)c2ccccc2-c2c1ccc1c2c2cc(-c3ccc4c5cc6oc7ccccc7c6cc5n(-c5ccccc5)c4c3)ccc2n1-c1ccccc1]

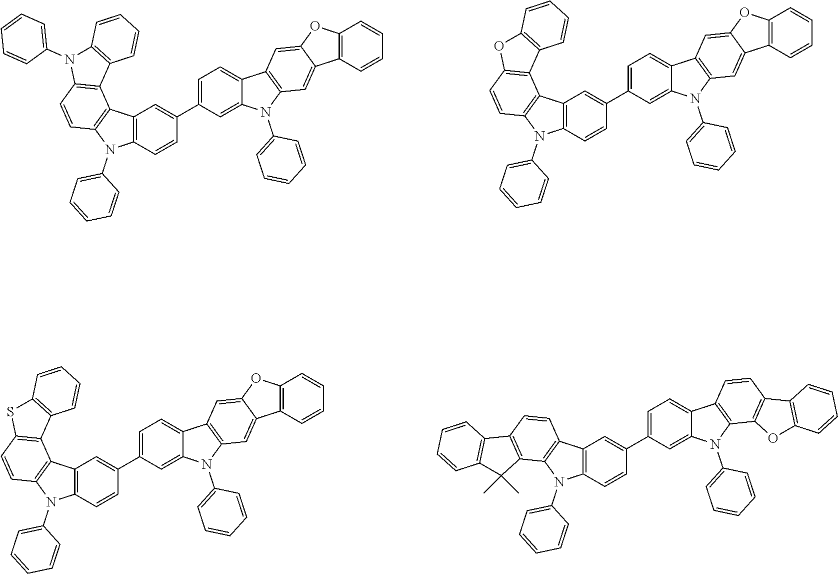 [SMILES: c1ccc(-n2c3cc(-c4ccc5c(c4)c4c6c7ccccc7n(-c7ccccc7)c6ccc4n5-c4ccccc4)ccc3c3cc4oc5ccccc5c4cc32)cc1 ; c1ccc(-n2c3cc(-c4ccc5c(c4)c4c6c(ccc4n5-c4ccccc4)oc4ccccc46)ccc3c3cc4oc5ccccc5c4cc32)cc1 ; c1ccc(-n2c3cc(-c4ccc5c(c4)c4c6c(ccc4n5-c4ccccc4)sc4ccccc46)ccc3c3cc4oc5ccccc5c4cc32)cc1 ; CC1(C)c2ccccc2-c2ccc3c4cc(-c5ccc6c7ccc8c9ccccc9oc8c7n(-c7ccccc7)c6c5)ccc4n(-c4ccccc4)c3c21]

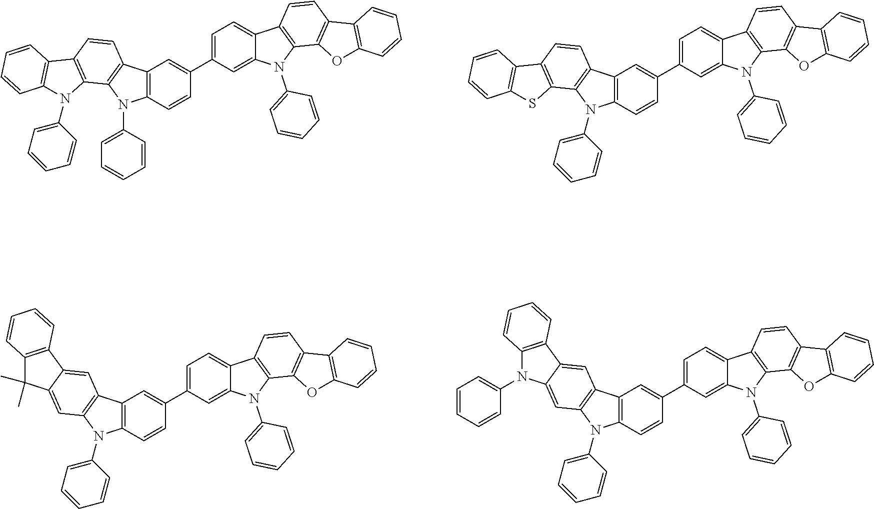 [SMILES: c1ccc(-n2c3cc(-c4ccc5c(c4)c4ccc6c7ccccc7n(-c7ccccc7)c6c4n5-c4ccccc4)ccc3c3ccc4c5ccccc5oc4c32)cc1 ; c1ccc(-n2c3cc(-c4ccc5c(c4)c4ccc6c7ccccc7sc6c4n5-c4ccccc4)ccc3c3ccc4c5ccccc5oc4c32)cc1 ; CC1(C)c2ccccc2-c2cc3c4cc(-c5ccc6c7ccc8c9ccccc9oc8c7n(-c7ccccc7)c6c5)ccc4n(-c4ccccc4)c3cc21 ; c1ccc(-n2c3ccccc3c3cc4c5cc(-c6ccc7c8ccc9c%10ccccc%10oc9c8n(-c8ccccc8)c7c6)ccc5n(-c5ccccc5)c4cc32)cc1]

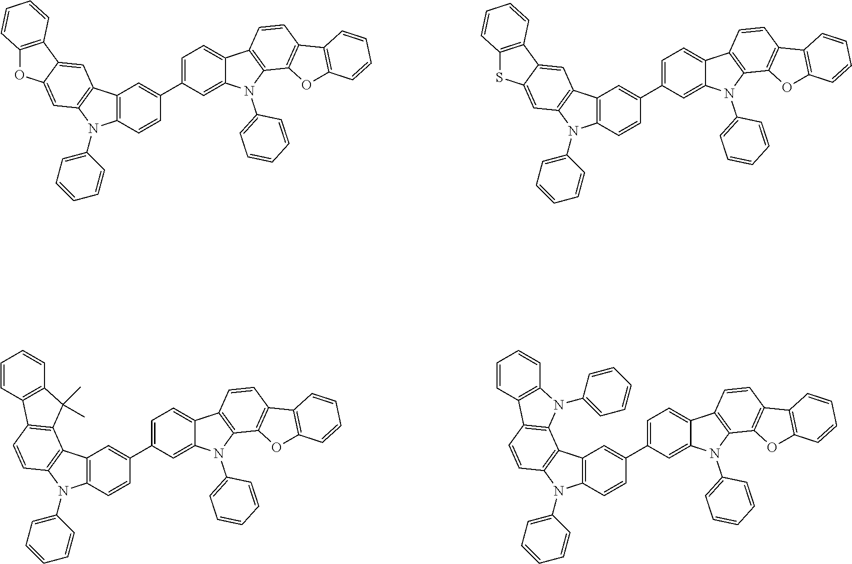 [SMILES: c1ccc(-n2c3ccc(-c4ccc5c6ccc7c8ccccc8oc7c6n(-c6ccccc6)c5c4)cc3c3cc4c(cc32)oc2ccccc24)cc1 ; c1ccc(-n2c3ccc(-c4ccc5c6ccc7c8ccccc8oc7c6n(-c6ccccc6)c5c4)cc3c3cc4c(cc32)sc2ccccc24)cc1 ; CC1(C)c2ccccc2-c2ccc3c(c21)c1cc(-c2ccc4c5ccc6c7ccccc7oc6c5n(-c5ccccc5)c4c2)ccc1n3-c1ccccc1 ; c1ccc(-n2c3ccc(-c4ccc5c6ccc7c8ccccc8oc7c6n(-c6ccccc6)c5c4)cc3c3c2ccc2c4ccccc4n(-c4ccccc4)c23)cc1]

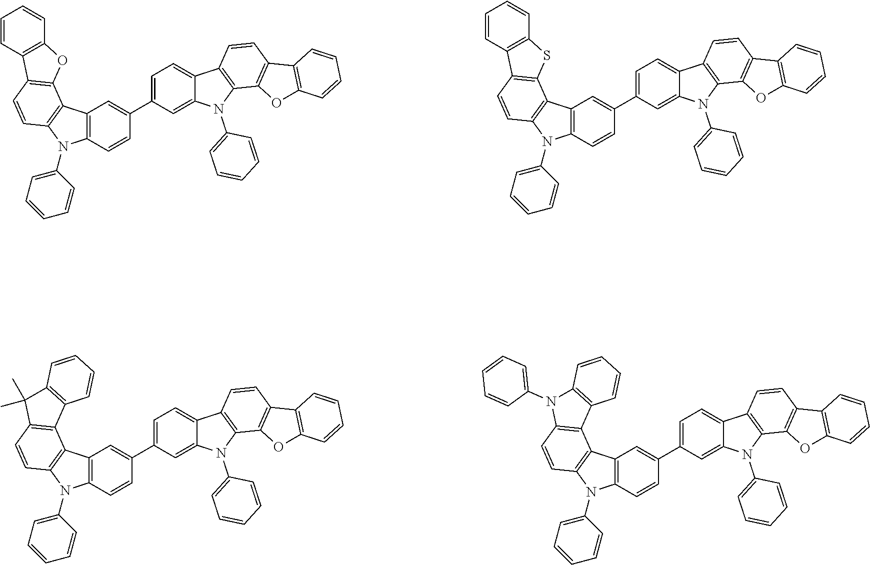 [SMILES: c1ccc(-n2c3ccc(-c4ccc5c6ccc7c8ccccc8oc7c6n(-c6ccccc6)c5c4)cc3c3c4oc5ccccc5c4ccc32)cc1 ; c1ccc(-n2c3ccc(-c4ccc5c6ccc7c8ccccc8oc7c6n(-c6ccccc6)c5c4)cc3c3c4sc5ccccc5c4ccc32)cc1 ; CC1(C)c2ccccc2-c2c1ccc1c2c2cc(-c3ccc4c5ccc6c7ccccc7oc6c5n(-c5ccccc5)c4c3)ccc2n1-c1ccccc1 ; c1ccc(-n2c3ccccc3c3c4c5cc(-c6ccc7c8ccc9c%10ccccc%10oc9c8n(-c8ccccc8)c7c6)ccc5n(-c5ccccc5)c4ccc32)cc1]

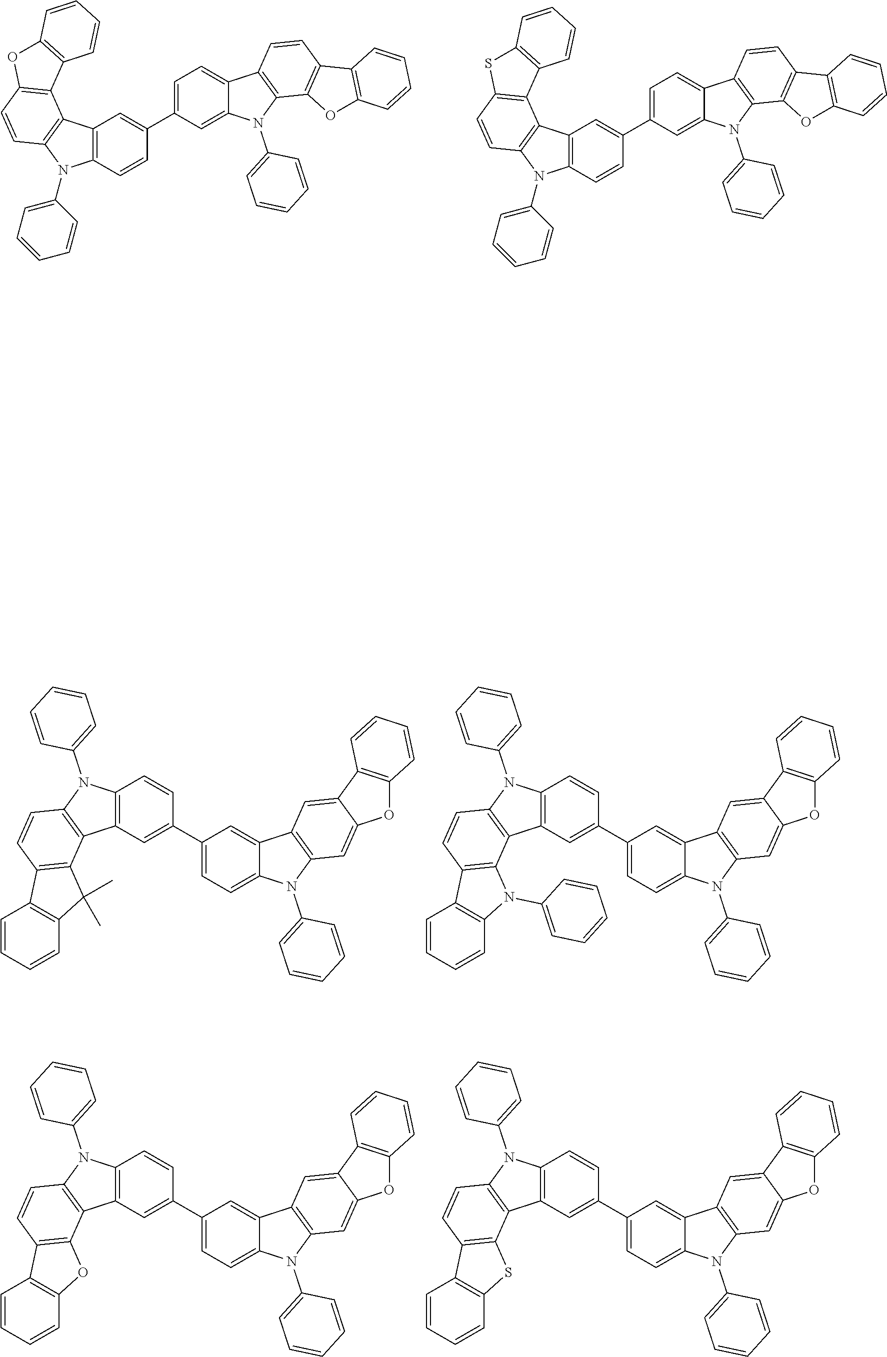

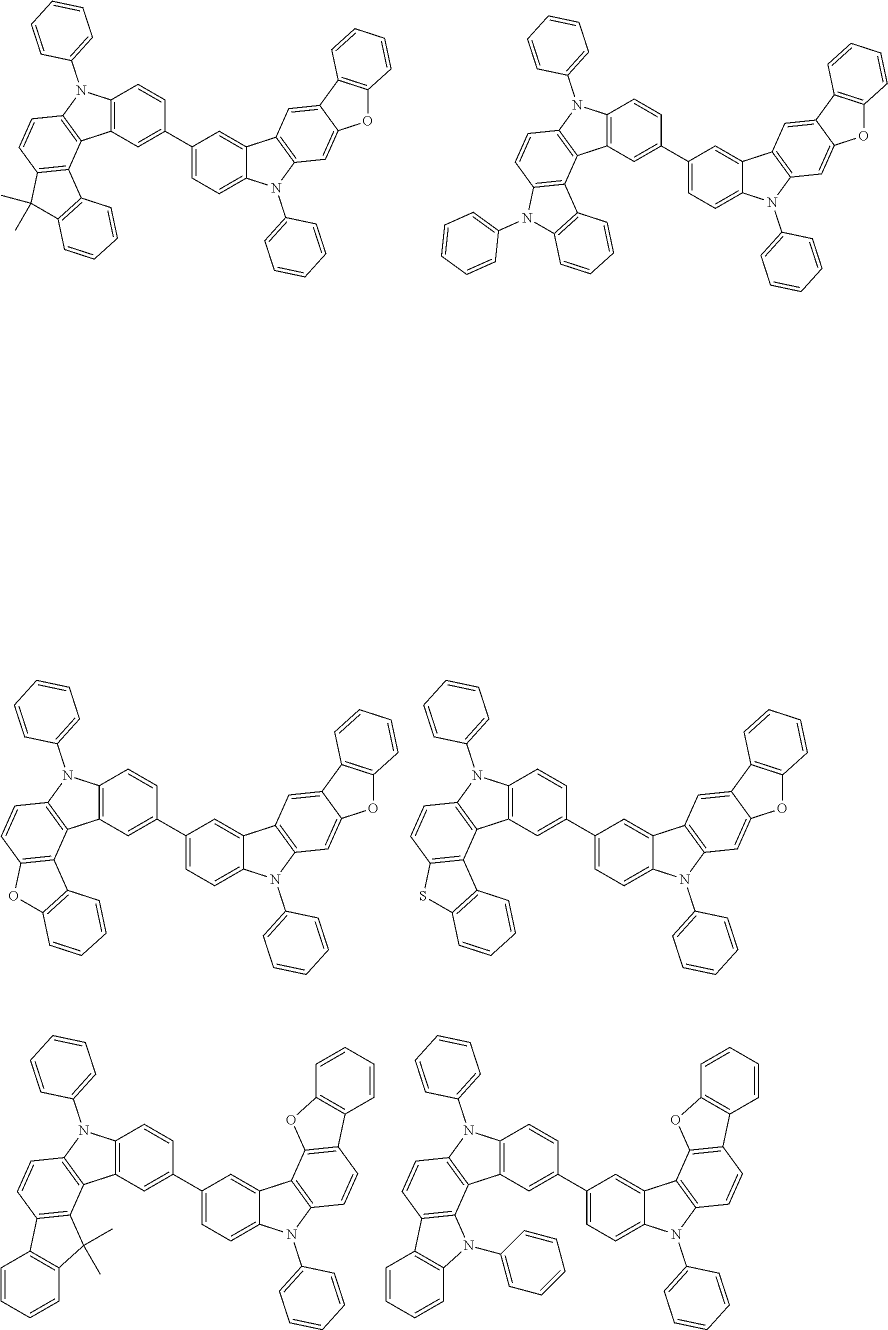

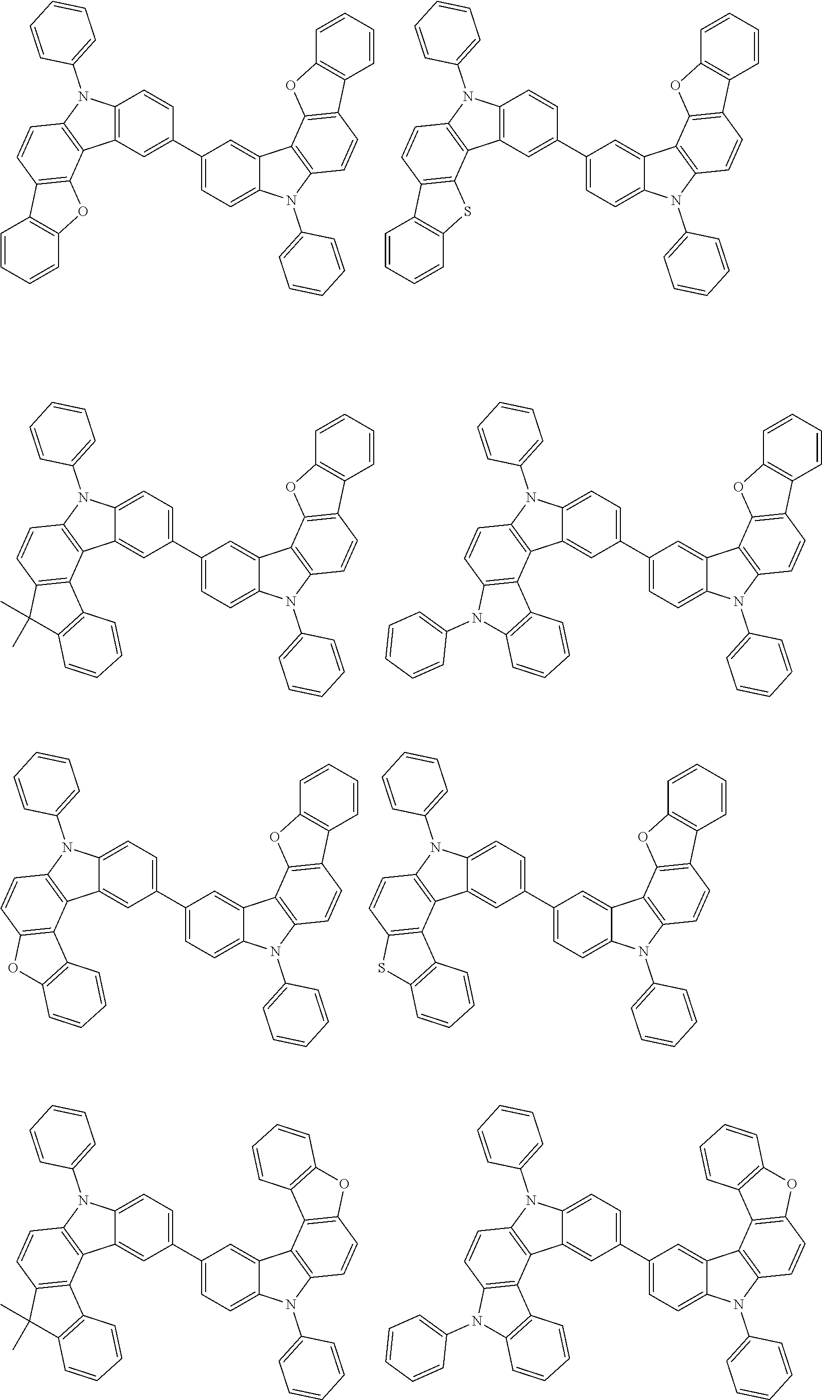

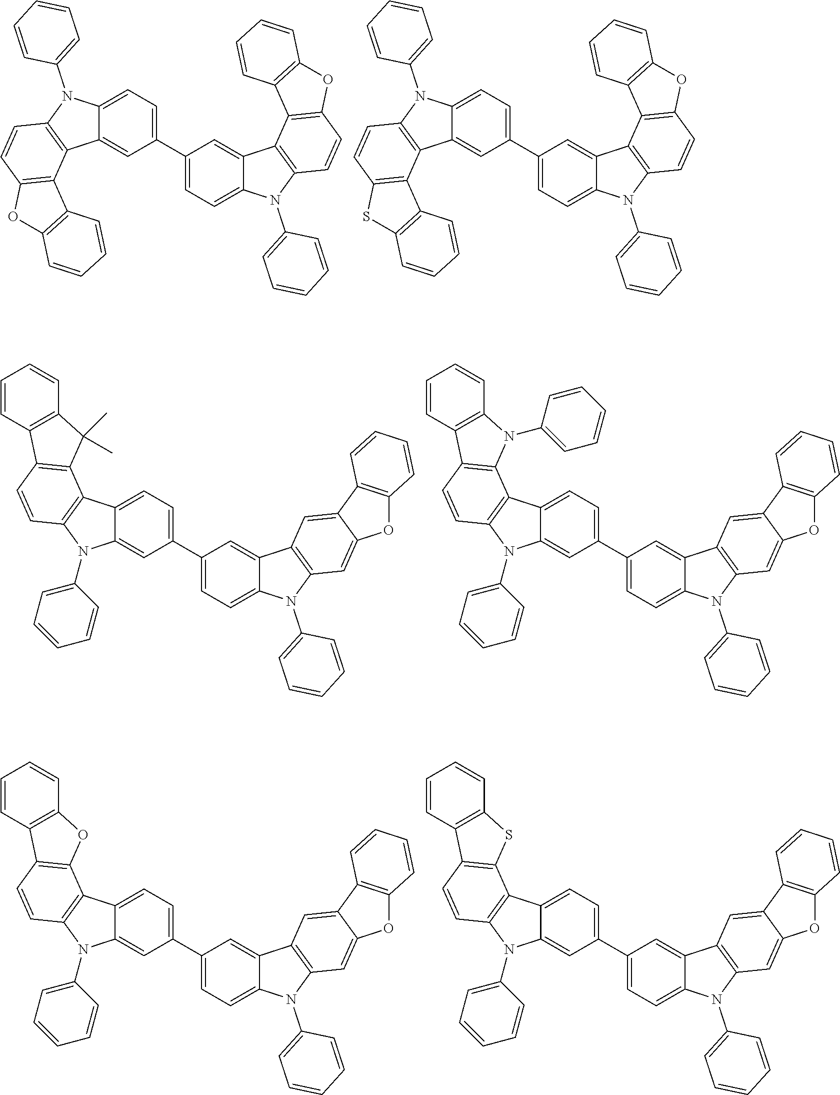

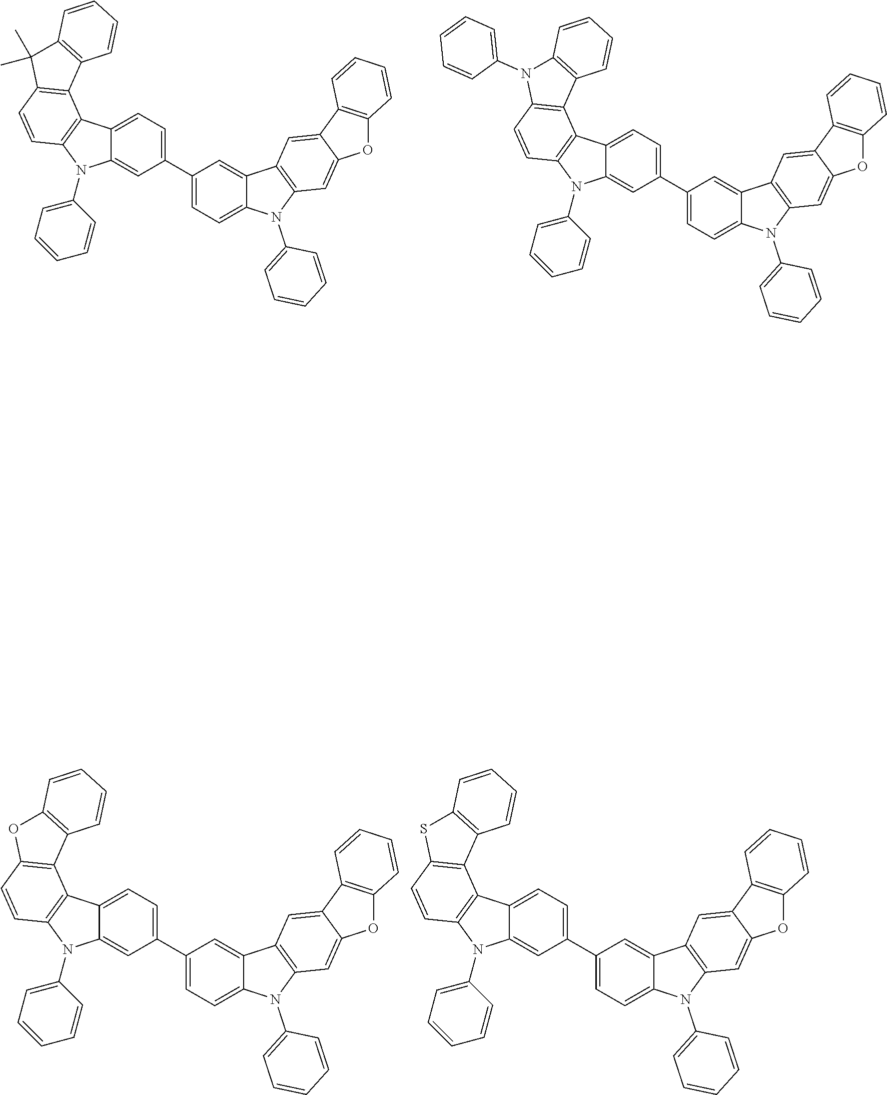

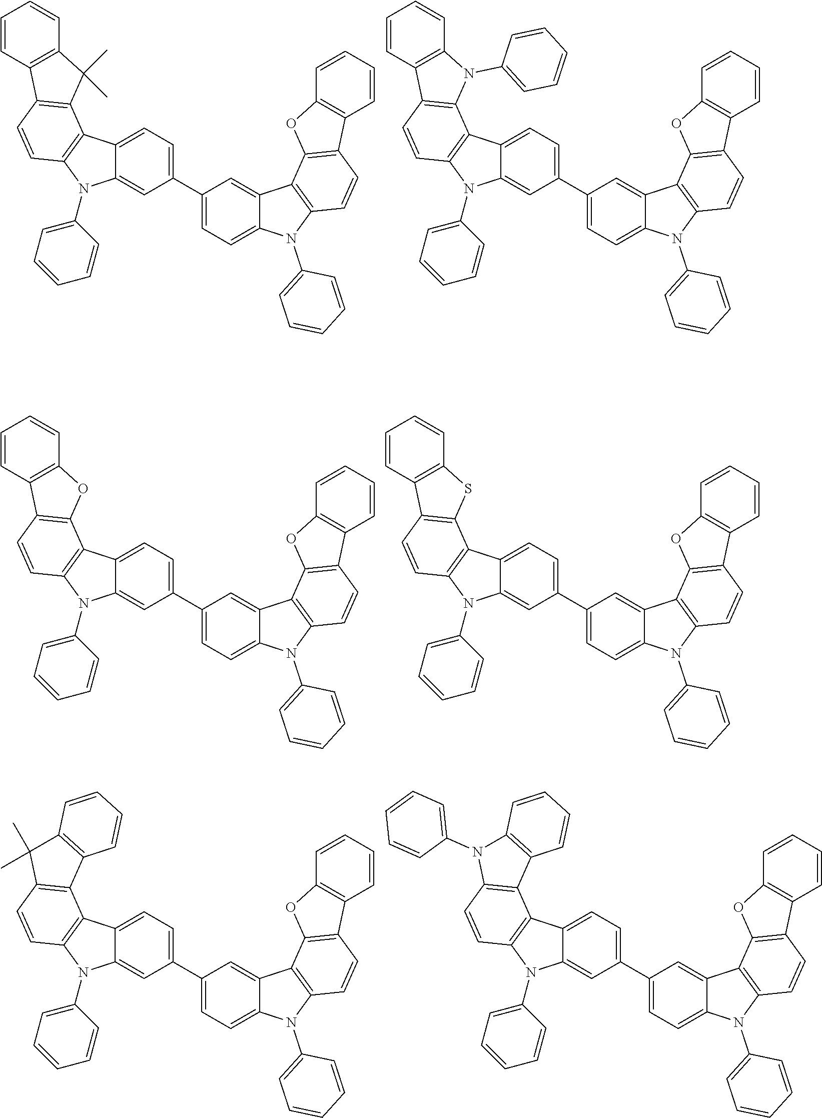

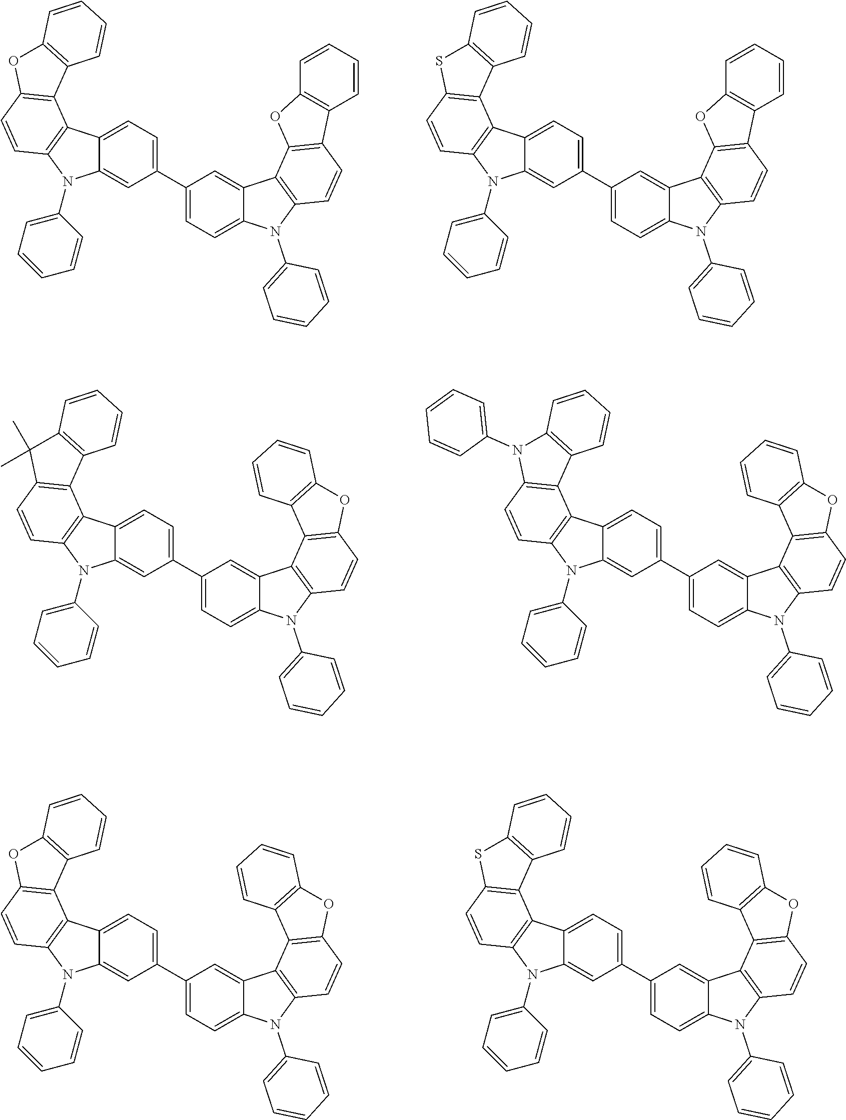

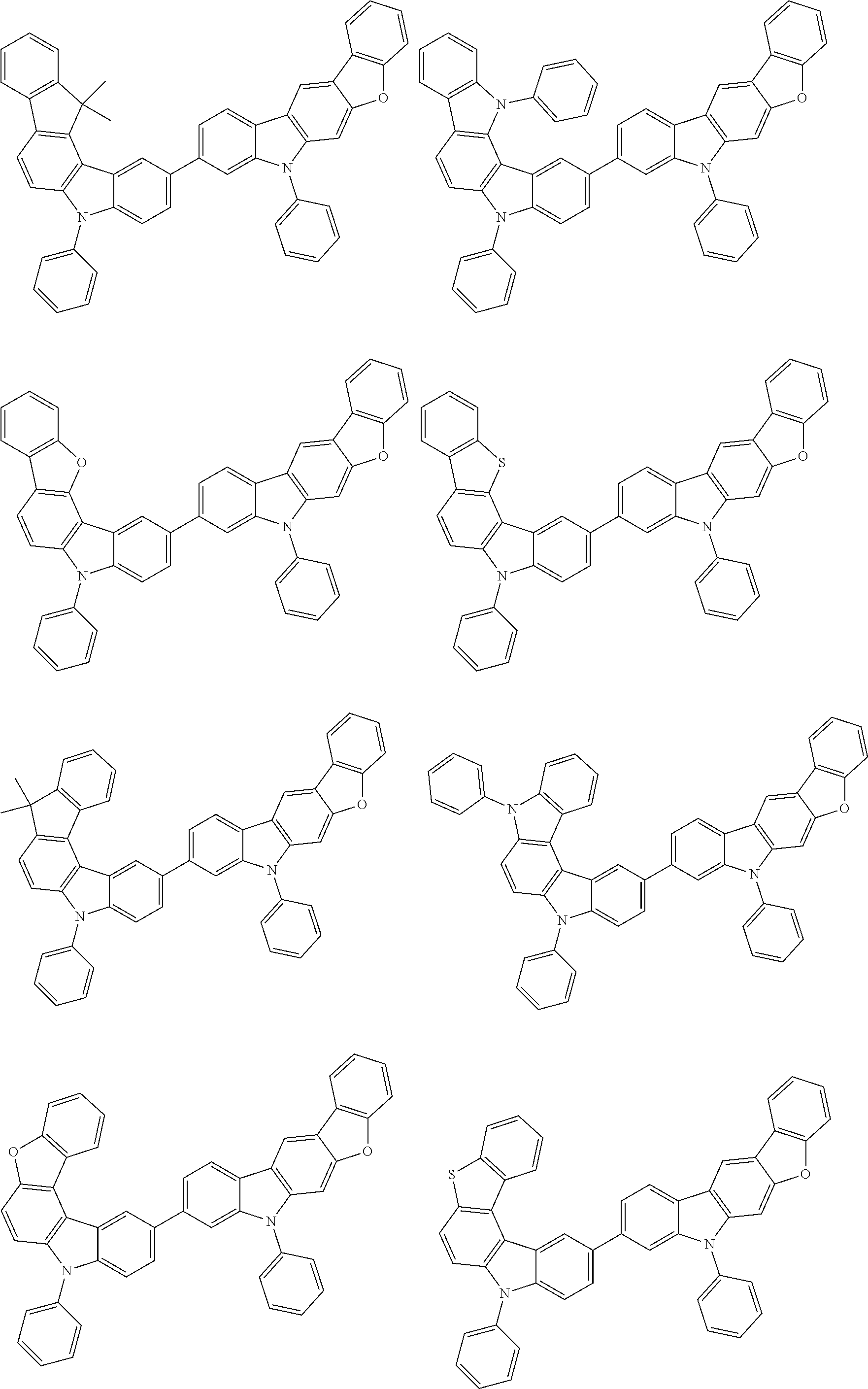

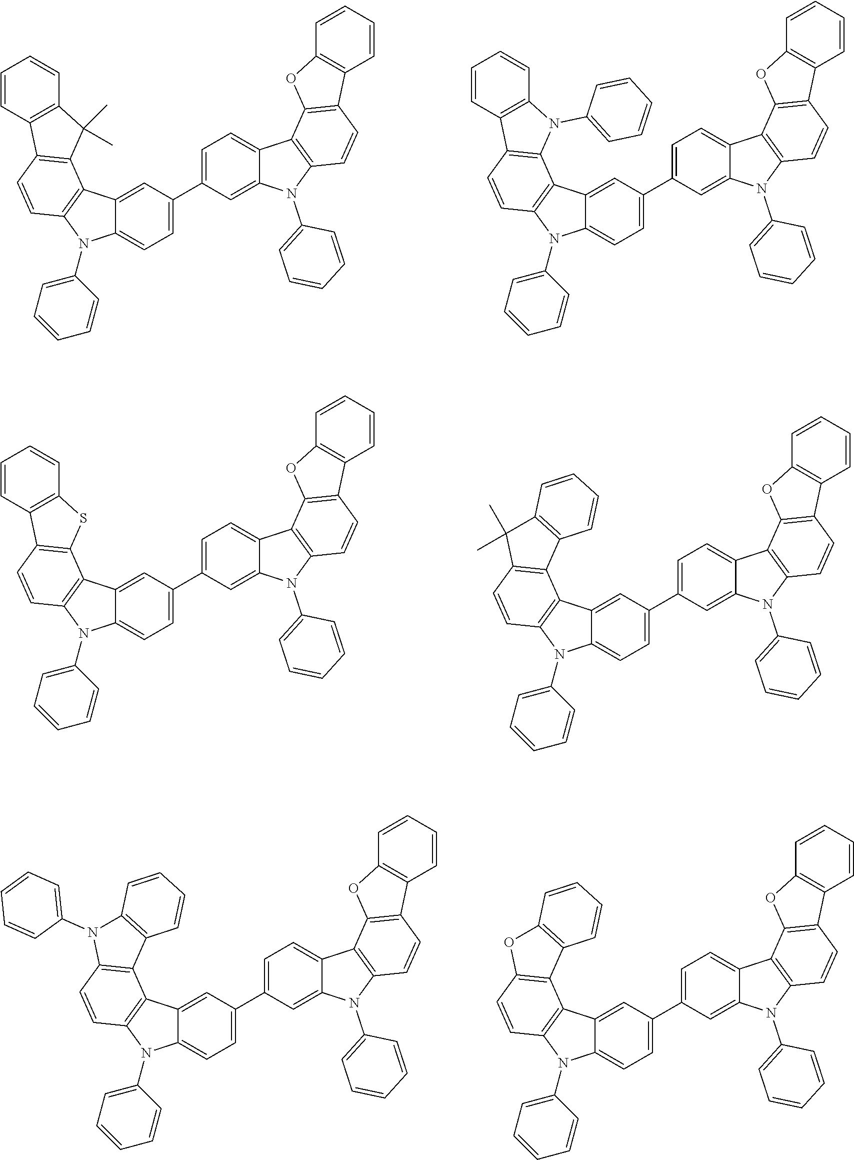

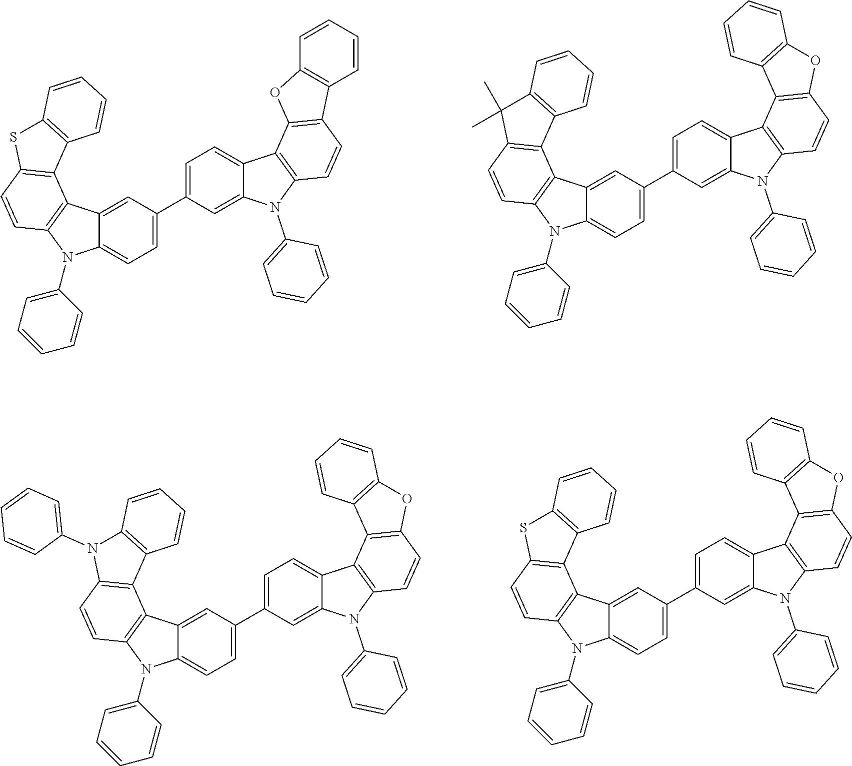 [SMILES: c1ccc(-n2c3cc(-c4ccc5c(c4)c4c6c(ccc4n5-c4ccccc4)sc4ccccc46)ccc3c3c4oc5ccccc5c4ccc32)cc1 ; CC1(C)c2ccccc2-c2c1ccc1c2c2cc(-c3ccc4c5c6c(ccc5n(-c5ccccc5)c4c3)oc3ccccc36)ccc2n1-c1ccccc1 ; c1ccc(-n2c3cc(-c4ccc5c(c4)c4c6c7ccccc7n(-c7ccccc7)c6ccc4n5-c4ccccc4)ccc3c3c4c(ccc32)oc2ccccc24)cc1 ; c1ccc(-n2c3cc(-c4ccc5c(c4)c4c6c(ccc4n5-c4ccccc4)sc4ccccc46)ccc3c3c4c(ccc32)oc2ccccc24)cc1]

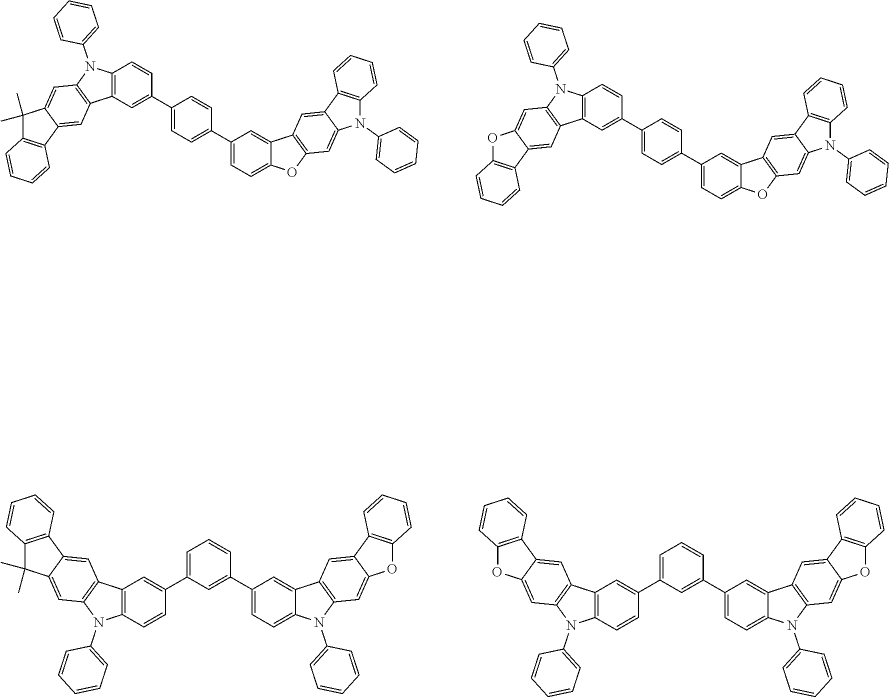 [SMILES: CC1(C)c2ccccc2-c2cc3c4cc(-c5ccc(-c6ccc7oc8cc9c(cc8c7c6)c6ccccc6n9-c6ccccc6)cc5)ccc4n(-c4ccccc4)c3cc21 ; c1ccc(-n2c3ccccc3c3cc4c(cc32)oc2ccc(-c3ccc(-c5ccc6c(c5)c5cc7c(cc5n6-c5ccccc5)oc5ccccc57)cc3)cc24)cc1 ; CC1(C)c2ccccc2-c2cc3c4cc(-c5cccc(-c6ccc7c(c6)c6cc8c(cc6n7-c6ccccc6)oc6ccccc68)c5)ccc4n(-c4ccccc4)c3cc21 ; c1ccc(-n2c3ccc(-c4cccc(-c5ccc6c(c5)c5cc7c(cc5n6-c5ccccc5)oc5ccccc57)c4)cc3c3cc4c(cc32)oc2ccccc24)cc1]

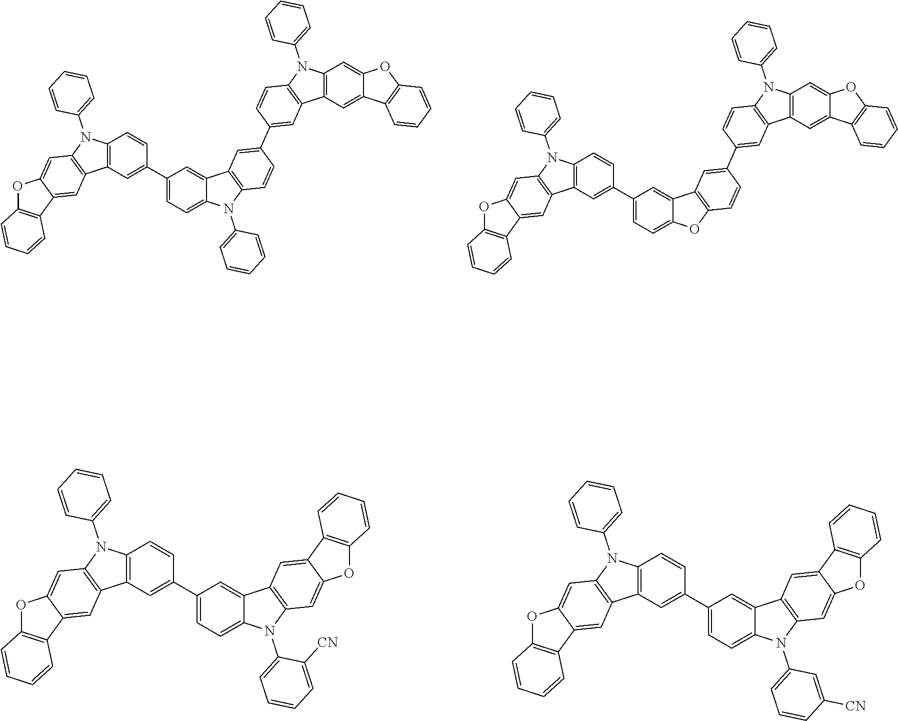 [SMILES: c1ccc(-n2c3ccc(-c4ccc5c(c4)c4cc6c(cc4n5-c4ccccc4)oc4ccccc46)cc3c3cc(-c4ccc5c(c4)c4cc6c(cc4n5-c4ccccc4)oc4ccccc46)ccc32)cc1 ; c1ccc(-n2c3ccc(-c4ccc5oc6ccc(-c7ccc8c(c7)c7cc9c(cc7n8-c7ccccc7)oc7ccccc79)cc6c5c4)cc3c3cc4c(cc32)oc2ccccc24)cc1 ; N#Cc1ccccc1-n1c2ccc(-c3ccc4c(c3)c3cc5c(cc3n4-c3ccccc3)oc3ccccc35)cc2c2cc3c(cc21)oc1ccccc13 ; N#Cc1cccc(-n2c3ccc(-c4ccc5c(c4)c4cc6c(cc4n5-c4ccccc4)oc4ccccc46)cc3c3cc4c(cc32)oc2ccccc24)c1]

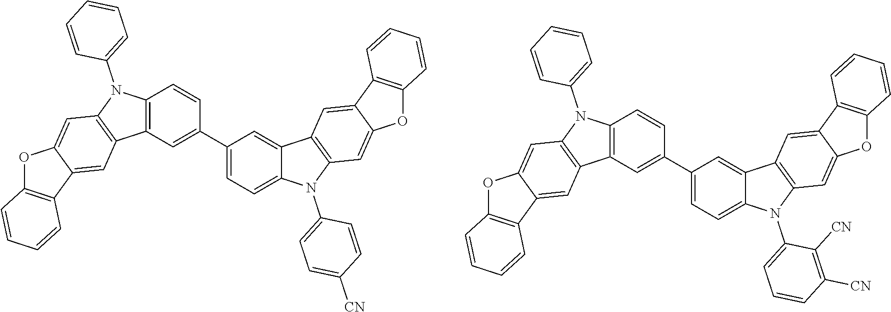 [SMILES: N#Cc1ccc(-n2c3ccc(-c4ccc5c(c4)c4cc6c(cc4n5-c4ccccc4)oc4ccccc46)cc3c3cc4c(cc32)oc2ccccc24)cc1 ; N#Cc1cccc(-n2c3ccc(-c4ccc5c(c4)c4cc6c(cc4n5-c4ccccc4)oc4ccccc46)cc3c3cc4c(cc32)oc2ccccc24)c1C#N]

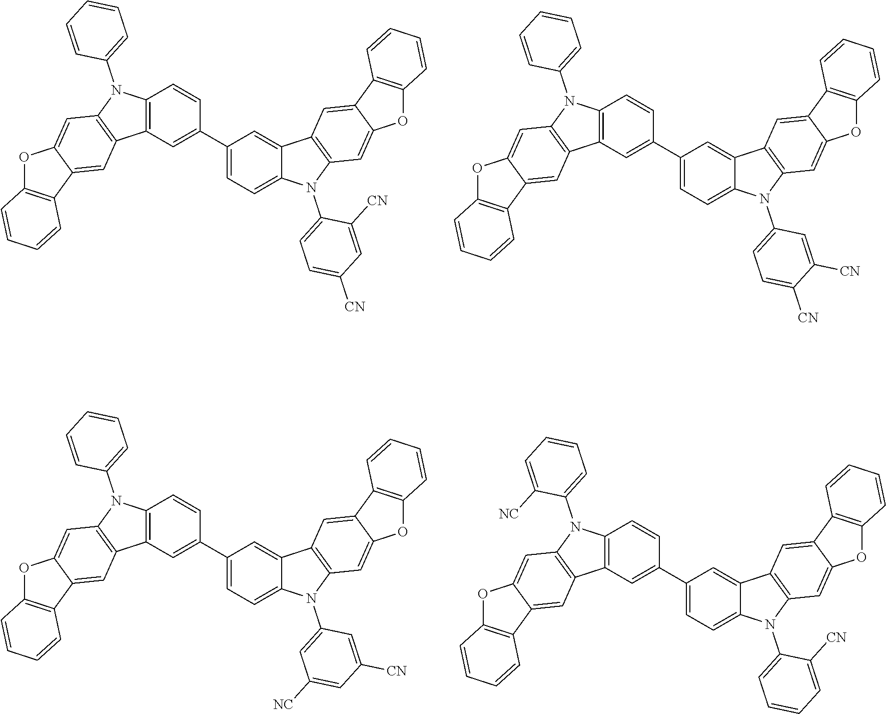 [SMILES: N#Cc1ccc(-n2c3ccc(-c4ccc5c(c4)c4cc6c(cc4n5-c4ccccc4)oc4ccccc46)cc3c3cc4c(cc32)oc2ccccc24)c(C#N)c1 ; N#Cc1ccc(-n2c3ccc(-c4ccc5c(c4)c4cc6c(cc4n5-c4ccccc4)oc4ccccc46)cc3c3cc4c(cc32)oc2ccccc24)cc1C#N ; N#Cc1cc(C#N)cc(-n2c3ccc(-c4ccc5c(c4)c4cc6c(cc4n5-c4ccccc4)oc4ccccc46)cc3c3cc4c(cc32)oc2ccccc24)c1 ; N#Cc1ccccc1-n1c2ccc(-c3ccc4c(c3)c3cc5c(cc3n4-c3ccccc3C#N)oc3ccccc35)cc2c2cc3c(cc21)oc1ccccc13]

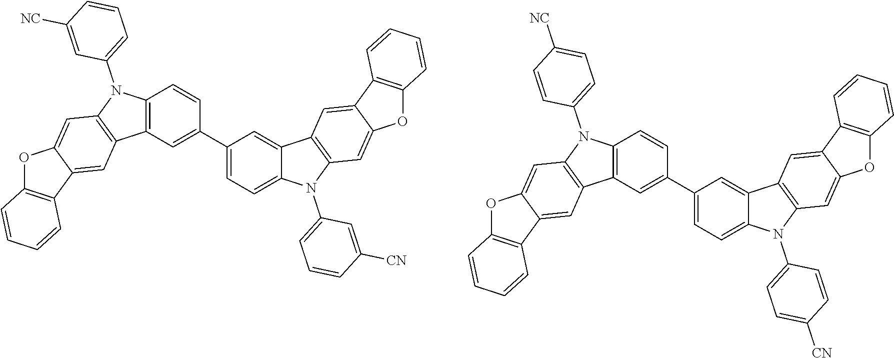 [SMILES: N#Cc1cccc(-n2c3ccc(-c4ccc5c(c4)c4cc6c(cc4n5-c4cccc(C#N)c4)oc4ccccc46)cc3c3cc4c(cc32)oc2ccccc24)c1 ; N#Cc1ccc(-n2c3ccc(-c4ccc5c(c4)c4cc6c(cc4n5-c4ccc(C#N)cc4)oc4ccccc46)cc3c3cc4c(cc32)oc2ccccc24)cc1]

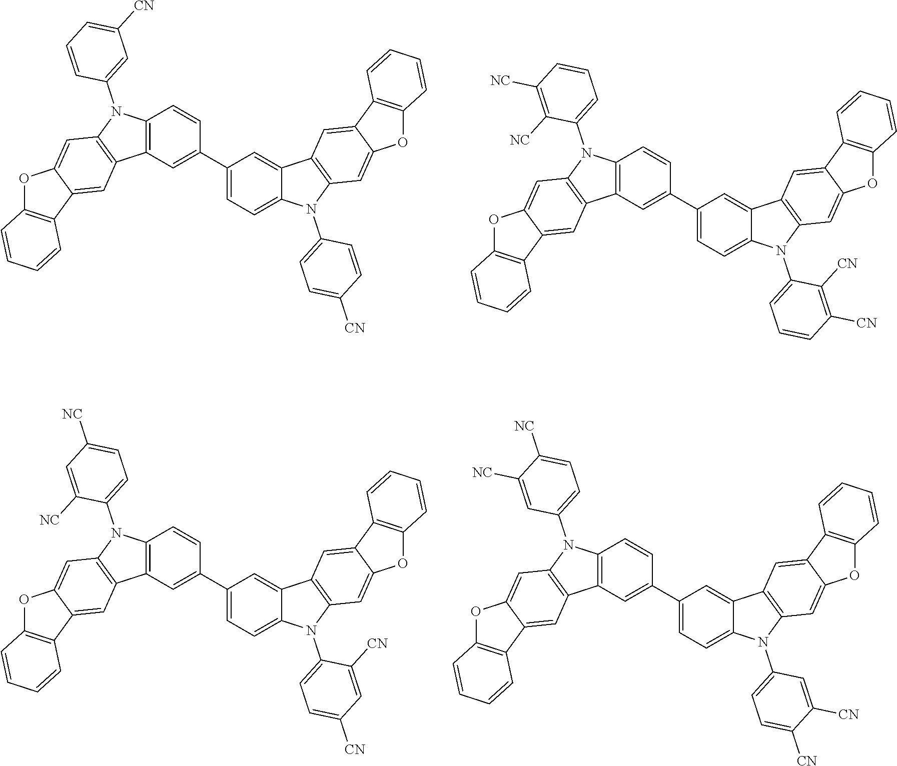 [SMILES: N#Cc1ccc(-n2c3ccc(-c4ccc5c(c4)c4cc6c(cc4n5-c4cccc(C#N)c4)oc4ccccc46)cc3c3cc4c(cc32)oc2ccccc24)cc1 ; N#Cc1cccc(-n2c3ccc(-c4ccc5c(c4)c4cc6c(cc4n5-c4cccc(C#N)c4C#N)oc4ccccc46)cc3c3cc4c(cc32)oc2ccccc24)c1C#N ; N#Cc1ccc(-n2c3ccc(-c4ccc5c(c4)c4cc6c(cc4n5-c4ccc(C#N)cc4C#N)oc4ccccc46)cc3c3cc4c(cc32)oc2ccccc24)c(C#N)c1 ; N#Cc1ccc(-n2c3ccc(-c4ccc5c(c4)c4cc6c(cc4n5-c4ccc(C#N)c(C#N)c4)oc4ccccc46)cc3c3cc4c(cc32)oc2ccccc24)cc1C#N]

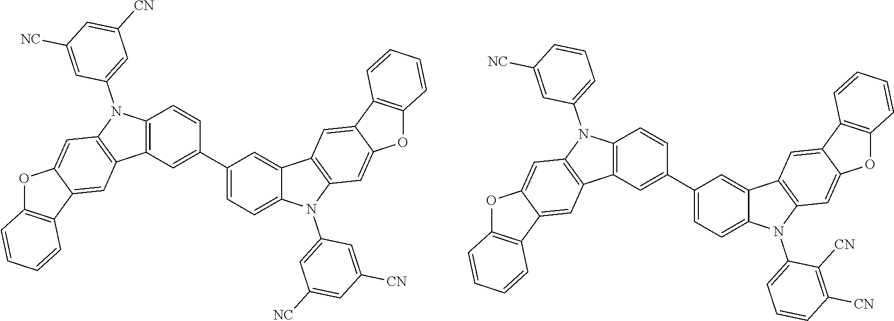 [SMILES: N#Cc1cc(C#N)cc(-n2c3ccc(-c4ccc5c(c4)c4cc6c(cc4n5-c4cc(C#N)cc(C#N)c4)oc4ccccc46)cc3c3cc4c(cc32)oc2ccccc24)c1 ; N#Cc1cccc(-n2c3ccc(-c4ccc5c(c4)c4cc6c(cc4n5-c4cccc(C#N)c4C#N)oc4ccccc46)cc3c3cc4c(cc32)oc2ccccc24)c1]

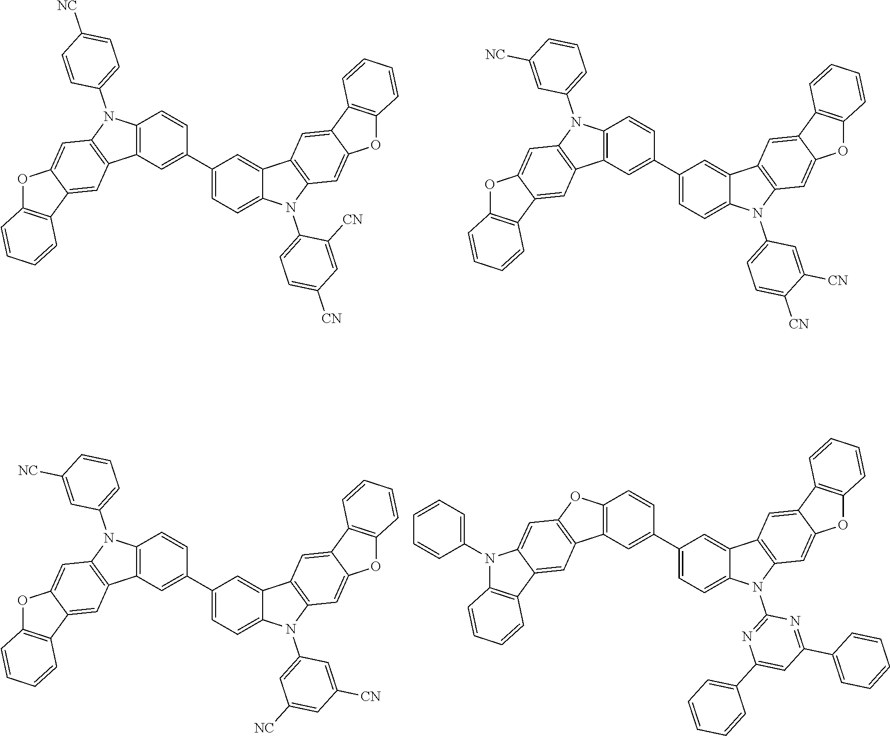

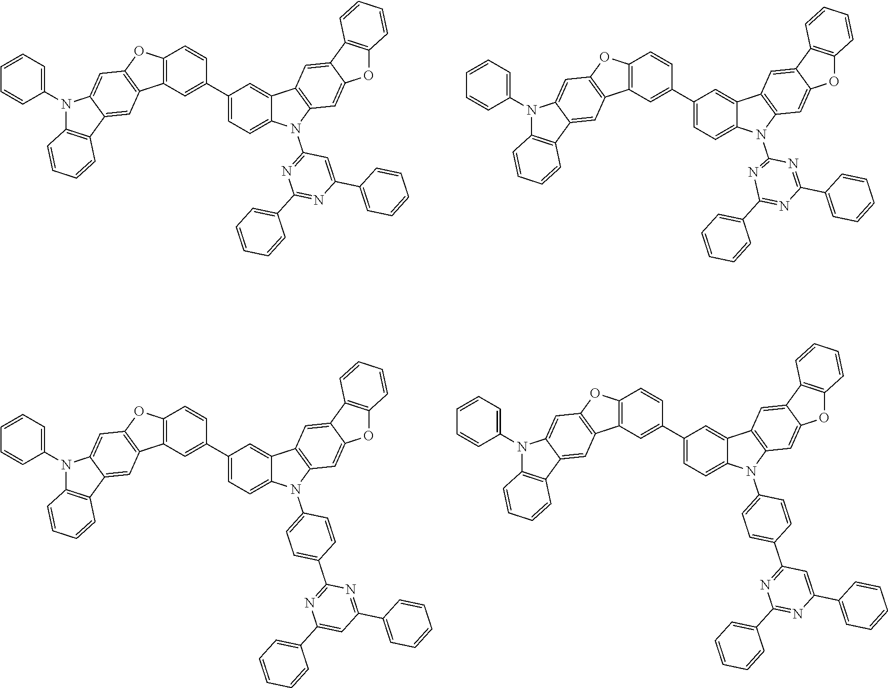 [SMILES: c1ccc(-c2cc(-n3c4ccc(-c5ccc6oc7cc8c(cc7c6c5)c5ccccc5n8-c5ccccc5)cc4c4cc5c(cc43)oc3ccccc35)nc(-c3ccccc3)n2)cc1 ; c1ccc(-c2nc(-c3ccccc3)nc(-n3c4ccc(-c5ccc6oc7cc8c(cc7c6c5)c5ccccc5n8-c5ccccc5)cc4c4cc5c(cc43)oc3ccccc35)n2)cc1 ; c1ccc(-c2cc(-c3ccccc3)nc(-c3ccc(-n4c5ccc(-c6ccc7oc8cc9c(cc8c7c6)c6ccccc6n9-c6ccccc6)cc5c5cc6c(cc54)oc4ccccc46)cc3)n2)cc1 ; c1ccc(-c2cc(-c3ccc(-n4c5ccc(-c6ccc7oc8cc9c(cc8c7c6)c6ccccc6n9-c6ccccc6)cc5c5cc6c(cc54)oc4ccccc46)cc3)nc(-c3ccccc3)n2)cc1]

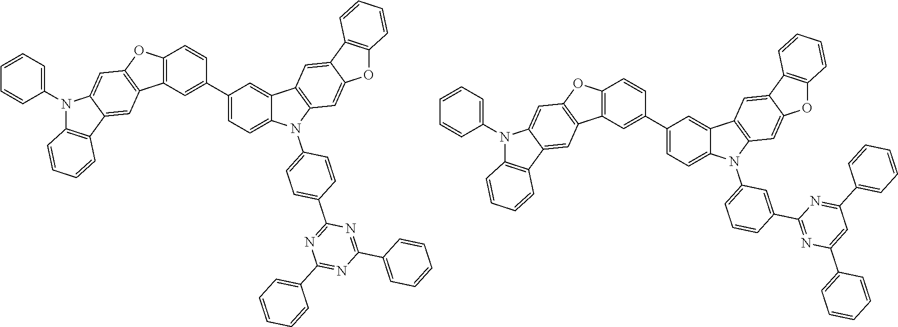 [SMILES: c1ccc(-c2nc(-c3ccccc3)nc(-c3ccc(-n4c5ccc(-c6ccc7oc8cc9c(cc8c7c6)c6ccccc6n9-c6ccccc6)cc5c5cc6c(cc54)oc4ccccc46)cc3)n2)cc1 ; c1ccc(-c2cc(-c3ccccc3)nc(-c3cccc(-n4c5ccc(-c6ccc7oc8cc9c(cc8c7c6)c6ccccc6n9-c6ccccc6)cc5c5cc6c(cc54)oc4ccccc46)c3)n2)cc1]

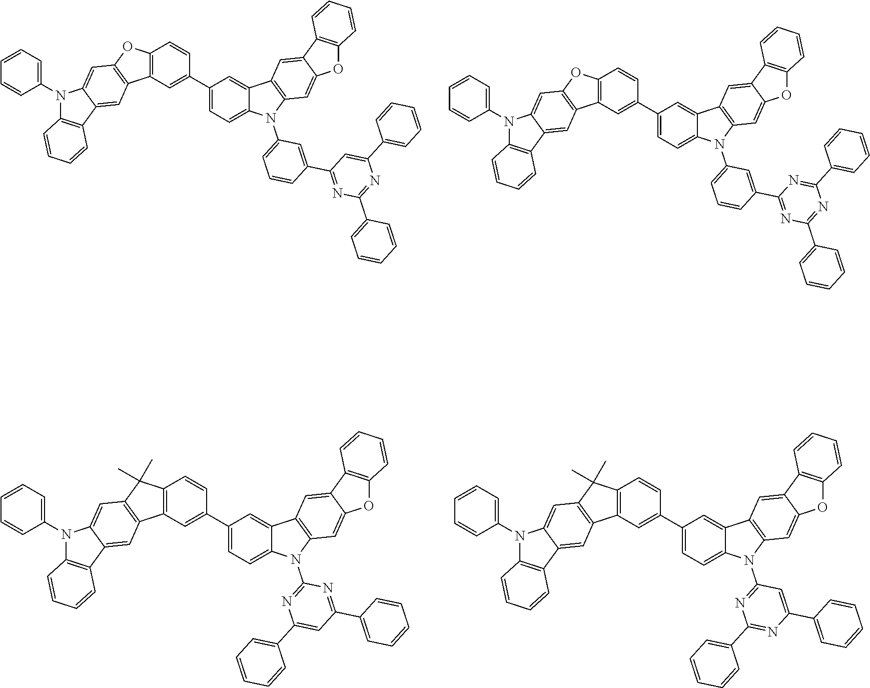 [SMILES: c1ccc(-c2cc(-c3cccc(-n4c5ccc(-c6ccc7oc8cc9c(cc8c7c6)c6ccccc6n9-c6ccccc6)cc5c5cc6c(cc54)oc4ccccc46)c3)nc(-c3ccccc3)n2)cc1 ; c1ccc(-c2nc(-c3ccccc3)nc(-c3cccc(-n4c5ccc(-c6ccc7oc8cc9c(cc8c7c6)c6ccccc6n9-c6ccccc6)cc5c5cc6c(cc54)oc4ccccc46)c3)n2)cc1 ; CC1(C)c2ccc(-c3ccc4c(c3)c3cc5c(cc3n4-c3nc(-c4ccccc4)cc(-c4ccccc4)n3)oc3ccccc35)cc2-c2cc3c4ccccc4n(-c4ccccc4)c3cc21 ; CC1(C)c2ccc(-c3ccc4c(c3)c3cc5c(cc3n4-c3cc(-c4ccccc4)nc(-c4ccccc4)n3)oc3ccccc35)cc2-c2cc3c4ccccc4n(-c4ccccc4)c3cc21]

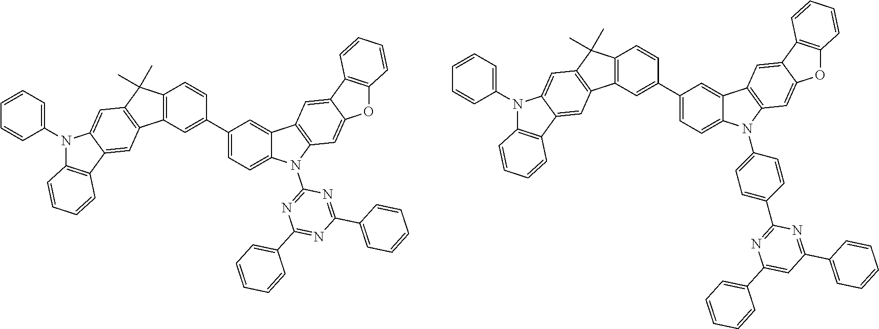 [SMILES: CC1(C)c2ccc(-c3ccc4c(c3)c3cc5c(cc3n4-c3nc(-c4ccccc4)nc(-c4ccccc4)n3)oc3ccccc35)cc2-c2cc3c4ccccc4n(-c4ccccc4)c3cc21 ; CC1(C)c2ccc(-c3ccc4c(c3)c3cc5c(cc3n4-c3ccc(-c4nc(-c6ccccc6)cc(-c6ccccc6)n4)cc3)oc3ccccc35)cc2-c2cc3c4ccccc4n(-c4ccccc4)c3cc21]

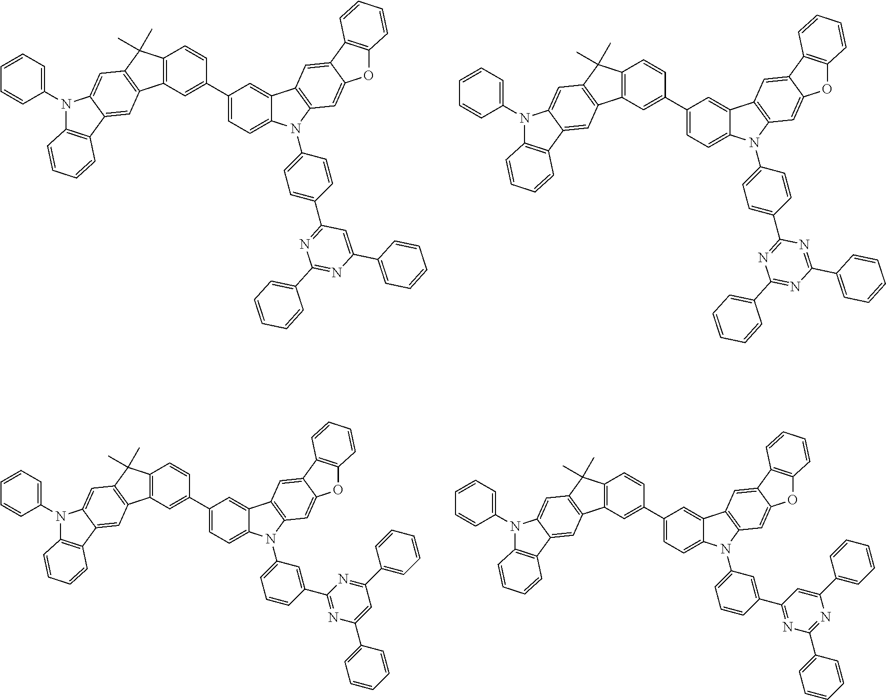

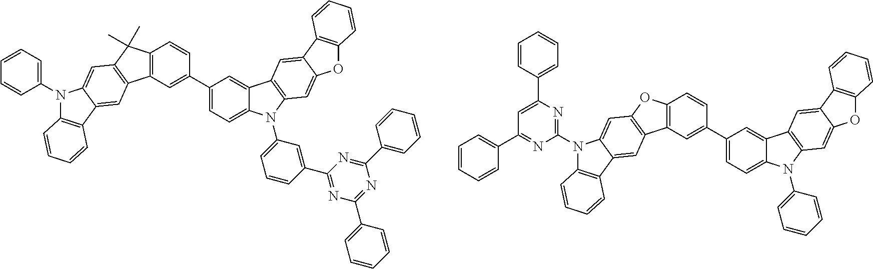 [SMILES: CC1(C)c2ccc(-c3ccc4c(c3)c3cc5c(cc3n4-c3cccc(-c4nc(-c6ccccc6)nc(-c6ccccc6)n4)c3)oc3ccccc35)cc2-c2cc3c4ccccc4n(-c4ccccc4)c3cc21 ; c1ccc(-c2cc(-c3ccccc3)nc(-n3c4ccccc4c4cc5c(cc43)oc3ccc(-c4ccc6c(c4)c4cc7c(cc4n6-c4ccccc4)oc4ccccc47)cc35)n2)cc1]

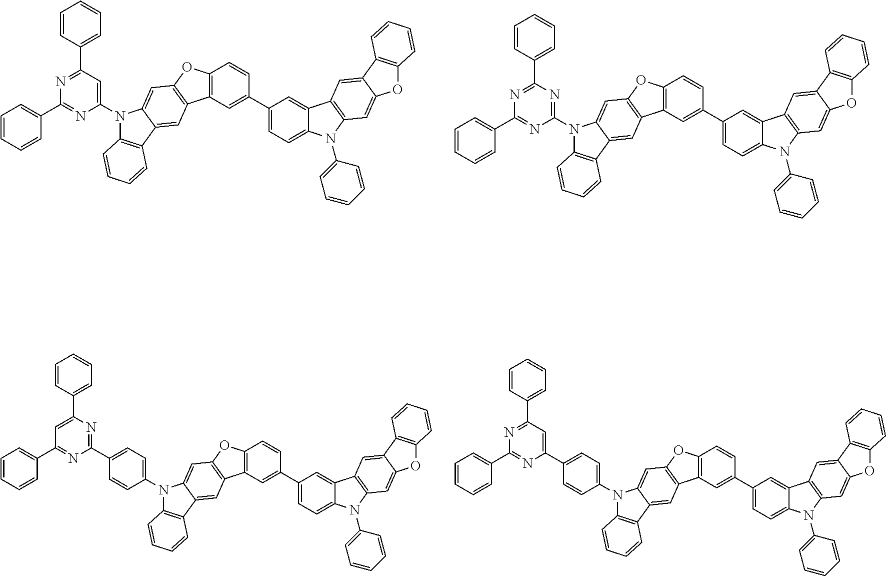 [SMILES: c1ccc(-c2cc(-n3c4ccccc4c4cc5c(cc43)oc3ccc(-c4ccc6c(c4)c4cc7c(cc4n6-c4ccccc4)oc4ccccc47)cc35)nc(-c3ccccc3)n2)cc1 ; c1ccc(-c2nc(-c3ccccc3)nc(-n3c4ccccc4c4cc5c(cc43)oc3ccc(-c4ccc6c(c4)c4cc7c(cc4n6-c4ccccc4)oc4ccccc47)cc35)n2)cc1 ; c1ccc(-c2cc(-c3ccccc3)nc(-c3ccc(-n4c5ccccc5c5cc6c(cc54)oc4ccc(-c5ccc7c(c5)c5cc8c(cc5n7-c5ccccc5)oc5ccccc58)cc46)cc3)n2)cc1 ; c1ccc(-c2cc(-c3ccc(-n4c5ccccc5c5cc6c(cc54)oc4ccc(-c5ccc7c(c5)c5cc8c(cc5n7-c5ccccc5)oc5ccccc58)cc46)cc3)nc(-c3ccccc3)n2)cc1]

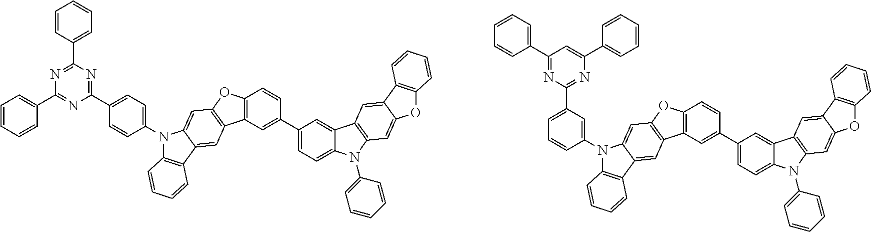 [SMILES: c1ccc(-c2nc(-c3ccccc3)nc(-c3ccc(-n4c5ccccc5c5cc6c(cc54)oc4ccc(-c5ccc7c(c5)c5cc8c(cc5n7-c5ccccc5)oc5ccccc58)cc46)cc3)n2)cc1 ; c1ccc(-c2cc(-c3ccccc3)nc(-c3cccc(-n4c5ccccc5c5cc6c(cc54)oc4ccc(-c5ccc7c(c5)c5cc8c(cc5n7-c5ccccc5)oc5ccccc58)cc46)c3)n2)cc1]

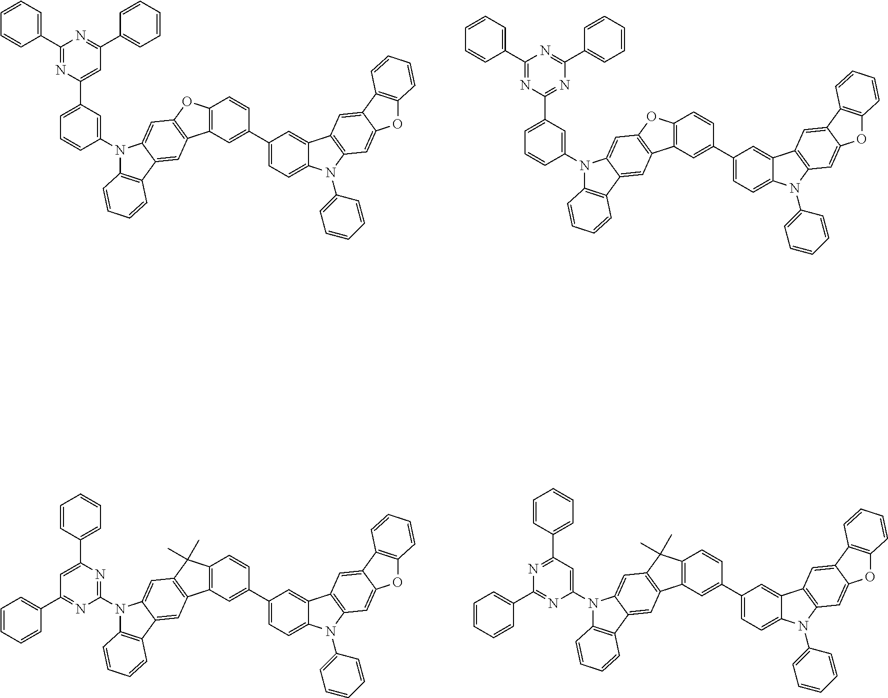 [SMILES: c1ccc(-c2cc(-c3cccc(-n4c5ccccc5c5cc6c(cc54)oc4ccc(-c5ccc7c(c5)c5cc8c(cc5n7-c5ccccc5)oc5ccccc58)cc46)c3)nc(-c3ccccc3)n2)cc1 ; c1ccc(-c2nc(-c3ccccc3)nc(-c3cccc(-n4c5ccccc5c5cc6c(cc54)oc4ccc(-c5ccc7c(c5)c5cc8c(cc5n7-c5ccccc5)oc5ccccc58)cc46)c3)n2)cc1 ; CC1(C)c2ccc(-c3ccc4c(c3)c3cc5c(cc3n4-c3ccccc3)oc3ccccc35)cc2-c2cc3c4ccccc4n(-c4nc(-c5ccccc5)cc(-c5ccccc5)n4)c3cc21 ; CC1(C)c2ccc(-c3ccc4c(c3)c3cc5c(cc3n4-c3ccccc3)oc3ccccc35)cc2-c2cc3c4ccccc4n(-c4cc(-c5ccccc5)nc(-c5ccccc5)n4)c3cc21]

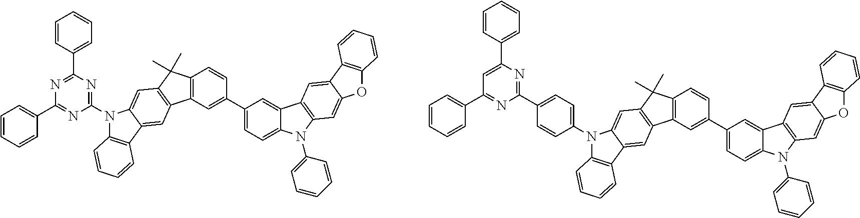 [SMILES: CC1(C)c2ccc(-c3ccc4c(c3)c3cc5c(cc3n4-c3ccccc3)oc3ccccc35)cc2-c2cc3c4ccccc4n(-c4nc(-c5ccccc5)nc(-c5ccccc5)n4)c3cc21 ; CC1(C)c2ccc(-c3ccc4c(c3)c3cc5c(cc3n4-c3ccccc3)oc3ccccc35)cc2-c2cc3c4ccccc4n(-c4ccc(-c5nc(-c6ccccc6)cc(-c6ccccc6)n5)cc4)c3cc21]

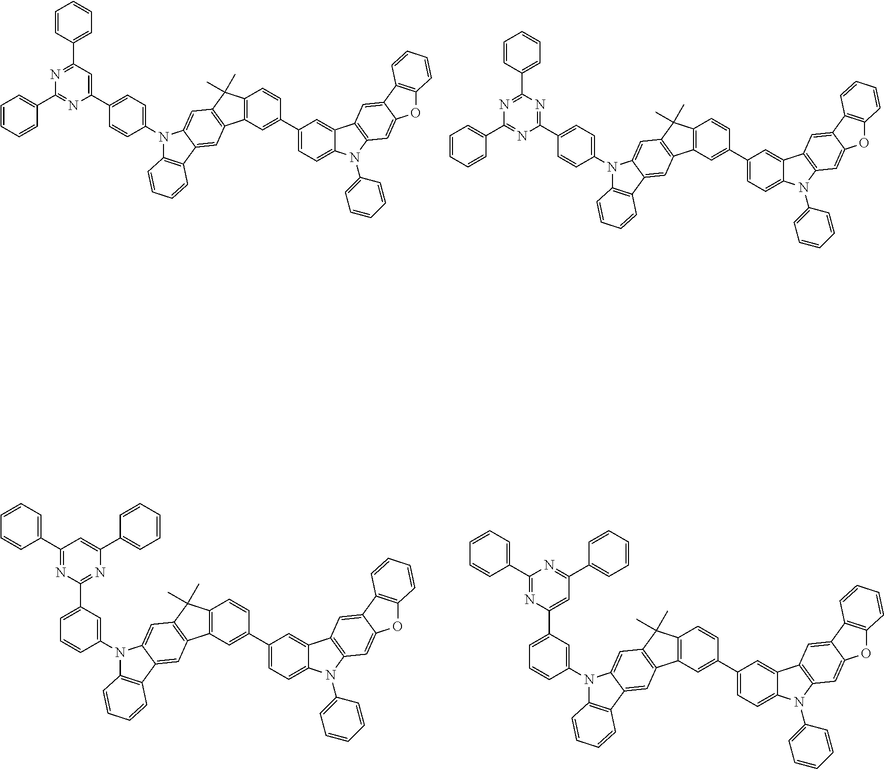

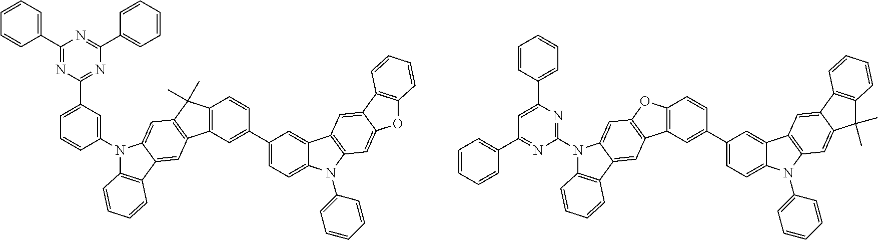 [SMILES: CC1(C)c2ccc(-c3ccc4c(c3)c3cc5c(cc3n4-c3ccccc3)oc3ccccc35)cc2-c2cc3c4ccccc4n(-c4cccc(-c5nc(-c6ccccc6)nc(-c6ccccc6)n5)c4)c3cc21 ; CC1(C)c2ccccc2-c2cc3c4cc(-c5ccc6oc7cc8c(cc7c6c5)c5ccccc5n8-c5nc(-c6ccccc6)cc(-c6ccccc6)n5)ccc4n(-c4ccccc4)c3cc21]

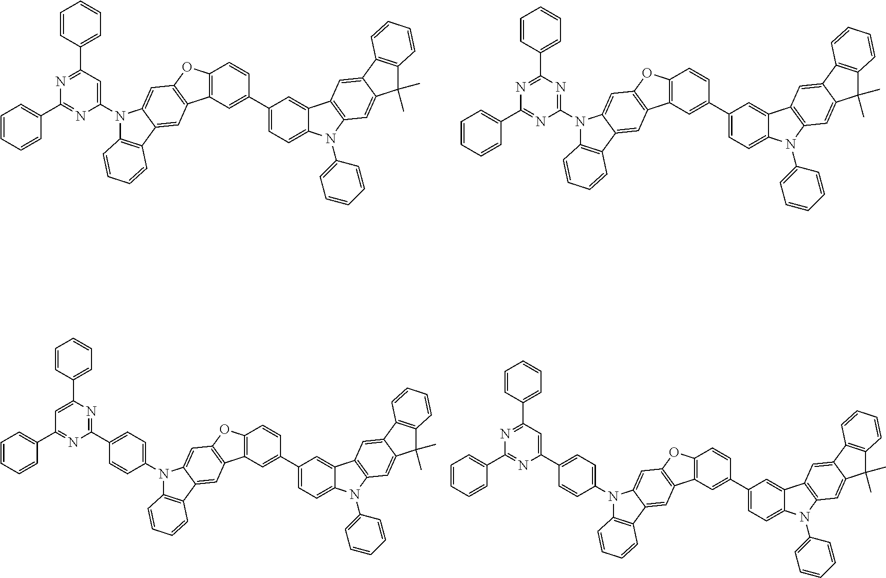

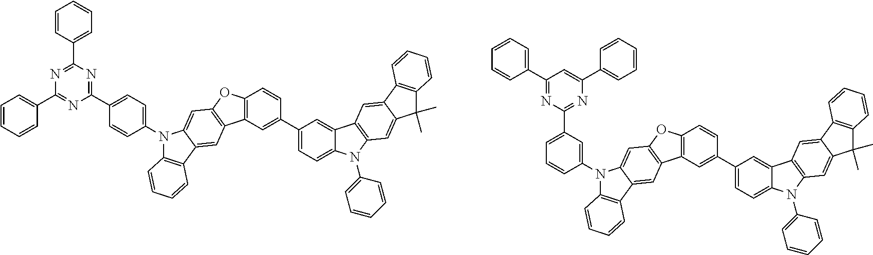 [SMILES: CC1(C)c2ccccc2-c2cc3c4cc(-c5ccc6oc7cc8c(cc7c6c5)c5ccccc5n8-c5ccc(-c6nc(-c7ccccc7)nc(-c7ccccc7)n6)cc5)ccc4n(-c4ccccc4)c3cc21 ; CC1(C)c2ccccc2-c2cc3c4cc(-c5ccc6oc7cc8c(cc7c6c5)c5ccccc5n8-c5cccc(-c6nc(-c7ccccc7)cc(-c7ccccc7)n6)c5)ccc4n(-c4ccccc4)c3cc21]

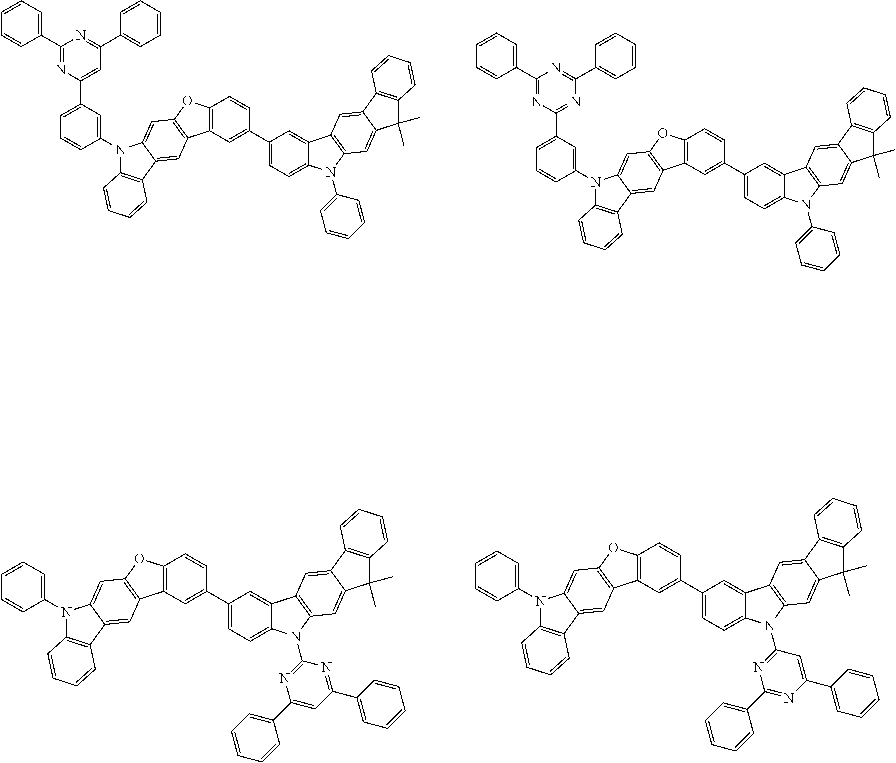 [SMILES: CC1(C)c2ccccc2-c2cc3c4cc(-c5ccc6oc7cc8c(cc7c6c5)c5ccccc5n8-c5cccc(-c6cc(-c7ccccc7)nc(-c7ccccc7)n6)c5)ccc4n(-c4ccccc4)c3cc21 ; CC1(C)c2ccccc2-c2cc3c4cc(-c5ccc6oc7cc8c(cc7c6c5)c5ccccc5n8-c5cccc(-c6nc(-c7ccccc7)nc(-c7ccccc7)n6)c5)ccc4n(-c4ccccc4)c3cc21 ; CC1(C)c2ccccc2-c2cc3c4cc(-c5ccc6oc7cc8c(cc7c6c5)c5ccccc5n8-c5ccccc5)ccc4n(-c4nc(-c5ccccc5)cc(-c5ccccc5)n4)c3cc21 ; CC1(C)c2ccccc2-c2cc3c4cc(-c5ccc6oc7cc8c(cc7c6c5)c5ccccc5n8-c5ccccc5)ccc4n(-c4cc(-c5ccccc5)nc(-c5ccccc5)n4)c3cc21]

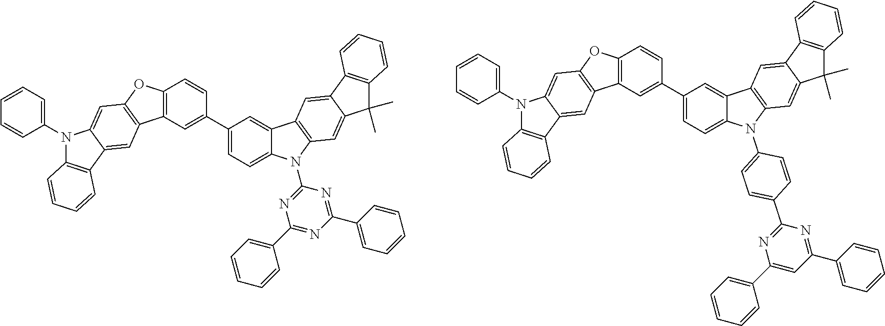 [SMILES: CC1(C)c2ccccc2-c2cc3c4cc(-c5ccc6oc7cc8c(cc7c6c5)c5ccccc5n8-c5ccccc5)ccc4n(-c4nc(-c5ccccc5)nc(-c5ccccc5)n4)c3cc21 ; CC1(C)c2ccccc2-c2cc3c4cc(-c5ccc6oc7cc8c(cc7c6c5)c5ccccc5n8-c5ccccc5)ccc4n(-c4ccc(-c5nc(-c6ccccc6)cc(-c6ccccc6)n5)cc4)c3cc21]

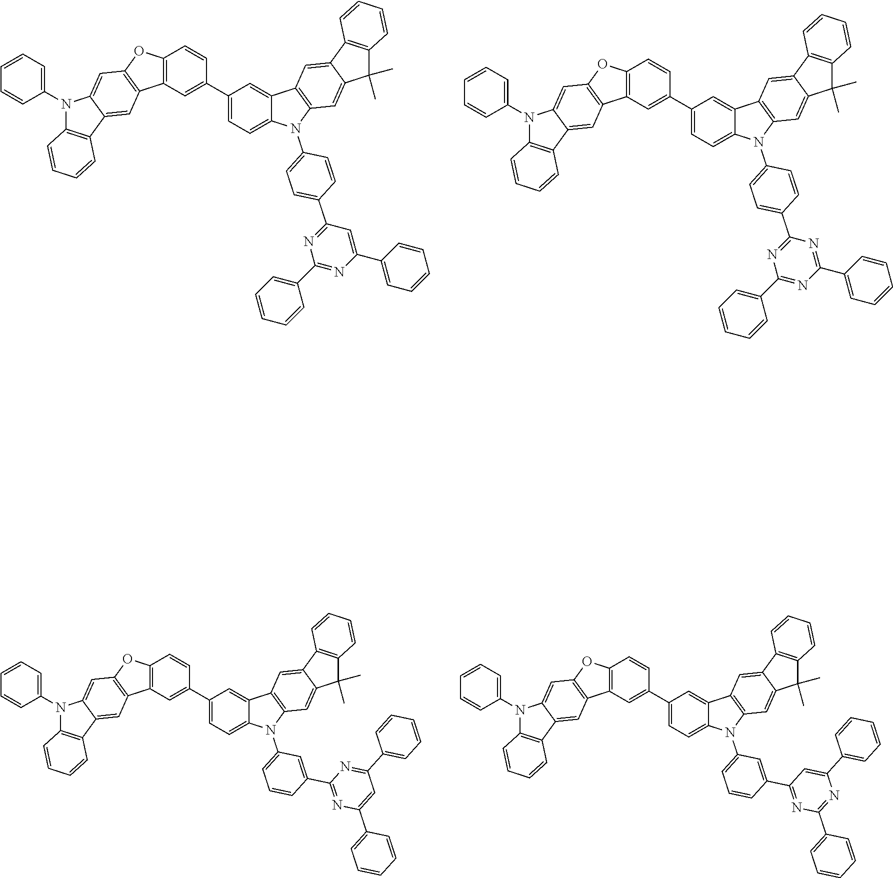

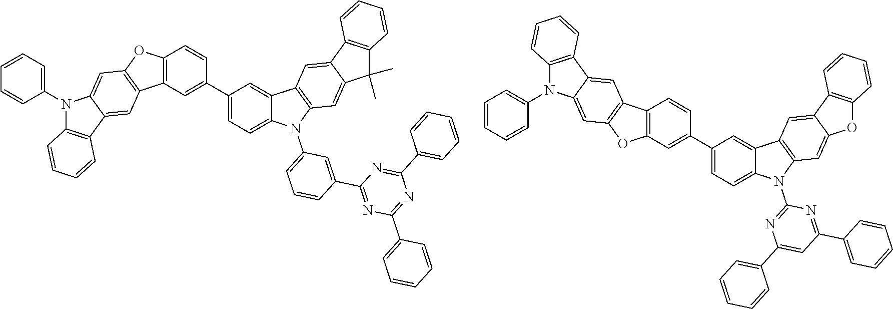

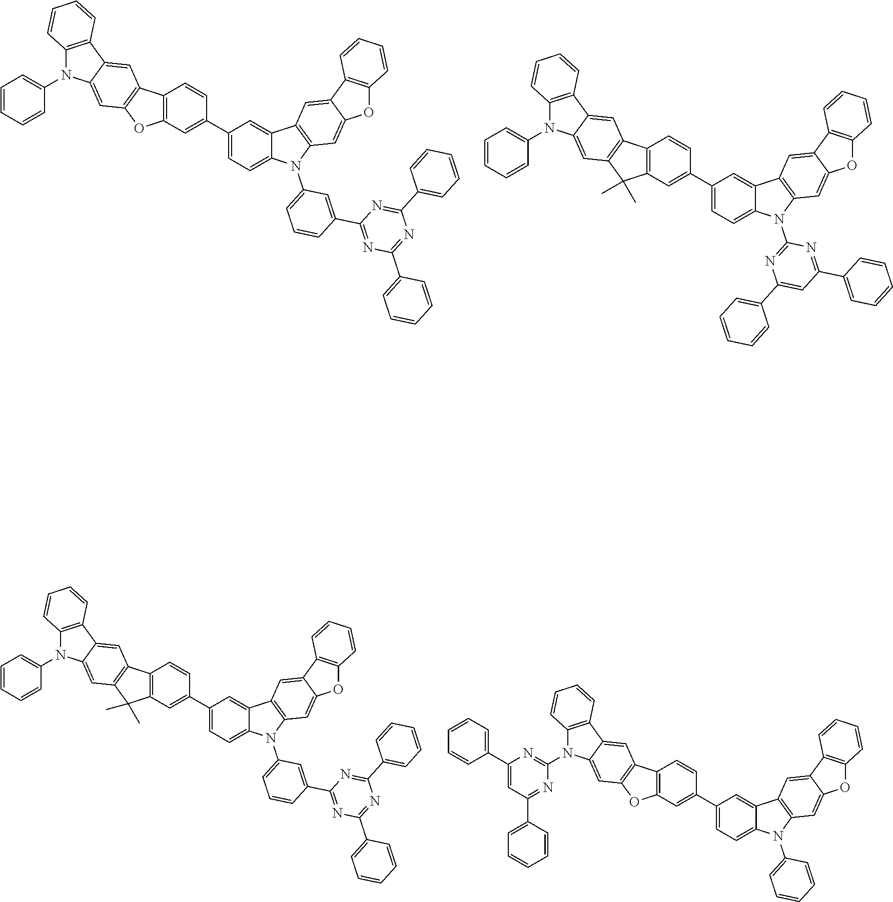

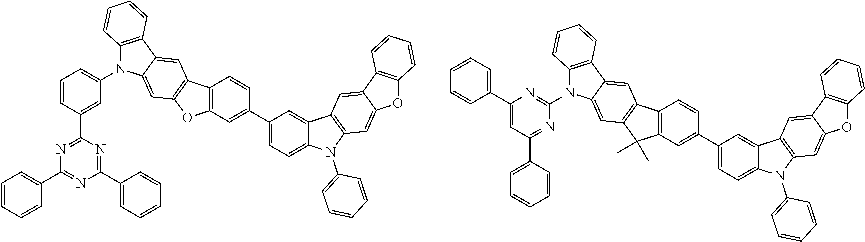 [SMILES: c1ccc(-c2nc(-c3ccccc3)nc(-c3cccc(-n4c5ccccc5c5cc6c(cc54)oc4cc(-c5ccc7c(c5)c5cc8c(cc5n7-c5ccccc5)oc5ccccc58)ccc46)c3)n2)cc1 ; CC1(C)c2cc(-c3ccc4c(c3)c3cc5c(cc3n4-c3ccccc3)oc3ccccc35)ccc2-c2cc3c4ccccc4n(-c4nc(-c5ccccc5)cc(-c5ccccc5)n4)c3cc21]

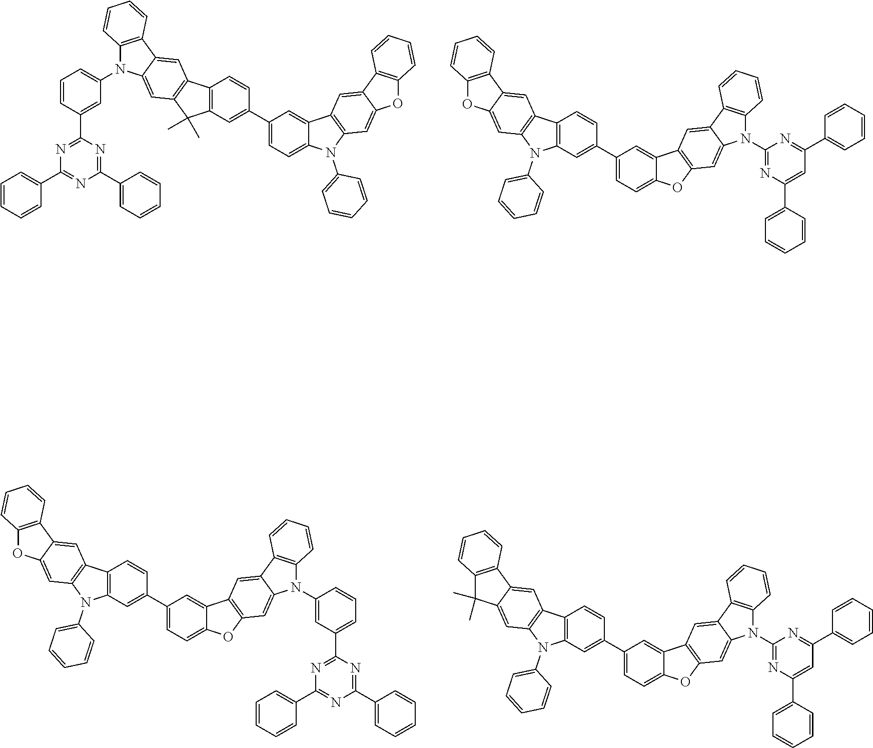 [SMILES: CC1(C)c2cc(-c3ccc4c(c3)c3cc5c(cc3n4-c3ccccc3)oc3ccccc35)ccc2-c2cc3c4ccccc4n(-c4cccc(-c5nc(-c6ccccc6)nc(-c6ccccc6)n5)c4)c3cc21 ; c1ccc(-c2cc(-c3ccccc3)nc(-n3c4ccccc4c4cc5c(cc43)oc3ccc(-c4ccc6c7cc8c(cc7n(-c7ccccc7)c6c4)oc4ccccc48)cc35)n2)cc1 ; c1ccc(-c2nc(-c3ccccc3)nc(-c3cccc(-n4c5ccccc5c5cc6c(cc54)oc4ccc(-c5ccc7c8cc9c(cc8n(-c8ccccc8)c7c5)oc5ccccc59)cc46)c3)n2)cc1 ; CC1(C)c2ccccc2-c2cc3c4ccc(-c5ccc6oc7cc8c(cc7c6c5)c5ccccc5n8-c5nc(-c6ccccc6)cc(-c6ccccc6)n5)cc4n(-c4ccccc4)c3cc21]

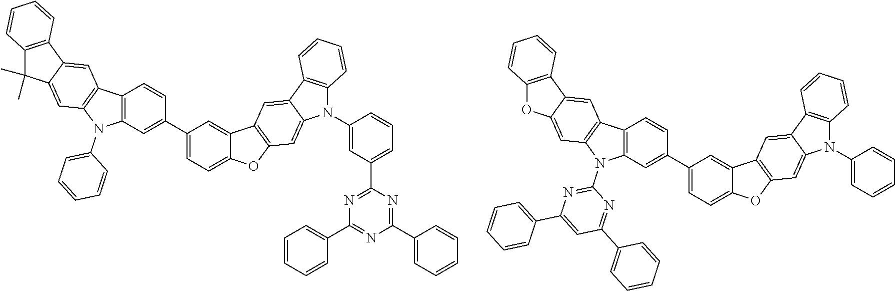

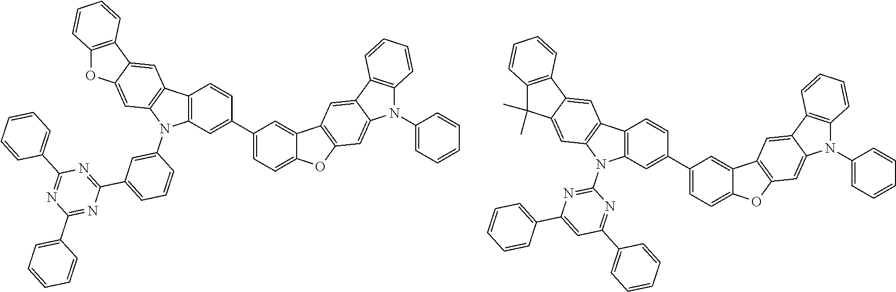 [SMILES: c1ccc(-c2nc(-c3ccccc3)nc(-c3cccc(-n4c5cc(-c6ccc7oc8cc9c(cc8c7c6)c6ccccc6n9-c6ccccc6)ccc5c5cc6c(cc54)oc4ccccc46)c3)n2)cc1 ; CC1(C)c2ccccc2-c2cc3c4ccc(-c5ccc6oc7cc8c(cc7c6c5)c5ccccc5n8-c5ccccc5)cc4n(-c4nc(-c5ccccc5)cc(-c5ccccc5)n4)c3cc21]

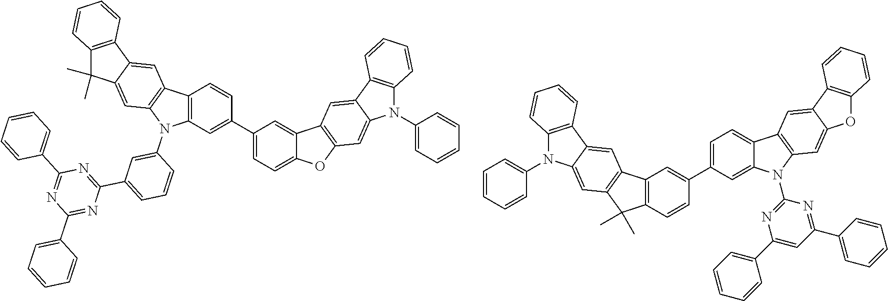

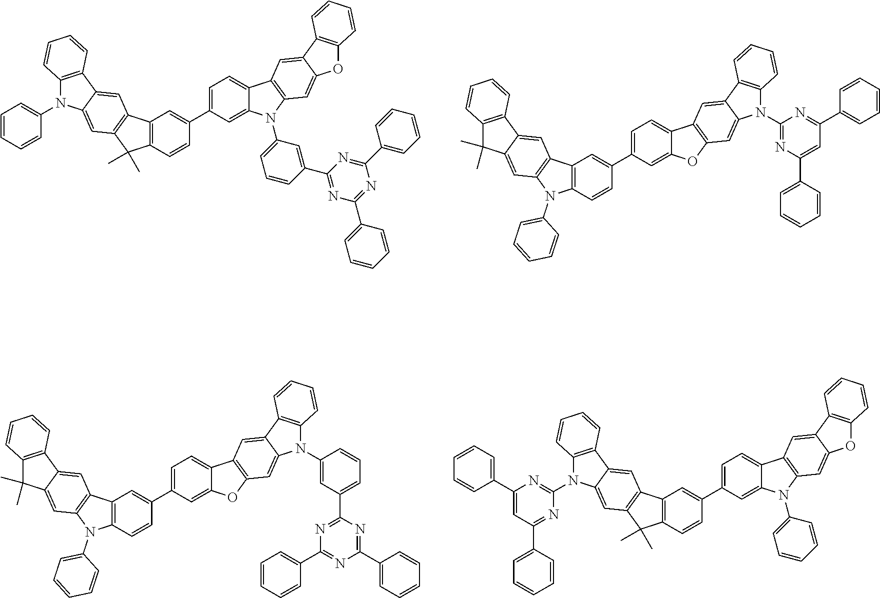 [SMILES: CC1(C)c2ccc(-c3ccc4c5cc6c(cc5n(-c5cccc(-c7nc(-c8ccccc8)nc(-c8ccccc8)n7)c5)c4c3)oc3ccccc36)cc2-c2cc3c4ccccc4n(-c4ccccc4)c3cc21 ; CC1(C)c2ccccc2-c2cc3c4cc(-c5ccc6c(c5)oc5cc7c(cc56)c5ccccc5n7-c5nc(-c6ccccc6)cc(-c6ccccc6)n5)ccc4n(-c4ccccc4)c3cc21 ; CC1(C)c2ccccc2-c2cc3c4cc(-c5ccc6c(c5)oc5cc7c(cc56)c5ccccc5n7-c5cccc(-c6nc(-c7ccccc7)nc(-c7ccccc7)n6)c5)ccc4n(-c4ccccc4)c3cc21 ; CC1(C)c2ccc(-c3ccc4c5cc6c(cc5n(-c5ccccc5)c4c3)oc3ccccc36)cc2-c2cc3c4ccccc4n(-c4nc(-c5ccccc5)cc(-c5ccccc5)n4)c3cc21]

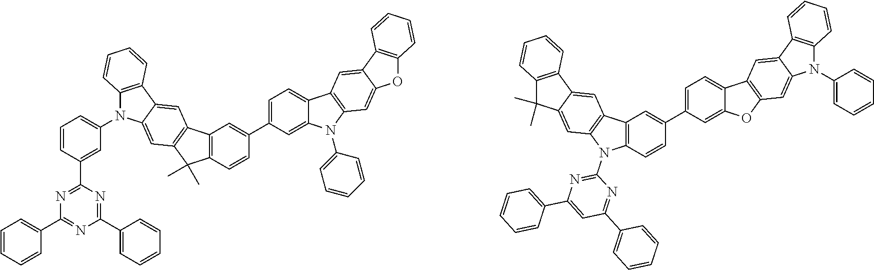 [SMILES: CC1(C)c2ccc(-c3ccc4c5cc6c(cc5n(-c5ccccc5)c4c3)oc3ccccc36)cc2-c2cc3c4ccccc4n(-c4cccc(-c5nc(-c6ccccc6)nc(-c6ccccc6)n5)c4)c3cc21 ; CC1(C)c2ccccc2-c2cc3c4cc(-c5ccc6c(c5)oc5cc7c(cc56)c5ccccc5n7-c5ccccc5)ccc4n(-c4nc(-c5ccccc5)cc(-c5ccccc5)n4)c3cc21]

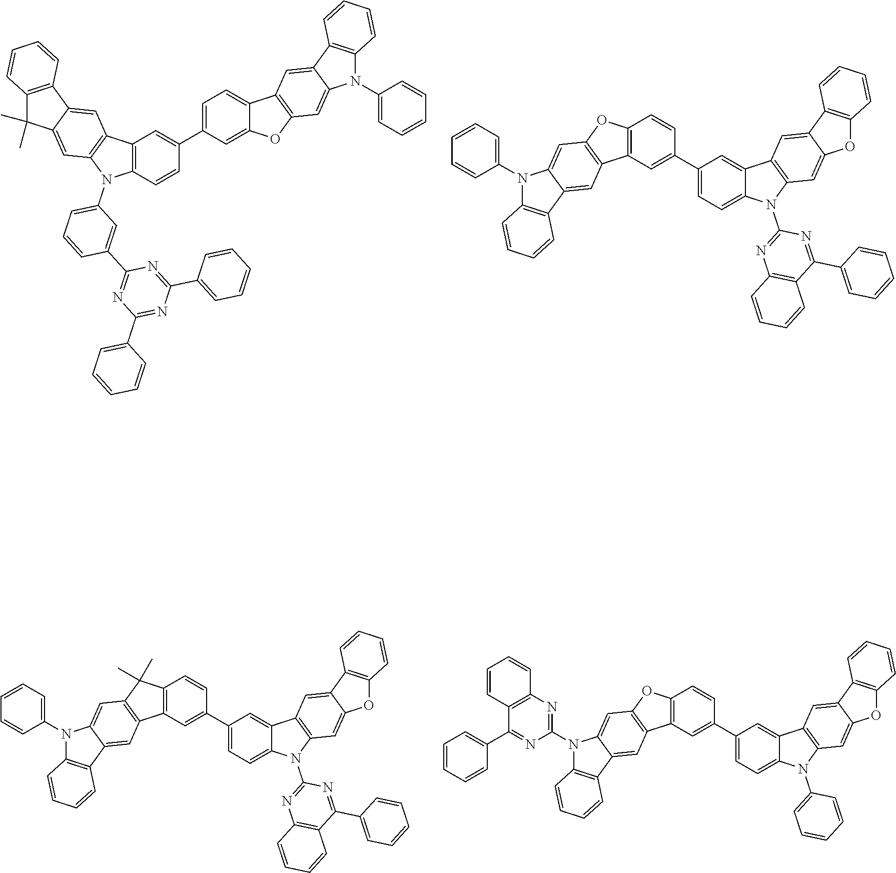

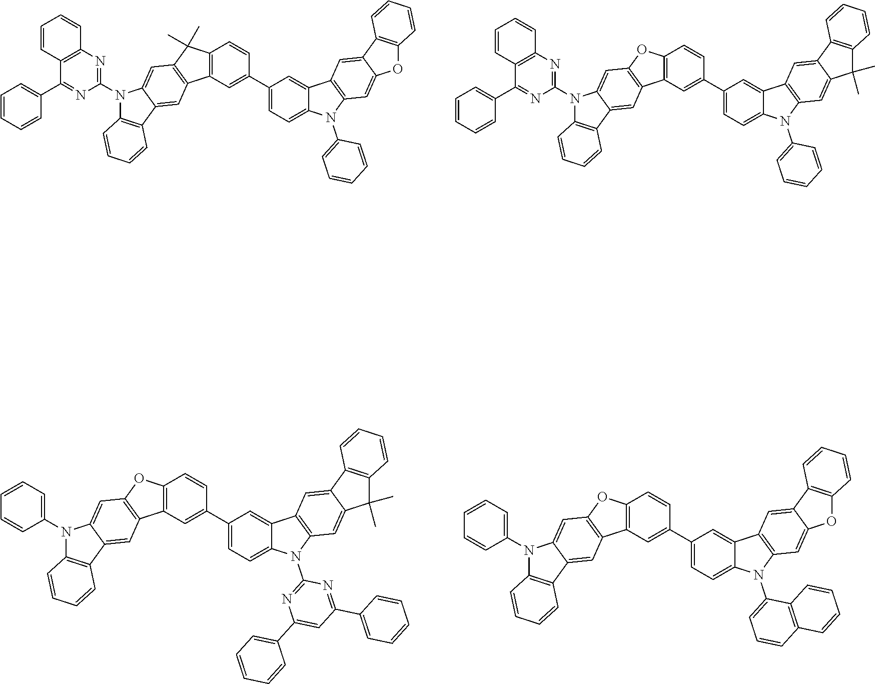 [SMILES: CC1(C)c2ccc(-c3ccc4c(c3)c3cc5c(cc3n4-c3ccccc3)oc3ccccc35)cc2-c2cc3c4ccccc4n(-c4nc(-c5ccccc5)c5ccccc5n4)c3cc21 ; CC1(C)c2ccccc2-c2cc3c4cc(-c5ccc6oc7cc8c(cc7c6c5)c5ccccc5n8-c5nc(-c6ccccc6)c6ccccc6n5)ccc4n(-c4ccccc4)c3cc21 ; CC1(C)c2ccccc2-c2cc3c4cc(-c5ccc6oc7cc8c(cc7c6c5)c5ccccc5n8-c5ccccc5)ccc4n(-c4nc(-c5ccccc5)cc(-c5ccccc5)n4)c3cc21 ; c1ccc(-n2c3ccccc3c3cc4c(cc32)oc2ccc(-c3ccc5c(c3)c3cc6c(cc3n5-c3cccc5ccccc35)oc3ccccc36)cc24)cc1]

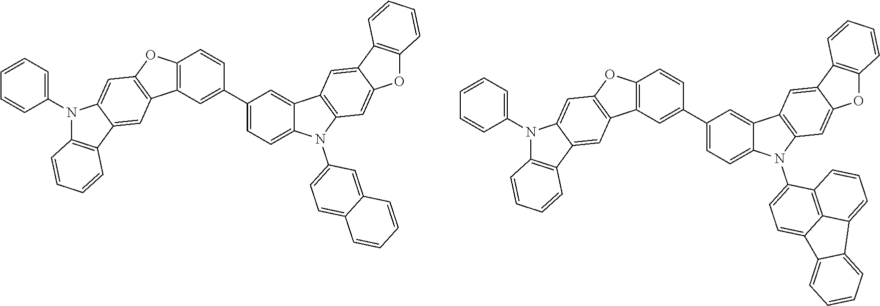 [SMILES: c1ccc(-n2c3ccccc3c3cc4c(cc32)oc2ccc(-c3ccc5c(c3)c3cc6c(cc3n5-c3ccc5ccccc5c3)oc3ccccc36)cc24)cc1 ; c1ccc(-n2c3ccccc3c3cc4c(cc32)oc2ccc(-c3ccc5c(c3)c3cc6c(cc3n5-c3ccc5c7c(cccc37)-c3ccccc3-5)oc3ccccc36)cc24)cc1]

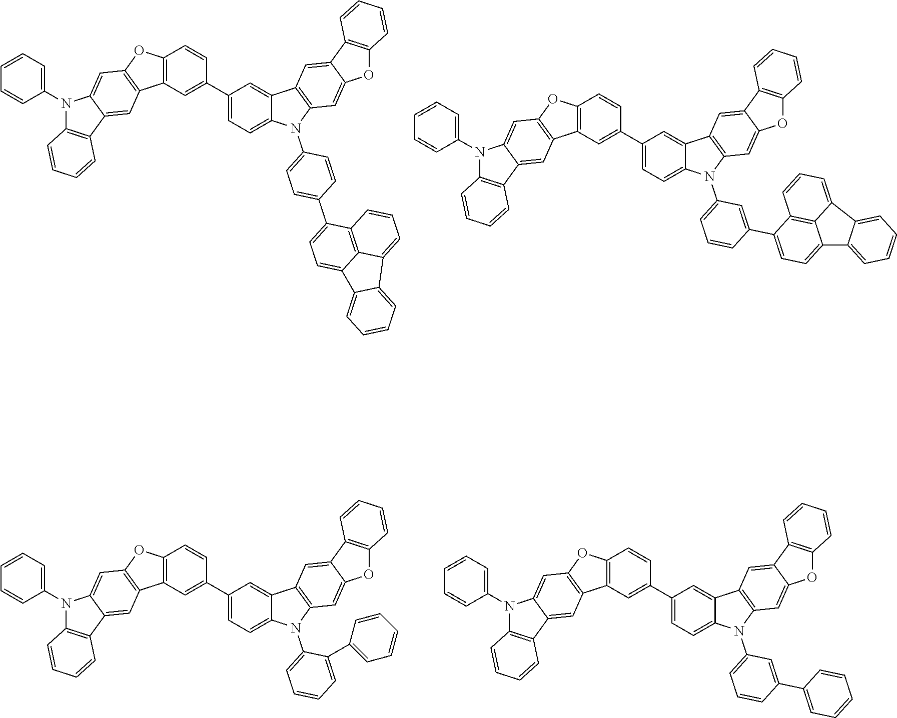 [SMILES: c1ccc(-n2c3ccccc3c3cc4c(cc32)oc2ccc(-c3ccc5c(c3)c3cc6c(cc3n5-c3ccc(-c5ccc7c8c(cccc58)-c5ccccc5-7)cc3)oc3ccccc36)cc24)cc1 ; c1ccc(-n2c3ccccc3c3cc4c(cc32)oc2ccc(-c3ccc5c(c3)c3cc6c(cc3n5-c3cccc(-c5ccc7c8c(cccc58)-c5ccccc5-7)c3)oc3ccccc36)cc24)cc1 ; c1ccc(-c2ccccc2-n2c3ccc(-c4ccc5oc6cc7c(cc6c5c4)c4ccccc4n7-c4ccccc4)cc3c3cc4c(cc32)oc2ccccc24)cc1 ; c1ccc(-c2cccc(-n3c4ccc(-c5ccc6oc7cc8c(cc7c6c5)c5ccccc5n8-c5ccccc5)cc4c4cc5c(cc43)oc3ccccc35)c2)cc1]

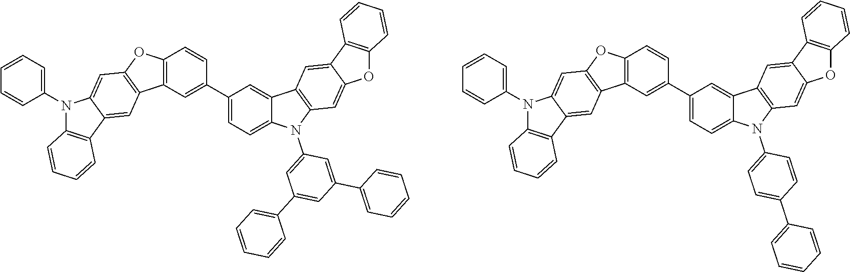 [SMILES: c1ccc(-c2cc(-c3ccccc3)cc(-n3c4ccc(-c5ccc6oc7cc8c(cc7c6c5)c5ccccc5n8-c5ccccc5)cc4c4cc5c(cc43)oc3ccccc35)c2)cc1 ; c1ccc(-c2ccc(-n3c4ccc(-c5ccc6oc7cc8c(cc7c6c5)c5ccccc5n8-c5ccccc5)cc4c4cc5c(cc43)oc3ccccc35)cc2)cc1]

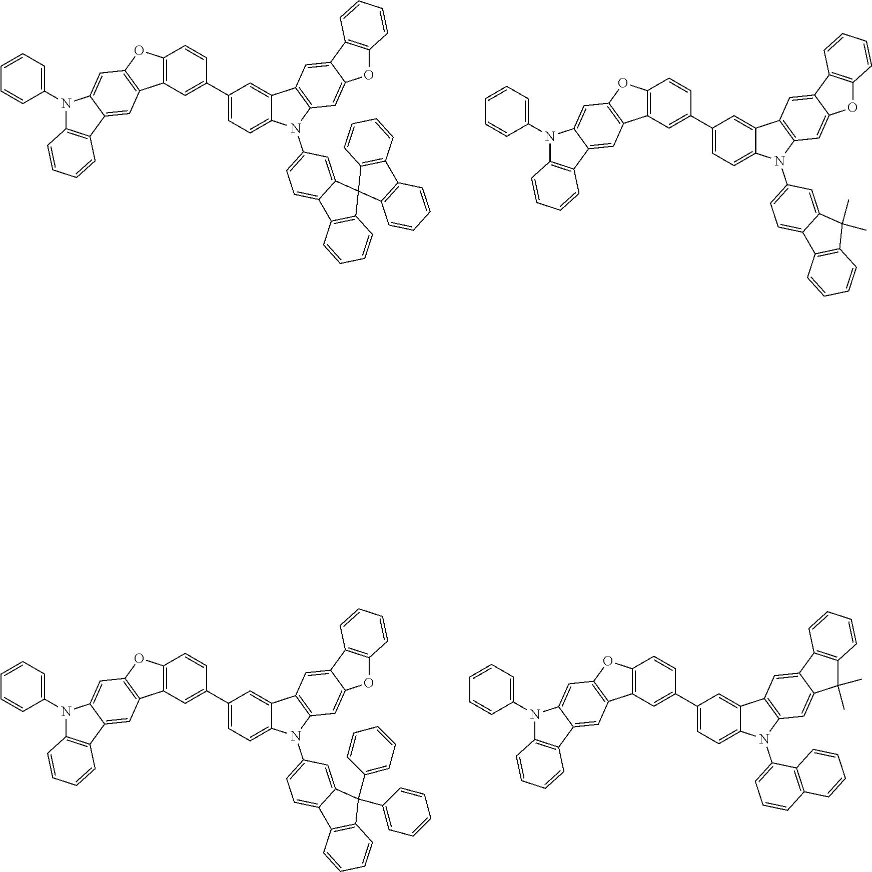 [SMILES: c1ccc(-n2c3ccccc3c3cc4c(cc32)oc2ccc(-c3ccc5c(c3)c3cc6c(cc3n5-c3ccc5c(c3)C3(c7ccccc7-c7ccccc73)c3ccccc3-5)oc3ccccc36)cc24)cc1 ; CC1(C)c2ccccc2-c2ccc(-n3c4ccc(-c5ccc6oc7cc8c(cc7c6c5)c5ccccc5n8-c5ccccc5)cc4c4cc5c(cc43)oc3ccccc35)cc21 ; c1ccc(-n2c3ccccc3c3cc4c(cc32)oc2ccc(-c3ccc5c(c3)c3cc6c(cc3n5-c3ccc5c(c3)C(c3ccccc3)(c3ccccc3)c3ccccc3-5)oc3ccccc36)cc24)cc1 ; CC1(C)c2ccccc2-c2cc3c4cc(-c5ccc6oc7cc8c(cc7c6c5)c5ccccc5n8-c5ccccc5)ccc4n(-c4cccc5ccccc45)c3cc21]

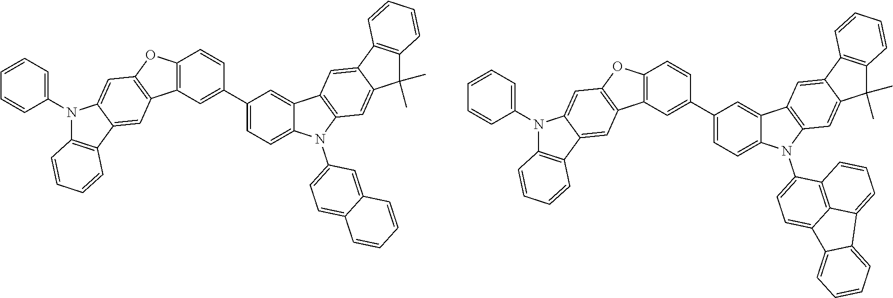 [SMILES: CC1(C)c2ccccc2-c2cc3c4cc(-c5ccc6oc7cc8c(cc7c6c5)c5ccccc5n8-c5ccccc5)ccc4n(-c4ccc5ccccc5c4)c3cc21 ; CC1(C)c2ccccc2-c2cc3c4cc(-c5ccc6oc7cc8c(cc7c6c5)c5ccccc5n8-c5ccccc5)ccc4n(-c4ccc5c6c(cccc46)-c4ccccc4-5)c3cc21]

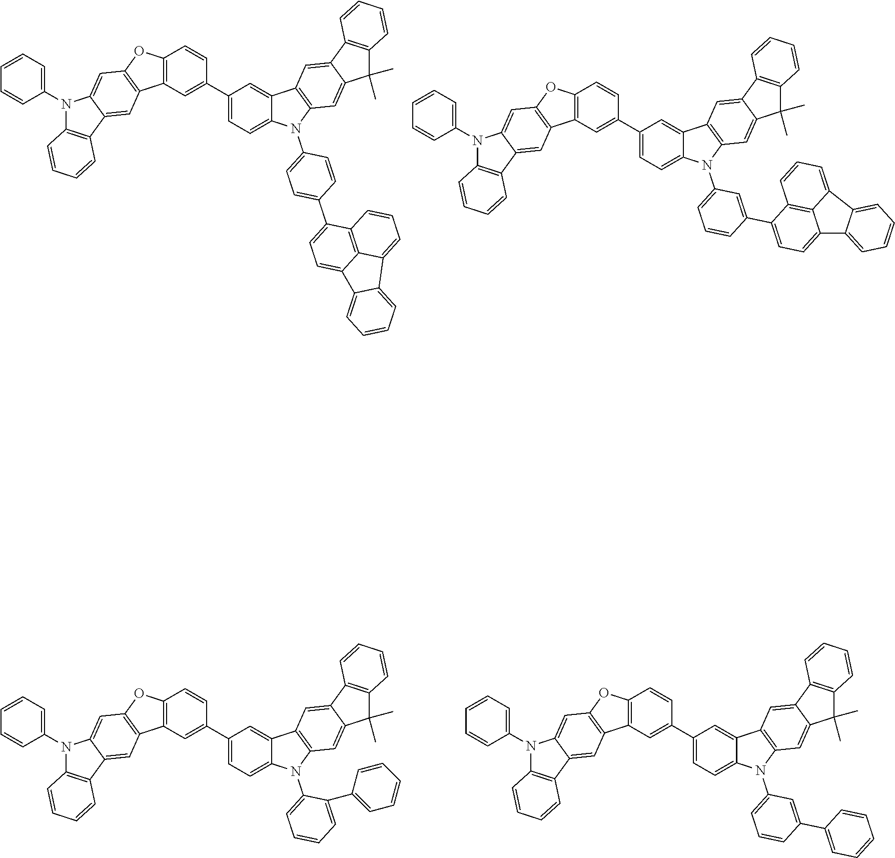 [SMILES: CC1(C)c2ccccc2-c2cc3c4cc(-c5ccc6oc7cc8c(cc7c6c5)c5ccccc5n8-c5ccccc5)ccc4n(-c4ccc(-c5ccc6c7c(cccc57)-c5ccccc5-6)cc4)c3cc21 ; CC1(C)c2ccccc2-c2cc3c4cc(-c5ccc6oc7cc8c(cc7c6c5)c5ccccc5n8-c5ccccc5)ccc4n(-c4cccc(-c5ccc6c7c(cccc57)-c5ccccc5-6)c4)c3cc21 ; CC1(C)c2ccccc2-c2cc3c4cc(-c5ccc6oc7cc8c(cc7c6c5)c5ccccc5n8-c5ccccc5)ccc4n(-c4ccccc4-c4ccccc4)c3cc21 ; CC1(C)c2ccccc2-c2cc3c4cc(-c5ccc6oc7cc8c(cc7c6c5)c5ccccc5n8-c5ccccc5)ccc4n(-c4cccc(-c5ccccc5)c4)c3cc21]

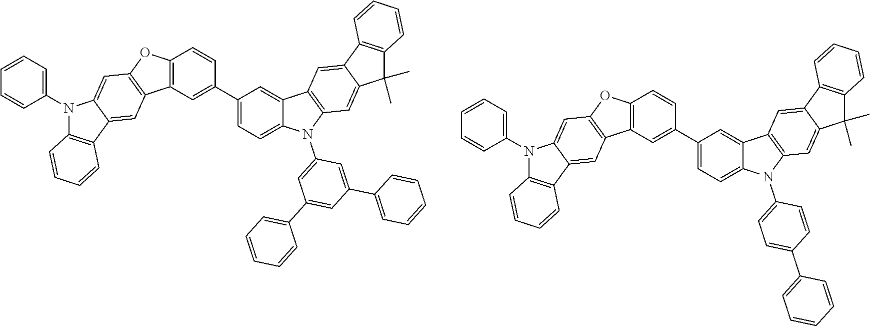 [SMILES: CC1(C)c2ccccc2-c2cc3c4cc(-c5ccc6oc7cc8c(cc7c6c5)c5ccccc5n8-c5ccccc5)ccc4n(-c4cc(-c5ccccc5)cc(-c5ccccc5)c4)c3cc21 ; CC1(C)c2ccccc2-c2cc3c4cc(-c5ccc6oc7cc8c(cc7c6c5)c5ccccc5n8-c5ccccc5)ccc4n(-c4ccc(-c5ccccc5)cc4)c3cc21]

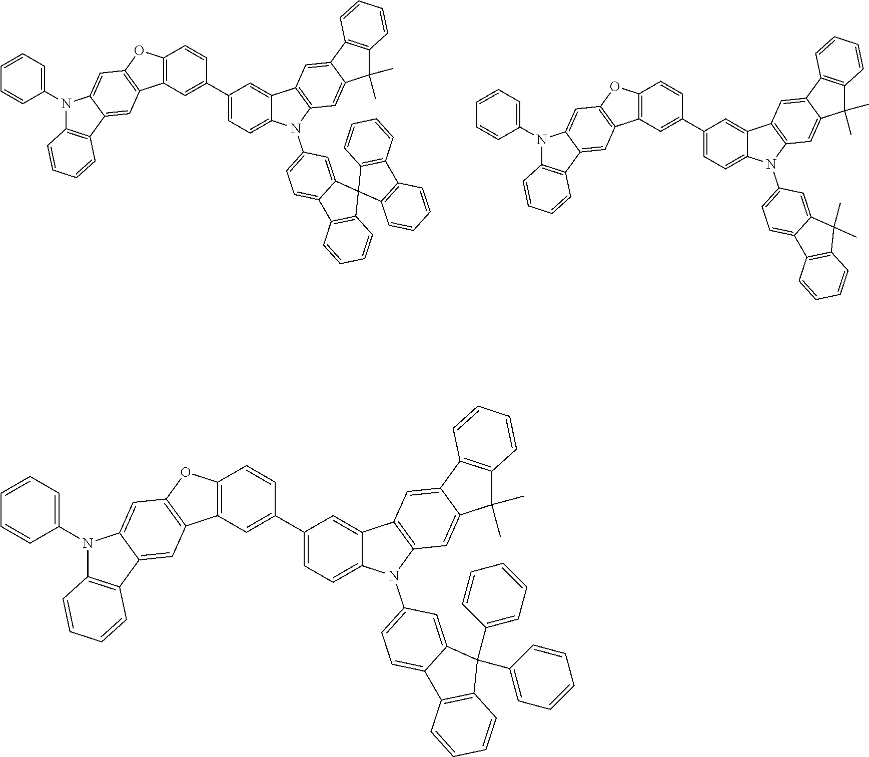 [SMILES: CC1(C)c2ccccc2-c2cc3c4cc(-c5ccc6oc7cc8c(cc7c6c5)c5ccccc5n8-c5ccccc5)ccc4n(-c4ccc5c(c4)C4(c6ccccc6-c6ccccc64)c4ccccc4-5)c3cc21 ; CC1(C)c2ccccc2-c2ccc(-n3c4ccc(-c5ccc6oc7cc8c(cc7c6c5)c5ccccc5n8-c5ccccc5)cc4c4cc5c(cc43)C(C)(C)c3ccccc3-5)cc21 ; CC1(C)c2ccccc2-c2cc3c4cc(-c5ccc6oc7cc8c(cc7c6c5)c5ccccc5n8-c5ccccc5)ccc4n(-c4ccc5c(c4)C(c4ccccc4)(c4ccccc4)c4ccccc4-5)c3cc21]

### Material for Organic EL Device

The material for organic EL device in an aspect of the invention comprises the compound (1) mentioned above. The content of the compound (1) is preferably 50% by mass or more, more preferably 70% by mass or more, still more preferably 80% by mass or more, and particularly preferably 90% by mass or more. The material for organic EL device may contain the compound (1) solely. The preferred compounds thereof are as described above. The following description made with respect to the compound (1) is equally applicable to the preferred compounds.

The material for organic EL device in an aspect of the invention is useful as a material for producing an organic EL device, for example, as a material for at least one organic thin film layer disposed between an anode and a cathode, particularly as a material for a hole transporting layer, a material of a hole injecting layer, or a host material (particularly a phosphorescent host material) for a light emitting layer.

### Organic EL Device

The organic EL device in an aspect of the invention will be described below.

Representative device structures (1) to (13) are shown below, although not limited thereto. The device structure (8) is preferably used.

(1) anode/light emitting layer/cathode;
(2) anode/hole injecting layer/light emitting layer/cathode;
(3) anode/light emitting layer/electron injecting layer/cathode;
(4) anode/hole injecting layer/light emitting layer/electron injecting layer/cathode;
(5) anode/organic semiconductor layer/light emitting layer/cathode;
(6) anode/organic semiconductor layer/electron blocking layer/light emitting layer/cathode;
(7) anode/organic semiconductor layer/light emitting layer/adhesion improving layer/cathode;
(8) anode/(hole injecting layer/)hole transporting layer/light emitting layer/electron transporting layer/electron injecting layer/cathode;
(9) anode/insulating layer/light emitting layer/insulating layer/cathode;
(10) anode/inorganic semiconductor layer/insulating layer/light emitting layer/insulating layer/cathode;
(11) anode/organic semiconductor layer/insulating layer/light emitting layer/insulating layer/cathode;
(12) anode/insulating layer/hole injecting layer/hole transporting layer/light emitting layer/insulating layer/cathode; and
(13) anode/insulating layer/hole injecting layer/hole transporting layer/light emitting layer/electron transporting layer/electron injecting layer/cathode.

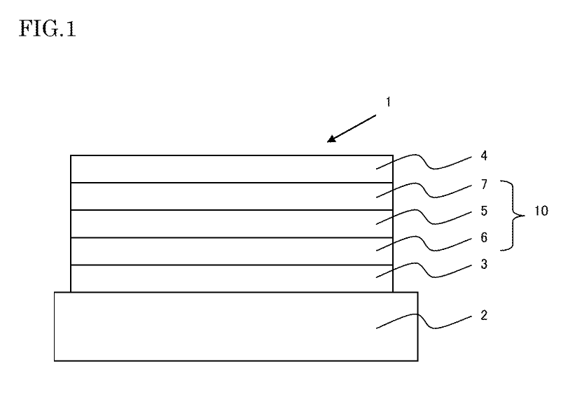

A schematic structure of an example of the organic EL device in an aspect of the invention is shown in FIG. 1, wherein the organic EL device 1 comprises a substrate 2, an anode 3, a cathode 4, and an emission unit 10 disposed between the anode 3 and the cathode 4. The emission unit 10 comprises a light emitting layer 5 which comprises a host material and a dopant (light emitting material). A hole injecting/transporting layer (anode-side organic thin film layer) 6, etc. may be disposed between the light emitting layer 5 and the anode 3, and an electron injecting/transporting layer (cathode-side organic thin film layer) 7, etc. may be disposed between the light emitting layer 5 and the cathode 4. An electron blocking layer may be disposed on the anode 3 side of the light emitting layer 5, and a hole blocking layer may be disposed on the cathode 4 side of the light emitting layer 5. With these blocking layers, electrons and holes are confined in the light emitting layer 5 to increase the exciton generation in the light emitting layer 5.

The organic EL device in an aspect of the invention comprises an organic thin film layer between a cathode and an anode, wherein the organic thin film layer comprises one or more layers comprising a light emitting layer and at least one layer of the organic thin film layer comprises the compound represented by formula (1) (compound (1)).

Examples of the organic thin film layer comprising the compound (1) include an anode-side organic thin film layer formed between an anode and a light emitting layer (hole transporting layer, hole injecting layer, etc.), a light emitting layer, a cathode-side organic thin film layer formed between a cathode and a light emitting layer (electron transporting layer, electron injecting layer, etc.), a space layer, and a blocking layer, although not limited thereto.

The compound (1) may be used in any layer of the organic thin film layer of an organic EL device. In view of the emission efficiency and the lifetime, the compound (1) is preferably used in a hole injecting layer, a hole transporting layer, or a light emitting layer.

In an embodiment of the invention, an organic EL device wherein a light emitting layer comprises the compound (1) is more preferred. In such an organic EL device, the light emitting layer preferably comprises a phosphorescent emitting material to be described below.

In an embodiment of the invention, an organic EL device wherein the organic thin film layer comprises at least one of a hole injecting layer and a hole transporting layer each of which comprises the compound (1) is more preferred. In such an organic EL device, the light emitting layer preferably comprises a fluorescent emitting material to be described below.

The content of the compound (1) in the organic thin film layer, preferably in a hole injecting layer or a hole transporting layer, is preferably 30 to 100 mol %, more preferably 50 to 100 mol %, still more preferably 80 to 100 mol %, and further more preferably 95 to 100 mol % each based on the total molar amount of the components in the organic thin film layer.

### Substrate

The substrate is a support for the emitting device and made of, for example, glass, quartz, and plastics. The substrate may be a flexible substrate, for example, a plastic substrate made of, for example, polycarbonate, polyarylate, polyether sulfone, polypropylene, polyester, polyvinyl fluoride, and polyvinyl chloride. An inorganic deposition film is also usable.

### Anode

The anode is formed on the substrate preferably from a metal, an alloy, an electrically conductive compound, and a mixture thereof, each having a large work function, for example, 4.5 eV or more. Examples of the material for the anode include indium oxide-tin oxide (ITO: indium tin oxide), indium oxide-tin oxide doped with silicon or silicon oxide, indium oxide-zinc oxide, indium oxide doped with tungsten oxide and zinc oxide, and graphene. In addition, gold (Au), platinum (Pt), nickel (Ni), tungsten (W), chromium (Cr), molybdenum (Mo), iron (Fe), cobalt (Co), copper (Cu), palladium (Pd), titanium (Ti), and a metal nitride (for example, titanium nitride) are also usable.

These materials are made into a film generally by a sputtering method. For example, a film of indium oxide-zinc oxide is formed by sputtering an indium oxide target doped with 1 to 10% by mass of zinc oxide, and a film of indium oxide doped with tungsten oxide and zinc oxide is formed by sputtering an indium oxide target doped with 0.5 to 5% by mass of tungsten oxide and 0.1 to 1% by mass of zinc oxide. In addition, a vacuum vapor deposition method, a coating method, an inkjet method, and a spin coating method are usable.

A hole injecting layer to be formed in contact with the anode is formed from a composite material which is capable of easily injecting holes independently of the work function of the anode. Therefore a material, for example, a metal, an alloy, an electroconductive compound, a mixture thereof, and a group 1 element and a group 2 element of the periodic table are usable as the electrode material.

A material having a small work function, for example, the group 1 element and the group 2 element of the periodic table, i.e., an alkali metal, such as lithium (Li) and cesium (Cs), an alkaline earth metal, such as magnesium (Mg), calcium (Ca), and strontium (Sr), and an alloy thereof, such as MgAg and AlLi, are also usable. In addition, a rare earth metal, such as europium (Eu) and ytterbium (Yb), and an alloy thereof are also usable. The alkali metal, the alkaline earth metal, and the alloy thereof can be made into the anode by a vacuum vapor deposition or a sputtering method. When a silver paste, etc. is used, a coating method and an inkjet method are usable.

### Hole Injecting Layer

The hole injecting layer comprises a highly hole-transporting material.

The hole injecting layer of the organic EL device in an aspect of the invention preferably contains the compound (1) solely or in combination with the compound mentioned below.

Examples of the highly hole-transporting material include molybdenum oxide, titanium oxide, vanadium oxide, rhenium oxide, ruthenium oxide, chromium oxide, zirconium oxide, hafnium oxide, tantalum oxide, silver oxide, tungsten oxide, and manganese oxide.

The following low molecular aromatic amine compound is also usable: 4,4′,4″-tris(N,N-diphenylamino)triphenylamine (TDATA), 4,4′,4″-tris[N-(3-methylphenyl)-N-phenylamino]triphenylamine (MTDATA), 4,4′-bis[N-(4-diphenylaminophenyl)-N-phenylamino]biphenyl (DPAB), 4,4′-bis(N-{4-[N′-(3-methylphenyl)-N′-phenylamino]phenyl}-N-phenylamino)biphenyl (DNTPD), 1,3,5-tris[N-(4-diphenylaminophenyl)-N-phenylamino]benzene (DPA3B), 3-[N-(9-phenylcarbazole-3-yl)-N-phenylamino]-9-phenylcarbazole (PCzPCA1), 3,6-bis[N-(9-phenylcarbazole-3-yl)-N-phenylamino]-9-phenylcarbazole (PCzPCA2), and 3-[N-(1-naphthyl)-N-(9-phenylcarbazole-3-yl)amino]-9-phenylcarbazole (PCzPCN1).

A polymeric compound, such as an oligomer, a dendrimer, a polymer, is also usable. Examples thereof include poly(N-vinylcarbazole) (PVK), poly(4-vinyltriphenylamine) (PVTPA), poly[N-(4-{N′-[4-(4-diphenylamino)phenyl]phenyl-N′-phenylamino}phenyl)methacrylamide] (PTPDMA), and poly[N,N′-bis(4-butylphenyl)-N,N′-bis(phenyl)benzidine] (Poly-TPD). An acid-added polymeric compound, such as poly(3,4-ethylenedioxythiophene)/poly(styrenesulfonic acid) (PEDOT/PSS) and polyalinine/poly(styrenesulfonic acid) (PAni/PSS), is also usable.

### Hole Transporting Layer

The hole transporting layer comprises a highly hole-transporting material.

The hole transporting layer of the organic EL device in an aspect of the invention may contain the compound (1) solely or in combination with the compound mentioned below.

The hole transporting layer may comprise an aromatic amine compound, a carbazole derivative, an anthracene derivative, etc. Examples the aromatic amine compound include 4,4′-bis[N-(1-naphthyl)-N-phenylamino]biphenyl (NPB), N,N′-bis(3-methylphenyl)-N,N′-diphenyl-[1,1′-biphenyl]-4,4′-diamine (TPD), 4-phenyl-4′-(9-phenylfluorene-9-yl)triphenylamine (BAFLP), 4,4′-bis[N-(9,9-dimethylfluorene-2-yl)-N-phenylamino]biphenyl (DFLDPBi), 4,4′,4″-tris(N,N-diphenylamino)triphenylamine (TDATA), 4,4′,4″-tris[N-(3-methylphenyl)-N-phenylamino]triphenylamine (MTDATA), and 4,4′-bis[N-(spiro-9,9′-bifluorene-2-yl)-N-phenylamino]biphenyl (BSPB). The above compounds have a hole mobility of mainly 10⁻⁶ cm²/Vs or more.

In addition, the hole transporting layer may contain a carbazole derivative, such as CBP, CzPA, and PCzPA, an anthracene derivative, such as t-BuDNA, DNA, and DPAnth, and a polymeric compound, such as poly(N-vinylcarbazole) (PVK) and poly(4-vinyltriphenylamine) (PVTPA).

Other materials are also usable if their hole transporting ability is higher than their electron transporting ability.

The layer comprising a highly hole-transporting material may be a single layer or a laminate of two or more layers each comprising the material mentioned above. For example, the hole transporting layer may be made into a two-layered structure of a first hole transporting layer (anode side) and a second hole transporting layer (light emitting layer side). In such a two-layered structure, the compound (1) may be used in either of the first hole transporting layer and the second hole transporting layer.

In the organic EL device in an aspect of the invention, a layer (acceptor layer) comprising an electron-accepting compound (acceptor material) may be formed on the anode-side of the hole transporting layer or the first hole transporting layer, because it is expected that the driving voltage is lowered and the production cost is reduced.

A compound represented by formula (A) or (B) is preferably used as the acceptor compound:

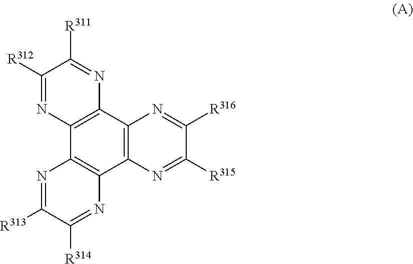

wherein R³¹¹ to R³¹⁶ may be the same or different and each independently represent a cyano group, —CONH₂, a carboxyl group, or —COOR³¹⁷ wherein R³¹⁷ represents an alkyl group having 1 to 20 carbon atoms or a cycloalkyl group having 3 to 20 carbon atoms; and one or more selected from R³¹¹ and R³¹², R³¹³ and R³¹⁴, or R³¹⁵ and R³¹⁶ may be bonded to each other to form a group represented by —CO—O—CO—.

R³¹⁷ is a methyl group, an ethyl group, a n-propyl group, an isopropyl group, a n-butyl group, an isobutyl group, a t-butyl group, a cyclopentyl group, or a cyclohexyl group.

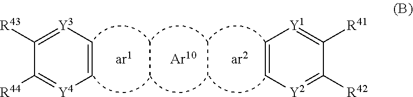

wherein:

R⁴¹ to R⁴⁴ may be the same or different and each represents a hydrogen atom, a substituted or unsubstituted alkyl group having 1 to 20 carbon atoms, a substituted or unsubstituted aryl group having 6 to 50 carbon atoms, a substituted or unsubstituted heterocyclic group having 5 to 50 ring atoms, a halogen atom, a substituted or unsubstituted fluoroalkyl group having 1 to 20 carbon atoms, a substituted or unsubstituted alkoxy group having 1 to 20 carbon atoms, a substituted or unsubstituted aryloxy group having 6 to 50 carbon atoms, or a cyano group;

adjacent groups of R²¹ to R²⁴ may be bonded to each other to form a ring;

Y¹ to Y⁴ may be the same or different and each represents —N═, —CH═, or —C(R⁴⁵)═, wherein R⁴⁵ represents a substituted or unsubstituted alkyl group having 1 to 20 carbon atoms, a substituted or unsubstituted aryl group having 6 to 50 carbon atoms, a substituted or unsubstituted heterocyclic group having 5 to 50 ring atoms, a halogen atom, a substituted or unsubstituted alkoxy group having 1 to 20 carbon atoms, a substituted or unsubstituted aryloxy group having 6 to 50 ring carbon atoms, or a cyano group;

Ar¹⁰ represents a fused ring having 6 to 24 ring carbon atoms or a hetero cyclic ring having 6 to 24 ring atoms; and

each of ar¹ and ar² independently represents a ring represented by formula (i) or (ii):

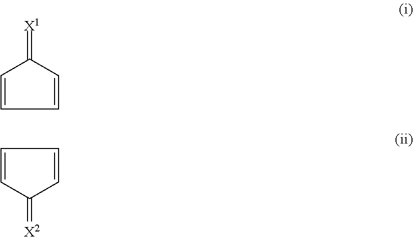

wherein X¹ and X² may be the same or different and each represents a divalent group represented by any one of formulae (a) to (g):

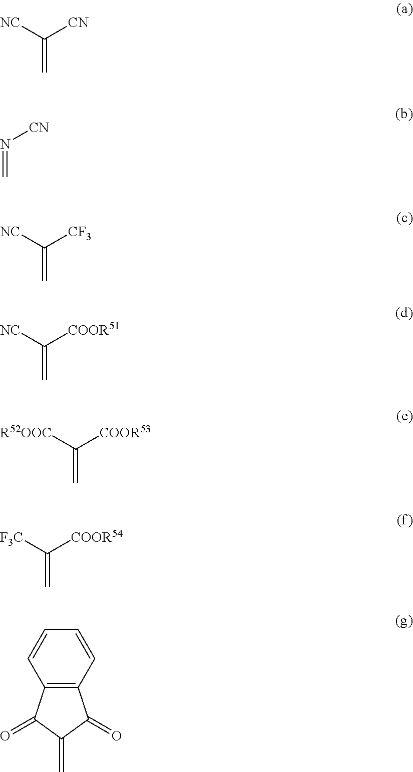

wherein:

R⁵¹ to R⁵⁴ may be the same or different and each represents a hydrogen atom, a substituted or unsubstituted alkyl group having 1 to 20 carbon atoms,

a substituted or unsubstituted aryl group having 6 to 50 carbon atoms, or a substituted or unsubstituted heterocyclic group having 5 to 50 ring atoms; and

R⁵² and R⁵³ may be bonded to each other to form a ring.

Examples of the groups for R⁴¹ to R⁴⁴ and R⁵¹ to R⁵⁴ are described below.

Examples of the alkyl group include a methyl group, an ethyl group, a n-propyl group, an isopropyl group, a n-butyl group, an isobutyl group, a tert-butyl group, a cyclopentyl group, and a cyclohexyl group.

Examples of the aryl group include a phenyl group, a biphenyl group, and a naphthyl group.

Examples of the heterocyclic group include residues of pyridine, pyrazine, furan, imidazole, benzimidazole, and thiophene.

Examples of the halogen atom include a fluorine atom, a chlorine atom, a bromine atom, and an iodine atom.

Examples of the alkoxy group include a methoxy group and an ethoxy group.

Example of the aryloxy group may include a phenyloxy group.

### Guest Material of Light Emitting Layer

The light emitting layer comprises a highly light-emitting material and may be formed from a various kind of materials. For example, a fluorescent emitting compound and a phosphorescent emitting compound are usable as the highly light-emitting material. The fluorescent emitting compound is a compound capable of emitting light from a singlet excited state, and the phosphorescent emitting compound is a compound capable of emitting light from a triplet excited state.

Examples of blue fluorescent emitting material for use in the light emitting layer include a pyrene derivative, a styrylamine derivative, a chrysene derivative, a fluoranthene derivative, a fluorene derivative, a diamine derivative, and a triarylamine derivative, such as N,N′-bis[4-(9H-carbazole-9-yl)phenyl]-N,N′-diphenylstilbene-4,4′-diamine (YGA2S), 4-(9H-carbazole-9-yl)-4′-(10-phenyl-9-anthryl)triphenylamine (YGAPA), and 4-(10-phenyl-9-anthryl)-4′-(9-phenyl-9H-carb azole-3-yl)triphenylamine (PCBAPA).

Examples of green fluorescent emitting material for use in the light emitting layer include an aromatic amine derivative, such as N-(9,10-diphenyl-2-anthryl)-N,9-diphenyl-9H-carbazole-3-amine (2PCAPA), N-[9,10-bis(1,1′-biphenyl-2-yl)-2-anthryl]-N,9-diphenyl-9H-carbazole-3-amine (2PCABPhA), N-(9,10-diphenyl-2-anthryl)-N,N′,N′-triphenyl-1,4-phenylenediamine (2DPAPA), N-[9,10-bis(1,1′-biphenyl-2-yl)-2-anthryl]-N,N′,N′-triphenyl-1,4-phenylenediamine (2DPABPhA), N-[9,10-bis(1, 1′-biphenyl-2-yl)]—N-[4-(9H-carbazole-9-yl)phenyl]-N-phenylanthracene-2-amine (2YGABPhA), and N,N,9-triphenylanthracene-9-amine (DPhAPhA).

Examples of red fluorescent emitting material for use in the light emitting layer include a tetracene derivative and a diamine derivative, such as N,N,N′,N′-tetrakis(4-methylphenyl)tetracene-5,11-diamine (p-mPhTD) and 7,14-diphenyl-N,N,N′,N′-tetrakis(4-methylphenyl)acenaphtho[1,2-a]fluoranthene-3,10-diamine (p-mPhAFD).

In an embodiment of the invention, the fluorescent emitting material is preferably comprises at least one selected from an anthracene derivative, a fluoranthene derivative, a styrylamine derivative, and an arylamine derivative.

Examples of blue phosphorescent emitting material for use in the light emitting layer include a metal complex, such as an iridium complex, an osmium complex, and a platinum complex. Examples thereof include bis[2-(4′,6′-difluorophenyl)pyridinato-N,C2′]iridium(III) tetrakis(1-pyrazolyl)borato (FIr₆), bis[2-(4′,6′-difluorophenyl)pyridinato-N,C2′]iridium(III) picolinato (FIrpic), bis[2-(3′,5′-bistrifluoromethylphenyl)pyridinato-N,C2′]iridium(III) picolinato (Ir(CF₃ppy)₂(pic)), and bis[2-(4′,6′-difluorophenyl)pyridinato-N,C2′]iridium(III) acetylacetonato (FIracac).

Examples of green phosphorescent emitting material for use in the light emitting layer include an iridium complex, such as tris(2-phenylpyridinato-N,C2′)iridium(III) (Ir(ppy)₃), bis(2-phenylpyridinato-N,C2′)iridium(III) acetylacetonato (Ir(ppy)₂(acac)), bis(1,2-diphenyl-1H-benzimidazolato)iridium(III) acetylacetonato (Ir(pbi)₂(acac)), and bis(benzo[h]quinolinato)iridium(III) acetylacetonato (Ir(bzq)₂(acac)).

Examples of red phosphorescent emitting material for use in the light emitting layer include a metal complex, such as an iridium complex, a platinum complex, a terbium complex, and a europium complex. Examples thereof include an organometallic complex, such as bis[2-(2′-benzo[4,5-α]thienyl)pyridinato-N,C3′]iridium(III) acetylacetonato (Ir(btp)₂(acac)), bis(1-phenylisoquinolinato-N,C2′)iridium(III) acetylacetonato (Ir(piq)₂(acac)), (acetylacetonato)bis[2,3-bis(4-fluorophenyl)quinoxalinato]iridium(III) (Ir(Fdpq)₂(acac)), and 2,3,7,8,12,13,17,18-octaethyl-21H,23H-porphyrin platinum(II) (PtOEP).

The following rare earth metal complex, such as tris(acetylacetonato) (monophenanthroline)terbium(III) (Tb(acac)₃(Phen)), tris(1,3-diphenyl-1,3-propanedionato)(monophenanthroline)europium(III) (Eu(DBM)₃(Phen)), and tris[1-(2-thenoyl)-3,3,3-trifluoroacetonato](monophenanthroline)europium(III) (Eu(TTA)₃(Phen)), emits light from the rare earth metal ion (electron transition between different multiple states), and therefore, usable as a phosphorescent emitting compound.

In an embodiment of the invention, the phosphorescent emitting material is an orthometalated complex of a metal atom selected from iridium (Ir), osmium (Os), and platinum (Pt).

### Host Material for Light Emitting Layer

The light emitting layer may be formed by dispersing the highly light-emitting material (guest material) mentioned above in another material (host material). The material in which the highly light-emitting material is to be dispersed may be selected from various kinds of materials and is preferably a material having a lowest unoccupied molecular orbital level (LUMO level) higher than that of the highly light-emitting material and a highest occupied molecular orbital level (HOMO level) lower than that of the highly light-emitting material.

In an embodiment of the invention, the compound of the invention is usable as the host material of light emitting layer.

The material in which the highly light-emitting material is to be dispersed may include, for example,

(1) a metal complex, such as an aluminum complex, a beryllium complex, and a zinc complex;
(2) a heterocyclic compound, such as an oxadiazole derivative, a benzimidazole derivative, and a phenanthroline derivative;
(3) a fused aromatic compound, such as a carbazole derivative, an anthracene derivative, a phenanthrene derivative, a pyrene derivative, and a chrysene derivative; and
(4) an aromatic amine compound, such as a triarylamine derivative and a fused aromatic polycyclic amine derivative.

Examples thereof include:

a metal complex, such as tris(8-quinolinolato)aluminum(III) (Alq), tris(4-methyl-8-quinolinolato)aluminum(III) (Almq₃), 8-quinolinolatolithium (Liq), bis(10-hydroxybenzo[h]quinolinato)beryllium(II) (BeBq₂), bis(2-methyl-8-quinolinolato)(4-phenylphenolato)aluminum(III) (BAlq), bis(8-quinolinolato)zinc(II) (Znq), bis[2-(2-benzoxazolyl)phenolato]zinc(II) (ZnPBO), and bis[2-(2-benzothiazolyl)phenolato]zinc(II) (ZnBTZ);

a heterocyclic compound, such as 2-(4-biphenylyl)-5-(4-tert-butylphenyl)-1,3,4-oxadiazole (PBD), 1,3-bis[5-(p-tert-butylphenyl)-1,3,4-oxadiazole-2-yl]benzene (OXD-7), 3-(4-biphenylyl)-4-phenyl-5-(4-tert-butylphenyl)-1,2,4-triazole (TAZ), 2,2′,2″-(1,3,5-benzenetriyl)tris(1-phenyl-1H-benzimidazole) (TPBI), bathophenanthroline (BPhen), and bathocuproin (BCP);

a fused aromatic compound, such as 9-[4-(10-phenyl-9-anthryl)phenyl]-9H-carbazole (CzPA), 3,6-diphenyl-9-[4-(10-phenyl-9-anthryl)phenyl]-9H-carbazole (DPCzPA), 9,10-bis(3,5-diphenylphenyl)anthracene (DPPA), 9,10-di(2-naphthyl)anthracene (DNA), 2-tert-butyl-9,10-di(2-naphthyl)anthracene (t-BuDNA), 9,9′-bianthryl (BANT), 9,9′-(stilbene-3,3′-diyl)diphenanthrene (DPNS), 9,9′-(stilbene-4,4′-diyl)diphenanthrene (DPNS2), 3,3′,3″-(benzene-1,3,5-triyl)tripyrene (TPB3), 9,10-diphenylanthracene (DPAnth), and 6,12-dimethoxy-5,11-diphenylchrysene; and

an aromatic amine compound, such as N,N-diphenyl-9-[4-(10-phenyl-9-anthryl)phenyl]-9H-carbazole-3-amine (CzA1PA), 4-(10-phenyl-9-anthryl)triphenylamine (DPhPA), N,9-diphenyl-N-[4-(10-phenyl-9-anthryl)phenyl]-9H-carbazole-3-amine (PCAPA), N,9-diphenyl-N-{4-[4-(10-phenyl-9-anthryl)phenyl]phenyl}-9H-carbazole-3-amine (PCAPBA), N-(9,10-diphenyl-2-anthryl)-N,9-diphenyl-9H-carbazole-3-amine (2PCAPA), NPB (or ca-NPD), TPD, DFLDPBi, and BSPB.

The material (host material) for dispersing the highly light-emitting material (guest material) may be used alone or in combination of two or more.

### Electron Transporting Layer

The electron transporting layer comprises a highly electron-transporting material, for example,

(1) a metal complex, such as an aluminum complex, a beryllium complex, and a zinc complex;
(2) a heteroaromatic compound, such as an imidazole derivative, a benzimidazole derivative, an azine derivative, a carbazole derivative, and a phenanthroline derivative; and
(3) a polymeric compound.

Examples of the low molecular organic compound include a metal complex, such as Alq, tris(4-methyl-8-quinolinolato)aluminum (Almq₃), bis(10-hydroxybenzo[h]quinolinato)beryllium (BeBq₂), BAlq, Znq, ZnPBO, and ZnBTZ; and a heteroaromatic compound, such as 2-(4-biphenylyl)-5-(4-tert-butylphenyl)-1,3,4-oxadiazole (PBD), 1,3-bis[5-(ptert-butylphenyl)-1,3,4-oxadiazole-2-yl]benzene (OXD-7), 3-(4-tert-butylphenyl)-4-phenyl-5-(4-biphenylyl)-1,2,4-triazole (TAZ), 3-(4-tert-butylphenyl)-4-(4-ethylphenyl)-5-(4-biphenylyl)-1,2,4-triazole (p-EtTAZ), bathophenanthroline (BPhen), bathocuproine (BCP), and 4,4′-bis(5-methylbenzoxazole-2-yl)stilbene (BzOs).

The above compounds have an electron mobility of mainly 10⁻⁶ cm²/Vs or more. Other materials are also usable in the electron transporting layer if their electron transporting ability is higher than their hole transporting ability. The electron transporting layer may be a single layer or a laminate of two or more layers each comprising the material mentioned above.

A polymeric compound is also usable in the electron transporting layer. Examples thereof include poly[(9,9-dihexylfluorene-2,7-diyl)-co-(pyridine-3,5-diyl)] (PF-Py), and poly[(9,9-dioctylfluorene-2,7-diyl)-co-(2,2′-bipyridine-6,6′-diyl)](PF-BPy).

In an embodiment of the invention, the electron transporting layer preferably comprises the heteroaromatic compound mentioned above, more preferably comprises the benzimidazole derivative.

### Electron Injecting Layer

The electron injecting layer comprises a highly electron-injecting material, for example, an alkali metal, an alkaline earth metal, and a compound of these metals, such as lithium (Li), cesium (Cs), calcium (Ca), lithium fluoride (LiF), cesium fluoride (CsF), calcium fluoride (CaF2), and lithium oxide (LiOx). In addition, an electron transporting material which is incorporated with an alkali metal, an alkaline earth metal or a compound thereof, for example, Alq doped with magnesium (Mg), is also usable. By using such a material, electrons are efficiently injected from the cathode.

A composite material obtained by mixing an organic compound and an electron donor is also usable in the electron injecting layer. Such a composite material is excellent in the electron injecting ability and the electron transporting ability, because the electron donor donates electrons to the organic compound. The organic compound is preferably a material excellent in transporting the received electrons. Examples thereof are the materials for the electron transporting layer mentioned above, such as the metal complex and the aromatic heterocyclic compound. Any material capable of giving its electron to another organic compound is usable as the electron donor. Preferred examples thereof are an alkali metal, an alkaline earth metal, and a rare earth metal, such as lithium, cesium, magnesium, calcium, erbium, and ytterbium; an alkali metal oxide and an alkaline earth metal oxide, such as, lithium oxide, calcium oxide, and barium oxide; a Lewis base, such as magnesium oxide; and an organic compound, such as tetrathiafulvalene (TTF).

### Cathode

The cathode is formed preferably from a metal, an alloy, an electrically conductive compound, and a mixture thereof, each having a small work function, for example, a work function of 3.8 eV or less. Examples of the material for the cathode include a metal of the group 1 or 2 of the periodic table, for example, an alkali metal, such as lithium (Li) and cesium (Cs), an alkaline earth metal, such as magnesium (Mg), an alloy containing these metals (for example, MgAg and AlLi), a rare earth metal, such as europium (Eu) and ytterbium (Yb), and an alloy containing a rare earth metal.

The alkali metal, the alkaline earth metal, and the alloy thereof can be made into the cathode by a vacuum vapor deposition or a sputtering method. When a silver paste, etc. is used, a coating method and an inkjet method are usable.

When the electron injecting layer is formed, the material for the cathode can be selected independently from the work function and various electroconductive materials, such as Al, Ag, ITO, graphene, and indium oxide-tin oxide doped with silicon or silicon oxide, are usable. These electroconductive materials are made into films by a sputtering method, an inkjet method, and a spin coating method.

Each layer of the organic EL device is formed by a dry film-forming method, such as vacuum vapor deposition, sputtering, plasma, and ion plating, and a wet film-forming method, such as spin coating, dip coating, and flow coating.

In the wet film-forming method, the material for each layer is dissolved or dispersed in a suitable solvent, such as ethanol, chloroform, tetrahydrofuran, and dioxane, and then the obtained solution or dispersion is made into a film. To improve the film-forming properties and prevent pin holes on the film, the solution and the dispersion may include a resin or an additive. Examples of the resin include an insulating resin and a copolymer thereof, such as polystyrene, polycarbonate, polyarylate, polyester, polyamide, polyurethane, polysulfone, polymethyl methacrylate, polymethyl acrylate, and cellulose; and a photoconductive resin, such as poly-N-vinylcarbazole and polysilane; and an electroconductive resin, such as polythiophene and polypyrrole. Examples of the additive include an antioxidant, an ultraviolet absorber, and a plasticizer.

The thickness of each layer is not particularly limited and selected so as to obtain a good device performance. If extremely thick, a large applied voltage is needed to obtain a desired emission output, thereby reducing the efficiency. If extremely thin, pinholes occur on the film to make it difficult to obtain a sufficient luminance even when applying an electric field. The thickness is generally 5 nm to 10 μm and preferably 10 nm to 0.2 μm. The thickness of the light emitting layer is, but not particularly limited to, preferably 5 to 100 nm, more preferably 7 to 70 nm, and still more preferably 10 to 50 nm. The thickness of the hole transporting layer is preferably 10 to 300 nm.

When the hole transporting layer is made into a two-layered structure as described above, the thickness of the first hole transporting layer (anode side) is preferably 50 to 300 nm, more preferably 50 to 250 nm, still more preferably 50 to 200 nm, and further preferably 50 to 150 nm, and the thickness of the second hole transporting layer (light emitting layer side) is preferably 5 to 100 nm, more preferably 5 to 80 nm, and if needed, 5 to 40 nm, preferably 5 to 20 nm.

### Electronic Device

The electronic device in an aspect of the invention comprises the organic EL device in an aspect of the invention mentioned above.

Examples of the electronic device include display parts, such as organic EL panel module; display devices of television sets, mobile phones, personal computer, etc.; and light emitting sources of lighting equipment and vehicle lighting equipment.

### EXAMPLES

The present invention will be described in more detail with respect to the examples and comparative examples. However, it should be noted that the scope of the invention is not limited to the following examples.

The compounds recited in the claims of this application can be synthesized by using a known reaction and a starting compound depending upon the target compound while referring to the following synthesis reactions.

### Intermediate Synthesis 1: Synthesis of Intermediate 1

Under an argon atmosphere, a mixture of 103.6 g (512.6 mmol) of 2-nitrobromobenzene, 108.7 g (512.7 mmol) of dibenzofuran-2-boronic acid, and 11.84 g (10.2 mmol) of Pd[PPh₃]₄ in 1200 ml of toluene and 770 ml (1540.0 mmol) of a 2 M aqueous solution of sodium carbonate was stirred and refluxed for 23 h under heating.

After the reaction, the mixture was cooled to room temperature, and the organic layer was separated in a separatory funnel. The organic layer was dried over anhydrous magnesium sulfate, filtered, and then concentrated. The concentrate residue was purified by silica gel column chromatography to obtain 106.2 g (yield: 72%) of a white solid, which was identified as the following intermediate 1 by FD-MS analysis (Field Desorption Mass Spectrometry).

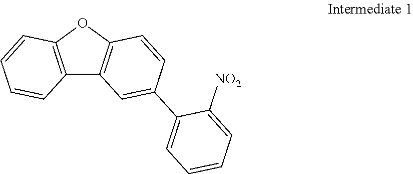 [SMILES: O=[N+]([O-])c1ccccc1-c1ccc2oc3ccccc3c2c1]

### Intermediate Synthesis 2: Synthesis of Intermediate 2

Under an argon atmosphere, a mixture of 106.2 g (367.3 mmol) of the intermediate 1 and 240.8 g (918.1 mmol) of triphenylphosphine in 710 ml of o-dichlorobenzene was stirred at 189° C. for 12 h under heating.

After the reaction, the mixture was cooled to room temperature, and the precipitated crystal was collected by filtration. The crystal was dissolved in tetrahydrofuran and treated with silica gel at room temperature. Then, the silica gel was removed by filtration, and the filtrate was concentrated. The concentrate residue was purified by recrystallization to obtain 52.4 g (yield: 55%) of a white solid, which was identified as the following intermediate 2 by FD-MS analysis.

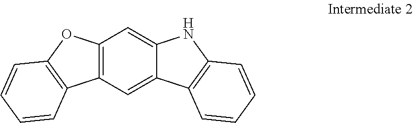 [SMILES: c1ccc2c(c1)[nH]c1cc3oc4ccccc4c3cc12]

### Intermediate Synthesis 3: Synthesis of Intermediate 3

Under an argon atmosphere, a mixture of 52.4 g (203.7 mmol) of the intermediate 2 in 700 ml of dimethylformamide was stirred. After adding 36.2 g (203.7 mmol) of N-bromosuccinimide, the mixture was further stirred at room temperature for 12 h.

After the reaction, the mixture was extracted with toluene in a separatory funnel. The organic layer was dried over anhydrous magnesium sulfate, filtered, and then concentrated. The concentrate residue was purified by silica gel column chromatography to obtain 43.1 g (yield: 63%) of a white solid, which was identified as the following intermediate 3 by FD-MS analysis.

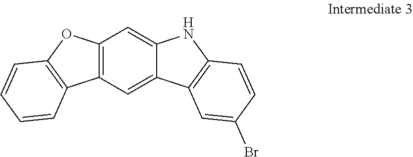 [SMILES: Brc1ccc2[nH]c3cc4oc5ccccc5c4cc3c2c1]

### Intermediate Synthesis 4: Synthesis of Intermediate 4

Under an argon atmosphere, a mixture of 2.79 g (14.6 mmol) of CuI and 2.5 g (22.0 mmol) of trans-1,2-cyclohexanediamine in 140 ml of dioxane was stirred for one hour at room temperature. After adding 13.6 g (40.6 mmol) of the intermediate 3, 9.9 g (48.5 mmol) of iodobenzene, and 21.7 g (102.3 mmol) of tripotassium phosphate, the mixture was stirred at 90° C. for 2 h under heating.

After the reaction, the mixture was cooled to room temperature, and the precipitated crystal was collected by filtration. The crystal was purified by silica gel column chromatography to obtain 13.4 g (yield: 80%) of a white solid, which was identified as the following intermediate 4 by FD-MS analysis.

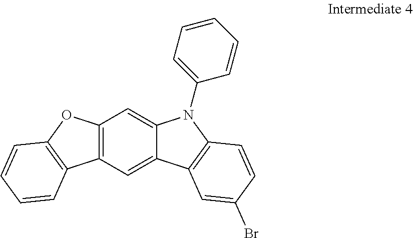 [SMILES: Brc1ccc2c(c1)c1cc3c(cc1n2-c1ccccc1)oc1ccccc13]

### Intermediate Synthesis 5: Synthesis of Intermediate 5

Under an argon atmosphere, a mixture of 10.7 g (26.0 mmol) of the intermediate 4, 8.07 g (31.8 mmol) of bispinacolatodiboron, 296 mg (1.32 mmol) of palladium acetate, 742 mg (2.64 mmol) of tricyclohexylphosphine, and 5.2 g (53.0 mmol) of potassium acetate in 100 ml of N-methylpyrrolidone was stirred at 65° C. for 30 min and further stirred at 90° C. for 3 h.

After the reaction, the mixture was cooled to room temperature, diluted with toluene, and filtered through celite. The mixture was extracted with toluene in a separatory funnel. The organic layer was dried over anhydrous magnesium sulfate, filtered, and then concentrated. The concentrate residue was purified by silica gel column chromatography to obtain 8.7 g (yield: 73%) of a white solid, which was identified as the following intermediate 5 by FD-MS analysis.

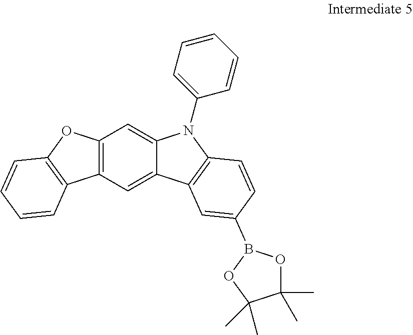 [SMILES: CC1(C)OB(c2ccc3c(c2)c2cc4c(cc2n3-c2ccccc2)oc2ccccc24)OC1(C)C]

### Intermediate Synthesis 6: Synthesis of Intermediate 6

Under an argon atmosphere, a mixture of 4.6 g (10.0 mmol) of the intermediate 5, 3.4 g (10.0 mmol) of the intermediate 3, and 163.3 mg (0.2 mmol) of Pd[dppf]Cl₂ in 50 ml of dioxane and 15 ml (30.0 mmol) of a 2 M aqueous solution of sodium carbonate was stirred and refluxed for 12 h under heating.

After the reaction, the mixture was cooled to room temperature, and the organic layer was separated in a separatory funnel. The organic layer was dried over anhydrous magnesium sulfate, filtered, and then concentrated. The concentrate residue was purified by silica gel column chromatography to obtain 3.2 g (yield: 55%) of a white solid, which was identified as the following intermediate 6 by FD-MS analysis.

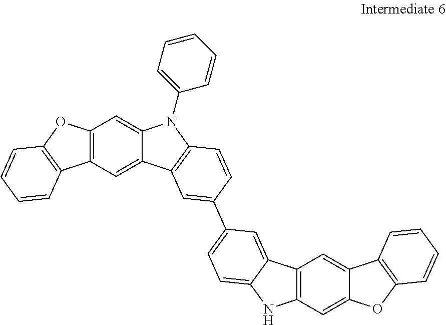 [SMILES: c1ccc(-n2c3ccc(-c4ccc5[nH]c6cc7oc8ccccc8c7cc6c5c4)cc3c3cc4c(cc32)oc2ccccc24)cc1]

### Intermediate Synthesis 7: Synthesis of Intermediate 7

In the same manner as in Intermediate Synthesis 1 except for using 108.7 g of dibenzofuran-4-boronic acid in place of dibenzofuran-2-boronic acid, 103.3 g (yield: 70%) of a white solid was obtained, which was identified as the following intermediate 7 by FD-MS analysis.

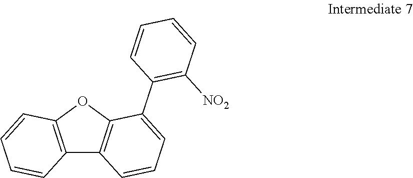 [SMILES: O=[N+]([O-])c1ccccc1-c1cccc2c1oc1ccccc12]

### Intermediate Synthesis 8: Synthesis of Intermediate 8

In the same manner as in Intermediate Synthesis 2 except for using 103.3 g of the intermediate 7 in place of the intermediate 1, 56.0 g (yield: 61%) of a white solid was obtained, which was identified as the following intermediate 8 by FD-MS analysis.

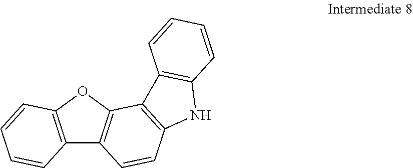 [SMILES: c1ccc2c(c1)[nH]c1ccc3c4ccccc4oc3c12]

### Intermediate Synthesis 9: Synthesis of Intermediate 9

In the same manner as in Intermediate Synthesis 3 except for using 56.0 g of the intermediate 8 in place of the intermediate 2, 40.2 g (yield: 55%) of a white solid was obtained, which was identified as the following intermediate 9 by FD-MS analysis.

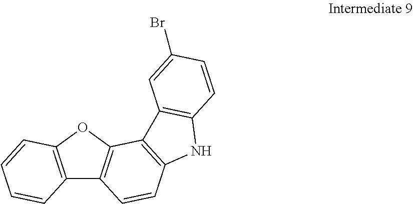 [SMILES: Brc1ccc2[nH]c3ccc4c5ccccc5oc4c3c2c1]

### Intermediate Synthesis 10: Synthesis of Intermediate 10

In the same manner as in Intermediate Synthesis 4 except for using 13.6 g of the intermediate 9 in place of the intermediate 3, 10.8 g (yield: 65%) of a white solid was obtained, which was identified as the following intermediate 10 by FD-MS analysis.

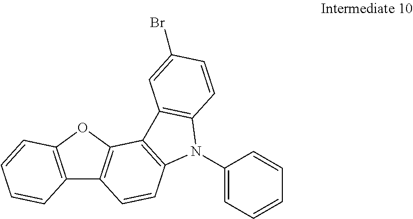 [SMILES: Brc1ccc2c(c1)c1c3oc4ccccc4c3ccc1n2-c1ccccc1]

### Intermediate Synthesis 11: Synthesis of Intermediate 11

In the same manner as in Intermediate Synthesis 5 except for using 10.8 g of the intermediate 10 in place of the intermediate 4, 7.3 g (yield: 61%) of a white solid was obtained, which was identified as the following intermediate 11 by FD-MS analysis.

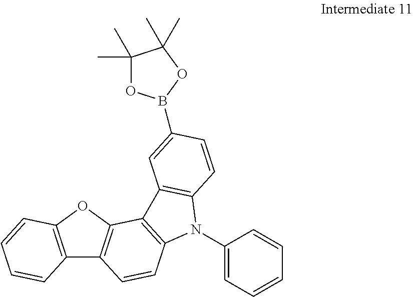 [SMILES: CC1(C)OB(c2ccc3c(c2)c2c4oc5ccccc5c4ccc2n3-c2ccccc2)OC1(C)C]

### Intermediate Synthesis 12: Synthesis of Intermediate 12

In the same manner as in Intermediate Synthesis 1 except for using 116.9 g of dibenzothiophene-2-boronic acid in place of dibenzofuran-2-boronic acid, 117.4 g (yield: 75%) of a white solid was obtained, which was identified as the following intermediate 12 by FD-MS analysis.

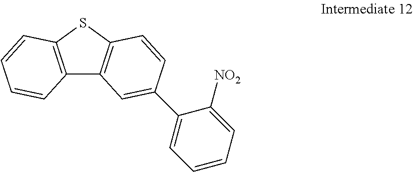 [SMILES: O=[N+]([O-])c1ccccc1-c1ccc2sc3ccccc3c2c1]

### Intermediate Synthesis 13: Synthesis of Intermediate 13

In the same manner as in Intermediate Synthesis 2 except for using 117.4 g of the intermediate 12 in place of the intermediate 1, 63.1 g (yield: 60%) of a white solid was obtained, which was identified as the following intermediate 13 by FD-MS analysis.

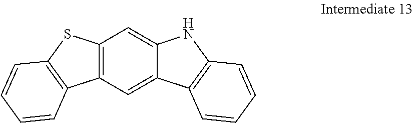 [SMILES: c1ccc2c(c1)[nH]c1cc3sc4ccccc4c3cc12]

### Intermediate Synthesis 14: Synthesis of Intermediate 14

In the same manner as in Intermediate Synthesis 3 except for using 63.1 g of the intermediate 13 in place of the intermediate 2, 46.3 g (yield: 57%) of a white solid was obtained, which was identified as the following intermediate 14 by FD-MS analysis.

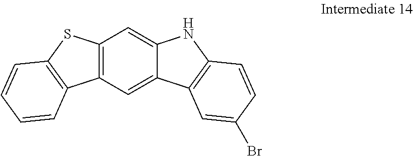 [SMILES: Brc1ccc2[nH]c3cc4sc5ccccc5c4cc3c2c1]

### Intermediate Synthesis 15: Synthesis of Intermediate 15

In the same manner as in Intermediate Synthesis 4 except for using 14.3 g of the intermediate 14 in place of the intermediate 3, 11.8 g (yield: 68%) of a white solid was obtained, which was identified as the following intermediate 15 by FD-MS analysis.

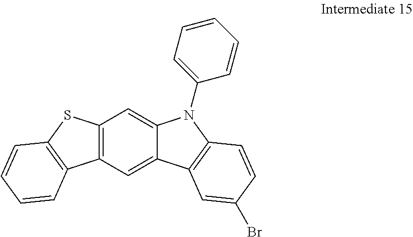 [SMILES: Brc1ccc2c(c1)c1cc3c(cc1n2-c1ccccc1)sc1ccccc13]

### Intermediate Synthesis 16: Synthesis of Intermediate 16

In the same manner as in Intermediate Synthesis 5 except for using 11.1 g of the intermediate 15 in place of the intermediate 4, 8.0 g (yield: 65%) of a white solid was obtained, which was identified as the following intermediate 16 by FD-MS analysis.

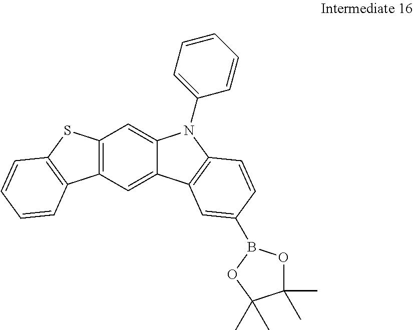 [SMILES: CC1(C)OB(c2ccc3c(c2)c2cc4c(cc2n3-c2ccccc2)sc2ccccc24)OC1(C)C]

### Intermediate Synthesis 17: Synthesis of Intermediate 17

In the same manner as in Intermediate Synthesis 6 except for using 4.8 g of the intermediate 16 in place of the intermediate 5 and using 3.5 g of the intermediate 14 in place of the intermediate 3, 2.8 g (yield: 45%) of a white solid was obtained, which was identified as the following intermediate 17 by FD-MS analysis.

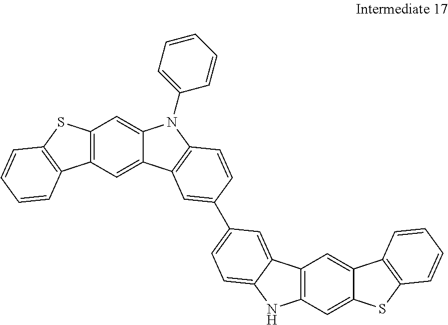 [SMILES: c1ccc(-n2c3ccc(-c4ccc5[nH]c6cc7sc8ccccc8c7cc6c5c4)cc3c3cc4c(cc32)sc2ccccc24)cc1]

### Intermediate Synthesis 18: Synthesis of Intermediate 18

Under an argon atmosphere, a mixture of 15 g (54.9 mmol) of 2-bromo-9,9-dimethylfluorene, 5 mL (54.9 mmol) of aniline, 0.754 g (0.82 mol) of tris(dibenzylideneacetone) dipalladium(0), 1.02 g (1.64 mmol) of 2,2′-bis(diphenylphosphino)-1,1′-binaphthyl (BINAP), and 10.5 g (109.3 mmol) of sodium t-butoxide in 270 mL of toluene was stirred and refluxed for 7 h under heating.

After the reaction, the mixture was cooled to room temperature and extracted with ethyl acetate in a separatory funnel. The obtained organic layer was washed with a sodium carbonate aqueous solution, dried over anhydrous sodium sulfate, filtered, and then concentrated. The obtained residue was purified by silica gel column chromatography and recrystallization to obtain 12 g (yield: 77%) of solid, which was identified as the following intermediate 18 by FD-MS analysis.

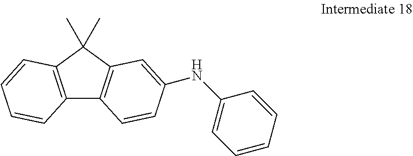 [SMILES: CC1(C)c2ccccc2-c2ccc(Nc3ccccc3)cc21]

### Intermediate Synthesis 19: Synthesis of Intermediate 19

Under the atmosphere, a mixture of 8.32 g (29.2 mmol) of the intermediate 18 in 29.2 mL of pivalic acid was heated to 120° C. After adding 0.404 g (2.92 mmol) of potassium carbonate and 7.87 g (35.1 mmol) of palladium acetate (II), the mixture was stirred for one hour.

After the reaction, the mixture was cooled to room temperature, diluted with ethyl acetate, and filtered to remove the impurities. The obtained filtrate washed with a sodium carbonate aqueous solution, dried over anhydrous magnesium sulfate, filtered, and then concentrated. The obtained residue was purified by silica gel column chromatography and recrystallization to obtain 5.15 g (yield: 62%) of solid, which was identified as the following intermediate 19 by FD-MS analysis.

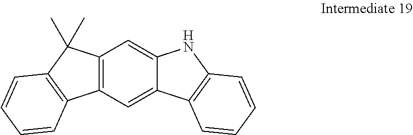 [SMILES: CC1(C)c2ccccc2-c2cc3c(cc21)[nH]c1ccccc13]

### Intermediate Synthesis 20: Synthesis of Intermediate 20

Under an argon atmosphere, into a mixture of 5.60 g (19.8 mmol) of the intermediate 19 in 99 mL of acetonitrile, 3.52 g (19.8 mmol) of N-bromosuccinimide was added under ice-cooling. The resultant mixture was stirred for one hour.

After the reaction, the mixture was heated to room temperature and the generated solid was collected by filtration. The filtrate was extracted with ethyl acetate, washed with water, dried over anhydrous magnesium sulfate, filtered, and then concentrated. The concentrate residue and the solid collected by filtration was combined and purified by silica gel column chromatography to obtain 6.93 g (yield: 97%) of solid, which was identified as the following intermediate 20 by FD-MS analysis.

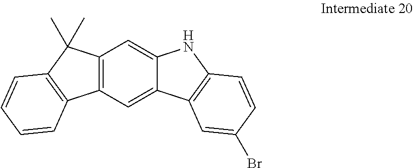 [SMILES: CC1(C)c2ccccc2-c2cc3c(cc21)[nH]c1ccc(Br)cc13]

### Intermediate Synthesis 21: Synthesis of Intermediate 21

In the same manner as in Intermediate Synthesis 6 except for using 3.0 g of the intermediate 20 in place of the intermediate 3, 2.65 g (yield: 52%) of a white solid was obtained, which was identified as the following intermediate 21 by FD-MS analysis.

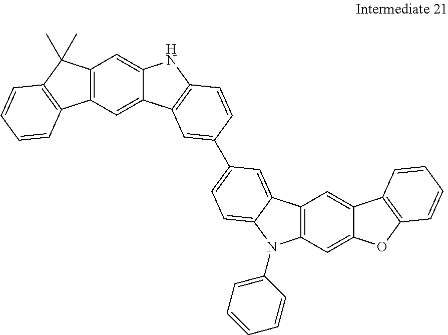 [SMILES: CC1(C)c2ccccc2-c2cc3c(cc21)[nH]c1ccc(-c2ccc4c(c2)c2cc5c(cc2n4-c2ccccc2)oc2ccccc25)cc13]

### Intermediate Synthesis 22: Synthesis of Intermediate 22

In the same manner as in Intermediate Synthesis 6 except for using 3.94 g of the intermediate 16 in place of the intermediate 5 and using 3.0 g of the intermediate 20 in place of the intermediate 3, 2.56 g (yield: 49%) of a white solid was obtained, which was identified as the following intermediate 22 by FD-MS analysis.

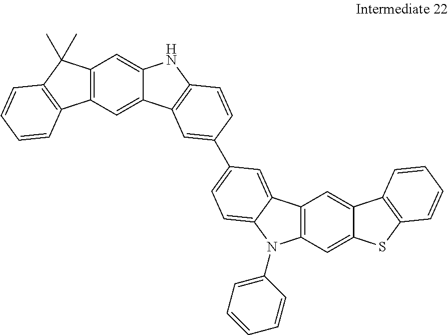 [SMILES: CC1(C)c2ccccc2-c2cc3c(cc21)[nH]c1ccc(-c2ccc4c(c2)c2cc5c(cc2n4-c2ccccc2)sc2ccccc25)cc13]

### Synthesis Example 1: Production of Aromatic Heterocyclic Derivative (PH1)

Under an argon atmosphere, a mixture of 2.9 g (5.0 mmol) of the intermediate 6, 1.4 g (5.25 mmol) of 2-chloro-4,6-diphenylpyrimidine, 140 mg (0.15 mmol) of Pd₂(dba)₃, 87 mg (0.3 mmol) of P(tBu)₃HBF₄, and 673 mg (7.0 mmol) of sodium t-butoxide in 30 ml of anhydrous xylene was refluxed for 8 h under heating.

After the reaction, the mixture was cooled to room temperature, and the precipitated crystal was collected by filtration. The crystal was purified by silica gel column chromatography to obtain 2.9 g (yield: 70%) of a white crystal, which was identified as the following compound (PH1) by FD-MS analysis: m/z=818 (theoretical value: 818 for C₅₈H₃₄N₄OL₂).

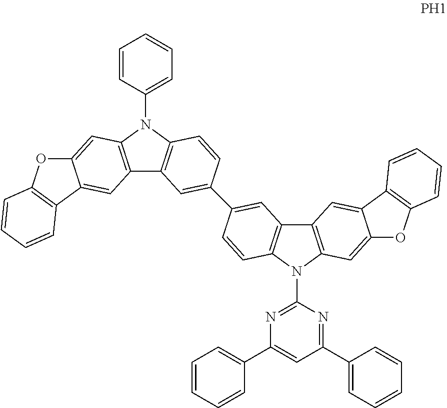 [SMILES: c1ccc(-c2cc(-c3ccccc3)nc(-n3c4ccc(-c5ccc6c(c5)c5cc7c(cc5n6-c5ccccc5)oc5ccccc57)cc4c4cc5c(cc43)oc3ccccc35)n2)cc1]

### Synthesis Example 2: Production of Aromatic Amine Derivative (PH2)

Under an argon atmosphere, a mixture of 4.1 g (10.0 mmol) of the intermediate 4, 4.6 g (10.0 mmol) of the intermediate 5, and 231 mg (0.2 mmol) of Pd[PPh₃]₄ in 50 ml of dioxane and 15 ml (30.0 mmol) of a 2 M aqueous solution of sodium carbonate was stirred and refluxed for 12 h under heating.

After the reaction, the mixture was cooled to room temperature, and the organic layer was separated in a separatory funnel. The organic layer was dried over anhydrous magnesium sulfate, filtered, and then concentrated. The concentrate residue was purified by silica gel column chromatography to obtain 4.3 g (yield: 65%) of a white solid, which was identified as the following compound (PH2) by FD-MS analysis: m/z=664 (theoretical value: 664 for C₄₈H₂₈N₂O₂).

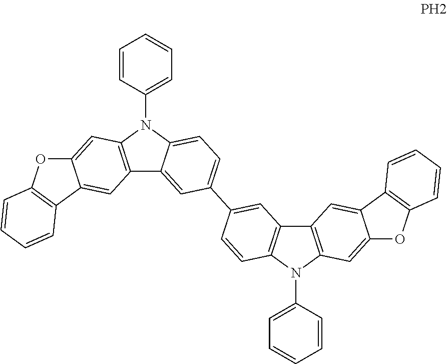 [SMILES: c1ccc(-n2c3ccc(-c4ccc5c(c4)c4cc6c(cc4n5-c4ccccc4)oc4ccccc46)cc3c3cc4c(cc32)oc2ccccc24)cc1]

### Synthesis Example 3: Production of Aromatic Amine Derivative (PH3)

In the same manner as in Synthesis Example 2 except for using 4.1 g of the intermediate 10 in place of the intermediate 4 and using 4.6 g of the intermediate 11 in place of the intermediate 5, 3.8 g (yield: 57%) of a white solid was obtained, which was identified as the following compound (PH3) by FD-MS analysis: m/z=664 (theoretical value: 664 for C₁₈H₂₈N₂O₂).

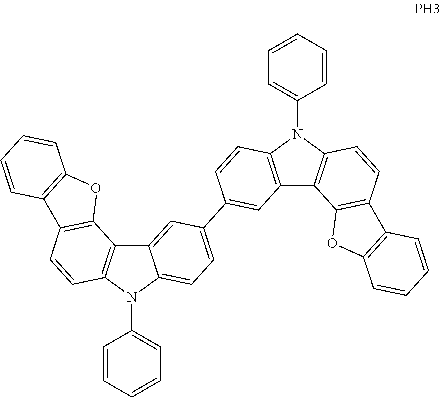 [SMILES: c1ccc(-n2c3ccc(-c4ccc5c(c4)c4c6oc7ccccc7c6ccc4n5-c4ccccc4)cc3c3c4oc5ccccc5c4ccc32)cc1]

### Synthesis Example 4: Production of Aromatic Amine Derivative (PH4)

In the same manner as in Synthesis Example 1 except for using 3.1 g of the intermediate 17 in place of the intermediate 6, 2.8 g (yield: 66%) of a white solid was obtained, which was identified as the following compound (PH4) by FD-MS analysis: m/z=851 (theoretical value: 851 for C₅₈H₃₄N₄S₂).

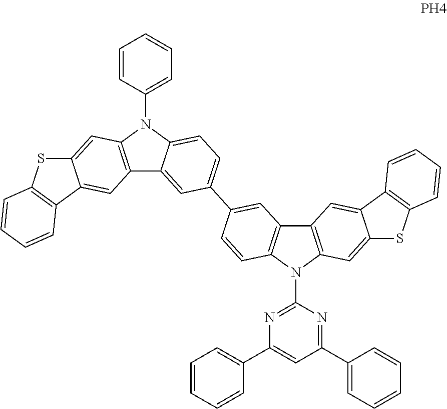 [SMILES: c1ccc(-c2cc(-c3ccccc3)nc(-n3c4ccc(-c5ccc6c(c5)c5cc7c(cc5n6-c5ccccc5)sc5ccccc57)cc4c4cc5c(cc43)sc3ccccc35)n2)cc1]

### Synthesis Example 5: Production of Aromatic Amine Derivative (PH5)

In the same manner as in Synthesis Example 2 except for using 4.3 g of the intermediate 15 in place of the intermediate 4 and using 4.8 g of the intermediate 16 in place of the intermediate 5, 4.2 g (yield: 60%) of a white solid was obtained, which was identified as the following compound (PH5) by FD-MS analysis: m/z=696 (theoretical value: 696 for C₄₈H₂₈N₂S₂).

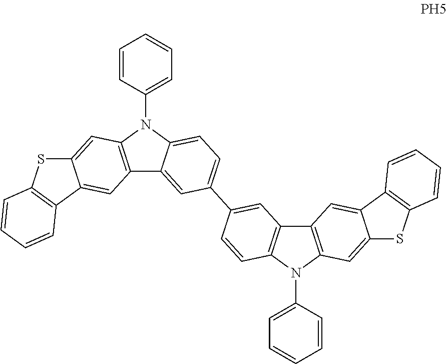 [SMILES: c1ccc(-n2c3ccc(-c4ccc5c(c4)c4cc6c(cc4n5-c4ccccc4)sc4ccccc46)cc3c3cc4c(cc32)sc2ccccc24)cc1]

### Synthesis Example 6: Production of Aromatic Amine Derivative (PH6)

In the same manner as in Synthesis Example 2 except for using 4.8 g of the intermediate 16 in place of the intermediate 5, 3.6 g (yield: 53%) of a white solid was obtained, which was identified as the following compound (PH6) by FD-MS analysis: m/z=680 (theoretical value: 680 for C₄₈H₂₈N₂O S).

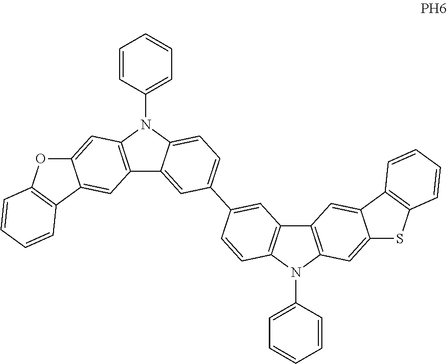 [SMILES: c1ccc(-n2c3ccc(-c4ccc5c(c4)c4cc6c(cc4n5-c4ccccc4)sc4ccccc46)cc3c3cc4c(cc32)oc2ccccc24)cc1]

### Synthesis Example 7: Production of Aromatic Amine Derivative (PH7)

In the same manner as in Synthesis Example 2 except for using 4.6 g of the intermediate 11 in place of the intermediate 5, 4.0 g (yield: 60%) of a white solid was obtained, which was identified as the following compound (PH7) by FD-MS analysis: m/z=664 (theoretical value: 664 for C₄₈H₂₈N₂O₂).

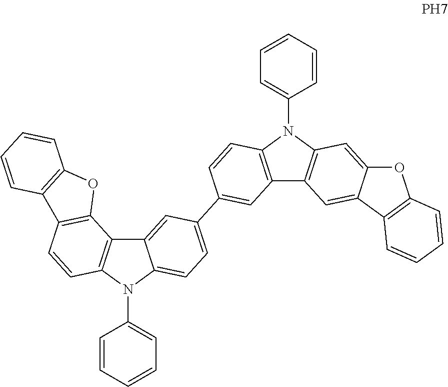 [SMILES: c1ccc(-n2c3ccc(-c4ccc5c(c4)c4c6oc7ccccc7c6ccc4n5-c4ccccc4)cc3c3cc4c(cc32)oc2ccccc24)cc1]

### Synthesis Example 8: Production of Aromatic Amine Derivative (PH8)

Under an argon atmosphere, a mixture of 2.20 g (3.58 mmol) of the intermediate 21, 0.675 g (4.30 mmol) of bromobenzene, 0.131 g (0.14 mol) of tris(dibenzylideneacetone) dipalladium(0), 0.072 g (0.36 mmol) of tri-tert-butylphosphine, and 0.69 g (7.16 mmol) of sodium t-butoxide in 17.9 mL of xylene was stirred and refluxed for 8 h under heating.

After the reaction, the reaction mixture was heated to room temperature and filtered, and the filtrate was concentrated. The obtained residue was purified by silica gel column chromatography and recrystallization to obtain 1.52 g (yield: 62%) of a white solid, which was identified as the following compound (PH8) by FD-MS analysis: m/z=690 (theoretical value: 690 for C₅₁H₃₄N₂O).

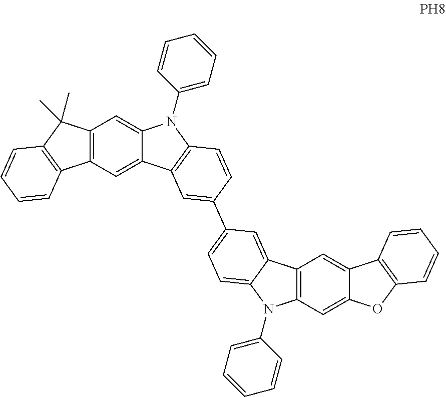 [SMILES: CC1(C)c2ccccc2-c2cc3c4cc(-c5ccc6c(c5)c5cc7c(cc5n6-c5ccccc5)oc5ccccc57)ccc4n(-c4ccccc4)c3cc21]

### Synthesis Example 9: Production of Aromatic Amine Derivative (PH9)

In the same manner as in Synthesis Example 8 except for using 2.26 g of the intermediate 22 in place of the intermediate 21, 1.32 g (yield: 52%) of a white solid was obtained, which was identified as the following compound (PH9) by FD-MS analysis: m/z=706 (theoretical value: 706 for C₅₁H₃₄N₂S).

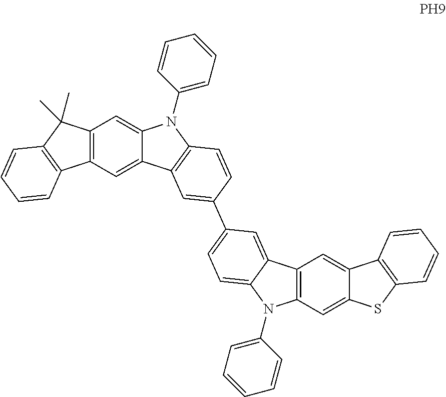 [SMILES: CC1(C)c2ccccc2-c2cc3c4cc(-c5ccc6c(c5)c5cc7c(cc5n6-c5ccccc5)sc5ccccc57)ccc4n(-c4ccccc4)c3cc21]

### Synthesis Example 10: Production of Aromatic Amine Derivative (PH10)

In the same manner as in Synthesis Example 8 except for using 2.11 g of the intermediate 6 in place of the intermediate 21 and using 1.00 g of 4-bromobiphenyl in place of bromobenzene, 1.49 g (yield: 56%) of a white solid was obtained, which was identified as the following compound (PH10) by FD-MS analysis: m/z=740 (theoretical value: 740 for C₅₄H₃₂N₂O₂).

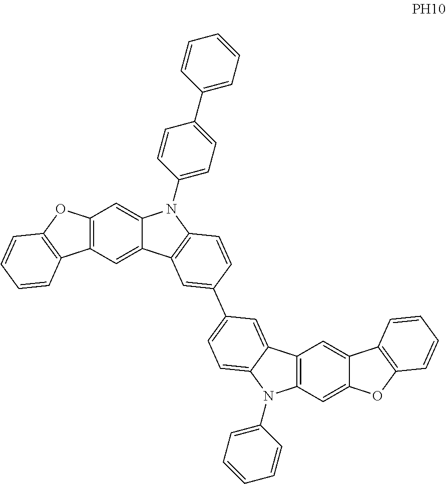 [SMILES: c1ccc(-c2ccc(-n3c4ccc(-c5ccc6c(c5)c5cc7c(cc5n6-c5ccccc5)oc5ccccc57)cc4c4cc5c(cc43)oc3ccccc35)cc2)cc1]

### Synthesis Example 11: Production of Aromatic Amine Derivative (PH11)

In the same manner as in Synthesis Example 8 except for using 2.11 g of the intermediate 6 in place of the intermediate 21 and using 1.00 g of 3-bromobiphenyl in place of bromobenzene, 1.30 g (yield: 49%) of a white solid was obtained, which was identified as the following compound (PH11) by FD-MS analysis: m/z=740 (theoretical value: 740 for C₅₄H₃₂N₂O₂).

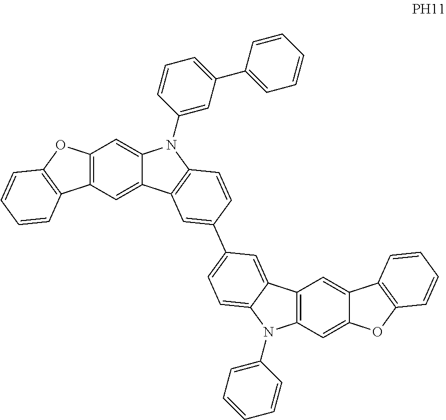 [SMILES: c1ccc(-c2cccc(-n3c4ccc(-c5ccc6c(c5)c5cc7c(cc5n6-c5ccccc5)oc5ccccc57)cc4c4cc5c(cc43)oc3ccccc35)c2)cc1]

### Synthesis Example 12: Production of Aromatic Amine Derivative (PH12)

In the same manner as in Synthesis Example 8 except for using 2.11 g of the intermediate 6 in place of the intermediate 21 and using 1.18 g of 2-bromo-9,9-dimethylfluorene in place of bromobenzene, 1.30 g (yield: 49%) of a white solid was obtained, which was identified as the following compound (PH12) by FD-MS analysis: m/z=780 (theoretical value: 780 for C₅₇H₃₆N₂O₂).

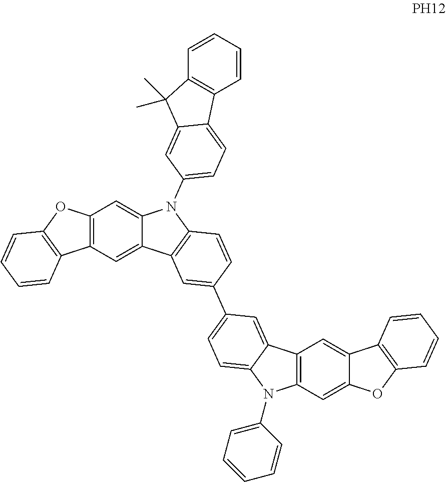 [SMILES: CC1(C)c2ccccc2-c2ccc(-n3c4ccc(-c5ccc6c(c5)c5cc7c(cc5n6-c5ccccc5)oc5ccccc57)cc4c4cc5c(cc43)oc3ccccc35)cc21]

### Example 1-1 (Production of Organic EL Device)

A glass substrate of 25 mm×75 mm×1.1 mm having an ITO transparent electrode (product of Geomatec Company) was cleaned by ultrasonic cleaning in isopropyl alcohol for 5 min and then UV (ultraviolet) ozone cleaning for 30 min.

The cleaned glass substrate having a transparent electrode line was mounted to a substrate holder of a vacuum vapor deposition apparatus. First, the electron-accepting compound (A1) was vapor-deposited on the surface having the transparent electrode line so as to cover the transparent electrode to form an acceptor layer with a thickness of 5 nm.

On the acceptor layer, the aromatic amine derivative (X1) as a first hole transporting material was vapor-deposited to form a first hole transporting layer with a thickness of 85 nm. Successively after forming the first hole transporting layer, the aromatic amine derivative (Y1) as a second hole transporting material was vapor-deposited to form a second hole transporting layer with a thickness of 10 nm.

On the hole transporting layer, the compound (PH1) as a phosphorescent host material and Ir(ppy)₃ as a phosphorescent dopant were vapor co-deposited into a thickness of 30 nm to form a phosphorescent emitting layer. The concentration of Ir(ppy)₃ was 15% by mass.

Thereafter, on the light emitting layer, the benzimidazole derivative (ET1) was vapor-deposited into a thickness of 30 nm and LiF was vapor-deposited into a thickness of 1 nm to form an electron transporting/injecting layer. Further, metallic Al was deposited into a thickness of 80 nm to form a cathode, thereby producing an organic EL device.

### Evaluation of Emission Performance of Organic EL Device

The organic EL device thus produced was allowed to emit light by driving at a constant current and measured for the luminance (L) and the current density. From the measured results, the current efficiency (L/J) and the driving voltage (V) at a current density of 10 mA/cm² were determined. In addition, the lifetime (80% lifetime) at a current density of 50 mA/cm² was determined. The 80% lifetime is the time taken until the luminance is reduced to 80% of the initial luminance when driving at a constant current. The results are shown in Table 1.

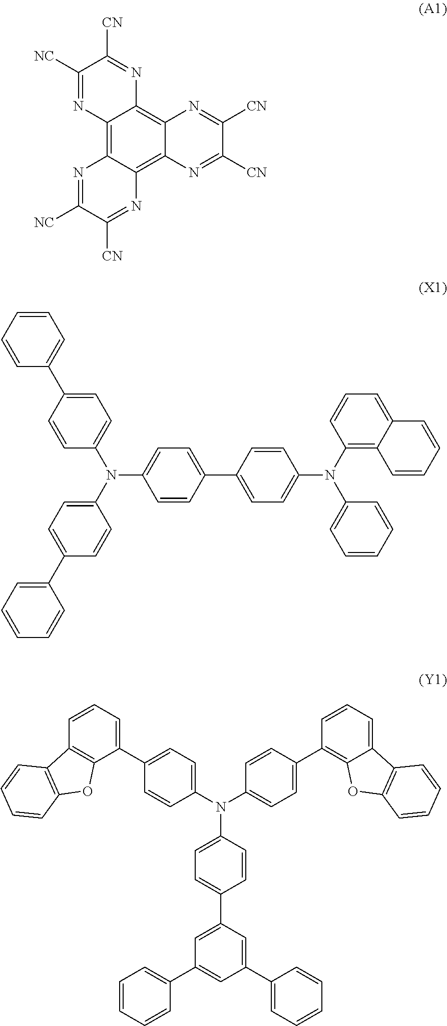 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccccc5)c5cccc6ccccc56)cc4)cc3)cc2)cc1 ; c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1]

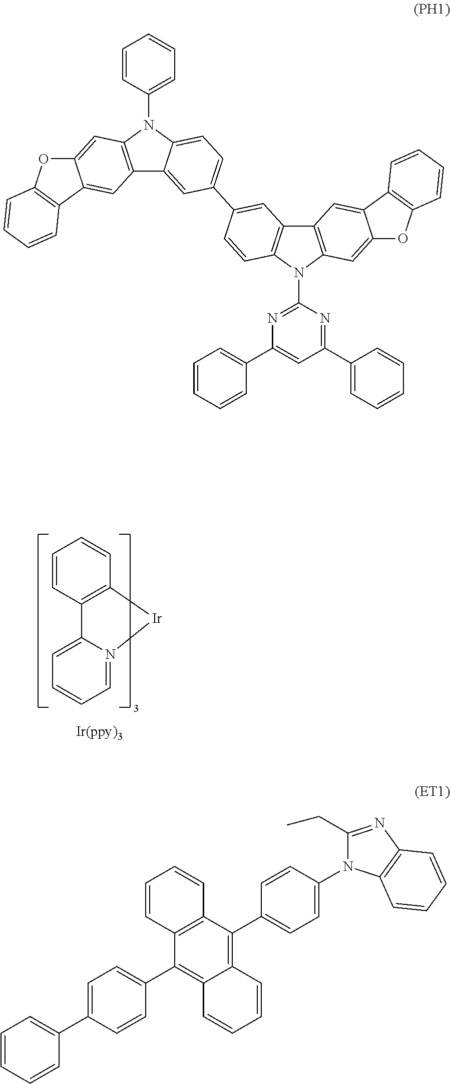

### Comparative Examples 1-1 to 1-4 (Production of Organic EL Device)

Each organic EL device of Comparative Examples 1-1 and 1-4 was produced and evaluated in the same manner as in Example 1-1 except for using each comparative compound described in Table 1 as the phosphorescent host material. The results are shown in Table 1.

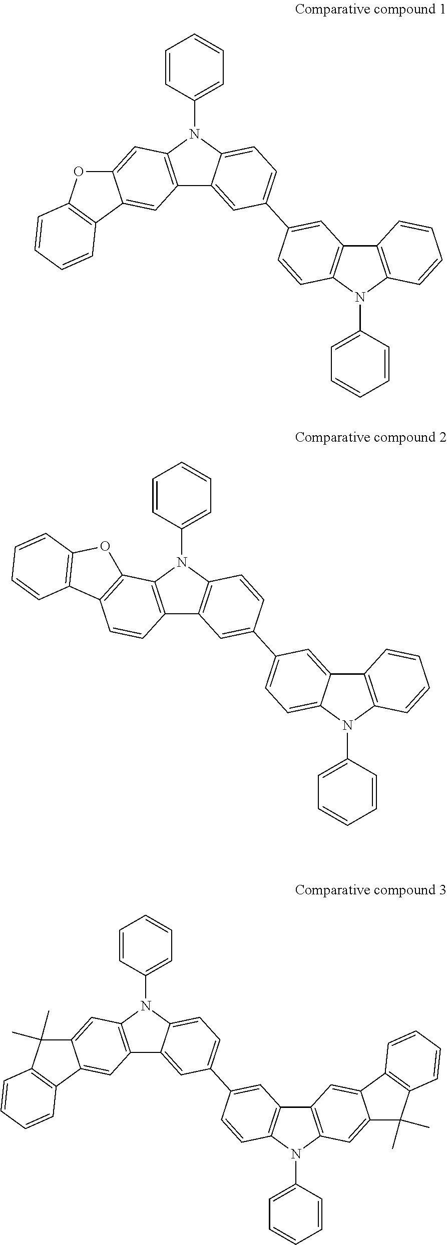 [SMILES: c1ccc(-n2c3ccccc3c3cc(-c4ccc5c(c4)c4cc6c(cc4n5-c4ccccc4)oc4ccccc46)ccc32)cc1 ; c1ccc(-n2c3ccccc3c3cc(-c4ccc5c(c4)c4ccc6c7ccccc7oc6c4n5-c4ccccc4)ccc32)cc1 ; CC1(C)c2ccccc2-c2cc3c4cc(-c5ccc6c(c5)c5cc7c(cc5n6-c5ccccc5)C(C)(C)c5ccccc5-7)ccc4n(-c4ccccc4)c3cc21]

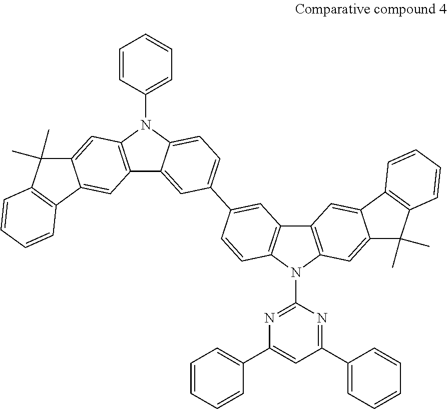 [SMILES: CC1(C)c2ccccc2-c2cc3c4cc(-c5ccc6c(c5)c5cc7c(cc5n6-c5nc(-c6ccccc6)cc(-c6ccccc6)n5)C(C)(C)c5ccccc5-7)ccc4n(-c4ccccc4)c3cc21]

TABLE 1

| | | Measured results | | |
|---|---|---|---|---|
| | | Emission | Driving | 80% |
| | | efficiency | voltage | life- |
| | Phosphorescent | (cd/A) | (V) | time |
| | host material | @10 mA/cm² | @10 mA/cm² | (h) |
| Example | | | | |
| 1-1 | PH1 | 50.1 | 3.5 | 210 |
| Comparative examples | | | | |
| 1-1 | Comparative compound 1 | 41.3 | 3.6 | 150 |
| 1-2 | Comparative compound 2 | 40.3 | 3.5 | 40 |
| 1-3 | Comparative compound 3 | 40.7 | 3.5 | 90 |
| 1-4 | Comparative compound 4 | 42.3 | 3.6 | 155 |

As seen from Table 1, the organic EL device having high emission efficiency and long lifetime was obtained by using the compound (PH1) as the phosphorescent host material.

### Example 2-1 (Production of Organic EL Device)

A glass substrate of 25 mm×75 mm×1.1 mm having an ITO transparent electrode (product of Geomatec Company) was cleaned by ultrasonic cleaning in isopropyl alcohol for 5 min and then UV (ultraviolet) ozone cleaning for 30 min. The cleaned glass substrate having a transparent electrode line was mounted to a substrate holder of a vacuum vapor deposition apparatus. First, the electron-accepting compound (A2) was vapor-deposited on the surface having the transparent electrode line so as to cover the transparent electrode to form an acceptor layer with a thickness of 5 nm.

On the acceptor layer, the aromatic amine derivative (X2) as a first hole transporting material was vapor-deposited to form a first hole transporting layer with a thickness of 90 nm. Successively after forming the first hole transporting layer, the aromatic amine derivative (Y2) as a second hole transporting material was vapor-deposited to form a second hole transporting layer with a thickness of 60 nm.

On the hole transporting layer, the compound (PH2) as a phosphorescent host material and Ir(ppy)₃ as a phosphorescent dopant were vapor co-deposited into a thickness of 40 nm to form a phosphorescent emitting layer. The concentration of Ir(ppy)₃ was 5% by mass.

Thereafter, on the light emitting layer, the carbazole derivative (ET2) and the metal complex Liq were vapor co-deposited into a thickness of 30 nm (concentration of metal complex Liq: 50% by mass) and then the metal complex Liq was vapor-deposited into a thickness of 1 nm to form an electron transporting/injecting layer. Further, metallic Al was deposited into a thickness of 80 nm to form a cathode, thereby producing an organic EL device.

### Evaluation of Emission Performance of Organic EL Device

The organic EL device thus produced was allowed to emit light by driving at a constant current and measured for the luminance (L) and the current density. From the measured results, the current efficiency (L/J) and the driving voltage (V) at a current density of 10 mA/cm² were determined. In addition, the lifetime (80% lifetime) at a current density of 50 mA/cm² was determined. The 80% lifetime is the time taken until the luminance is reduced to 80% of the initial luminance when driving at a constant current. The results are shown in Table 2.

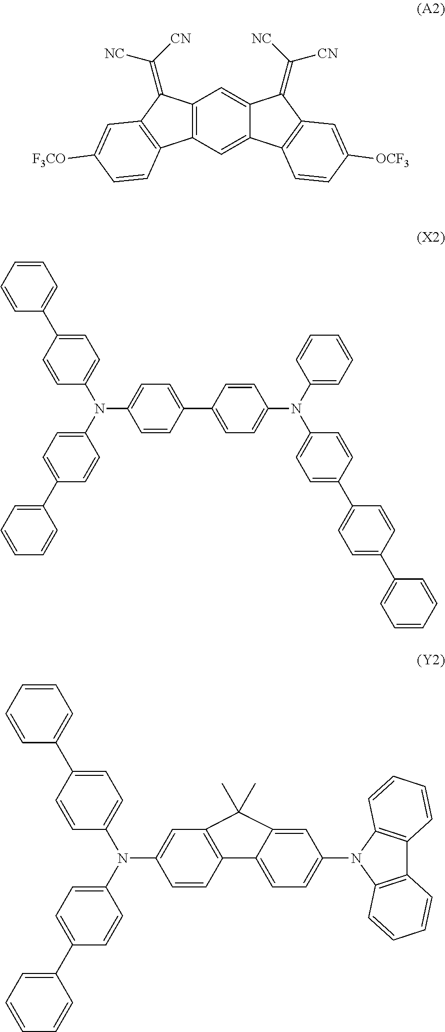 [SMILES: N#CC(C#N)=C1c2cc(OC(F)(F)F)ccc2-c2cc3c(cc21)C(=C(C#N)C#N)c1cc(OC(F)(F)F)ccc1-3 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21]

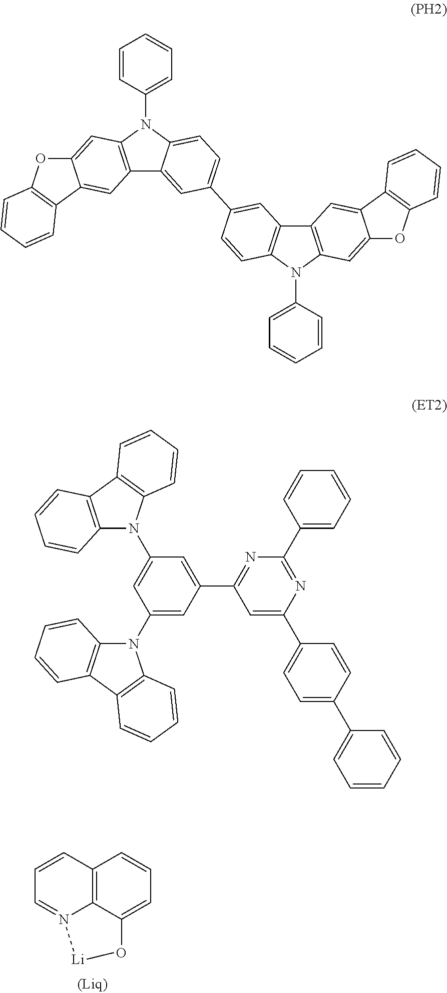

### Example 2-2 (Production of Organic EL Device)

The organic EL device of Example 2-2 was produced and evaluated in the same manner as in Example 2-1 except for using the compound (PH3) described in Table 2 as the phosphorescent host material. The results are shown in Table

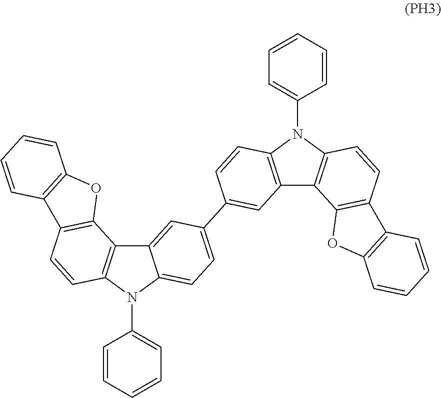 [SMILES: c1ccc(-n2c3ccc(-c4ccc5c(c4)c4c6oc7ccccc7c6ccc4n5-c4ccccc4)cc3c3c4oc5ccccc5c4ccc32)cc1]

### Comparative Examples 2-1 to 2-5 (Production of Organic EL Device)

Each organic EL device of Comparative Examples 2-1 to 2-5 was produced and evaluated in the same manner as in Example 2-1 except for using each comparative compound described in Table 2 as the phosphorescent host material. The results are shown in Table 2.

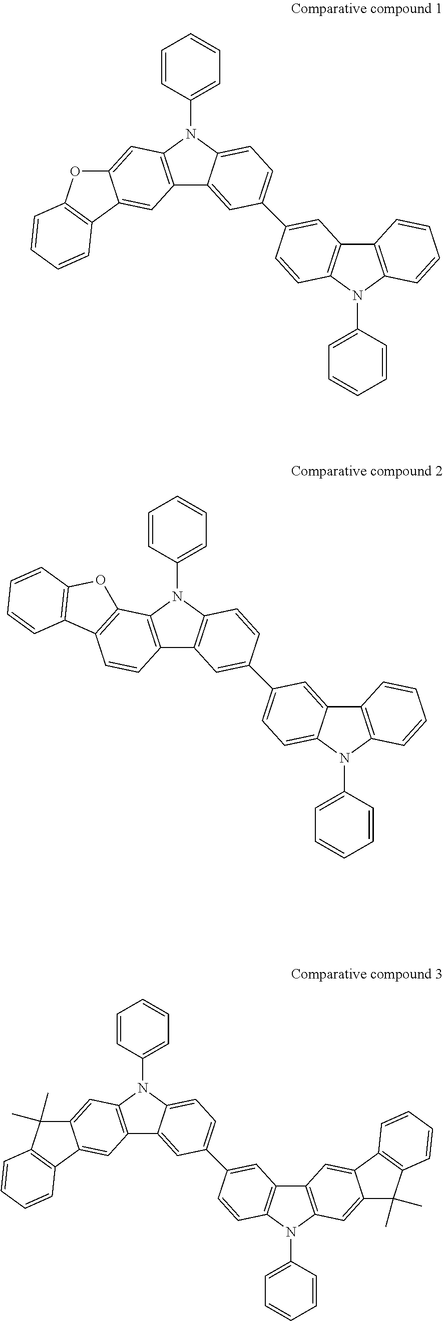 [SMILES: c1ccc(-n2c3ccccc3c3cc(-c4ccc5c(c4)c4cc6c(cc4n5-c4ccccc4)oc4ccccc46)ccc32)cc1 ; c1ccc(-n2c3ccccc3c3cc(-c4ccc5c(c4)c4ccc6c7ccccc7oc6c4n5-c4ccccc4)ccc32)cc1 ; CC1(C)c2ccccc2-c2cc3c4cc(-c5ccc6c(c5)c5cc7c(cc5n6-c5ccccc5)C(C)(C)c5ccccc5-7)ccc4n(-c4ccccc4)c3cc21]

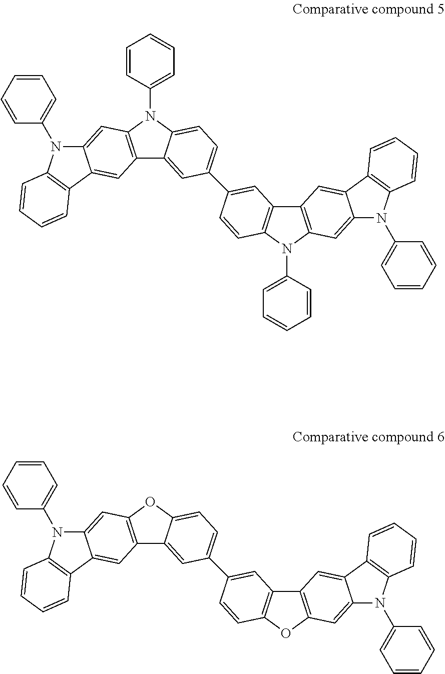 [SMILES: c1ccc(-n2c3ccccc3c3cc4c5cc(-c6ccc7c(c6)c6cc8c9ccccc9n(-c9ccccc9)c8cc6n7-c6ccccc6)ccc5n(-c5ccccc5)c4cc32)cc1 ; c1ccc(-n2c3ccccc3c3cc4c(cc32)oc2ccc(-c3ccc5oc6cc7c(cc6c5c3)c3ccccc3n7-c3ccccc3)cc24)cc1]

TABLE 2

| | | Measured results | | |
|---|---|---|---|---|
| | | Emission | Driving | 80% |
| | | efficiency | voltage | life- |
| | Phosphorescent | (cd/A) | (V) | time |
| | host material | @10 mA/cm² | @10 mA/cm² | (h) |
| Examples | | | | |
| 2-1 | PH2 | 67.9 | 4.7 | 150 |
| 2-2 | PH3 | 69.3 | 4.8 | 100 |
| Comparative examples | | | | |
| 2-1 | Comparative compound 1 | 57.8 | 4.8 | 100 |
| 2-2 | Comparative compound 2 | 55.7 | 4.6 | 35 |
| 2-3 | Comparative compound 3 | 55.3 | 4.6 | 90 |
| 2-4 | Comparative compound 5 | 40.8 | 5.4 | 35 |
| 2-5 | Comparative compound 6 | 40.2 | 5.8 | 45 |

As seen from Table 2, the organic EL devices having high emission efficiency and long lifetime were obtained by using the compound (PH2) or (PH3) as the phosphorescent host material. In addition, the organic EL devices capable of operating at low driving voltage as compared with the organic EL device using the comparative compound 5 or 6 were obtained by using the compound (PH2) or (PH3) as the phosphorescent host material.

### Example 3-1 (Production of Organic EL Device)

A glass substrate of 25 mm×75 mm×1.1 mm having an ITO transparent electrode (product of Geomatec Company) was cleaned by ultrasonic cleaning in isopropyl alcohol for 5 min and then UV (ultraviolet) ozone cleaning for 30 min.

The cleaned glass substrate having a transparent electrode line was mounted to a substrate holder of a vacuum vapor deposition apparatus. First, the electron-accepting compound (A2) was vapor-deposited on the surface having the transparent electrode line so as to cover the transparent electrode to form an acceptor layer with a thickness of 5 nm.

On the acceptor layer, the aromatic amine derivative (X2) as a first hole transporting material was vapor-deposited to form a first hole transporting layer with a thickness of 80 nm. Successively after forming the first hole transporting layer, the compound (PH2) as a second hole transporting material was vapor-deposited to form a second hole transporting layer with a thickness of 10 nm.

On the hole transporting layer, the host compound (BH) and the dopant compound (BD) were vapor co-deposited into a thickness of 25 nm to form a light emitting layer. The concentration of the dopant compound (BD) was 4% by mass.

Thereafter, on the light emitting layer, the compound (ET3) was vapor-deposited into a thickness of 10 nm, the compound (ET1) was vapor-deposited into a thickness of 15 nm, and then LiF was vapor-deposited into a thickness of 1 nm to form an electron transporting/injecting layer. Further, metallic Al was deposited into a thickness of 80 nm to form a cathode, thereby producing an organic EL device.

### Evaluation of Emission Performance of Organic EL Device

The organic EL device thus produced was allowed to emit light by driving at a constant current and measured for the luminance (L) and the current density. From the measured results, the current efficiency (L/J) and the driving voltage (V) at a current density of 10 mA/cm² were determined. In addition, the lifetime (80% lifetime) at a current density of 50 mA/cm² was determined. The 80% lifetime is the time taken until the luminance is reduced to 80% of the initial luminance when driving at a constant current. The results are shown in Table 3.

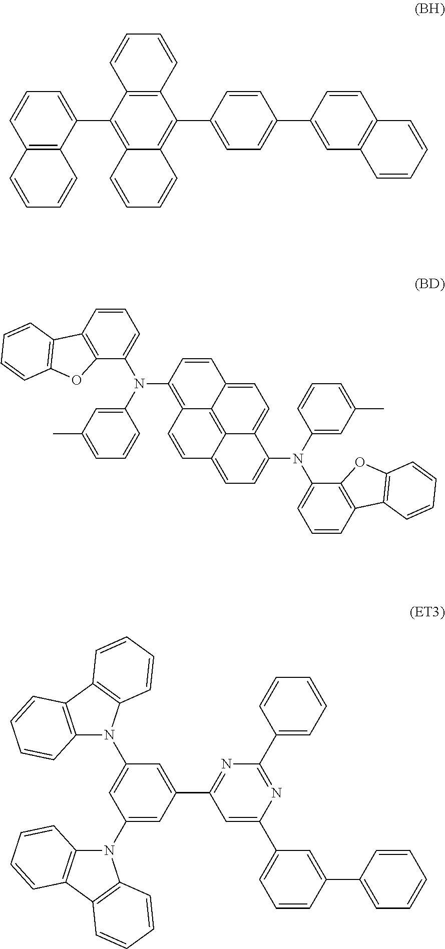

### Examples 3-2 to 3-10

Each organic EL device of Examples 3-2 to 3-10 was produced and evaluated in the same manner as in Example 3-1 except for using each compound described in Table 3 as the second hole transporting material. The results are shown in Table 3.

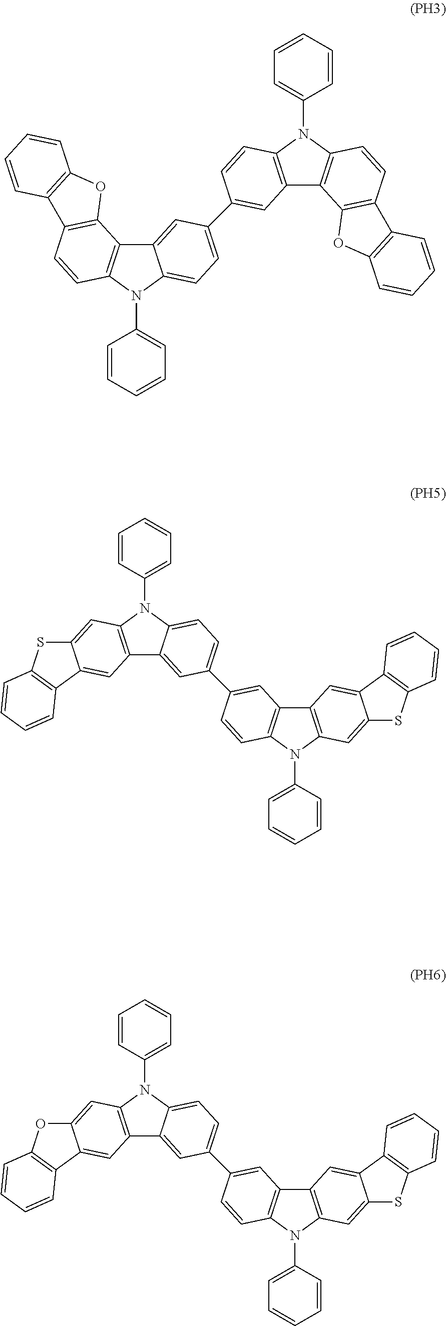 [SMILES: c1ccc(-n2c3ccc(-c4ccc5c(c4)c4c6oc7ccccc7c6ccc4n5-c4ccccc4)cc3c3c4oc5ccccc5c4ccc32)cc1 ; c1ccc(-n2c3ccc(-c4ccc5c(c4)c4cc6c(cc4n5-c4ccccc4)sc4ccccc46)cc3c3cc4c(cc32)sc2ccccc24)cc1 ; c1ccc(-n2c3ccc(-c4ccc5c(c4)c4cc6c(cc4n5-c4ccccc4)sc4ccccc46)cc3c3cc4c(cc32)oc2ccccc24)cc1]

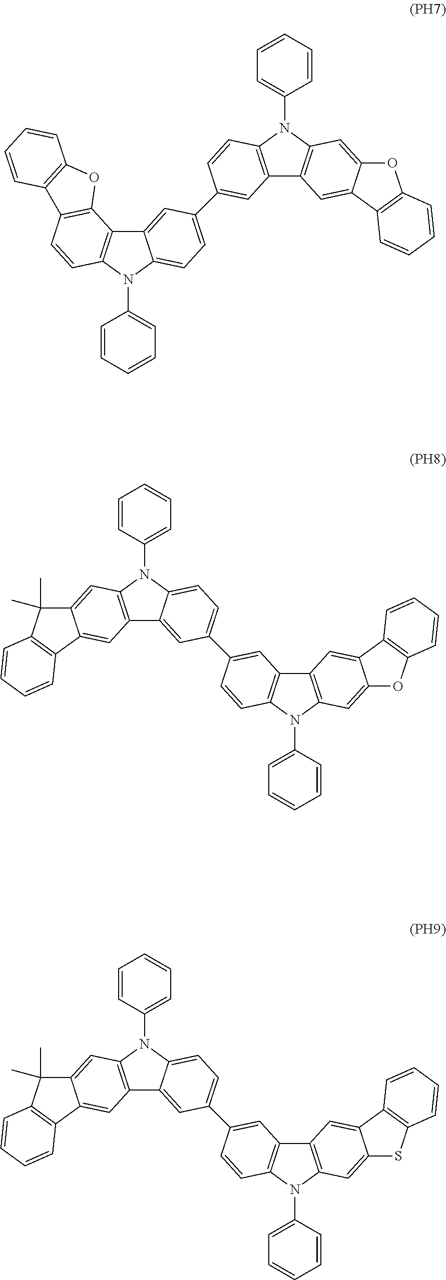 [SMILES: c1ccc(-n2c3ccc(-c4ccc5c(c4)c4c6oc7ccccc7c6ccc4n5-c4ccccc4)cc3c3cc4c(cc32)oc2ccccc24)cc1 ; CC1(C)c2ccccc2-c2cc3c4cc(-c5ccc6c(c5)c5cc7c(cc5n6-c5ccccc5)oc5ccccc57)ccc4n(-c4ccccc4)c3cc21 ; CC1(C)c2ccccc2-c2cc3c4cc(-c5ccc6c(c5)c5cc7c(cc5n6-c5ccccc5)sc5ccccc57)ccc4n(-c4ccccc4)c3cc21]

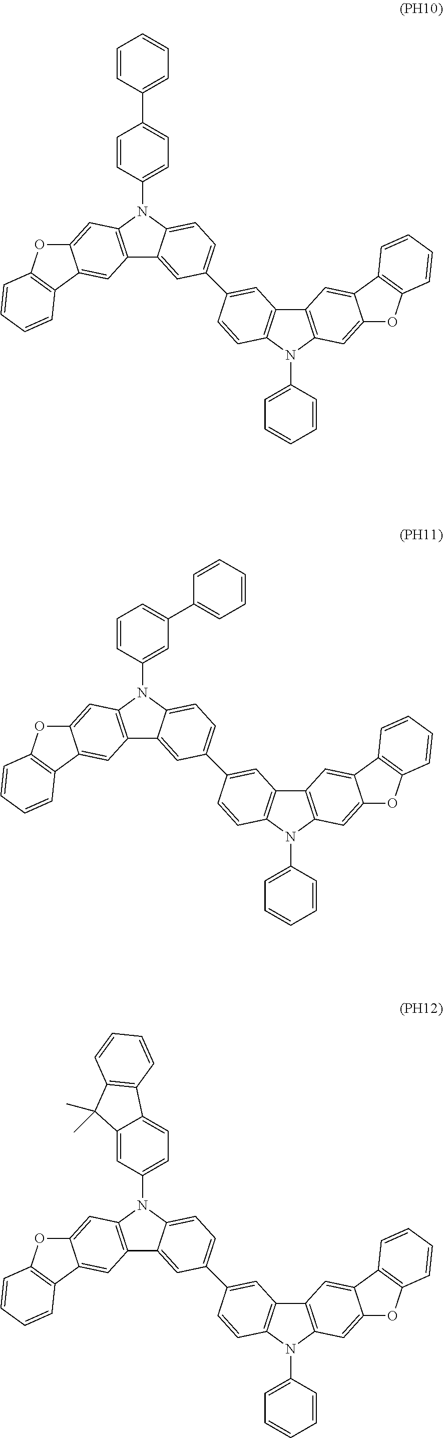 [SMILES: c1ccc(-c2ccc(-n3c4ccc(-c5ccc6c(c5)c5cc7c(cc5n6-c5ccccc5)oc5ccccc57)cc4c4cc5c(cc43)oc3ccccc35)cc2)cc1 ; c1ccc(-c2cccc(-n3c4ccc(-c5ccc6c(c5)c5cc7c(cc5n6-c5ccccc5)oc5ccccc57)cc4c4cc5c(cc43)oc3ccccc35)c2)cc1 ; CC1(C)c2ccccc2-c2ccc(-n3c4ccc(-c5ccc6c(c5)c5cc7c(cc5n6-c5ccccc5)oc5ccccc57)cc4c4cc5c(cc43)oc3ccccc35)cc21]

### Comparative Examples 3-1 to 3-5

Each organic EL device of Comparative Examples 3-1 to 3-5 was produced and evaluated in the same manner as in Example 3-1 except for using each comparative compound described in Table 3 as the second hole transporting material. The results are shown in Table 3.

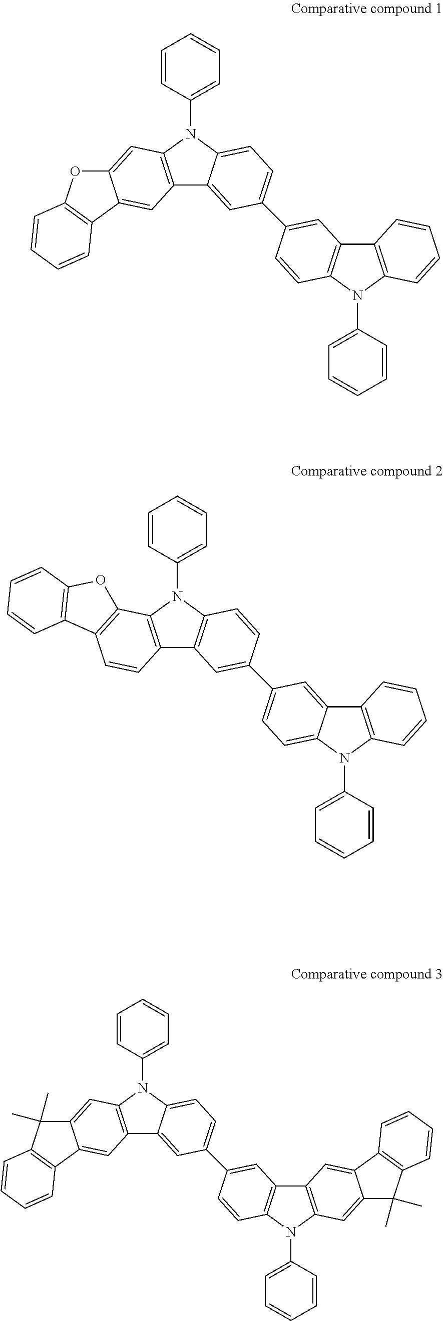 [SMILES: c1ccc(-n2c3ccccc3c3cc(-c4ccc5c(c4)c4cc6c(cc4n5-c4ccccc4)oc4ccccc46)ccc32)cc1 ; c1ccc(-n2c3ccccc3c3cc(-c4ccc5c(c4)c4ccc6c7ccccc7oc6c4n5-c4ccccc4)ccc32)cc1 ; CC1(C)c2ccccc2-c2cc3c4cc(-c5ccc6c(c5)c5cc7c(cc5n6-c5ccccc5)C(C)(C)c5ccccc5-7)ccc4n(-c4ccccc4)c3cc21]

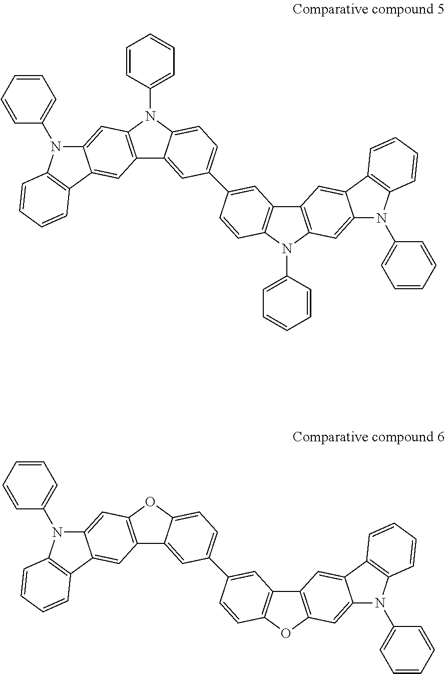 [SMILES: c1ccc(-n2c3ccccc3c3cc4c5cc(-c6ccc7c(c6)c6cc8c9ccccc9n(-c9ccccc9)c8cc6n7-c6ccccc6)ccc5n(-c5ccccc5)c4cc32)cc1 ; c1ccc(-n2c3ccccc3c3cc4c(cc32)oc2ccc(-c3ccc5oc6cc7c(cc6c5c3)c3ccccc3n7-c3ccccc3)cc24)cc1]

TABLE 3

| | | | Measured results | | |
|---|---|---|---|---|---|
| | First hole | | Emission | Driving | 80% |
| | trans- | Second hole | efficiency | voltage | life |
| | porting | transporting | (cd/A) | (V) | time |
| | material | material | @10 mA/cm² | @10 mA/cm² | (h) |
| Examples | | | | | |
| 3-1 | X2 | Comparative | 7.8 | 3.7 | 280 |
| 3-1 | X2 | PH2 | 9.0 | 3.5 | 350 |
| 3-2 | X2 | PH3 | 9.2 | 3.7 | 300 |
| 3-3 | X2 | PH5 | 9.4 | 3.5 | 300 |
| 3-4 | X2 | PH6 | 9.3 | 3.5 | 310 |
| 3-5 | X2 | PH7 | 9.1 | 3.6 | 320 |
| 3-6 | X2 | PH8 | 9.1 | 3.4 | 340 |
| 3-7 | X2 | PH9 | 9.3 | 3.4 | 310 |
| 3-8 | X2 | PH10 | 9.1 | 3.4 | 330 |
| 3-9 | X2 | PH11 | 9.1 | 3.5 | 340 |
| 3-10 | X2 | PH12 | 9.0 | 3.3 | 300 |
| Comparative examples | | | | | |
| 3-1 | X2 | Comparative | 7.8 | 3.7 | 280 |
| | | compound 1 | | | |
| 3-2 | X2 | Comparative | 7.5 | 3.8 | 75 |
| | | compound 2 | | | |
| 3-3 | X2 | Comparative | 7.5 | 3.8 | 270 |
| | | compound 3 | | | |
| 3-4 | X2 | Comparative | 6.8 | 3.5 | 70 |
| | | compound 5 | | | |
| 3-5 | X2 | Comparative | 2.7 | 4.9 | 10 |
| | | compound 6 | | | |

As seen from Table 3, the organic EL devices having high emission efficiency and long lifetime were obtained by using any of the compounds (PH2), (PH3), and (PH5) to (PH12) as the second hole transporting material. In addition, the organic EL devices capable of operating at extremely low driving voltage as compared with the organic EL device using the comparative compound 6 were obtained by using any of the compounds (PH2), (PH3), and (PH5) to (PH12) as the second hole transporting material.

### REFERENCE SIGNS LIST

1: Organic EL device
2: Substrate
3: Anode
4: Cathode
5: Light emitting layer
6: Anode-side organit thin film layer
7: Cathode-side organit thin film layer
10: Emission unit

1: A compound represented by formula (1):
A-L-B (1)
wherein:
A is a group represented by formula (1-A);
B is a group represented by formula (1-B);
L is a single bond, a substituted or unsubstituted arylene group having 6 to 50 ring carbon atoms, or a substituted or unsubstituted heteroarylene group having 5 to 50 ring atoms;
L is bonded to one of R¹ to R⁴ of formula (1-A) and R¹¹ to R¹⁴ of formula (1-B); 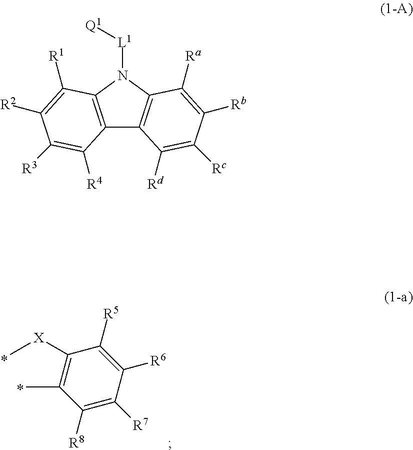
R^{a} and R^{b}, R^{b} and R^{c}, or R^{c} and R^{d} are direct bonds which are respectively bonded to sites * of formula (1-a) to form a ring structure, and the others of R^{a} to R^{d} are each independently a hydrogen atom or a substituent, or adjacent two thereof are bonded to each other to form a ring structure;
one of R¹ to R⁸ is bonded to L;
R¹ to R⁸ not bonded to L are each independently a hydrogen atom or a substituent, or adjacent two thereof are bonded to each other to form a ring structure;
L¹ is a single bond, a substituted or unsubstituted arylene group having 6 to 50 ring carbon atoms, or a substituted or unsubstituted heteroarylene group having 5 to 50 ring atoms;
Q¹ is a substituted or unsubstituted aryl group having 6 to 50 ring carbon atoms or a substituted or unsubstituted heteroaryl group having 5 to 40 ring atoms;
X is an oxygen atom or a sulfur atom; 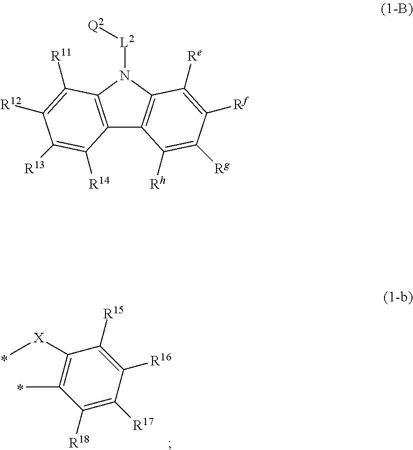
R^{e} and R^{f}, R^{f} and R^{g}, or R^{g} and R^{h} are direct bonds which are respectively bonded to sites * of formula (1-b) to form a ring structure, and the others of R^{e} to R^{h} are each independently a hydrogen atom or a substituent, or adjacent two thereof are bonded to each other to form a ring structure;
one of R¹¹ to R¹⁸ is bonded to L;
R¹¹ to R¹⁸ not bonded to L are each independently a hydrogen atom or a substituent, or adjacent two thereof are bonded to each other to form a ring structure;
L² is a single bond, a substituted or unsubstituted arylene group having 6 to 50 ring carbon atoms, or a substituted or unsubstituted heteroarylene group having 5 to 50 ring atoms;
Q² is a substituted or unsubstituted aryl group having 6 to 50 ring carbon atoms, or a substituted or unsubstituted heteroarylene group having 5 to 40 ring atoms;
Y is C(R¹⁹)(R²⁰), N(R²¹), an oxygen atom, or a sulfur atom;
each of R¹⁹ and R²⁰ is independently a hydrogen atom, a substituted or unsubstituted alkyl group having 1 to 20 carbon atoms, a substituted or unsubstituted aryl group having 6 to 50 ring carbon atoms, a substituted or unsubstituted heteroaryl group having 5 to 50 ring atoms, a substituted or unsubstituted fluoroalkyl group having 1 to 20 carbon atoms, or a cyano group, or adjacent two thereof are bonded to each other to form a ring structure; and
R²¹ is a substituted or unsubstituted alkyl group having 1 to 20 carbon atoms, a substituted or unsubstituted aryl group having 6 to 50 ring carbon atoms, a substituted or unsubstituted heteroaryl group having 5 to 50 ring atoms, or a substituted or unsubstituted fluoroalkyl group having 1 to 20 carbon atoms.

2: The compound according to claim 1, wherein A is represented by any of formulae (1-A-1) to (1-A-6): 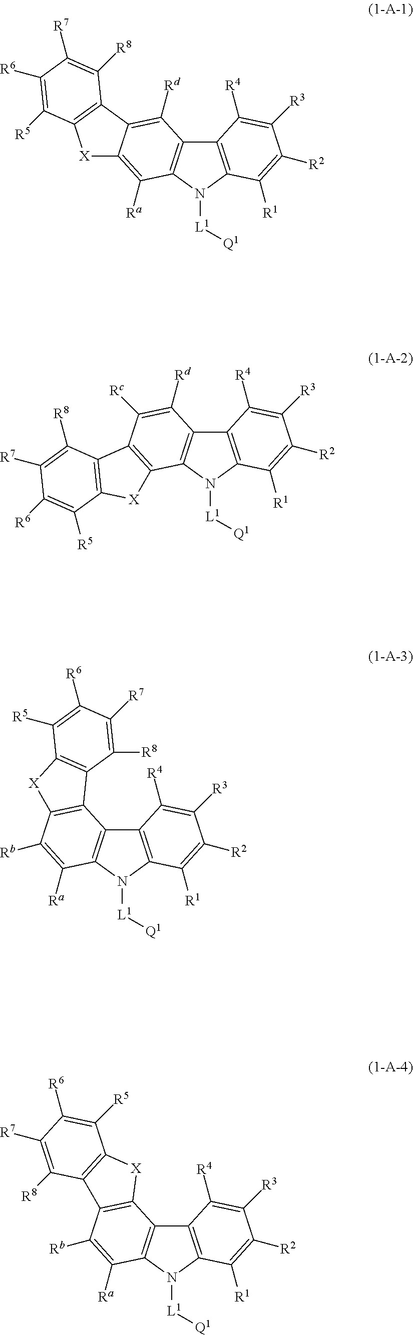 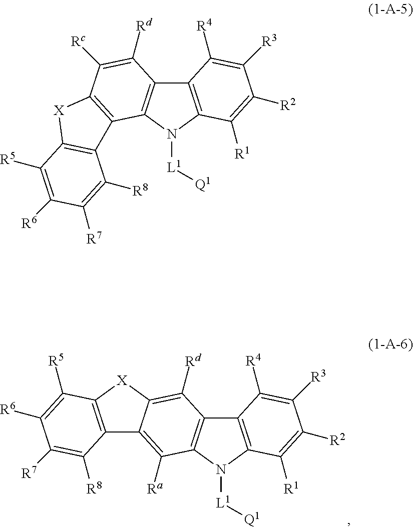
wherein R¹ to R⁸, X, L, and Q¹ are as defined in claim 1, and each of R^{a} to R^{d} is independently a hydrogen atom or a substituent.

3: The compound according to claim 1, wherein B is represented by any of formulae (1-B-1) to (1-B-6): 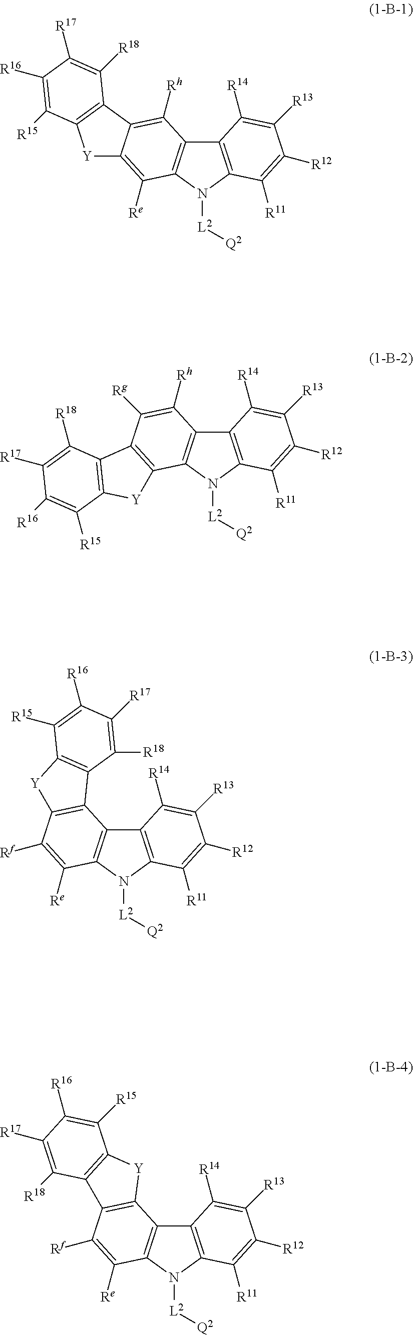 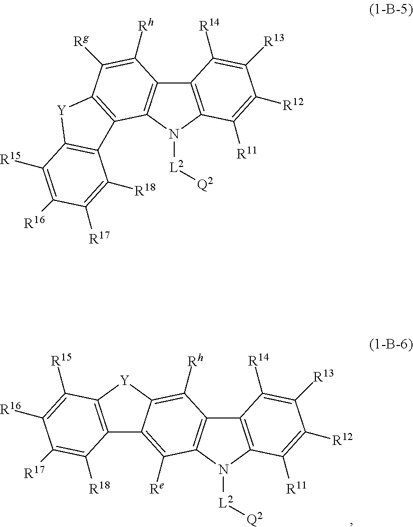
wherein R¹¹ to R¹⁸, Y, L², and Q² are as defined in claim 1, and each of R^{e} to R^{h} is independently a hydrogen atom or a substituent.

4: The compound according to claim 1, wherein the substituent is independently a substituted or unsubstituted alkyl group having 1 to 50 carbon atoms, a substituted or unsubstituted aryl group having 6 to 50 ring carbon atoms, a substituted or unsubstituted heteroaryl group having 5 to 50 ring atoms, a halogen atom, a cyano group, a substituted or unsubstituted fluoroalkyl group having 1 to 50 carbon atoms, a substituted or unsubstituted alkoxy group having 1 to 50 carbon atoms, a substituted or unsubstituted fluoroalkoxy group having 1 to 50 carbon atoms, a substituted or unsubstituted aryloxy group having 6 to 50 ring carbon atoms, a disubstituted amino group, wherein the substituent is selected from the group consisting of an alkyl group having 1 to 50 carbon atoms and an aryl group having 6 to 50 ring carbon atoms, or a trisubstituted silyl group, wherein the substituent is selected from the group consisting of an alkyl group having 1 to 50 carbon atoms and an aryl group having 6 to 50 ring carbon atoms.

5: The compound according to claim 1, wherein one of R¹ to R⁴ is bonded to L.

6: The compound according to claim 1, wherein one of R¹¹ to R¹⁴ is bonded to L.

7: The compound according to claim 1, wherein one of R¹⁵ to R¹⁸ is bonded to L.

8: The compound according to claim 1, wherein the compound is represented by formula (2): 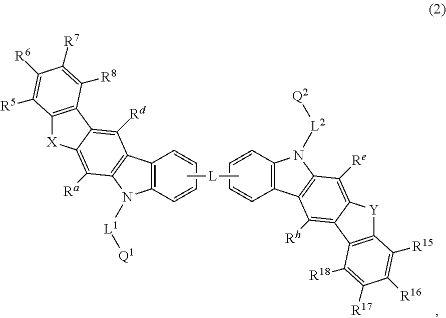
wherein:
R^{a}, R^{d}, R^{e}, R^{h}, R⁵ to R⁸, R¹⁵ to R¹⁸, X, Y, L, L¹, L², Q¹, and Q² are as defined in claim 1;
two benzene rings to which L is bonded may have a substituent;
the substituent is as defined above with respect to R¹ to R⁴ and R¹¹ to R¹⁴; and
the substituents may be boded to each other to form a ring structure.

9: The compound according to claim 1, wherein the compound is represented by any of formulae (2-1) to (2-4): 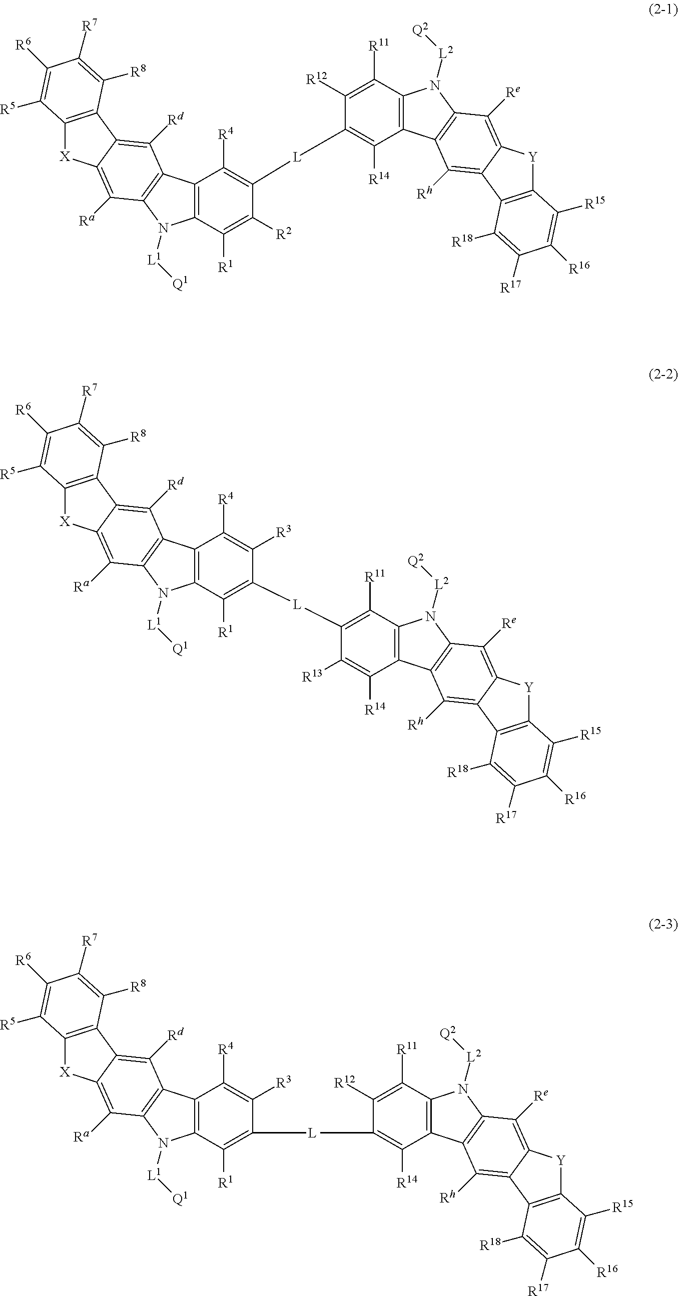 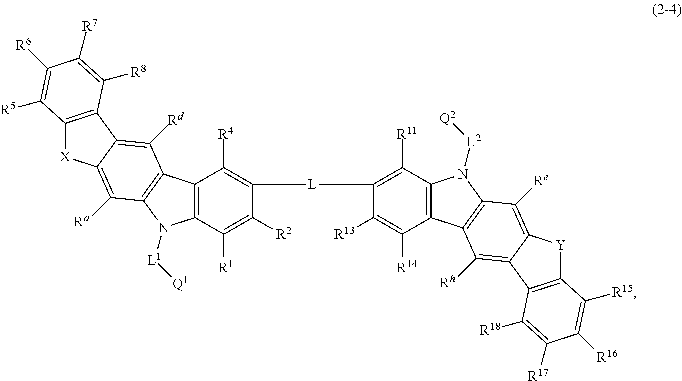
wherein:
R^{a}, R^{d}, R^{e}, R^{h}, R⁵ to R⁸, R¹⁵ to R¹⁸, X, Y, L, L¹, L², Q¹, and Q² are as defined in claim 1,
each of R¹ to R⁴ and R¹¹ to R¹⁴ is independently a hydrogen atom or a substituent; and
adjacent substituents may be bonded to each other to form a ring structure.

10: The compound according to claim 1, wherein the compound is represented by any of formulae (2-1′), (2-3′), and (2-4′): 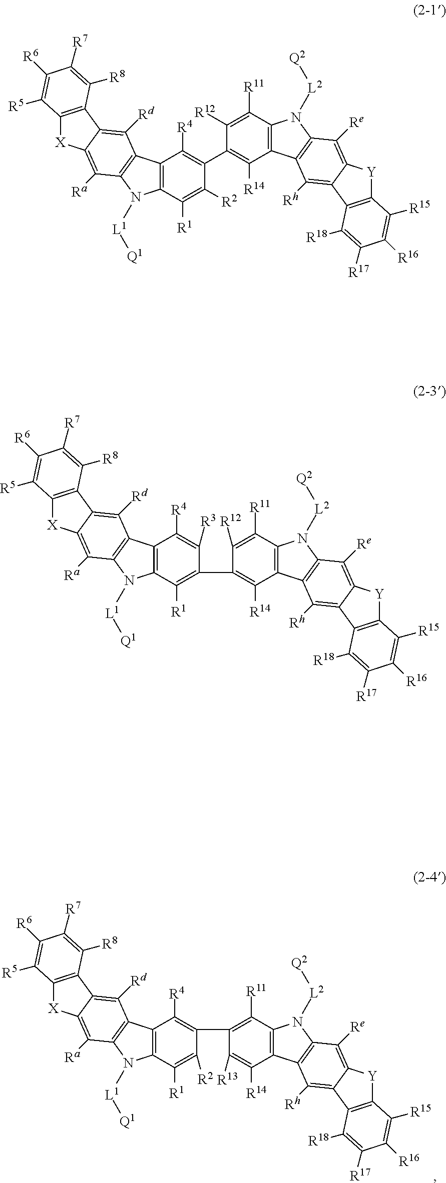
wherein:
R^{a}, R^{d}, R^{e}, R^{h}, R⁵ to R⁸, R¹⁵ to R¹⁸, X, Y, L¹, L², Q¹, and Q² are as defined in claim 1;
each of R¹ to R⁴ and R¹¹ to R¹⁴ is independently a hydrogen atom or a substituent; and
adjacent substituents may be bonded to each other to form a ring structure.

11: The compound according to claim 1, wherein the compound is represented by formula (2-1′-i) or (2-4′-i): 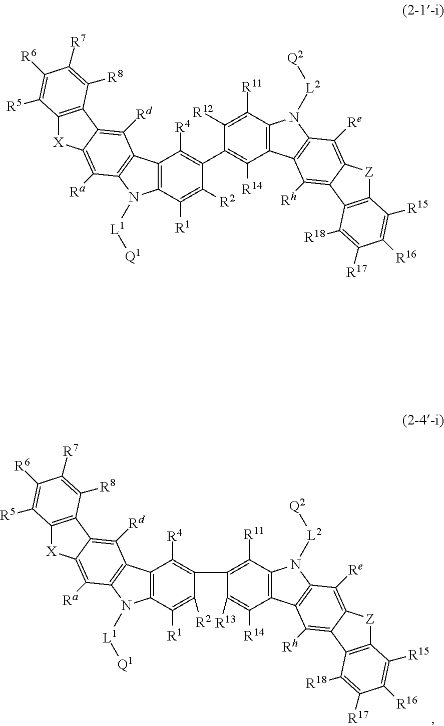
wherein:
R^{a}, R^{d}, R^{e}, R^{h}, R⁵ to R⁸, R¹⁵ to R¹⁸, X, L¹, L², Q¹, and Q² are as defined in claim 1;
each of R¹ to R⁴ and R¹¹ to R¹⁴ is independently a hydrogen atom or a substituent;
adjacent substituents may be bonded to each other to form a ring structure; and
Z is an oxygen atom or a sulfur atom.

12: The compound according to claim 1, wherein the compound is represented by formula (2-1′-i-O) or (2-4′-i-O): 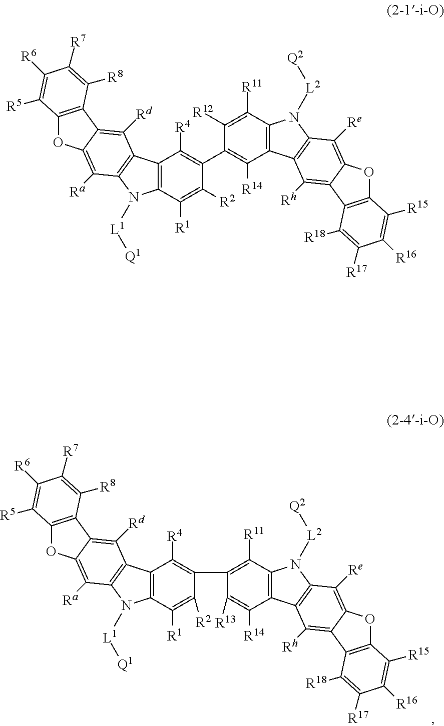
wherein:
R^{a}, R^{d}, R^{c}, R^{h}, R⁵ to R⁸, R¹⁵ to R¹⁸, L¹, L², Q¹, and Q² are as defined in claim 1;
each of R¹ to R⁴ and R¹¹ to R¹⁴ is independently a hydrogen atom or a substituent; and
adjacent substituents may be bonded to each other to form a ring structure.

13: The compound according to claim 1, wherein at least one selected from the group consisting of Q¹ and Q² is a substituted or unsubstituted aryl group having 6 to 50 ring carbon atoms.

14: The compound according to claim 1, wherein each of Q¹ and Q² is independently a substituted or unsubstituted aryl group having 6 to 50 ring carbon atoms.

15: The compound according to claim 1, wherein Q¹ is represented by formula (3): 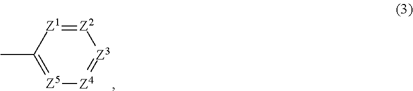
wherein:
at least one selected from the group consisting of Z¹ to Z⁵ is a nitrogen atom and the others are each independently C(R³⁰), wherein R³⁰ is a hydrogen atom or a substituent;
when two or more of Z¹ to Z⁵ are C(R³⁰), variables R³⁰ may be the same or different; and
variables R³⁰ may be bonded to each other to form a ring structure.

16: The compound according to claim 15, wherein Q¹ is represented by formula (3-1): 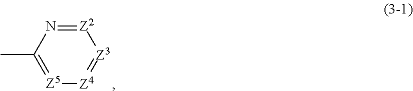
wherein:
each of Z² to Z⁵ is independently a nitrogen atom or C(R³⁰), wherein R³⁰ is a hydrogen atom or a substituent;
when two or more of Z² to Z⁵ are C(R³⁰), variables R³⁰ may be the same or different; and
variables R³⁰ may be bonded to each other to form a ring structure.

17: The compound according to claim 1, wherein Q¹ is represented by any of formulae (3-2) to (3-4): 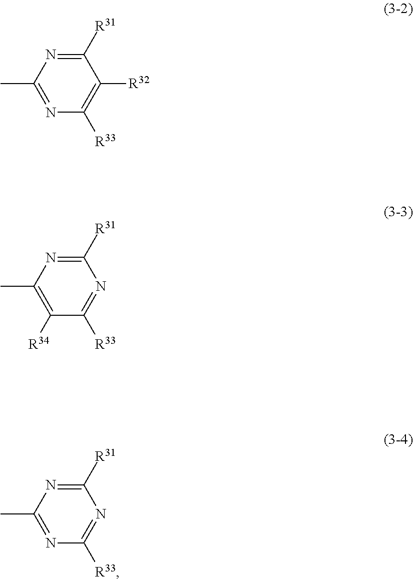
wherein:
each of R³¹ to R³⁴ is independently a hydrogen atom or a substituent;
R³¹ and R³², and R³² and R³³ in formula (3-2) may be bonded to each other to form a ring structure; and
R³³ and R³⁴ in formula (3-3) may be bonded to each other to form a ring structure.

18: The compound according to claim 1, wherein the substituent is independently a substituted or unsubstituted aryl group having 6 to 50 ring carbon atoms, a substituted or unsubstituted heteroaryl group having 5 to 50 ring atoms, a halogen atom, a cyano group, a substituted or unsubstituted fluoroalkyl group having 1 to 50 carbon atoms, a substituted or unsubstituted alkoxy group having 1 to 50 carbon atoms, a substituted or unsubstituted fluoroalkoxy group having 1 to 50 carbon atoms, or a substituted or unsubstituted aryloxy group having 6 to 50 ring carbon atoms.

19: The compound according to claim 1, wherein R¹ to R⁸ not bonded to L, R¹¹ to R¹⁸ not bonded to L, R^{a} to R^{d} not bonded to the site * of formula (1-a), thereby failing to form a ring structure, and R^{e} to R^{h} not bonded to the site * of formula (1-b), thereby failing to form a ring structure are all hydrogen atoms.

20: A material for organic electroluminescence device, the material comprising the compound of claim 1.

21: An organic electroluminescence device, comprising an organic thin film layer between a cathode and an anode, wherein:
the organic thin film layer comprises one or more layers comprising a light emitting layer; and
at least one layer of the organic thin film layer comprises the compound of claim 1.

22: The organic electroluminescence device according to claim 21, wherein the light emitting layer comprises the compound.

23: The organic electroluminescence device according to claim 21, wherein the organic thin film layer comprises at least one of a hole injecting layer and a hole transporting layer each of which comprises the compound.

24: The organic electroluminescence device according to claim 21, wherein the light emitting layer comprises a fluorescent emitting material.

25: The organic electroluminescence device according to claim 24, wherein the fluorescent emitting material comprises at least one selected from the group consisting of an anthracene derivative, a fluoranthene derivative, a styrylamine derivative, and an arylamine derivative.

26: The organic electroluminescence device according to claim 21, wherein the light emitting layer comprises a phosphorescent emitting material.

27: The organic electroluminescence device according to claim 26, wherein the phosphorescent emitting material is an orthometalated complex of a metal atom selected from the group consisting of iridium (Ir), osmium (Os), and platinum (Pt).

28: An electronic device, comprising the organic electroluminescence device according to claim 21.
